SEQUENCE LISTING (14 sequences)

SEQ ID NO: 1 - protein, 106 aa, Unknown
  Description of Unknown Last bacterial common ancestor (LBCA) thioredoxin polypeptide
MSVIEINDENFEEEVLKSDKPVLVDFWAPWCGPCRMIAPIIEELAEEYEGKVKFAKVNVDENPETAAKYGIMSIPTLLLFKNGEVVDKLVGARPKEALKERIEKHL

SEQ ID NO: 2 - protein, 106 aa, Unknown
  Description of Unknown Last archaeal common ancestor (LACA) thioredoxin polypeptide
MSVVQLNDENFDEVIKKNNKVVVVDFWAEWCGPCRMIAPIIEELAKEYAGKVVFGKLNVDENPETAAKYGIMSIPTLLFFKNGKVVDQLVGAMPKEALKERIKKYL

SEQ ID NO: 3 - protein, 106 aa, Unknown
  Description of Unknown Archaeal/eukaryotic common ancestor (AECA) thioredoxin polypeptide
MSVIEINDENFDEVIKKSDKVVVVDFWAEWCGPCRMIAPIIEELAEEYAGKVVFGKVNVDENPEIAAKYGIMSIPTLLFFKNGKVVDQLVGARPKEALKERIKKYL

SEQ ID NO: 4 - protein, 106 aa, Unknown
  Description of Unknown Last eukaryotic common ancestor (LECA) thioredoxin polypeptide
MVIQVTNKEEFEAILSEADKLVVVDFFATWCGPCKMIAPFFEELSEEYPDKVVFIKVDVDEVPDVAAKYGITSMPTFKFFKNGKKVDELVGANQEKLKQMILKHAP

SEQ ID NO: 5 - protein, 106 aa, Unknown
  Description of Unknown Last common ancestor of cyanobacterial and deinococcus/thermus groups (LPBCA) thioredoxin polypeptide
MSVIEVTDENFEQEVLKSDKPVLVDFWAPWCGPCRMIAPIIEELAKEYEGKVKVVKVNVDENPNTAAQYGIRSIPTLLLFKNGQVVDRLVGAQPKEALKERIDKHL

SEQ ID NO: 6 - protein, 106 aa, Unknown
  Description of Unknown Last common ancestor of gamma-proteobacteria, 1.61 Gyr old (LGPCA) thioredoxin polypeptide
MSIIHVTDDSFDQDVLKADKPVLVDFWAEWCGPCKMIAPILDEIAEEYEGKLKVAKVNIDENPETAAKYGIRGIPTLMLFKNGEVAATKVGALSKSQLKEFLDANL

SEQ ID NO: 7 - protein, 106 aa, Unknown
  Description of Unknown Last common ancestor of animals and fungi (LAFCA) thioredoxin polypeptide
MVIQVTNKDEFESILSEADKLVVVDFTATWCGPCKMIAPKFEELSEEYPDNVVFLKVDVDEVEDVAAEYGISAMPTFQFFKNGKKVDELTGANQEKLKAMIKKHAA

SEQ ID NO: 8 - protein, 4 aa, Unknown
  Description of Unknown Thioredoxin active site peptide
CGPC

SEQ ID NO: 9 - protein, 6 aa, Artificial Sequence
  Description of Artificial Sequence Synthetic 6xHis tag
HHHHHH

SEQ ID NO: 10 - protein, 6 aa, Artificial Sequence
  Description of Artificial Sequence Synthetic peptide
DDDDKX

SEQ ID NO: 11 - protein, 104 aa, Unknown
  Description of Unknown Ancestral Enzyme thioredoxin AECA polypeptide
SVIEINDENFDEVIKKDKVVVVDFWAEWCGPCRMIAPIIEELAEEYAGKVVFGKVNVDENPEIAAKYGIMSIPTLLFFKNGKVVDQLVGARPKEALKERIKKYL

SEQ ID NO: 12 - protein, 106 aa, Unknown
  Description of Unknown Last archaeal common ancestor (LACA) thioredoxin polypeptide
MSVVQLNDENFDEVIKKNNKVVVVDFWAEWCGPCRMIAPIIEELAKEYAGKVVFGKLNVDENPEIAAKYGIMSIPTLLFFKNGKVVDQLVGAMPKEALKERIKKYL

SEQ ID NO: 13 - protein, 108 aa, Escherichia coli
SDKIIHLTDDSFDTDVLKADGAILVDFWAEWCGPCKMIAPILDEIADEYQGKLTVAKVNIDQNPGTAPKYGIRGIPTLLLFKNGEVAATKVGALSKGQLKEFLDANLA

SEQ ID NO: 14 - protein, 105 aa, Homo sapiens
MVKQIESKTAFQEALDAAGDKLVVVDFSATWCGPCKMIKPFFHSLSEKYSNVIFLEVDVDDCQDVASECEVKCTPTFQFFKKGQKVGEFSGANKEKLEATINELV

(19) United States
(12) Patent Application Publication
(10) Pub. No.: US 2013/0252307 A1
(43) Pub. Date: Sep. 26, 2013

(54) ANCESTRAL PROTEINS

(71) Applicants: Julio M. Fernandez, New York, NY (US); Raul Perez-Jimenez, New York, NY (US); Eric Gaucher, Atlanta, GA (US); Pallav Kosuri, New York, NY (US)

(72) Inventors: Julio M. Fernandez, New York, NY (US); Raul Perez-Jimenez, New York, NY (US); Eric Gaucher, Atlanta, GA (US); Pallav Kosuri, New York, NY (US)

(73) Assignee: GEORGIA TECH RESEARCH CORPORATION, Atlanta, GA (US)

(21) Appl. No.: 13/810,420

(22) PCT Filed: Jul. 15, 2011
(86) PCT No.: PCT/US11/44275

§ 371 Date: May 29, 2013

Related U.S. Application Data
(60) Provisional application No. 61/364,640, filed on Jul. 15, 2010.

(51) Int. Cl. C12N 9/00

(52) U.S. Cl. CPC C12N 9/0036
USPC 435/189; 530/389.8; 536/23.2; 435/320.1; 435/419; 435/252.3; 435/254.2; 435/348; 435/363; 435/366; 435/352; 435/354; 435/349; 435/358; 435/369; 435/350; 703/2

(57) ABSTRACT

The invention provides a method for increasing the stability and/or activity of a polypeptide at low pH and/or elevated temperatures. The invention further provides a method for increasing the melting temperature of a polypeptide. Also provided are paleoenzymologically reconstructed thioredoxin polypeptides having activity at higher temperatures and/or lower pH than extant thioredoxin polypepetides, as well as paleoenzymologically reconstructed thioredoxin polypeptides having higher melting temperatures than extant thioredoxin polypepetides.

This application claims priority to U.S. Provisional Application No. 61/364,640, filed on Jul. 15, 2010, and also claims priority to PCT/US11/44084, filed on Jul. 14, 2011, which are herein incorporated by reference in their entirety.

This invention was made with government support under HL66030 and HL61228 awarded by NIH. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

To conform to the requirements for International Patent Applications, many of the figures presented herein are black and white representations of images originally created in color. The original color versions can be viewed in Perez-Jimenez et al., 2011, Nat Struct Mol. Biol., 18(5):592-6 (including the accompanying Supplementary Information available in the on-line version of the manuscript available on the Nature Structural & Molecular Biology web site) and Perez-Jimenez, et al., 2009, Nat Struct Mol Biol 16: 890-6, and Alegre-Cebollada et al., 2010, J Biol Chem, 285(25):18961-6. The contents of Perez-Jimenez et al., 2011, Nat Struct Mol. Biol., May; 18(5):592-6 (including the accompanying “Supplementary Information,”), Perez-Jimenez et al., 2009, Nat Struct Mol Biol 16:890-6 and Alegre-Cebollada et al., 2010, J Biol Chem, 285(25):18961-6, are herein incorporated by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

The market for industrial enzymes has exploded in the past decades, with applications now including biotech, pharma, detergents, textile production, food processing, wine making, paper manufacturing, beauty products and many other areas. This has created an increasing need for enzymes that are stable at a wider range of temperatures and pH. As of today, there is no reliable method to achieve this while not simultaneously affecting the activity. A common practice nowadays is to randomly insert mutations in existing enzymes and screen for variants that exhibit the desired characteristics. However, due to the enormous combinatorial possibilities, this often becomes a costly and work-intense endeavor, and never guarantees success. Still, this has been the preferred method to discover most of the presently used industrial enzymes, many of which are patented.

Little is known about how the chemistry of primitive enzymes arose and how the environmental conditions affected the evolution of their chemistry (Zalatan et al., Nat. Chem. Biol., 5:516-520 (2009)); however since these organisms lived on the primordial earth and in an environment that was much hotter and more acidic than today, their enzymes would have been optimized to have a higher thermal and acidic stability than their modern counterparts. Experimental paleogenetics and paleobiochemistry (e.g. the study of resurrected proteins) can reveal valuable information regarding the adaptation of extinct forms of life to climatic, ecological and physiological alterations (Thornton, Science 301, 1714-7 (2003); Thomson et al., Nat Genet. 37, 630-5 (2005); Boussau et al., Nature 456, 942-5 (2008); Chang et al., Mol Biol Evol 19, 1483-9 (2002)). Unfortunately, previous reconstruction and resurrection provide a journey back in time on the order of a only few millions years (Myr) (Benner et al., Adv Enzymol Relat Areas Mol Biol 75, 1-132, xi (2007); Thornton, Nat Rev Genet. 5, 366-75 (2004); Gaucher et al., Nature 425, 285-8 (2003)). Consequently, many hypotheses about ancient life remain untested and cannot be directly answered by examining fossil records (Nisbet and Sleep, Nature 409, 1083-91 (2001)). There is a need for reliable methods for optimizing the pH and temperature stabilities of existing enzymes. There is also a need for methods useful for developing enzymes in a predictable and cost effective manner that are more effective and work in a wider range of environments. This invention addresses these needs.

### SUMMARY OF THE INVENTION

In one aspect, the invention relates to an isolated polypeptide having a sequence selected from the group consisting of: SEQ ID NO: 1-7. In another aspect, the invention relates to an isolated polypeptide having at least about 75% identity to SEQ ID NO: 1-7. In still another aspect, the invention relates to an isolated polypeptide comprising at least about 10, at least about 20, at least about 30, at least about 50 at least about 60, at least about 70, at least about 80, at least about 90 or at least about 100 consecutive amino acids from any of SEQ ID NOs: 1-7. In one embodiment, the isolated polypeptide does not have 100% identity with any extant polypeptide. In another embodiment, the variant has at least about 85.5%, at least about 90.5%, at least about 92.5%, at least about 95%, about 96%, about 96.5%, about 97%, about 97.5%, about 98%, about 98.5%, about 99%, about 99.5% or about 99.9% amino acid sequence identity to any one of SEQ ID NO: 1-7.

In still a further embodiment, the isolated polypeptide has enzymatic activity. In still another embodiment, the isolated polypeptide has thioredoxin activity.

In yet another embodiment, the isolated polypeptide is labeled. In one embodiment, the label is colorimetric, radioactive, chemiluminescent, or fluorescent. In still a further embodiment, the isolated polypeptide is chemically modified. In one embodiment, the chemical modification comprises covalent modification of an amino acid. In another embodiment, the covalent modification comprises methylation, acetylation, phosphorylation, ubiquitination, sumoylation, citrullination, or ADP ribosylation.

In one aspect, the invention relates to an isolated antibody that specifically binds to a polypeptide of any of SEQ ID NO: 1-7.

In another aspect, the invention relates to an isolated nucleic acid comprising a nucleic acid sequence which encodes a polypeptide having a sequence selected from the group consisting of: SEQ ID NO: 1-7. In another aspect, the invention relates to an isolated nucleic acid comprising a nucleic acid sequence which encodes a polypeptide having at least about 75% identity to SEQ ID NO: 1-7. In another aspect, the invention relates to an isolated nucleic acid comprising a nucleic acid sequence which encodes a polypeptide comprising at least about 10, at least about 20, at least about 30, at least about 50 at least about 60, at least about 70, at least about 80, at least about 90 or at least about 100 consecutive amino acids from any of SEQ ID NOs: 1-7.

In one embodiment, the nucleic acid sequence is optimized for expression in a mammalian expression system. In another embodiment, the nucleic acid sequence is optimized for expression in a bacterial expression system. In one embodiment, the bacterial expression system is E. coli. In another embodiment, the isolated nucleic acid is operably linked to one or more control sequences that direct the production of the polypeptide in a suitable expression host.

In another aspect, the invention relates to a recombinant expression vector comprising an isolated nucleic acid comprising a nucleic acid sequence which encodes a polypeptide having a sequence selected from the group consisting of: SEQ ID NO: 1-7.

In another aspect, the invention relates to a recombinant expression vector comprising an isolated nucleic acid comprising a nucleic acid sequence which encodes a polypeptide having at least about 75% identity to SEQ ID NO: 1-7.

In another aspect, the invention relates to a recombinant expression vector comprising an isolated nucleic acid comprising a nucleic acid sequence which encodes a polypeptide comprising at least about 10, at least about 20, at least about 30, at least about 50 at least about 60, at least about 70, at least about 80, at least about 90 or at least about 100 consecutive amino acids from any of SEQ ID NOs: 1-7.

In another aspect, the invention relates to a host cell comprising a recombinant expression vector comprising an isolated nucleic acid comprising a nucleic acid sequence which encodes a polypeptide having a sequence selected from the group consisting of: SEQ ID NO: 1-7.

In another aspect, the invention relates to a host cell comprising a recombinant expression vector comprising an isolated nucleic acid comprising a nucleic acid sequence which encodes a polypeptide having at least about 75% identity to SEQ ID NO: 1-7.

In another aspect, the invention relates to a host cell comprising a recombinant expression vector comprising an isolated nucleic acid comprising a nucleic acid sequence which encodes a polypeptide comprising at least about 10, at least about 20, at least about 30, at least about 50 at least about 60, at least about 70, at least about 80, at least about 90 or at least about 100 consecutive amino acids from any of SEQ ID NOs: 1-7.

In still a further aspect, the invention relates to a method for producing a polypeptide having a sequence selected from the group consisting of: SEQ ID NO: 1-7, the method comprising cultivating a host cell comprising a nucleic acid construct comprising a polynucleotide encoding the polypeptide under conditions suitable for production of the polypeptide; and recovering the polypeptide.

In still a further aspect, the invention relates to a method for producing a polypeptide having at least about 75% identity to SEQ ID NO: 1-7, the method comprising cultivating a host cell comprising a nucleic acid construct comprising a polynucleotide encoding the polypeptide under conditions suitable for production of the polypeptide; and recovering the polypeptide.

In still a further aspect, the invention relates to a method for producing a polypeptide comprising at least about 10, at least about 20, at least about 30, at least about 50 at least about 60, at least about 70, at least about 80, at least about 90 or at least about 100 consecutive amino acids from any of SEQ ID NOs: 1-7, the method comprising cultivating a host cell comprising a nucleic acid construct comprising a polynucleotide encoding the polypeptide under conditions suitable for production of the polypeptide; and recovering the polypeptide.

In still another aspect, the invention relates to a method of generating a reconstructed ancestral polypeptide having greater activity or stability at low pH than an extant polypeptide, the method comprising (a) aligning a plurality of sequences corresponding to homologues of the extant polypeptide, (b) generating a phylogenetic tree of the plurality of sequences corresponding homologues of the extant polypeptide, (c) using bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position and wherein a polypeptide comprising the reconstructed ancestral polypeptide sequence has increased activity or stability at low pH relative to the extant polypeptide.

In still another aspect, the invention relates to a method generating a reconstructed ancestral polypeptide having greater activity or stability at high temperature than an extant polypeptide, the method comprising (a) aligning a plurality of sequences corresponding to homologues of the extant polypeptide, (b) generating a phylogenetic tree of the plurality of sequences corresponding homologues of the extant polypeptide, (c) using bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position and wherein a polypeptide comprising the reconstructed ancestral polypeptide sequence has increased activity or stability at high temperature relative to the extant polypeptide.

In still another aspect, the invention relates to a method generating a reconstructed ancestral polypeptide having a higher melting temperature than an extant polypeptide, the method comprising (a) aligning a plurality of sequences corresponding to homologues of the extant polypeptide, (b) generating a phylogenetic tree of the plurality of sequences corresponding homologues of the extant polypeptide, (c) using bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position and wherein a polypeptide comprising the reconstructed ancestral polypeptide sequence has a higher melting temperature than an extant polypeptide.

In one embodiment, the extant polypeptide is a thioredoxin polypeptide.

In another aspect, the invention relates to a polypeptide produced according to the methods described herein.

### BRIEF DESCRIPTION OF THE FIGURES

FIG. 1. Single molecule assay of Trx catalysis. FIG. 1A A pair of vicinal cysteines (positions 32 and 75 in the sequence; yellow) are engineered into the I27 protein structure, dividing the protein mechanically in two parts. The two cysteines spontaneously form a disulfide bond. A polypeptide made of eight repeats of such engineered I27 proteins, (I27_{S-S})₈, is mechanically stretched at constant force. FIG. 1B Unfolding of a single protein in the chain causes a step elongation by ˜11 nm. Unfolding also removes the steric constraints on the disulfide bond exposing it to a nucleophilic attack by a Trx enzyme present in the surrounding solution. FIG. 1C A successful nucleophilic attack reduces the disulfide bond and allows for a further extension of the protein by ˜14 nm. FIG. 1D Experimental force clamp trace showing the stepwise elongation of a (I27_{S-S})₈ polypeptide at a constant force of 100 pN. The first step marks the unfolding of a single I27_{S-S} module in the chain and the second the reduction of its disulfide bond. The rate of reduction at any given force is easily measured from a collection of such traces. FIG. 1E Force dependency of the rate of reduction of disulfide bonds by different reducing agents. Human Trx shows a negative force dependency that reaches a force independent minimum. By contrast, L-cysteine shows a simple exponential increase in the rate of reduction with the applied force. Bacterial thioredoxins show a combination of mechanisms giving a characteristic V shaped force dependency.

FIG. 2. Molecular mechanisms of Trx catalysis. FIG. 2A Trx enzymes main structural features are a prominent binding groove marked by the shaded light green area, and the catalytic cysteine located on the rim of the groove (human; PDB code 3Trx). FIG. 2B A Trx enzyme collides and binds a substrate protein that contains a disulfide bond. Once the disulfide bonded substrate binds to the groove, the sulfur atoms of the catalytic cysteine (#1, inset) and the substrate disulfide (#2,3, inset) must align 180° from each other in order to acquire the correct S_{N}2 geometry for disulfide bond reduction to occur. This alignment takes place inside the binding groove.

FIG. 3. Structural characteristics of the binding groove in Trx enzymes. FIG. 3A Geometric characteristics of the peptide-binding groove in human Trx. FIG. 3B A clear structural difference can be observed when comparing bacterial and eukaryotic origin Trxs. In the case of eukaryotic Trxs the binding groove is much deeper and hindered than in the case of bacterial Trxs. FIG. 3C Comparison of the force dependency of the reduction rate for human and E. coli Trx enzymes. Human Trx (10 μM, red squares) shows two distinct mechanisms. A first mechanism is exponentially inhibited by force (I), and a second mechanism is force independent (II). A third mechanism is apparent in E. coli Trx (10 μM, green triangles) whereby at high forces, the rate of catalysis increases exponentially (III).

FIG. 4. Resurrected Trx from the Last Bacterial Common Ancestor (LBCA). FIG. 4A Differential scanning calorimetry measure the melting temperatures of LBCA (113° C.) and modern E. Coli Trx (87° C.). FIG. 4B LBCA is active at pH 5, by contrast modern E. coli and human thioredoxin show ˜20 fold lower rates at this pH. FIG. 4C The rate of reduction of LBCA shows a maximum at 100 pN, suggesting changes in the way the substrate fits into the binding groove. By contrast, all extant Trx enzymes show a maximal rate at zero force.

FIG. 5. Schematic of the combined TIRF-AFM (Total Internal Reflection Fluorescence-Atomic Force Microscope) experiment. FIG. 5A A fluorescently labeled Trx enzyme binds to an exposed disulfide bond in an unfolded polypeptide. When bound, the enzyme is localized in the TIRF field and can consequently be detected as a bright fluorescence spot localized exactly underneath of the AFM tip. The catalysis event is independently detected by the AFM as a stepwise extension of the substrate. The final dissociation event is detected as the disappearance of the fluorescent spot from the base of the AFM cantilever. FIG. 5B Schematic drawing showing the expected data from a combined TIRF-AFM experiment. The fluorescence intensity data comes from the pixels on the CCD corresponding to the area under the tip of the AFM. The extension trace shows the surface-tip distance for the AFM during force-clamp. Three relevant dwell times to be measured are marked 1, 2 and 3 respectively. The force dependency of all three dwell times will be measured.

FIG. 6. Force spectroscopy reveals the dynamic rearrangement of the substrate during Trx catalysis. FIG. 6A An Atomic Force Microscopy (AFM) based assay of Trx catalysis. A disulfide bonded polypeptide is picked up by an AFM cantilever and mechanically stretched at constant force. The cartoons on the right show the detection scheme. The polypeptide is first extended by unfolding, right up to the disulfide bond. The exposed disulfide then undergoes a nucleophilic attack by the Trx enzyme. Reduction of the substrate disulfide bond allows for an extra extension that is easily detected by the AFM. The rate of reduction is measured from the kinetics of the step increases in length that mark each reduction event. FIG. 6B A key observation made using the single molecule assay was that a sufficiently high mechanical force applied to the substrate disulfide bond inhibited the enzymatic reaction. The sequence of cartoons explains the effect of a pulling force in inhibiting the rotation of the disulfide bond that is needed to acquire the configuration for the S_{N}2 reaction.

FIG. 7. A putative search mechanism for Trx enzymes. (1) A Trx enzyme undergoing a 3-D diffusion search randomly binds the exposed polypeptide. (2) The enzyme then undergoes a 1-D diffusion search for the exposed disulfide, over a sliding distance dₛₗ. This mechanism greatly reduces the time necessary for finding the target.

FIG. 8. Phylogenetic Tree used for the ancestral sequence reconstruction of Trx enzymes. A total of 203 sequences were used (see Table 1). The nodes of interest are indicated with red arrows. Last bacterial common ancestors (LCBA), last archaeal common ancestor (LACA), archaea/eukaryota common ancestor (AECA), last common ancestor cyanobacterial and deinococcus/thermus groups (LPBCA) that represents the origin of photosynthetic bacteria; last eukaryotic common ancestor (LECA), last common ancestor of γ-proteobacteria (LGPCA) and last common ancestor of animals and fungi (LAFCA).

FIG. 9. Phylogenetic analysis of Trx enzymes and ancestral sequences reconstruction. FIG. 9A Schematic phylogenetic tree showing the geological time in which different extinct organisms lived, i.e., last bacterial common ancestors (LBCA); last archaeal common ancestor (LACA); archaea/eukaryota common ancestor (AECA) and last eukaryotic common ancestor (LECA). Other internal nodes are: the last common ancestor of photosynthetic bacteria (LPBCA), the last common ancestor of γ-proteobacteria (LGPCA), and the last common ancestor of animals and fungi (LAFCA). The dashed lines represent further bifurcations. Divergence times are compiled from multiple sources (see Hedges and Kumar, The Timetree of life, xxi, 551 p. (Oxford University Press, Oxford, 2009)). FIG. 9B Posterior probability distribution of the inferred amino acids across 106 sites for the interested internal nodes. The inferred amino acid at each site for the interested internal node is the residue with the highest posterior probability. FIG. 9C Denaturation temperatures (Tₘ) vs. geological time for ancestral Trx enzymes. Modern E. coli and Human Trx enzymes are also indicated. The inset shows experimental DSC thermograms for E. coli Trx and LBCA Trx.

FIG. 10 M-PASs for Trx enzymes belonging to representative extinct organisms: The sequences are calculated using maximum likelihood methods. Also included are E. coli and human Trx sequences for comparative purposes. A high degree of conservation around the active site CGPC is observed (red residues marked with asterisks).

FIG. 11. Single-molecule disulfide reduction assay. FIG. 11A Schematic representation of the singe-molecule disulfide reduction assay. A first pulse of force rapidly unfolds the I27_{G32C-A75C} domains (Unf.). When the disulfide bond is exposed to the solvent a single Trx molecule can reduce it (Red.) FIG. 11B Experimental force-clamp trace showing single disulfide reductions of a (I27_{G32C-A75C})₈ polypeptide. The unfolding pulse was set at 185 pN for 0.2 s and the test-pulse force at 500 pN. FIG. 11C Probability of reduction (P_{red}(t)) resulted from summing and normalizing the reduction test pulse at different forces for AECA Trx (3.5 μM). FIG. 11D Force-dependency of disulfide reduction by AECA Trx; human Trx is also shown for comparison. Both Trx enzymes show a similar pattern: a negative force-dependency of the reduction rate, from 30-200 pN, consistent with a Michaelis-Menten mechanisms and a force-independent mechanism, from 200 pN and up, described by an electron transfer reaction (Perez-Jimenez et al., Nat Struct Mol Biol 16, 890-6 (2009)). Notice the higher activity for AECA Trx (3.5 μM for AECA Trx vs. 10 μM for human TRX). The lines represent fittings to the kinetic model.

FIG. 12. Force-clamp experiment for detection of single disulfide reduction events. A first pulse of force (175 pN, 0.3 s) unfolds the I27_{G32C-A75C} domains up to the disulfide bond. The unfolding events can be monitored as a series of step of ˜11 nm per module (bottom panel). A second pulse of force (100 pN) is applied to monitor single disulfide reduction by Trx enzymes. In this case the release of the trapped residues behind the disulfide bond gives rise to a length increment of ˜14 nm per module (top panel).

FIG. 13A-F. Experimental traces of single disulfide reductions by ancestral Trxs. Both, the unfolding pulse (175 pN) and the test pulse at different forces are shown. Individual reduction events can be observed in the test-pulse force. Numerous traces like these (15-80) are used at every force to complete the full force-dependency of disulfide bond reduction by Trx enzymes, as shown in FIG. 14.

FIG. 14. Force-dependence of disulfide reduction by ancestral Trx enzymes. The reduction rate at a given force is obtained by summing, averaging and fitting to a single exponential numerous traces (15-80) like the one shown in FIG. 11B. The solid lines are fitting to the kinetic model. The grey circles and dashed lines represent the rate vs. force dependence for modern Trxs: Pea Trxm from chloroplast (FIG. 14C), P. falciparum Trx (FIG. 14D), E. coli Trx (FIGS. 14A and 14E) and Human Trx (FIG. 14F) (all extracted from Perez-Jimenez et al. Nat Struct Mol Biol 16, 890-6 (2009)). These modern Trxs are descendants of the ancestral Trxs in the same plot.

FIG. 15. Rate constants for disulfide bond reduction by ancestral Trxs. These values are obtained by extrapolating to zero force the fitting of the reduction rate vs. force data (FIG. 8) to the three-state kinetic model described in the methods section.

FIG. 16. Rate constants of disulfide bond reduction at pH 5. FIG. 16A A high activity for AECA (black squared) and LACA (circles) Trxs can be observed at pH 5 when the substrate is pulled at low forces (50-150 pN). LBCA Trx (triangles) shows similar activity to that at pH 7.2 with a similar trend (FIG. 14A). The solid lines are exponential fit to the experimental data. FIG. 16B The rate constants for disulfide reduction by ancestral Trxs at F=100 pN are remarkably high when compared with the rate constants measured for modern Trxs, E. coli and human at the same force.

FIG. 17. Functional assay of fluorescently labeled Trx enzymes. FIG. 17A Ensemble average of reduction events obtained with labeled E. coli Trx enzymes (10 μM). FIG. 17B TIRF image capturing a labeled enzyme entering the evanescent field. The trace shows the time course of one such visit. Stepwise bleaching events mark the multiple labels of the enzyme (arrows).

FIG. 18. A single molecule assay for oxidative folding. FIG. 18A Under a denaturing force of 110 pN, each initial (I27_{S-S})₈ unfolding event is measured as an 11 nm extension of the polypeptide, followed by reduction events catalyzed by human thioredoxin (10 μM wild-type hTrx), yielding additional 14 nm extensions (inset). Refolding of the fully denatured polypeptide is subsequently initiated by switching off the stretching force. After some time Δt, folding is stopped and the state of the substrate is probed by again applying a stretching force. During the probe stage we only observed 25 nm steps, indicating that while the (I27_{S-S})₈ polypeptide had refolded, the disulfide bonds did not reoxidize. FIG. 18B A histogram of the step sizes observed during the probe pulse from different traces confirms the absence of reoxidized proteins. FIG. 18C By contrast if the exact same experiment is repeated in the presence of a mutant form of human thioredoxin (hTrx^{C35S}), all disulfide bonds reduced during the denature pulse, become reoxidized as demonstrated by the presence of an equal number of 11 nm and 14 nm steps during the probe pulse.

FIG. 19. Cross-linking reaction to generate cleavable substrates. FIG. 19A Two distant cysteines are introduced in the I27 protein at positions A and B (positions 27 and 55). We covalently link the exposed cysteines with bifunctional molecules containing a cleavable bond (green bar). FIG. 19B If the I27 protein is left open, the unfolding step size is that of a full length protein with ΔL˜29 nm. FIG. 19C If the cysteines are bridged by a bifunctional reagent (here shown with BMDB), many I27 proteins now extend by only ΔL˜20 nm, limited by the covalent bridge. FIG. 19D Cleavage of a bridge by an enzyme will result into a further extension by ΔL˜9 nm, identifying the reaction.

FIG. 20. Rate constants for disulfide bond reduction by ancestral and modern Trxs enzymes. These values are obtained by extrapolating to zero force the fitting of the reduction rate vs. force data (FIG. 14) to the three-state kinetic model described herein.

FIG. 21. Insulin activity assay for ancestral and modern Trx enzymes. Activity determined with the turbidity insulin bulk enzymatic assay (Benner et al., Adv Enzymol Relat Areas Mol Biol 75, 1-132, xi (2007)). The turbidity assay is less sensitive in detecting differences in activity amongst the different enzymes. This assay cannot be used to probe the activity of the enzymes at pH 5 due to the precipitation of insulin at pH below 6 (Benner et al., Adv Enzymol Relat Areas Mol Biol 75, 1-132, xi (2007); Thornton, Nat Rev Genet. 5, 366-75 (2004)).

FIG. 22. Rate constants for disulfide reduction by ancestral Trx enzymes at pH 5 are higher than for modern E. coli and human Trx. Thioredoxin from the acidophile Acetobater aceti shows activity at pH 5, enzymes from the thermophilic Sulfolobus tokodaii do not show a detectable rate of reduction at the same pH. All experiments were conducted at a pulling force of 100 pN. Error bars represent s.e.m. obtained using the bootstrap method.

FIG. 23. Activity of ancestral Trxs and modern E. coli Trx measured using DTNB as substrate at pH 5 and determined by monitoring spectrophotometrically the formation of TNB at 412 nm. Error bars represent s.d. from three different measurements.

FIG. 24. Experimental DSC thermogram for Sulfolubus tokodaii Trx (Archaea). The solid line represents fit to the two-state thermodynamic model (Liberles, Ancestral sequence reconstruction, xiii, 252 p. (Oxford University Press, Oxford; New York, 2007)). A Tₘ of 122.6° C. is obtained from the fit.

FIG. 25. Structural representation of the ancestral enzyme thioredoxin AECA.

### DETAILED DESCRIPTION OF THE INVENTION

The issued patents, applications, and other publications that are cited herein are hereby incorporated by reference to the same extent as if each was specifically and individually indicated to be incorporated by reference.

Industry has a large demand of pH stable and temperature polypeptides for use in a number of industrial applications. Methods to alter polypeptide pH and temperature stability without eliminating function of the polypeptide are highly needed. The methods described herein are related in part to the finding that it is possible to predict, synthesize and characterize enzymes from extinct organisms that lived on earth as long as 4 billion years ago. In certain aspects, the methods described herein are relate to the understanding that because these organisms lived on the primordial earth (i.e. in an environment that was much hotter and more acidic than today), their enzymes were necessarily optimized through selective pressure to have a higher thermal and acidic stability than their modern counterparts. In some aspects, the methods described herein are relate to the finding that because enzyme homologues exist different species, Bayesian statistics can be used to predict the ancestral gene encoding for a version of the enzyme that was present in the common ancestor of these organisms.

In certain aspects, the methods described herein can be used to substitute amino acids according to their presence in resurrected protein sequences from extinct organisms. In one embodiment, the methods described herein are useful for altering (e.g increasing) the stability of a recombinant polypeptide at low pH and/or high temperatures by making one or more conservative substitutions in the amino acid sequence of the polypeptide. In one embodiment, the methods described herein are useful for altering (e.g increasing) the activity of a recombinant polypeptide at low pH and/or high temperatures by making one or more conservative substitutions in the amino acid sequence of the polypeptide.

In certain aspects, the invention described herein relates to the finding that single molecule force-clamp spectroscopy can be used to study protein dynamics under a mechanical force. The experimental resurrection of ancestors of these universal enzymes together with the sensitivity of single-molecule techniques can be a powerful tool towards understanding the origin and evolution of life on Earth. As described herein, the force-dependency of a reaction can be a sensitive probe of substrate nanomechanics during catalysis. This type of protein spectroscopy can also be useful for obtaining details of enzyme active site dynamics. The methods described herein can also complement structural x-ray and NMR data and provide benchmarks for molecular dynamics simulations

### DEFINITIONS

The singular forms “a,” “an,” and “the” include plural references unless the content clearly dictates otherwise.

As used herein, “sequence identity” means the percentage of identical nucleotide or amino acid residues at corresponding positions in two or more sequences when the sequences are aligned to maximize sequence matching, i.e., taking into account gaps and insertions. “Percent identity” in the context of two or more nucleic acids or polypeptide sequences, refers to the percentage of nucleotides or amino acids that two or more sequences or subsequences contain which are the same. A specified percentage of amino acid residues or nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for correspondence over a comparison window, or designated region as measured using one of the following sequence comparison algorithms or by manual alignment and visual inspection.

As used herein, the term “extant” refers to taxa (such as species, genera or families) that are still in existence (living). The term extant contrasts with extinct. As used herein, the terms “extant protein”, “extant polypeptide”, “extant amino acid sequence”, “extant gene” and “extant nucleic acid sequence” refer to proteins, polypeptides, amino acid sequences, genes, and nucleic acid sequences from extant taxa.

Other definitions are provided throughout the specification.

A journey back in time is possible at the molecular level by resurrecting proteins from extinct organisms. Laboratory resurrection of these ancestral proteins enables exploration of aspects of ancient life that cannot be inferred from fossil records alone (Benner et al., Adv Enzymol Relat Areas Mol Biol 75, 1-132, xi (2007); Thornton, Nat Rev Genet. 5, 366-75 (2004); Liberles, Ancestral sequence reconstruction, xiii, 252 p. (Oxford University Press, Oxford; New York, 2007); Hall, Proc Natl Acad Sci USA 103, 5431-6 (2006). Such time traveling is largely limited by the ambiguity in the historical models used for ancestral sequence inference. (Pollock and Chang, in Ancestral sequence reconstruction, pages 85-94 (ed. Liberles. D. A., Oxford University Press, Oxford; New York, 2007); Gaucher et al., Nature 425, 285-8 (2003); Gaucher et al., Nature 451, 704-7 (2008)). For instance, uncertainties in databases, sequence alignments, failures in evolutionary theories and uncertainty in the construction of phylogenetic trees are common sources of ambiguity.

Understanding the molecular mechanisms of enzyme function presents unique challenges in biophysics. In certain aspects, the invention described herein relates to computational methods for resuscitating ancestral genes. In some embodiments, the methods described herein can be used to reconstruct the amino acid sequence of ancient proteins. Reconstructed proteins can be expressed in an expression system and, in certain applications, examined for their activity, pH stability or thermal stability (Gaucher et al. Nature, 2008. 451(7179): p. 704-U2; Gaucher et al, Nature, 2003. 425(6955): p. 285-8).

The pH and temperature stability of polypeptides can depend in part on the distribution of amino acid residues throughout the three dimensional structure of the polypeptide. In one aspect, the methods described herein are relate to findings from the resurrection of seven Precambrian thioredoxin enzymes (Trx), dating back between ˜1.4 and ˜4 billion years ago (Gyr). These findings relate to the evolution of enzymatic reactions of thioredoxin enzymes (Trx) from extinct organisms that lived in the Precambrian. Their mechanism of reduction was probed using single molecule force-spectroscopy which can readily distinguish simple nucleophiles from the more complex chemistry of the active site of Trx enzymes. As described herein, differential scanning calorimetry (DSC) showed that these resurrected enzymes have melting temperatures up to ˜32° C. higher than those of extant Trx, following a trend with a slope of ˜6 K/Gyr. From the force-dependency of the rate of reduction of an engineered substrate can be used to determine whether the ancient Trxs utilized chemical mechanisms of reduction similar to those of modern enzymes. As described herein, the most ancient enzymes showed high activity at low pH, where the extant Trxs became inactive under in low pH environments. The results described herein show that, while Trx enzymes have maintained their reductase chemistry unchanged, they have adapted over a 4 Gyr time span to the changes in temperature and ocean acidity that characterize the evolution of the environment from ancient to modern Earth.

The results described herein also show that the chemical mechanisms observed in modern Trx enzymes were already present in Trxs from Precambrian organisms. Ancestral Trx enzymes from LBCA, AECA and LACA that lived in the mid-to-late Hadean were highly resistant to temperature and active in relatively acidic conditions. These findings are consistent with the hypothesis that in early life Trx enzymes were present in hot environments and these environments have progressively cooled from 4 to 0.5 Gyr (Nisbet and Sleep, Nature 409, 1083-91 (2001); Gaucher et al., Nature 451, 704-7 (2008); Knauth et al., Geo. Soc. Am. Bull., 115: 566-580 (2003); Schulte, M., Oceanography 20, 42-49 (2007)). However, it is also possible that a much cooler early Earth was populated by psychrophiles, mesophiles and thermophiles and that the latter could have been the only survivors of cataclysmic events (e.g., the late heavy bombardment or global glaciations on Early Earth (Nisbet and Sleep, Nature 409, 1083-91 (2001); Gogarten-Boekels et al., Orig. Life Evol. Biosph., 25: 251-264 (1995)). Thus, these findings indicate that important biochemical pathways in the modern biosphere were already established by 3.5 Gyr ago (Nisbet and Sleep, Nature 409, 1083-91 (2001)). For instance, metabolism is one of the most conserved cellular processes. Important pathways like energy production, sugar degradation, cofactor biosynthesis or amino acids processing are highly conserved from bacteria to human and were likely present in LUCA (Peregrin-Alvarez et al., Genome Res 13, 422-7 (2003)). Thus, in some aspects, the present invention is directed to a nucleic acid encoding a recombinant thioredoxin or to recombinant thioredoxin amino acid sequences, such as for example a thioredoxin polypeptide optimized to have greater stability and/or activity at high temperature and/or low pH, that has been modified to change amino acids where the one or more modified are pH optimizing or temperature optimizing modifications.

Evolution operates at multiple levels of biological organization; however, enzymatic mechanisms accompanying adaptive changes seem to be highly conserved. The ability of enzymes to maintain specific chemical reactivities and mechanisms in disparate environments is necessary for the diversification of life. While this ability is exemplified by Trx enzymes, it can also be universal to all proteins (e.g., ubiquitin, RNase, ATPase or other metabolic enzymes that have been maintained in nearly all organisms throughout the history of life). Thus, although some of compositions and methods described herein relate to the activity of resurrected thioredoxin, the paleoenzymological methods described herein can be used to generate polypeptides optimized to have greater stability and/or activity at high temperature and/or low pH. The experimental resurrection of ancestors of these universal proteins together with the sensitivity of single-molecule techniques can be a powerful tool towards understanding the origin and evolution of life on Earth.

In one aspect, the invention relates to computational methods for determining ancestral sequences. Such methods can be used, for example, to determine ancestral sequences for an extant polypeptide (e.g. thioredoxin). In another aspect, the invention relates to methods for increasing the stability and/or activity of a polypeptide (e.g. a thioredoxin) at low pH or at elevated temperature. Methods for determining ancestral sequences can be based on amino acid sequences or on nucleic acid sequences encoding (or predicted to encode) proteins.

In some embodiments, the computational methods described herein are based on the principle of maximum likelihood. The sequences of polypeptides used in the methods described herein can be selected on the basis of a common feature (e.g. a threshold sequence identity, common enzymatic activity, or common modular domain architecture). The methods may involve the construction of a phylogeny using an evolutionary model of the probabilities of amino acid or nucleic acid substitutions polypeptide among different organisms.

Where the sequences differ (e.g. due to mutation), the maximum likelihood methodology can be used to assigns an amino acid or nucleic acid residue to the node a phylogenetic trees (i.e., the branch point of the lineages). Generally, a model of sequence substitutions and then a maximum likelihood phylogeny can be determined for multiple data sets. The sequence at the base node of the maximum likelihood phylogeny is referred to as the ancestral sequence (or most recent common ancestor).

In certain embodiments, the invention is directed to methods for generating an ancestral polypeptide (e.g. thioredoxin) sequences through reconstruction of phylogenetic trees. The ancestral polypeptide sequence may be any polypeptide sequence which contains at least homolog in another organism.

In one aspect, the invention described herein relates to a method for increasing the temperature stability of a recombinant polypeptide produced from a nucleic acid in an expression system, the method comprising replacing one or more temperature stability decreasing amino acids of the recombinant polypeptide with one or more temperature stability increasing amino acids. In another aspect, the invention described herein relates to a method for increasing the pH stability of a recombinant polypeptide produced from a nucleic acid in an expression system, the method comprising replacing one or more temperature pH decreasing amino acids of the recombinant polypeptide with one or more pH stability increasing amino acids.

In certain aspects, the present invention relates to the finding that it is possible to predict, synthesize and characterize polypeptides from extinct organisms. Thus, one embodiment the stability of a extant polypeptide at low pH (e.g. a pH lower than the pH at which the extant polypeptide is expressed in an organism, or the pH at which the polypeptide displays its greatest stability and/or activity) can be increased by reconstructing an ancestral polypeptide of the extant polypeptide by (a) aligning a plurality of sequences corresponding homologues of the extant polypeptide, (b) generating a phylogenetic tree of the plurality of sequences corresponding homologues of the extant polypeptide, (c) using Bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position.

Thus, one embodiment the stability of a extant polypeptide at high temperature (e.g. a temperature higher than the temperature at which the extant polypeptide is expressed in an organism, or the temperature at which the polypeptide displays its greatest stability and/or activity) can be increased by reconstructing an ancestral polypeptide of the extant polypeptide by (a) aligning a plurality of sequences corresponding homologues of the extant polypeptide, (b) generating a phylogenetic tree of the plurality of sequences corresponding homologues of the extant polypeptide, (c) using Bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position.

In another embodiment the activity of a extant polypeptide at low pH (e.g. a pH lower than the pH at which the extant polypeptide is expressed in an organism, or the pH at which the polypeptide displays its greatest stability and/or activity) can be increased by reconstructing an ancestral polypeptide of the extant polypeptide by (a) aligning a plurality of sequences corresponding homologues of the extant polypeptide, (b) generating a phylogenetic tree of the plurality of sequences corresponding homologues of the extant polypeptide, (c) using Bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position.

In another embodiment the activity of a extant polypeptide at high temperature (e.g. a temperature higher than the temperature at which the extant polypeptide is expressed in an organism, or the temperature at which the polypeptide displays its greatest stability and/or activity) can be increased by reconstructing an ancestral polypeptide of the extant polypeptide by (a) aligning a plurality of sequences corresponding homologues of the extant polypeptide, (b) generating a phylogenetic tree of the plurality of sequences corresponding homologues of the extant polypeptide, (c) using Bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position.

In another embodiment the melting temperature of a extant polypeptide can be increased by reconstructing an ancestral polypeptide of the extant polypeptide by (a) aligning a plurality of sequences corresponding homologues of the extant polypeptide, (b) generating a phylogenetic tree of the plurality of sequences corresponding homologues of the extant polypeptide, (c) using Bayesian statistical analysis to generate inferred sequences of one or more ancestral genes encoding a version of the polypeptide that was present in a common ancestor of at least two or more organisms in the phylogenetic tree, (d) calculating posterior probabilities for all 20 amino acids in each inferred sequence, (e) generating a reconstructed ancestral polypeptide sequence by assigning to each position in the inferred sequence the amino acid residue having the highest posterior probability for that position.

In one embodiment, the sequence of a reconstructed protein can be generated by contracting a phylogenetic tree from a plurality of extant (modern) sequences of the enzyme to be reconstructed. The phylogenetic tree can be used to predict the sequences corresponding to every node of the tree. In one embodiment, the enzyme to be reconstructed can be a thioredoxin enzyme and the extant enzymes of a plurality of extant thioredoxin enzymes can be used to construct a phylogenetic tree and predict the sequences of every node of the tree.

Generally, polypeptide sequences corresponding homologues of the extant polypeptide can be obtained from publicly available databases (e.g., GenBank). Sequence comparison and alignment can be performed according to different analytical parameters. For example, in some cases, one sequence can be used are a reference against which all other sequences are compared. In the case of sequence comparison algorithms, test and reference sequences can be input into a computer and sequence algorithm program parameters can be designate for analysis. Alignment of the sequences can be performed using any method, algorithm or program known in the art. Examples of suitable alignment programs include, but are not limited to, MUSCLE (Edgar, Nucleic Acids Res 32, 1792-7 (2004)), Clustal W, the BioEdit program available from North Carolina State University (available at http://www mbio.ncsu.edu/BioEdit/bioedit.html), and the SegEd program.

The terms “homologous” or “homologue” refer to related sequences that share a common ancestor or arise from gene duplication and are determined based on degree of sequence identity. Alternatively, a related sequence may be a sequence having homology, which has arisen by convergent evolution. These terms describe the relationship between a gene found in one species, subspecies, variety, cultivar or strain and the corresponding or equivalent gene in another species, subspecies, variety, cultivar or strain or, in the case of paralogous genes, two related sequences within a species, subspecies, variety, cultivar or strain. “Homologous sequences” are thought, believed, or known to be functionally related. A functional relationship may be indicated in a number of ways, including, but not limited to: (a) the degree of sequence identity; and/or (b) the same or similar biological function. Homology can be determined using software programs readily available in the art, such as those discussed in Current Protocols in Molecular Biology (F. M. Ausubel et al., eds., 1987).

The term “homolog” is also used to refer to proteins with amino acid sequences sharing at least about 60%, 70%, 80%, 90% or more identity with the amino acid sequences of an ancestral protein, such as the ancestral Trx proteins described herein. The term “homolog” is also used to refer to gene sequences with nucleic acid sequences sharing at least about 60%, 70%, 80%, 90% or more identity with nucleic acid sequences capable of encoding an ancestral protein, such as the ancestral Trx proteins described herein.

In certain embodiments of the methods described herein, the sequences and/or sequence alignments can be further subjected to manual correction. Other suitable alignment algorithms include, but are not limited to the local homology algorithm of Smith and Waterman (Adv. Appl. Math. 2:482 (1981)), by the homology alignment algorithm of Needleman and Wunsch (J. Mol. Biol. 48:443 (1970)), by the search for identity method of Pearson and Lipman (Proc. Natl. Acad. Sci. USA 85:2444 (1988)), by the progressive alignment method of Feng and Doolittle (J. Mol. Evol. 35:351-60 (1987)) (e.g. PILUP), by the CLUSTAL method described by Higgins and Sharp (Gene 73:237-44 (1988); CABIOS 5:151-53 (1989)), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally Ausubel et al., Current Protocols in Molecular Biology, John Wiley and Sons, New York (1996)). Analysis of the percent sequence identity between the test sequence(s) and the reference sequence can be performed on the basis of designated program parameters. For example, a reference sequence can be compared to other test sequences to determine the percent sequence identity relationship using the following parameters different gap weights, different gap length weights, and weighted end gaps. Appropriate parameters can be identified by one skilled in the art. In some embodiments, the number of sequences can also be reduced by treating conservative substitutions occupying a position in a sequence as being identical to a single residue occupying that position. The choice of residue representing the members of one or more conservative substitution groups may be selected based on the physio-chemical properties of the amino acid, the frequency of occurrence in the sequence alignment or any other criteria known in the art.

A “conservative substitution,” when describing a protein, refers to a change in the amino acid composition of the protein that is less likely to substantially alter the protein's activity. Thus, “conservatively modified variations” of a particular amino acid sequence refers to amino acid substitutions of those amino acids that are less likely to be critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing amino acids that are often functionally similar are well known in the art (see, e.g., Creighton, Proteins, W. H. Freeman and Company (1984)). Conservative amino acid substitutions can be made at one or more non-essential amino acid residues. A conservative amino acid substitution can be a substitution in which an amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), aliphatic side chains (e.g., glycine, alanine, valine, leucine, isoleucine), and sulfur-containing side chains (methionine, cysteine). Substitutions can also be made between acidic amino acids and their respective amides (e.g., asparagine and aspartic acid, or glutamine and glutamic acid).

Conservative amino acid substitutions can be utilized in making variants of the Trx enzymes described herein. For example, replacement of a leucine with an isoleucine or valine, an aspartate with a glutamate, a threonine with a serine, or a similar replacement of an amino acid with a structurally related amino acid, may not have a major effect on the properties of the resulting polypeptide or fusion polypeptide. Whether an amino acid change results in a functional polypeptide or fusion polypeptide can readily be determined by assaying the specific activity of the polypeptide or fusion polypeptide.

One skilled in the art will also be able to remove sequences below a particular size cut-off, subject the sequences to split decomposition analysis to remove any phylogenetic noise. A phylogenetic tree can then be constructed by heuristic search using a maximum likelihood (ML) approach. In one embodiment, one or more phylogenetic trees can be generated a suitable program known in the art. Examples of suitable programs include, but are not limited to PAUP (e.g. PAUP 4.0 beta) and PHYML. In one embodiment, the phylogenetic analysis and the phylogenetic tree can be performed using PAUP by the minimum evolution distance criterion with 1000 bootstrap replicates. Once phylogenetic trees are generated, one skilled in the art will appreciate that such tree can be rooted according to different parameters. In certain embodiments, the phylogenetic tree can be used to predict the sequences corresponding to every node of the tree. Parameters suitable for use with the methods described herein include, but are not limited to, strict or relaxed molecular clock model (Lai, Microbiol. Rev., 56:61-79, 1992; Lee et al., J. Virol., 73:11-18, 1999), non-reversible models of substitution, midpoint rooting, and/or outgroup criterion (Gao et al., J. Virol., 79:1154-1163, 2005; Higgins and Sharp, Gene, 73:237-244, 1988; Lai, Microbiol. Rev., 56:61-79, 1992; Lee et al., J. Virol., 73:11-18, 1999; Logvinoff et al., Proc. Natl. Acad. Sci. USA, 101:10149-10154, 2004; Mink et al., Virology, 200:246-255, 1994). The rooted tree can then be used as a template to simulate an ancestral sequence. Simulation of ancestral sequences at internal nodes as well as at common ancestor can be inferred using a reconstruction program using Bayesian statistical analysis. An exemplary reconstruction program for Bayesian statistical analysis is PAML (e.g. PAML version 3.14). In one embodiment, the Bayesian statistical analysis is performed using PAML and the gamma distribution for variable replacement rates across sites is incorporated (Yang, Comput Appl Biosci 13, 555-556 (1997)). In another embodiment, the Bayesian statistical analysis is performed using MrBayes (mrbayes csit.fsu.edu). For each site of the inferred sequences, posterior probabilities can be calculated for all 20 amino acids and the amino acid residue with the highest posterior probability can be assigned at each site of an inferred sequence.

Sequences corresponding homologues of the recombinant polypeptide can be nucleic acid sequences, amino acid sequences, confirmed sequences, predicted sequences or hypothetical sequences. Where conversion of nucleic acid sequences to amino acid sequences is required (e.g. for alignment purposes), one skilled in the art will readily be able to convert the nucleic acid sequences to amino acid sequences using appropriate codon translation tables and/or algorithms for identifying protein coding regions in nucleic acids. In certain embodiments, the sequences corresponding homologues of the recombinant polypeptide can be selected such that at least one sequence is from an organism of the archaea domain, at least one sequence is from an organism of the bacteria domain and at least one sequence is from an organism of the eukarya domain.

Phylogenetically related sequences may be divided according to any criteria known to a person of skill in the art. Exemplary subdivisions include, but are not limited to subdivisions according to phylogenetic distance, function, motif organization, or the like.

The methods of the present invention can be performed using a computer. In one embodiment, the invention involves the use of a computer system which is adapted to allow input of one or more sequences and which includes computer code for performing one or more of the steps of the various methods described herein. For example, the present invention encompasses a computer program that includes code for performing one or more of generating protein sequences, generating gene sequences, aligning gene or polypeptide sequences, generating phylogenetic relationships, performing maximum likelihood and/or Bayesian statistical analysis and for computing any of the methods described herein sequentially or simultaneously.

The computer systems of the invention can comprise a means for inputting data such as the sequence of proteins, a processor for performing the various calculations described herein, and a means for outputting or displaying the result of the calculations.

One of skill in the art can readily create computer code for executing the methods of the invention, using any suitable computer code language or system known in the art, such as “C” for example.

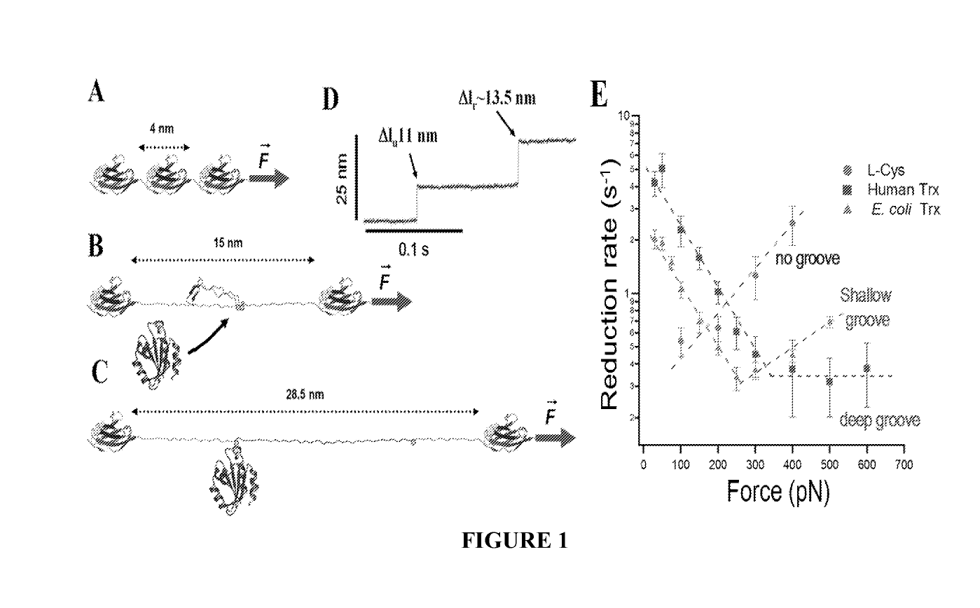

Thioredoxins belong to a broad family of oxidoreductase enzymes ubiquitous in all living organisms (Holmgren, Thioredoxin Annu Rev Biochem 54, 237-71 (1985)). In one aspect, the methods described herein relate to the evolution of thioredoxin (Trx) enzymes. In certain aspects, the methods and compositions described herein relate to the finding that the chemical mechanisms of reduction by thioredoxin enzymes have evolved over time and where the earliest forms thioredoxin enzymes had capabilities that were only comparable to those of simple reducing agents like glutathione or cysteine (FIG. 1E) (Ainavarapu et al., Journal of the American Chemical Society, 2008. 130(20): p. 6479-6487). Such evolutionary pressures can have driven the enzymes towards developing unique and efficient mechanisms of reduction (Wiita, A. P., et al., Nature, 2007. 450(7166): p. 124-7).

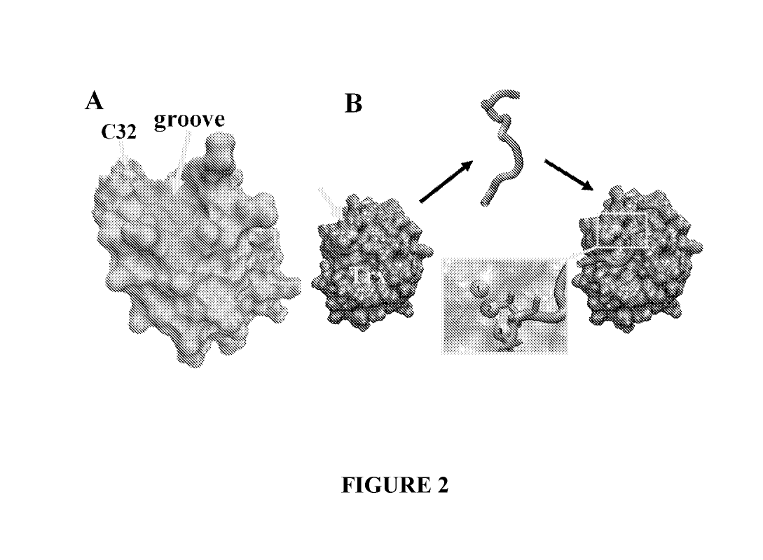

The archetypical active site (CXXC) and the Trx fold are well conserved throughout evolution, indicating that Trxs enzymes were present in primitive forms of life. By using single molecule force-clamp spectroscopy the chemical mechanisms of disulfide reduction by Trx enzymes can be examined in detail at the sub-Ångström scale (Wiita et al., Nature 450, 124-7 (2007); Perez-Jimenez et al., Nat Struct Mol Biol 16, 890-6 (2009)). Hence, the combination of single-molecule force spectroscopy and the resurrection of ancestral proteins can reveal novel insights into the reductase activity of these sulfur-based enzymes. Thioredoxin (Trx) enzymes reduce disulfide bonds in a myriad of target proteins in both intracellular and extracellular compartments (Amer and Holmgren, Eur J Biochem, 2000. 267(20): p. 6102-9; Kumar et al., Proc Natl Acad Sci USA, 2004. 101(11): p. 3759-64; Powis and Montfort, Annu Rev Biophys Biomol Struct, 2001. 30: p. 421-55). In addition to its role as an important cellular antioxidant, the reduction of disulfide bonds by Trx can activate signaling cascades by triggering conformational changes in transcription factors (e.g. NF-κB) (Lillig and Holmgren, Antioxid Redox Signal, 2007. 9(1): p. 25-47) or ion channel activation (Xu et al., TRPC channel activation by extracellular thioredoxin. Nature, 2008. 451(7174): p. 69-72). Trx plays essential roles in the life cycle of viruses (Holmgren, A., Thioredoxin and glutaredoxin systems. J Biol Chem, 1989. 264(24): p. 13963-6) and can be an activator of viral entry into cells. Trx catalyzes the reduction of disulfide bonds in the second domain of the extracellular receptor CD4 as an important step in HIV entry into cells (Matthias, et al., Nat Immunol, 2002. 3(8): p. 727-32; Matthias and Hogg, Antioxid Redox Signal, 2003. 5(1): p. 133-8). Trx is also involved in DNA replication and repair by keeping the essential enzyme ribonucleotide reductase in its reduced state (Avval and Holmgren, J Biol Chem, 2009. 284(13): p. 8233-40). Trx enzymes share a highly conserved amino acid motif, Cys-X-X-Cys, in their active sites as well as a characteristic structural motif called the Trx fold (FIG. 2). There are over 5,000 known DNA sequences that contain this motif and are classified as Trxs by Pfam database (http://pfam.sanger.ac.uk/).

Thioredoxin enzymes have structural features that help positioning the participating sulfur atoms, such that an attack through an S_{N}2 reaction is favored, resulting in disulfide bond reduction. An important structural feature in the Trx family of enzymes is the presence of a hydrophobic binding groove that abuts the active site of the enzyme (FIG. 2A).

The mode of action of Trx catalysis occurs through two conserved cysteine residues of the active site which play complementary roles during the reduction of a target disulfide bond. First, the catalytic Cys32 attacks the target disulfide bond resulting in a mixed disulfide between the enzyme and the substrate. Catalysis is resolved by a subsequent nucleophilic attack by Cys35 (Carvalho, et al., J Phys Chem B, 2008. 112(8): p. 2511-23; Chivers and Raines, Biochemistry, 1997. 36(50): p. 15810-6). After this cycle, the two cysteines in the active site are disulfide bonded and the enzyme is rendered inactive. Another enzyme called Trx reductase (TrxR) draws electrons from NADPH to reduce and reactivate Trx, allowing this cycle to be repeated indefinitely (Williams et al., Eur J Biochem, 2000. 267(20): p. 6110-7; Mustacich, Powis, Biochem J, 2000. 346 Pt 1: p. 1-8). The catalytic activity of Trx enzymes relies on an active cysteine thiolate (FIG. 2; Cys32) that reduces target disulfide bonds by acting as a potent nucleophile.

A structural feature of thioredoxin enzymes is a polypeptide binding groove adjacent to the active site of the enzyme. The groove also serves to orient the substrate with respect to the catalytic cysteine, creating signatures that can be detected by force-clamp spectroscopy. The target binds into the binding groove and the target is then reduced by the exposed thiol of the catalytic cysteine. At least four different types of force-dependent reactions can be distinguished. As described herein, a variety of extant and ancient thioredoxins with different groove characteristics, like depth and width, can be used to examine how groove characteristics determine the force-dependency of the reaction. In certain embodiments, the methods described herein can be used to identify groove-free forms of thioredoxin by using evolutionary trees to resuscitate ancient forms of the enzyme and study their catalytic mechanisms. As described herein, molecular dynamics simulations can be used to examine the relationship between the groove characteristics and the mechanisms observed.

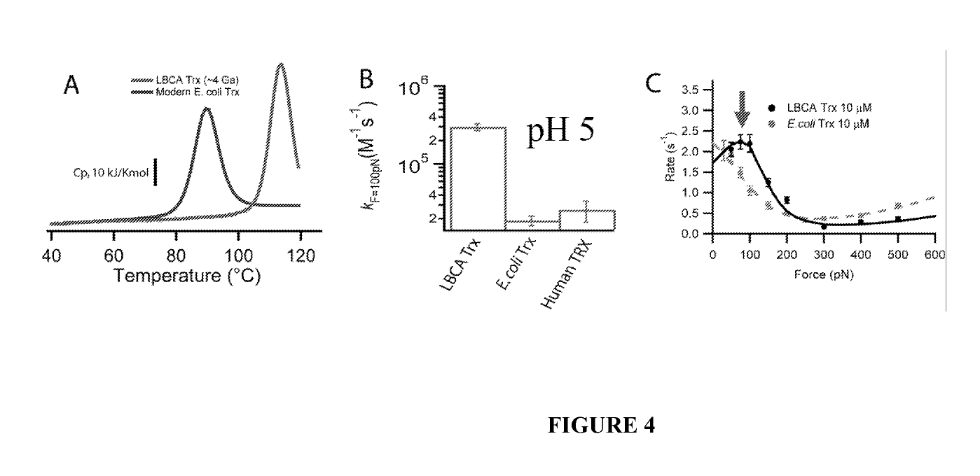

A fundamental step in the evolution of thioredoxin chemistry may have been the formation of this binding groove. Thus, by resurrecting ancient forms of thioredoxins, the methods described herein can be used to identify early versions of these enzymes where groove binding was either absent or shallow and poorly evolved (FIG. 4C). Such findings can be used to establish a detailed correlate between the binding groove and the observed force-dependent catalysis.

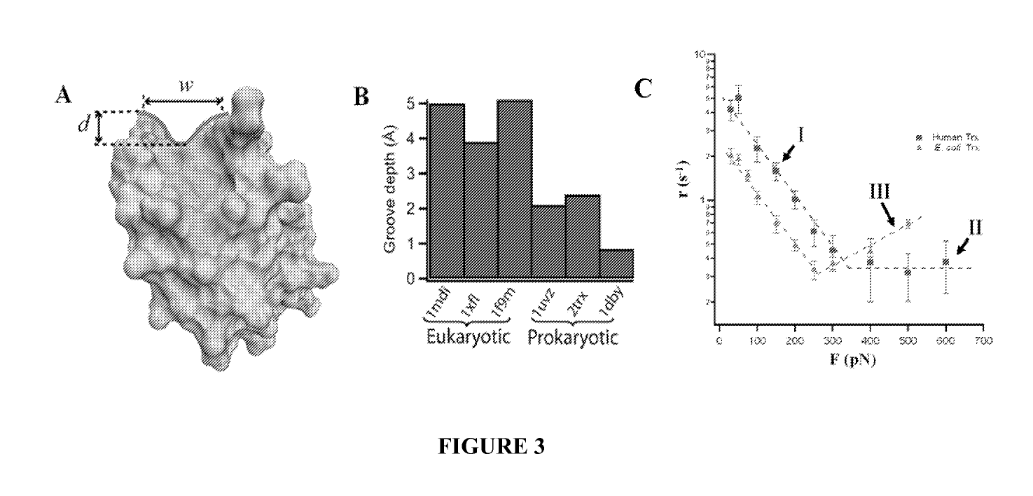

Several structural features of the binding groove can be directly measured from X-ray or NMR structures of Trx enzymes and by correlating them with observed chemical mechanisms of action. For example, structural axes can be defined to measure the depth and width of the binding groove in the region surrounding the catalytic cysteine (FIG. 3A) (Perez-Jimenez, et al., Nature Structural & Molecular Biology, 2009. 16(8): p. 890-U120). FIG. 3B shows the depth of the groove of three Eukaryotic Trx: spinach Trxf (PDB code: 1f9m) (Capitani et al., J Mol Biol, 2000. 302: p. 135-154), human Trx (1mdi) (Qin et al., Structure, 1995. 3: p. 289-297), A. thaliana Trxh1 (1xfl) (Peterson et al., Protein Sci., 2005. 14: p. 2195-2200); and three bacterial-origin Trx: human Trx2 (1uvz) (Smeets et al., Protein Sci., 2005. 14: p. 2610-2621), C. reinhardtii Trxm (1dby) (Lancelin et al., Proteins 2000. 41: p. 334-349), and E. coli Trx (2trx) (Katti et al., J Mol Biol, 1990. 212(1): p. 167-84). A difference in the structural characteristics of the groove is apparent between these selected prokaryotic and eukaryotic Trx enzymes. Trx enzymes with deeper grooves may limit the mobility of the substrate, and thereby restrict the type of chemical mechanisms available for reduction of the substrate, resulting in different force dependencies of catalysis (FIG. 3C).

The binding groove becomes evident by studying mixed disulfide complexes between a mutant form of Trx lacking C35 and disulfide bonded target such as Nf-kB and Ref-1 derived polypeptides (FIG. 2B) (Qin et al., Structure, 1995. 3: p. 289-297; Qin et al., Structure 1996. 4: p. 613-620). The enzyme can be prevented from resolving the mixed disulfide stage by mutating C35 and the substrate gets trapped in the groove, disulfide bonded to the catalytic cysteine. The structure of such mixed disulfide complexes indicates that both van der Waals contacts and specific intermolecular hydrogen bonds play roles in the recognition and binding of substrates in the Trx groove (Maeda et al. Structure, 2006. 14(11): p. 1701-10).

As described herein, ancient thioredoxin enzymes can be reconstructed that are functional and show greatly altered properties. Further, as described herein, Trx enzymes from different kingdoms can be reconstructed to identify thioredoxin enzymes showing unique features in their force-dependent rate of catalysis. Such findings can be related to their binding groove. Many x-ray structures of Trx enzymes are known (e.g. PDB: 1ZZY, 2FCH, 2FD3, etc). Similarly, x-ray structures of resurrected enzymes can also be resolved (e.g. LBCA; FIG. 4) and the characteristics of the groove can be correlated with observed force-dependent catalysis data. The methods described herein can also be used to develop detailed molecular models for the substrate-enzyme interactions for the thioredoxin family. These models can be tested by completing molecular dynamic simulations of the studied enzyme-substrate complexes (Wiita, A. P., et al., Nature, 2007. 450(7166): p. 124-7). Such analysis can be used to gain information about the mobility of the substrate disulfide related to the different chemical mechanisms (Perez-Jimenez, et al., Nature Structural & Molecular Biology, 2009. 16(8): p. 890-U120).

In on aspect, the invention relates to Trx ancestral proteins having the Trx amino acid sequence of SEQ ID NO: 1-7. Such ancestor proteins include, for example, full-length protein, polypeptides, fragments, derivatives and analogs thereof. In one aspect, the invention provides amino acid sequences of ancestor proteins in SEQ ID NOs: 1-7. In some embodiments, the ancestor protein is functionally active.

In one embodiment, the invention is directed to a last bacterial common ancestor (LBCA) Trx amino acid having the sequence

(SEQ ID NO: 1)
MSVIEINDENFEEEVLKSDKPVLVDFWAPWCGPCRMIAPIIEELAEEYE
GKVKFAKVNVDENPETAAKYGIMSIPTLLLFKNGEVVDKLVGARPKEAL
KERIEKHL.

In another embodiment, the invention is directed to a last archaeal common ancestor (LACA) Trx amino acid having the sequence

(SEQ ID NO: 2)
MSVVQLNDENFDEVIKKNNKVVVVDFWAEWCGPCRMIAPIIEELAKEYA
GKVVFGKLNVDENPETAAKYGIMSIPTLLFFKNGKVVDQLVGAMPKEAL
KERIKKYL.

In another embodiment, the invention is directed to an archaeal/eukaryotic common ancestor (AECA) Trx amino acid having the sequence

(SEQ ID NO: 3)
MSVIEINDENFDEVIKKSDKVVVVDFWAEWCGPCRMIAPIIEELAEEYA
GKVVFGKVNVDENPEIAAKYGIMSIPTLLFFKNGKVVDQLVGARPKEAL
KERIKKYL.

In another embodiment, the invention is directed to a last eukaryotic common ancestor (LECA) Trx amino acid having the sequence

(SEQ ID NO: 4)
MVIQVTNKEEFEAILSEADKLVVVDFFATWCGPCKMIAPFFEELSEEYP
DKVVFIKVDVDEVPDVAAKYGITSMPTFKFFKNGKKVDELVGANQEKLK
QMILKHAP.

In another embodiment, the invention is directed to a last common ancestor of cyanobacterial and deinococcus/thermus groups (LPBCA) Trx amino acid having the sequence

(SEQ ID NO: 5)
MSVIEVTDENFEQEVLKSDKPVLVDFWAPWCGPCRMIAPIIEELAKEYE
GKVKVVKVNVDENPNTAAQYGIRSIPTLLLFKNGQVVDRLVGAQPKEAL
KERIDKHL.

In another embodiment, the invention is directed to the last common ancestor of γ-proteobacteria, ˜1.61 Gyr old (LGPCA) Trx amino acid having the sequence

(SEQ ID NO: 6)
MSIIHVTDDSFDQDVLKADKPVLVDFWAEWCGPCKMIAPILDEIAEEYE
GKLKVAKVNIDENPETAAKYGIRGIPTLMLFKNGEVAATKVGALSKSQL
KEFLDANL.

In another embodiment, the invention is directed to the last common ancestor of animals and fungi (LAFCA) Trx amino acid having the sequence

(SEQ ID NO: 7)
MVIQVTNKDEFESILSEADKLVVVDFTATWCGPCKMIAPKFEELSEEYP
DNVVFLKVDVDEVEDVAAEYGISAMPTFQFFKNGKKVDELTGANQEKLK
AMIKKHAA.

A specific embodiment relates to an ancestor protein, fragment, derivative or analog that can be bound by an antibody. Such ancestor proteins, fragments, derivatives or analogs can be tested for the desired immunogenicity by procedures known in the art. (See e.g., Harlow and Lane).

In another aspect, a polypeptide is provided which consists of or comprises a fragment that has at least 8-10 contiguous amino acids of the Trx amino acid sequence as provided in any one of SEQ ID NO: 1-7. In other embodiments, the fragment comprises at least 20 or 50 contiguous amino acids of the Trx amino acid sequence as provided in any one of SEQ ID NO: 1-7.

In one aspect, the invention is directed to polypeptide variants of any one of SEQ ID NO: 1-7. Contemplated variants of any one of SEQ ID NO: 1-7 include but are not limited to polypeptide sequences having at least from about 50% to about 55% identity to that of any one of SEQ ID NO: 1-7. Contemplated variants of any one of SEQ ID NO: 1-7 include but are not limited to polypeptide sequences having at least from about 55.1% to about 60% identity to that of any one of SEQ ID NO: 1-7. Contemplated variants of any one of SEQ ID NO: 1-7 include but are not limited to polypeptide sequences having at least from about 60.1% to about 65% identity to that of any one of SEQ ID NO: 1-7. Contemplated variants of any one of SEQ ID NO: 1-7 include but are not limited to polypeptide sequences having at least from about 65.1% to about 70% identity to that of any one of SEQ ID NO: 1-7. Contemplated variants of any one of SEQ ID NO: 1-7 include but are not limited to polypeptide having at least from about 70.1% to about 75% identity to that of any one of SEQ ID NO: 1-7. Contemplated variants of any one of SEQ ID NO: 1-7 include but are not limited to polypeptide sequences having at least from about 75.1% to about 80% identity to that of any one of SEQ ID NO: 1-7. Contemplated variants of any one of SEQ ID NO: 1-7 include but are not limited to polypeptide sequences having at least from about 80.1% to about 85% identity to that of any one of SEQ ID NO: 1-7. Contemplated variants of any one of SEQ ID NO: 1-7 include but are not limited to polypeptide sequences having at least from about 85.1% to about 90% identity to that of any one of SEQ ID NO: 1-7. Contemplated variant of any one of SEQ ID NO: 1-7 include but are not limited to polypeptide sequences having at least from about 90.1% to about 95% identity to that of any one of SEQ ID NO: 1-7. Contemplated variants of any one of SEQ ID NO: 1-7 include but are not limited to polypeptide sequences having at least from about 95.1% to about 97% identity to that of any one of SEQ ID NO: 1-7. Contemplated variant of any one of SEQ ID NO: 1-7 include but are not limited to polypeptide sequences having at least from about 97.1% to about 99% identity to that of any one of SEQ ID NO: 1-7.

In certain aspects, the invention is directed to a Trx amino acid sequence as provided in any one of SEQ ID NO: 1-7. In another embodiment of the above aspect of the invention, the nucleic acid comprises consecutive nucleotides having a sequence substantially identical to any one of SEQ ID NO: 1-7.

In certain aspects, the invention is directed to an isolated nucleic acid encoding, or capable of encoding, a Trx amino acid sequence as provided in any one of SEQ ID NO: 1-7. In certain aspects, the invention is directed to an isolated nucleic acid complementary to an isolated nucleic acid encoding, or capable of encoding, Trx amino acid sequences as provided in any one of SEQ ID NO: 1-7.

In certain aspects, the invention is directed to isolated amino acid sequence variants of any one of SEQ ID NO: 1-7. Variants of SEQ ID NO: 1-7 include, but are not limited to, amino acid sequences having at least from about 50% to about 55% identity to that of SEQ ID NO: 1-7. Variants of SEQ ID NO: 1-7 include, but are not limited to, amino acid sequences having at least from about 55.1% to about 60% identity to that of SEQ ID NO: 1-7. Variants of SEQ ID NO: 1-7 include, but are not limited to, amino acid sequences having at least from about 60.1% to about 65% identity to that of SEQ ID NO: 1-7. Variants of SEQ ID NO: 1 include, but are not limited to, amino acid sequences having at least from about 65.1% to about 70% identity to that of SEQ ID NO: 1-7. Variants of SEQ ID NO: 1 include, but are not limited to, amino acid sequences having at least from about 70.1% to about 75% identity to that of SEQ ID NO: 1-7. Variants of SEQ ID NO: 1-7 include, but are not limited to, amino acid sequences having at least from about 75.1% to about 80% identity to that of SEQ ID NO: 1-7. Variants of SEQ ID NO: 1-7 include, but are not limited to, amino acid sequences having at least from about 80.1% to about 85% identity to that of SEQ ID NO: 1-7. Variants of SEQ ID NO: 1-7 include, but are not limited to, amino acid sequences having at least from about 85.1% to about 90% identity to that of SEQ ID NO: 1-7. Variants of SEQ ID NO: 1-7 include, but are not limited to, amino acid sequences having at least from about 90.1% to about 95% identity to that of SEQ ID NO: 1-7. Variants of SEQ ID NO: 1-7 include, but are not limited to, amino acid sequences having at least from about 95.1% to about 97% identity to that of SEQ ID NO: 1-7. Variants of SEQ ID NO: 1-7 include, but are not limited to, amino acid sequences having at least from about 97.1% to about 99% identity to that of SEQ ID NO: 1-7.

In one embodiment invention is directed to a polypeptide sequence comprising from about 10 to about 50 consecutive amino acids from any one of SEQ ID NO: 1-7. The invention is directed to a polypeptide sequence comprising from about 10 to about 15 consecutive amino acids from any one of SEQ ID NO: 1-7. The invention is directed to a polypeptide sequence comprising from about 10 to about 20 consecutive amino acids from any one of SEQ ID NO: 1-7. The invention is directed to a polypeptide sequence comprising from about 10 to about 25 consecutive amino acids from any one of SEQ ID NO: 1-7. The invention is directed to a polypeptide sequence comprising from about 10 to about 30 consecutive amino acids from any one of SEQ ID NO: 1-7. The invention is directed to a polypeptide sequence comprising from about 10 to about 35 consecutive amino acids from any one of SEQ ID NO: 1-7. The invention is directed to a polypeptide sequence comprising from about 10 to about 40 consecutive amino acids from any one of SEQ ID NO: 1-7. The invention is directed to a polypeptide sequence comprising from about 10 to about 45 consecutive amino acids from any one of SEQ ID NO: 1-7. The invention is directed to a polypeptide sequence comprising from about 10 to about 50 consecutive amino acids from any one of SEQ ID NO: 1-7. The invention is directed to a polypeptide sequence comprising from about 10 to about 55 consecutive amino acids from any one of SEQ ID NO: 1-7. The invention is directed to a polypeptide sequence comprising from about 10 to about 60 consecutive amino acids from any one of SEQ ID NO: 1-7. The invention is directed to a polypeptide sequence comprising from about 10 to about 65 consecutive amino acids from any one of SEQ ID NO: 1-7. The invention is directed to a polypeptide sequence comprising from about 10 to about 70 consecutive amino acids from any one of SEQ ID NO: 1-7.

The invention is further directed to polypeptide sequences having from about 50% to about 99% identity to a polypeptide sequence comprising from about 8 to about 75 consecutive amino acids from any one of SEQ ID NO: 1-7. The invention is further directed to polypeptide sequences having from about 50% to about 99% identity to a polypeptide sequence comprising from about 8 to about 80 consecutive amino acids from any one of SEQ ID NO: 1-7. The invention is further directed to polypeptide sequences having from about 50% to about 99% identity to a polypeptide sequence comprising from about 8 to about 85 consecutive amino acids from any one of SEQ ID NO: 1-7. The invention is further directed to polypeptide sequences having from about 50% to about 99% identity to a polypeptide sequence comprising from about 8 to about 90 consecutive amino acids from any one of SEQ ID NO: 1-7. The invention is further directed to polypeptide sequences having from about 50% to about 99% identity to a polypeptide sequence comprising from about 8 to about 95 consecutive amino acids from any one of SEQ ID NO: 1-7. The invention is further directed to polypeptide sequences having from about 50% to about 99% identity to a polypeptide sequence comprising from about 8 to about 80 consecutive amino acids from any one of SEQ ID NO: 1-7. The invention is further directed to polypeptide sequences having from about 50% to about 99% identity to a polypeptide sequence comprising from about 8 to about 85 consecutive amino acids from any one of SEQ ID NO: 1-7. The invention is further directed to polypeptide sequences having from about 50% to about 99% identity to a polypeptide sequence comprising from about 8 to about 110 consecutive amino acids from any one of SEQ ID NO: 1-7.

In one embodiment, the invention is directed to an isolated nucleic acid sequence comprising from about 10 to about 50 consecutive nucleotides of a nucleic acid encoding, or capable of encoding any one of SEQ ID NO: 1-7. In another embodiment, the invention is directed to an isolated nucleic acid sequence comprising from about 10 to about 100 consecutive nucleotides of a nucleic acid encoding, or capable of encoding any one of SEQ ID NO: 1-7. In another embodiment, the invention is directed to an isolated nucleic acid sequence comprising from about 10 to about 200 consecutive nucleotides of a nucleic acid encoding, or capable of encoding any one of SEQ ID NO: 1-7. In another embodiment, the invention is directed to an isolated nucleic acid sequence comprising from about 10 to about 300 consecutive nucleotides of a nucleic acid encoding, or capable of encoding any one of SEQ ID NO: 1-7. In another embodiment, the invention is directed to an isolated nucleic acid sequence comprising from about 10 to about 320 consecutive nucleotides of a nucleic acid encoding, or capable of encoding any one of SEQ ID NO: 1-7.

In other aspects the invention is directed to isolated nucleic acid sequences such as primers and probes, comprising nucleic acid sequences derived from of a nucleic acid encoding, or capable of encoding any one of SEQ ID NO: 1-7. The isolated nucleic acids which can be used as primer and/probes are of sufficient length to allow hybridization with, i.e. formation of duplex with a corresponding target nucleic acid sequence, or a nucleic acid encoding, or capable of encoding any one of SEQ ID NO: 1-7, or a variant thereof.

To be expressed, the DNA segment encoding a gene can be coupled to one or more cis acting regulatory elements that regulate the expression profile of the gene. Such regulatory elements comprise, but are not limited to, elements that promote transcription, enhance transcription, silence transcription, modulate transcription such that it is responsive to extracellular and intracellular cues, regulate stability of the encoded RNA, regulate splicing of the encoded RNA, regulate export of the encoded RNA, regulate localization of the encoded RNA, regulate translation from the encoded RNA. Also apparent to those skilled in the art is that the expression profile of a given gene in one organism is frequently a reliable indicator of the expression pattern of homologs in phylogenetically related organisms.

Ancestor protein derivatives and analogs can be produced by various methods known in the art. The manipulations which result in their production can occur at the gene or protein level. For example, a nucleic acid encoding an ancestor protein can be modified by any of numerous strategies known in the art (see, e.g., Sambrook), such as by making conservative substitutions, deletions, insertions, and the like. The nucleic acid sequence can be cleaved at appropriate sites with restriction endonuclease(s), followed by further enzymatic modification, if desired, isolated, and ligated in vitro. In the production of nucleic acids encoding a fragment, derivative or analog of an ancestor protein, the modified nucleic acid typically remains in the proper translational reading frame, so that the reading frame is not interrupted by translational stop signals or other signals that interfere with the synthesis of the fragment, derivative or analog. The ancestral sequence nucleic acid can also be mutated in vitro or in vivo to create and/or destroy translation, initiation and/or termination sequences. The ancestral sequence-encoding nucleic acid can also be mutated to create variations in coding regions and/or to form new restriction endonuclease sites or destroy preexisting ones and to facilitate further in vitro modification. Any technique for mutagenesis known in the art can be used, including but not limited to chemical mutagenesis, in vitro site-directed mutagenesis, and the like. In one embodiment, genes encoding the ancestral Trxs enzymes can be synthesized and codon-optimized for expression in an expression system (e.g. E. coli cells). One skilled in the art will be able generate codon-optimized variants of the nucleic acid sequences encoding the ancestral Trx proteins described herein for expression in a desired expression system.

The ancestral polypeptides described herein can be produced in a host expression system. Exemplary host expression systems include but not limited to, eukaryotic expression systems, prokaryotic expression systems, plant expression systems, animal expression systems, bacterial expression systems, yeast cell expression systems, insect cell expression systems, mammalian cell expression systems, primate cell expression systems, human cell expression systems, hamster cell expression systems, mouse cell expression systems, goat cell expression systems, sheep cell expression systems, bird cell expression systems, chicken cell expression systems, and the like. The host expression system may also be any cell line suitable for recombinant protein expression, including, but not limited to, Chinese hamster ovary (CHO) cells, mouse myeloma NS0 cells, baby hamster kidney cells (BHK), human embryo kidney 293 cells (HEK-293), human C6 cells, Madin-Darby canine kidney cells (MDCK) and Sf9 insect cells. The expression system may also be an entire organism, such as a transgenic plant or animal. For example, the expression system may be a transgenic sheep or cow that capable of expression of recombinant proteins that are secreted into the milk, or a recombinant plant capable of expressing recombinant proteins. Any suitable host system for recombinant protein expression known in the art can be used in accordance with the methods of the present invention.

Expression of nucleic acid sequences can be regulated by a second nucleic acid sequence so that the encoded nucleic acid is expressed in a host transformed with the recombinant DNA molecule. For example, expression of an ancestral sequence can be controlled by any suitable promoter/enhancer element known in the art. Suitable promoters include, for example, the SV40 early promoter region (Benoist and Chambon, Nature 290:304-10 (1981)), the promoter contained in the 3′ long terminal repeat of Rous sarcoma virus (Yamamoto et al., Cell 22:787-97 (1980)), the herpes thymidine kinase promoter (Wagner et al., Proc. Natl. Acad. Sci. USA 78:1441-45 (1981)), the Cytomegalovirus promoter, the translational elongation factor EF-1.alpha. promoter, the regulatory sequences of the metallothionein gene (Brinster et al., Nature 296:39-42 (1982)), prokaryotic promoters such as, for example, the .beta.-lactamase promoter (Villa-Komaroff et al., Proc. Natl. Acad. Sci. USA 75:3727-31 (1978)) or the tac promoter (deBoer et al., Proc. Natl. Acad. Sci. USA 80:21-25 (1983)), plant expression vectors including the cauliflower mosaic virus 35S RNA promoter (Gardner et al., Nucl. Acids Res. 9:2871-88 (1981)), and the promoter of the photosynthetic enzyme ribulose biphosphate carboxylase (Herrera-Estrella et al., Nature 310:115-20 (1984)), promoter elements from yeast or other fungi such as the GAL7 and GAL4 promoters, the ADH (alcohol dehydrogenase) promoter, the PGK (phosphoglycerol kinase) promoter, the alkaline phosphatase promoter, and the like.

In a specific embodiment, a vector is used that comprises a promoter operably linked to the ancestral sequence encoding nucleic acid, one or more origins of replication, and, optionally, one or more selectable markers (e.g., an antibiotic resistance gene). Suitable selectable markers include, for example, those conferring resistance to ampicillin, tetracycline, neomycin, G418, and the like. An expression construct can be made, for example, by subcloning a nucleic acid encoding an ancestral sequence into a restriction site of the pRSECT expression vector. Such a construct allows for the expression of the ancestral sequence under the control of the T7 promoter with a histidine amino terminal flag sequence for affinity purification of the expressed polypeptide.

Expression systems suitable for use with the methods described herein include, but are not limited to in-vitro expression systems and in vivo expression systems. Exemplary in vitro expression systems include, but are not limited to, cell-free transcription/translation systems (e.g. ribosome based protein expression systems). Several such systems are known in the art (see, for example, Tymms (1995) In vitro Transcription and Translation Protocols: Methods in Molecular Biology Volume 37, Garland Publishing, NY).

Exemplary in vivo expression systems include, but are not limited to prokaryotic expression systems such as bacteria (e.g., E. coli and B. subtilis), yeast expression systems (e.g., Saccharomyces cerevisiae), worm expression systems (e.g. Caenorhabditis elegans), insect expression systems (e.g. Sf9 cells), plant expression systems, and amphibian expression systems (e.g. melanophore cells).

Manipulations of the ancestral sequence can also be made at the protein level. Included within the scope of the invention are ancestor protein fragments, derivatives or analogs that are differentially modified during or after synthesis (e.g., in vivo or in vitro translation). Such modifications include conservative substitution, glycosylation, acetylation, phosphorylation, amidation, derivatization by known protecting/blocking groups, proteolytic cleavage, linkage to an antibody molecule or other cellular ligand, and the like. Any of numerous chemical modifications can be carried out by known techniques, including, but not limited to, specific chemical cleavage (e.g., by cyanogen bromide); enzymatic cleavage (e.g., by trypsin, chymotrypsin, papain, V8 protease, and the like); modification by, for example, NaBH.sub.4 acetylation, formylation, oxidation and reduction; metabolic synthesis in the presence of tunicamycin; and the like. Amino acids can be modified, for example, co-translationally or post-translationally during recombinant production (e.g., N-linked glycosylation at N-X-S/T motifs during expression in mammalian cells) or modified by synthetic means. Examples of modified amino acids suitable for use with the methods described herein include, but are not limited to, glycosylated amino acids, sulfated amino acids, prenlyated (e.g., farnesylated, geranylgeranylated) amino acids, acetylated amino acids, PEG-ylated amino acids, biotinylated amino acids, carboxylated amino acids, phosphorylated amino acids, and the like. Exemplary protocol and additional amino acids can be found in Walker (1998) Protein Protocols on CD-ROM Human Press, Towata, N.J.

In addition, fragments, derivatives and analogs of ancestor proteins can be chemically synthesized. For example, a peptide corresponding to a portion, or fragment, of an ancestor protein, which comprises a desired domain, can be synthesized by use of chemical synthetic methods using, for example, an automated peptide synthesizer. (See also Hunkapiller et al., Nature 310:105-11 (1984); Stewart and Young, Solid Phase Peptide Synthesis, 2nd ed., Pierce Chemical Co., Rockford, Ill., (1984).) Furthermore, if desired, nonclassical amino acids or chemical amino acid analogs can be introduced as a substitution or addition into the polypeptide sequence. Non-classical amino acids include, but are not limited to, the D-isomers of the common amino acids, .alpha.-amino isobutyric acid, 4-aminobutyric acid, 2-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, .beta.-alanine, selenocysteine, fluoro-amino acids, designer amino acids such as .beta.-methyl amino acids, C .alpha.-methyl amino acids, N .alpha.-methyl amino acids, and other amino acid analogs. Furthermore, the amino acid can be D (dextrorotary) or L (levorotary).

The ancestral protein, fragment, derivative or analog can also be a chimeric, or fusion, protein-comprising an ancestor protein, fragment, derivative or analog thereof (typically consisting of at least a domain or motif of the ancestor protein, or at least 10 contiguous amino acids of the ancestor protein) joined at its amino- or carboxy-terminus via a peptide bond to an amino acid sequence of a different protein. In one embodiment, such a chimeric protein is produced by recombinant expression of nucleic acid encoding the chimeric protein. The chimeric nucleic acid can be made by ligating the appropriate nucleic acid sequences to each other in the proper reading frame and expressing the chimeric product by methods commonly known in the art. Alternatively, the chimeric protein can be made by protein synthetic techniques (e.g., by use of an automated peptide synthesizer).

The nucleic acids encoding ancestral sequences can be inserted into an appropriate expression vector (i.e., a vector which contains the necessary elements for the transcription and translation of the inserted polypeptide-coding sequence). A variety of host-vector systems can be utilized to express the polypeptide-coding sequence(s). These include, for example, mammalian cell systems infected with virus (e.g., vaccinia virus, adenovirus, sindbis virus, Venezuelan equine encephalitis (VEE) virus, and the like), insect cell systems infected with virus (e.g., baculovirus), microorganisms such as yeast containing yeast vectors, or bacteria transformed with bacteriophage DNA, plasmid DNA, or cosmid DNA. The expression elements of vectors vary in their strengths and specificities. Depending on the host-vector system utilized, any one of a number of suitable transcription and translation elements can be used. In specific embodiments, the ancestral sequence is expressed in human cells, other mammalian cells, yeast or bacteria. In yet another embodiment, a fragment of an ancestral sequence comprising an immunologically active region of the sequence is expressed. In one embodiment, the ancestral genes can be cloned into a pQE80L vector and transformed in E. coli BL21 (DE3) cells. For expression, the cells can be incubated overnight in LB medium at 37° C. and protein expression can be induced with 1 mM IPTG. Expressed protein can be recovered by pelleting and sonicated the cells.

Upon expression, ancestral proteins can be isolated and purified by standard methods including chromatography (e.g., ion exchange, affinity, sizing column chromatography, high pressure liquid chromatography), centrifugation, differential solubility, or by any other standard technique for the purification of proteins. In one embodiment, the ancestral proteins can be His 6-tagged. Upon recovery, the proteins can be purified by loading cell lysates onto a His GraviTrap affinity column. The purified protein can be verified by SDS-PAGE. The proteins can then loaded into PD-10 desalting column and finally dialyzed against a buffer (e.g. 50 mM HEPES, pH 7.0 buffer).

Conditions for Trx enzymatic activity can vary according to the Trx enzyme because thioredoxins are in a reduced state to be active. Reduced state Trx enzymes can be generated by any method known in the art, including but not limited to the use of a complementary bacterial or eukaryotic Trx reductase (TrxR) enzyme. Where Trx enzymes are from extant sources or are resurrected enzymes, their accompanying reductases may be unknown or unavailable. In such cases small amounts of dithiothreitol (DTT) (e.g. 50-100 μM) or Tris(2-carboxyethyl)phosphine HCl (TCEP hydrochloride) can be used to maintain the enzymes in the reduced state. The amount of DTT of TCEP can be selected such that it is sufficient to maintain the enzymes in the reduced state but low enough as to not trigger the reduction of disulfide bonds by themselves. Such conditions can to be established for each individual enzyme.

Enzymes can be exceptional catalysts useful for accelerating chemical reaction rates by several orders of magnitude. The mechanisms of numerous enzymatic reactions can be studied using any number of protein biochemistry as well as structural biology approaches, including, but not limited to X-ray crystallography and NMR. Such studies can be used to identify structural features and conformational changes necessary for the catalytic activity of enzymes. Single molecule techniques can also be useful for studying enzyme dynamics in solution at the Ångström scale. In certain aspects, single molecule techniques are useful where observation of rearrangements in the participating atoms necessary for catalysis is important. Such approaches generate data that, combined together with structural information as well as molecular dynamics simulations, can provide a more complete view of enzyme dynamics.

Several methods, some of which are based on spectrophotometry, can be used to determine the activity of Trx enzymes. Exemplary methods include, but are not limited to monitoring the oxidation of NADPH in the presence of Trx reductase or ribonucleotide reductase (Holmgren, J Biol Chem, 1979. 254(18): p. 9113-9; Holmgren, J Biol Chem, 1979. 254(19): p. 9627-32); the observation of the turbidity of solutions containing insulin, which readily aggregates after reduction of its disulfide bonds (Holmgren, J Biol Chem, 1979. 254(19): p. 9627-32) or the use of Ellman's reagent (DTNB), where upon reduction by thiol groups generates products that can be easily detected with a spectrophotometer (Holmgren, Thioredoxin. Annu Rev Biochem, 1985. 54: p. 237-71). Changes in tryptophan fluorescence have also been used to measure rates of Trx oxidation and reduction (Holmgren, J Biol Chem, 1972. 247(7): p. 1992-8). Although effective in monitoring the overall activity of thioredoxin, these methods are not sensitive enough to probe the substrate-enzyme interactions that take place in the binding groove of the enzyme. Such methods can be important because binding grooves are common in enzymes and enzymatic reactions. In such cases, examination of the enzymatic mechanisms and/or activity can be facilitated by single molecule techniques.

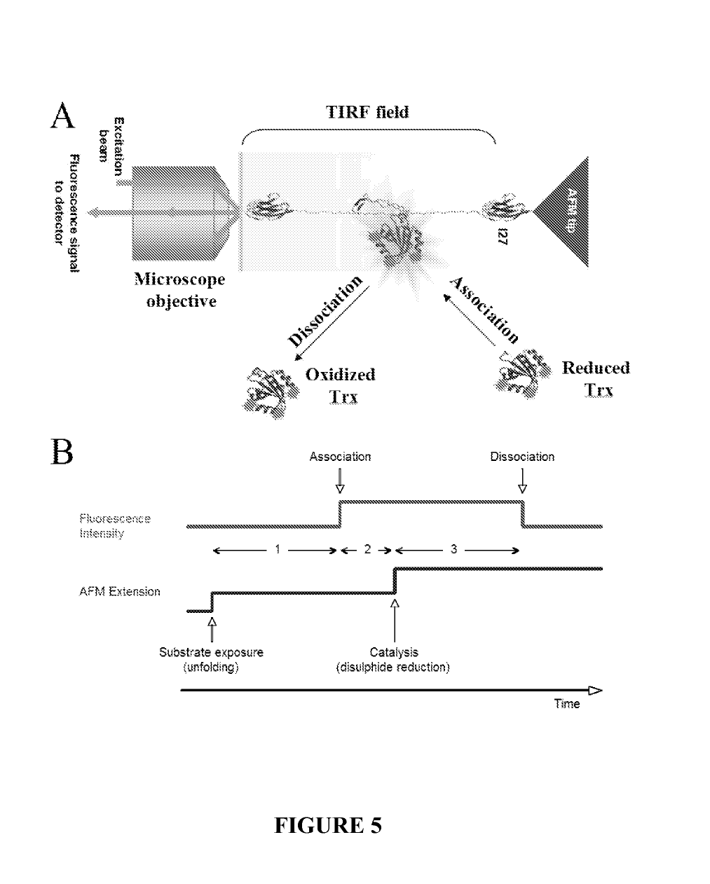

Described herein is a force-clamp spectrometer built on top of a “through the lens” Total Internal Reflection Fluorescence (TIRF) microscope. This experimental setup enables the application of force to a single protein while at the same time measuring a fluorescent signal. The force-spectrometer can be either an AFM (Sarkar et al., Proc Natl Acad Sci USA, 2004. 101(35): p. 12882-6), or an electromagnet (Liu et al., Biophysical Journal, 2009. 96(9): p. 3810-3821). Both of these can readily pick up and stretch a single engineered polypeptide. The design takes advantage of the stability and high spatial sensitivity of the evanescent field of the TIRF microscope. As a result of total internal reflection, an evanescent wave is formed on the surface of the microscope slide. The amplitude of the evanescent wave decays exponentially, with a space constant that can be set to be as short as ˜90 nm and up to ˜300 nm. The evanescent wave can excite any fluorophore that enters this field, and its fluorescence can readily be measured by a high performance CCD camera. The rapidly decaying evanescent field on the surface of the microscope slide can be used either to measure displacement in the z direction or to capture single molecule fluorescence without any background emanating from the solution buffer. The combined AFM/TIRF microscope to can be used to demonstrate that a calibrated evanescent field can be used to track the mechanical unfolding of a single polypeptide with sub-nanometer resolution (Sarkar et al., Proc Natl Acad Sci USA, 2004. 101(35): p. 12882-6). The same TIRF microscope equipped with magnetic tweezers can track the unfolding of a polypeptide at very low forces and for very long periods of time (Liu et al., Biophysical Journal, 2009. 96(9): p. 3810-3821). However, the simplest application of the AFM/TIRF microscope is in detecting fluorescence over a very short distance of a mechanically stretched protein, without interference from the bulk. This technique has been demonstrated by mechanically stretching and unfolding the protein talin, a key player in coupling the cytoskeleton of a cell to the extracellular matrix (del Rio et al., Science, 2009. 323(5914): p. 638-41). These experiments demonstrate the versatility of combining force-spectroscopy with TIRF microscopy. As described herein, this technique can be used to monitor the association/dissociation reactions of single thioredoxin enzymes as they reduce disulfide bonds in substrate proteins. Trx enzymes can be labeled while remaining active, for example, exposed lysines of Trx enzymes can be labeled with Alexa Fluor 488 fluorophore such to allow monitoring when the enzyme binds to the exposed disulfide bond. The experimental design is shown in FIG. 5. This approach can be used to measure the time course of association and dissociation of fluorescently labeled thioredoxins, while simultaneously observing the reduction of the substrate and to characterize the dynamics of the enzyme-substrate interactions at the single molecule level and develop kinetic models for catalysis (Wiita, A. P., et al., Nature, 2007. 450(7166): p. 124-7).

The association and dissociation of fluorescently labeled thioredoxin enzymes can be measured while simultaneously monitoring reduction events using force-spectroscopy/TIRF instrumentation. The force dependency of association and dissociation can also be measured as can the dwell times between association and reduction. These data can be used to examine the mechanisms by which thioredoxin enzymes find their target disulfide bonds. As described herein, the single molecule AFM detection of disulfide bond reduction can be combined with simultaneous Total Internal Reflection (TIRF) detection of fluorescently labeled thioredoxin enzymes to follow them as they bind and unbind to the disulfide bond being reduced. This instrument enables real time visualization of the entire association, reduction and dissociation cycle of a single enzyme as it catalyzes the reduction of its target. The combined AFM/TIRF instrument can be used to study the search mechanism, and to measure association and dissociation rates as a function of the mechanical force applied to the substrate.

In one aspect, the invention described herein relates to the use of single molecule force-clamp spectroscopy techniques for investigating the chemical mechanisms of catalysis of thioredoxins, a broad class of enzymes that specialize in reducing disulfide bonds and that can also function as oxidases and isomerases. Thioredoxin enzymes are present in all known organisms from bacteria to human and play crucial roles in a wide variety of cellular functions. Thioredoxins have been implicated in pathological processes such as vascular damage caused by oxidative injury, virus entry into cells, and a wide variety of immune related disorders, but also have found practical use in biotechnology.

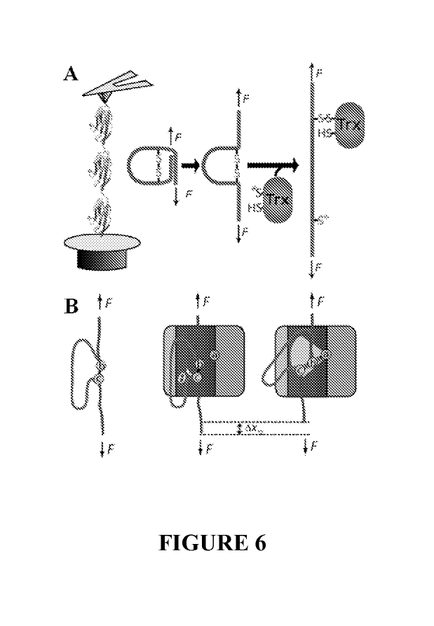

The single molecule assay for the reduction of disulfide bonds by thioredoxin can be performed by detecting the step elongation of a protein under force, which results from the cleavage of a covalent bond (FIG. 6). This scheme can be generalized to other types of enzymes that catalyze the cleavage of covalent bonds such as proteases. Proteases are a vast group of proteins that efficiently catalyze the hydrolysis of peptide bonds (Beynon and Bond, Proteolytic enzymes: a practical approach. 2001, New York: Oxford University Press). Alterations in their physiological activities are responsible for the occurrence or exacerbation of numerous pathologies, such as cancer or inflammatory and cardiovascular diseases (Lopez-Otin and Bond, J Biol Chem, 2008. 283(45): p. 30433-7). Proteases are regarded as potential drug targets or biomarkers by the pharmaceutical industry (Turk, Nat Rev Drug Discov, 2006. 5(9): p. 785-99). Pharmacological interventions on protease activity benefit from detailed knowledge of their mechanism of catalysis (Walker and Lynas Cell Mol Life Sci, 2001. 58(4): p. 596-624). Single molecule techniques to study protease enzymes and uncover substrate dynamics during proteolysis, thereby enabling pharmacological intervention on protease activity from detailed knowledge of their mechanism of catalysis.

By applying a calibrated force the conformations of a disulfide bond substrate can be controlled, and the effect of this restriction on the activity of thioredoxin enzymes can be measured. This assay is a highly sensitive probe of the sub-Ångström level rearrangement of the sulfur atoms at the catalytic center of Trx enzymes (Wiita, A. P., et al., Nature, 2007. 450(7166): p. 124-7). By combining this new form of spectroscopy together with structural data and molecular dynamics simulations we obtain novel insights into catalysis. These studies can be generalized and understood in relation to the structure of other enzymes to evaluate of the range of chemical mechanisms available to thioredoxin as well as other enzymes and how such mechanisms can be controlled by structural features such as binding grooves.

Single molecule assays can also be used to detect the oxidase activity of thioredoxin enzymes. For example, if the stretching force is quenched after a substrate disulfide bond has been reduced, the substrate protein folds, however the disulfide bond does not reform spontaneously. By introducing a mutant form of thioredoxin, efficient re-oxidation can be obtained during folding.

Force spectroscopy can also be used to examine other covalent bond cleaving enzymes. For example, proteases share structural features in common with thioredoxins such as a binding groove adjacent to the catalytic nucleophile. A steric-switch approach, where a bond cleavage event is translated into an easily identified stepwise elongation of the substrate protein, can be adapted to detect the activity of proteases, and study their catalytic mechanisms.

As described herein, single molecule force-spectroscopy experiments demonstrate that the application of a mechanical force to a substrate disulfide bond can regulate the catalytic activity of thioredoxin enzymes, thereby revealing distinct chemical mechanisms of reduction that can be distinguished by their sensitivity to an applied force. Thus, single molecule assay of thioredoxin catalysis provides with a novel and useful new approach to study the chemical mechanisms of catalysis in this important class of enzymes.

One advantage of the single molecule approach is that individual conformations, which can otherwise be averaged out in bulk experiments, can be observed directly and then correlated with the known structural features of the molecule. This approach can also be used for ion channels, where it was possible to provide a detailed account of the structure-function relationship for this class of membrane proteins. As described herein, single molecule assays for substrate dynamics in thioredoxin and protease catalysis can be used to study enzyme dynamics.

In single molecule force clamp spectroscopy experiments, a mechanical force is applied to a substrate protein containing a target disulfide bond, and the effect of the resulting stiffening on the rate of reduction or oxidation by thioredoxin enzymes is measured. The applied force restricts the movement of the enzymatic substrate in the binding groove of the enzyme, acting as a form of spectroscopy that can be used to investigate the types of substrate motions that occur during enzymatic catalysis. As described herein, this form of spectroscopy can be used to study the catalytic mechanisms of enzymes, including, but not limited to thioredoxin enzymes and proteases.

The application of force to a substrate disulfide bond can be used to modulate conformational dynamics in the binding groove of Trx (FIG. 6), thereby regulating the catalytic activity of the enzyme (Wiita, A. P., et al., Nature, 2007. 450(7166): p. 124-7). This form of molecular spectroscopy can resolve substrate motions in the active site of the Trx enzyme with sub-Ångström resolution. Force-spectroscopy of Trx catalysis indicates that the chemical mechanism of reduction is characterized by its rapid inhibition by a force applied to the substrate disulfide bond. When compared with other reducing agents, this chemical mechanism is specific to Trx enzymes. After binding to the enzymatic groove, the reaction occurs by rotation of the target disulfide bond against the pulling force in order to acquire the correct geometry for the S_{N}2 chemical reaction to occur (FIG. 6B). Other chemical mechanisms of reduction also operate simultaneously (Perez-Jimenez, et al., Nature Structural & Molecular Biology, 2009. 16(8): p. 890-U120). The force-clamp spectroscopy approach is validated by the fact that the rates of reduction extrapolated to zero force agree with those measured using spectrophotometric methods (Perez-Jimenez, et al., Nature Structural & Molecular Biology, 2009. 16(8): p. 890-U120). Hence, force spectroscopy of Trx catalysis can be used to study the dynamics of a substrate in the binding groove of an enzyme. Indeed, the single molecule reduction assay (as shown in FIG. 6A) is readily able to distinguish the chemistry of simple nucleophiles, such as cysteine and glutathione, from more elaborate pathways for the reduction of disulfide bonds, which are unique to groove based thioredoxin enzymes (FIG. 1E) (Wiita, A. P., et al., Nature, 2007. 450(7166): p. 124-7; Perez-Jimenez, et al., Nature Structural & Molecular Biology, 2009. 16(8): p. 890-U120; Ainavarapu et al., Journal of the American Chemical Society, 2008. 130(20): p. 6479-6487). Furthermore, the force-clamp spectroscopy assay is able to combine the observation of protein folding, together with reduction-oxidation cycles.

During protein disulfide bond reduction, thioredoxin binds to the substrate in a catalytically favorable configuration (Qin et al., Structure, 1995. 3: p. 289-297). The mechanisms by which thioredoxin finds a substrate disulfide bond can be examined by measuring the association and dissociation of single enzymes as they find and reduce a disulfide bond. Thioredoxin enzymes may find and position the two bonded sulfur atoms out of the thousands of atoms of the host protein by utilizing a “reduced dimensionality” approach (Adam and Delbruck, Structural Chemistry and Molecular Biology, ed. A. Rich and N. Davidson. 1968, New York: W. H. Freeman and Co. 198-215; von Hippel and Berg, J Biol Chem, 1989. 264(2): p. 675-8), similar to enzymes that target DNA (Gorman et al., Mol Cell, 2007. 28(3): p. 359-70; Stanford et al., Embo J, 2000. 19(23): p. 6546-57). A reduced dimensionality search consists of at least two distinct steps: a nonspecific association with the substrate macromolecule followed by some form of processivity along the coordinates of the substrate (Riggs, et al, Lac Repressor-Operator Interaction 0.3. Kinetic Studies. Journal of Molecular Biology, 1970. 53(3): p. 401-7).

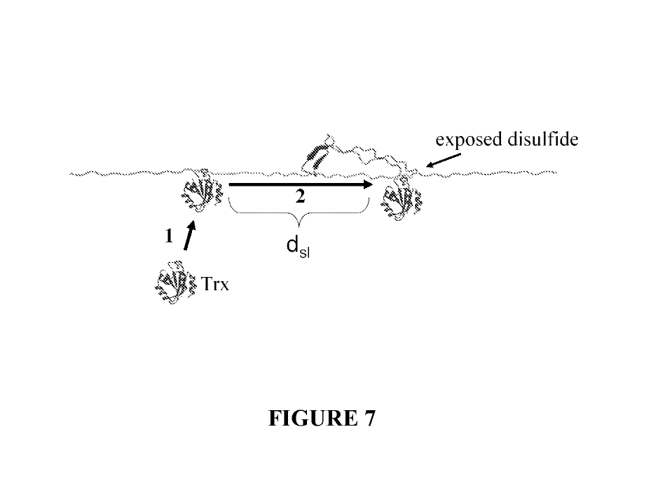

In the case of DNA binding enzymes, the principle of reduced dimensionality has been well established as a widespread mechanism (Halford et al., Nucleic Acids Res, 2004. 32(10): p. 3040-52). For enzymes acting on macromolecular substrates, reduced dimensionality may be important for facilitating the target search (Adam and Delbruck, Structural Chemistry and Molecular Biology, ed. A. Rich and N. Davidson. 1968, New York: W. H. Freeman and Co. 198-215; Riggs, et al, Lac Repressor-Operator Interaction 0.3. Kinetic Studies. Journal of Molecular Biology, 1970. 53(3): p. 401-7; Berg and Blomberg, Biophysical Chemistry, 1978. 8(4): p. 271-280; Berg et al., Biochemistry, 1981. 20(24): p. 6929-6948; von Hippel and Berg, J Biol Chem, 1989. 264(2): p. 675-8). In the case of Trx enzymes, Trx enzymes may first bind to a substrate and then diffusing along the extended polypeptide until finding the disulfide bond. The polypeptide stays loosely bound to the enzymatic groove, and slides randomly towards the disulfide. The simplest expression for the mean time to target is given by

$$〈 t 〉 \approx \frac{〈 {d}_{st}^{2} 〉}{2 D} ,$$

where D is the diffusion coefficient for the enzyme sliding along the polypeptide and dₛₗ is the sliding distance between the place where Trx was first bound to the polypeptide and the exposed disulfide bond (FIG. 7). This simple scenario can be examined by directly measuring the distribution of dwell times between binding and reduction. The time to target can depend on the square of the sliding distance dₛ₁, which we will vary using protein engineering (Stanford et al., Embo Journal, 2000. 19(23): p. 6546-6557; Halford et al., Nucleic Acids Research, 2004. 32(10): p. 3040-3052).

Although several different ancestral Trx polypeptides are described herein, one of skill in the art will recognize that other types of ancestral polypeptides can also be produced using the methods described herein. Ancestral sequences can be generated for any polypeptide using the methods described herein, including, but not limited to therapeutic proteins and proteins susceptible to industrial use.

The stability and/or activity of any polypeptide at low pH or elevated temperature can be modified according to the methods described herein. Polypeptides having increased stability and/or activity of any polypeptide at low pH or elevated temperature that can be produced according to the methods described herein can be from any source or origin and can include a polypeptide found in prokaryotes, viruses, and eukaryotes, including fungi, plants, yeasts, insects, and animals, including mammals (e.g. humans). Polypeptides having increased stability and/or activity of any polypeptide at low pH or elevated temperature that can be produced according to the methods described herein include, but are not limited to any polypeptide sequences, known or hypothetical or unknown, which can be identified using common sequence repositories. Example of such sequence repositories include, but are not limited to GenBank EMBL, DDBJ and the NCBI. Other repositories can easily be identified by searching on the internet. Polypeptides that can be produced using the methods described herein also include polypeptides have at least about 60%, 70%, 75%, 80%, 90%, 95%, or at least about 99% or more identity to any known or available polypeptide (e.g., a therapeutic polypeptide, a diagnostic polypeptide, an industrial enzyme, or portion thereof, and the like).

Polypeptides having increased stability and/or activity of any polypeptide at low pH or elevated temperature that can be produced according to the methods described herein also include polypeptides comprising one or more non-natural amino acids. As used herein, a non-natural amino acid can be, but is not limited to, an amino acid comprising a moiety where a chemical moiety is attached, such as an aldehyde- or keto-derivatized amino acid, or a non-natural amino acid that includes a chemical moiety. A non-natural amino acid can also be an amino acid comprising a moiety where a saccharide moiety can be attached, or an amino acid that includes a saccharide moiety.

Polypeptides having increased stability and/or activity of any polypeptide at low pH or elevated temperature can also comprise peptide derivatives (for example, that contain one or more non-naturally occurring amino acids). In specific embodiments, the library members contain one or more non-natural or non-classical amino acids or cyclic peptides. Non-classical amino acids include but are not limited to the D-isomers of the common amino acids, -amino isobutyric acid, 4-aminobutyric acid, Abu, 2-amino butyric acid; .-Abu, -Ahx, 6-amino hexanoic acid; Aib, 2-amino isobutyric acid; 3-amino propionic acid; ornithine; norleucine; norvaline, hydroxyproline, sarcosine, citrulline, cysteic acid, t-butylglycine, t-butylalanine, phenylglycine, cyclohexylalanine, .beta.-alanine, designer amino acids such as .beta.-methyl amino acids, C-methyl amino acids, N-methyl amino acids, fluoro-amino acids and amino acid analogs in general. Furthermore, the amino acid can be D (dextrorotary) or L (levorotary).

Also inclusive are derivative polypeptides having an amino acid sequence selected from the group consisting of a polypeptide of SEQ ID NOs: 1-7 and which has been acetylated, carboxylated, phosphorylated, glycosylated, ubiquitinated or other post-translational modifications. In another embodiment, the derivative has been labeled with, e.g., radioactive isotopes such as ¹²⁵I, ³²P, ³⁵S, and ³H. In another embodiment, the derivative has been labeled with fluorophores, chemiluminescent agents, enzymes, and antiligands that can serve as specific binding pair members for a labeled ligand.

Polypeptide modifications are well known to those of skill and have been described in detail in the scientific literature. Several common modifications, such as glycosylation, lipid attachment, sulfation, gamma-carboxylation of glutamic acid residues, hydroxylation and ADP-ribosylation, for instance, are described in most basic texts, such as, for instance Creighton, Protein Structure and Molecular Properties, 2nd ed., W. H. Freeman and Company (1993). Many detailed reviews are available on this subject, such as, for example, those provided by Wold, in Johnson (ed.), Posttranslational Covalent Modification of Proteins, pgs. 1-12, Academic Press (1983); Seifter et al., Meth. Enzymol. 182: 626-646 (1990) and Rattan et al., Ann. N.Y. Acad. Sci. 663: 48-62 (1992).

One can determine whether a polypeptide of the invention will be post-translationally modified by analyzing the sequence of the polypeptide to determine if there are peptide motifs indicative of sites for post-translational modification. There are a number of computer programs that permit prediction of post-translational modifications. See, e.g., expasy with the extension .org of the world wide web (accessed Nov. 11, 2002), which includes PSORT, for prediction of protein sorting signals and localization sites, SignalP, for prediction of signal peptide cleavage sites, MITOPROT and Predotar, for prediction of mitochondrial targeting sequences, NetOGlyc, for prediction of type O-glycosylation sites in mammalian proteins, big-PI Predictor and DGPI, for prediction of prenylation-anchor and cleavage sites, and NetPhos, for prediction of Ser, Thr and Tyr phosphorylation sites in eukaryotic proteins. Other computer programs, such as those included in GCG, also can be used to determine post-translational modification peptide motifs.

Examples of types of post-translational modifications include, but are not limited to: (Z)-dehydrobutyrine; 1-chondroitin sulfate-L-aspartic acid ester; l′-glycosyl-L-tryptophan; 1′-phospho-L-histidine; 1-thioglycine; 2′-(S-L-cysteinyl)-L-histidine; 2′-[3-carboxamido (trimethylammonio)propyl]-L-histidine; 2′-alpha-mannosyl-L-tryptophan; 2-methyl-L-glutamine; 2-oxobutanoic acid; 2-pyrrolidone carboxylic acid; 3′-(1′-L-histidyl)-L-tyrosine; 3′-(8alpha-FAD)-L-histidine; 3′-(S-L-cysteinyl)-L-tyrosine; 3′,3″,5′-triiodo-L-thyronine; 3′-4′-phospho-L-tyrosine; 3-hydroxy-L-proline; 3′-methyl-L-histidine; 3-methyl-L-lanthionine; 3′-phospho-L-histidine; 4′-(L-tryptophan)-L-tryptophyl quinone; 42 N-cysteinyl-glycosylphosphatidylinositolethanolamine; 43-(T-L-histidyl)-L-tyrosine; 4-hydroxy-L-arginine; 4-hydroxy-L-lysine; 4-hydroxy-L-proline; 5′-(N-6-L-lysine)-L-topaquinone; 5-hydroxy-L-lysine; 5-methyl-L-arginine; alpha-1-microglobulin-Ig alpha complex chromophore; bis-L-cysteinyl bis-L-histidino diiron disulfide; bis-L-cysteinyl-L-N3′-histidino-L-serinyl tetrairon' tetrasulfide; chondroitin sulfate D-glucuronyl-D-galactosyl-D-galactosyl-D-xylosyl-L-serine; D-alanine; D-allo-isoleucine; D-asparagine; dehydroalanine; dehydrotyrosine; dermatan 4-sulfate D-glucuronyl-D-galactosyl-D-galactosyl-D-xylosyl-L-serine; D-glucuronyl-N-glycine; dipyrrolylmethanemethyl-L-cysteine; D-leucine; D-methionine; D-phenylalanine; D-serine; D-tryptophan; glycine amide; glycine oxazolecarboxylic acid; glycine thiazolecarboxylic acid; heme P450-bis-L-cysteine-L-tyrosine; heme-bis-L-cysteine; hemediol-L-aspartyl ester-L-glutamyl ester; hemediol-L-aspartyl ester-L-glutamyl ester-L-methionine sulfonium; heme-L-cysteine; heme-L-histidine; heparan sulfate D-glucuronyl-D-galactosyl-D-galactosyl-D-xylosyl-L-serine; heme P450-bis-L-cysteine-L-lysine; hexakis-L-cysteinyl hexairon hexasulfide; keratan sulfate D-glucuronyl-D-galactosyl-D-galactosyl-D-xylosyl-L-threonine; L oxoalanine-lactic acid; L phenyllactic acid; 1′-(8alpha-FAD)-L-histidine; L-2′,4′,5′-topaquinone; L-3′,4′-dihydroxyphenylalanine; L-3′,4′,5′-trihydroxyphenylalanine; L-4′-bromophenylalanine; L-6′-bromotryptophan; L-alanine amide; L-alanyl imidazolinone glycine; L-allysine; L-arginine amide; L-asparagine amide; L-aspartic 4-phosphoric anhydride; L-aspartic acid 1-amide; L-beta-methylthioaspartic acid; L-bromohistidine; L-citrulline; L-cysteine amide; L-cysteine glutathione disulfide; L-cysteine methyl disulfide; L-cysteine methyl ester; L-cysteine oxazolecarboxylic acid; L-cysteine oxazolinecarboxylic acid; L-cysteine persulfide; L-cysteine sulfenic acid; L-cysteine sulfinic acid; L-cysteine thiazolecarboxylic acid; L-cysteinyl homocitryl molybdenum-heptairon-nonasulfide; L-cysteinyl imidazolinone glycine; L-cysteinyl molybdopterin; L-cysteinyl molybdopterin guanine dinucleotide; L-cystine; L-erythro-beta-hydroxyasparagine; L-erythro-beta-hydroxyaspartic acid; L-gamma-carboxyglutarnic acid; L-glutamic acid 1-amide; L-glutamic acid 5-methyl ester; L-glutamine amide; L-glutamyl 5-glycerylphosphorylethanolarnine; L-histidine amide; L-isoglutamyl-polyglutamic acid; L-isoglutamyl-polyglycine; L-isoleucine amide; L-lanthionine; L-leucine amide; L-lysine amide; L-lysine thiazolecarboxylic acid; L-lysinoalanine; L-methionine amide; L-methionine sulfone; L-phenyalanine thiazolecarboxylic acid; L-phenylalanine amide; L-proline amide; L-selenocysteine; L-selenocysteinyl molybdopterin guanine dinucleotide; L-serine amide; L-serine thiazolecarboxylic acid; L-seryl imidazolinone glycine; L-T-bromophenylalanine; L-T-bromophenylalanine; L-threonine amide; L-thyroxine; L-tryptophan amide; L-tryptophyl quinone; L-tyrosine amide; L-valine amide; meso-lanthionine; N-(L-glutamyl)-L-tyrosine; N-(L-isoaspartyl)-glycine; N-(L-isoaspartyl)-L-cysteine; N,N,N-trimethyl-L-alanine; N,N-dimethyl-L-proline; N2-acetyl-L-lysine; N2-succinyl-L-tryptophan; N4-(ADP-ribosyl)-L-asparagine; N4-glycosyl-L-asparagine; N4-hydroxymethyl-L-asparagine; N4-methyl-L-asparagine; N5-methyl-L-glutamine; N6-1-carboxyethyl-L-lysine; N6-(4-amino hydroxybutyl)-L-lysine; N6-(L-isoglutamyl)-L-lysine; N6-(phospho-5′-adenosine)-L-lysine; N6-(phospho-5′-guanosine)-L-lysine; N6,N6,N6-trimethyl-L-lysine; N6,N6-dimethyl-L-lysine; N6-acetyl-L-lysine; N6-biotinyl-L-lysine; N6-carboxy-L-lysine; N6-formyl-L-lysine; N6-glycyl-L-lysine; N6-lipoyl-L-lysine; N6-methyl-L-lysine; N6-methyl-N-6-poly(N-methyl-propylamine)-L-lysine; N6-mureinyl-L-lysine; N6-myristoyl-L-lysine; N6-palmitoyl-L-lysine; N6-pyridoxal phosphate-L-lysine; N6-pyruvic acid 2-iminyl-L-lysine; N6-retinal-L-lysine; N-acetylglycine; N-acetyl-L-glutamine; N-acetyl-L-alanine; N-acetyl-L-aspartic acid; N-acetyl-L-cysteine; N-acetyl-L-glutamic acid; N-acetyl-L-isoleucine; N-acetyl-L-methionine; N-acetyl-L-proline; N-acetyl-L-serine; N-acetyl-L-threonine; N-acetyl-L-tyrosine; N-acetyl-L-valine; N-alanyl-glycosylphosphatidylinositolethanolamine; N-asparaginyl-glycosylphosphatidylinositolethanolamine; N-aspartyl-glycosylphosphatidylinositolethanolamine; N-formylglycine; N-formyl-L-methionine; N-glycyl-glycosylphosphatidylinositolethanolamine; N-L-glutamyl-poly-L-glutamic acid; N-methylglycine; N-methyl-L-alanine; N-methyl-L-methionine; N-methyl-L-phenylalanine; N-myristoyl-glycine; N-palmitoyl-L-cysteine; N-pyruvic acid 2-iminyl-L-cysteine; N-pyruvic acid 2-iminyl-L-valine; N-seryl-glycosylphosphatidylinositolethanolamine; N-seryl-glycosyOSPhingolipidinositolethanolamine; O-(ADP-ribosyl)-L-serine; O-(phospho-5′-adenosine)-L-threonine; O-(phospho-5′-DNA)-L-serine; O-(phospho-5′-DNA)-L-threonine; O-(phospho-5′rRNA)-L-serine; O-(phosphoribosyl dephospho-coenzyme A)-L-serine; O-(sn-1-glycerophosphoryl)-L-serine; O4′-(8alpha-FAD)-L-tyrosine; O4′-(phospho-5′-adenosine)-L-tyrosine; O4′-(phospho-5′-DNA)-L-tyrosine; O4′-(phospho-5′-RNA)-L-tyrosine; O4′-(phospho-5′-uridine)-L-tyrosine; O4-glycosyl-L-hydroxyproline; O4′-glycosyl-L-tyrosine; O4′-sulfo-L-tyrosine; O5-glycosyl-L-hydroxylysine; O-glycosyl-L-serine; O-glycosyl-L-threonine; omega-N-(ADP-ribosyl)-L-arginine; omega-N-omega-N′-dimethyl-L-arginine; omega-N-methyl-L-arginine; omega-N-omega-N-dimethyl-L-arginine; omega-N-phospho-L-arginine; O′ octanoyl-L-serine; O-palmitoyl-L-serine; O-palmitoyl-L-threonine; O-phospho-L-serine; O-phospho-L-threonine; O-phosphopantetheine-L-serine; phycoerythrobilin-bis-L-cysteine; phycourobilin-bis-L-cysteine; pyrroloquinoline quinone; pyruvic acid; S hydroxycinnamyl-L-cysteine; S-(2-aminovinyl)methyl-D-cysteine; S-(2-aminovinyl)-D-cysteine; S-(6-FW-L-cysteine; S-(8alpha-FAD)-L-cysteine; S-(ADP-ribosyl)-L-cysteine; 5-(L-isoglutamyl)-L-cysteine; S-12-hydroxyfarnesyl-L-cysteine; S-acetyl-L-cysteine; S-diacylglycerol-L-cysteine; S-diphytanylglycerot diether-L-cysteine; S-farnesyl-L-cysteine; S-geranylgeranyl-L-cysteine; S-glycosyl-L-cysteine; S-glycyl-L-cysteine; S-methyl-L-cysteine; S-nitrosyl-L-cysteine; S-palmitoyl-L-cysteine; S-phospho-L-cysteine; S-phycobiliviolin-L-cysteine; S-phycocyanobilin-L-cysteine; S-phycoerythrobilin-L-cysteine; S-phytochromobilin-L-cysteine; S-selenyl-L-cysteine; S-sulfo-L-cysteine; tetrakis-L-cysteinyl diiron disulfide; tetrakis-L-cysteinyl iron; tetrakis-L-cysteinyl tetrairon tetrasulfide; trans-2,3-cis 4-dihydroxy-L-proline; tris-L-cysteinyl triiron tetrasulfide; tris-L-cysteinyl triiron trisulfide; tris-L-cysteinyl-L-aspartato tetrairon tetrasulfide; tris-L-cysteinyl-L-cysteine persulfido-bis-L-glutamato-L-histidino tetrairon disulfide trioxide; tris-L-cysteinyl-L-N3′-histidino tetrairon tetrasulfide; tris-L-cysteinyl-L-NM'-histidino tetrairon tetrasulfide; and tris-L-cysteinyl-L-serinyl tetrairon tetrasulfide.

Additional examples of post translational modifications can be found in web sites such as the Delta Mass database based on Krishna, R. G. and F. Wold (1998). Posttranslational Modifications. Proteins—Analysis and Design. R. H. Angeletti. San Diego, Academic Press. 1: 121-206.; Methods in Enzymology, 193, J. A. McClosky (ed) (1990), pages 647-660; Methods in Protein Sequence Analysis edited by Kazutomo Imahori and Fumio Sakiyama, Plenum Press, (1993) “Post-translational modifications of proteins” R. G. Krishna and F. Wold pages 167-172; “GlycoSuiteDB: a new curated relational database of glycoprotein glycan structures and their biological sources” Cooper et al. Nucleic Acids Res. 29; 332-335 (2001) “O-GLYCBASE version 4.0: a revised database of O-glycosylated proteins” Gupta et al. Nucleic Acids Research, 27: 370-372 (1999); and “PhosphoBase, a database of phosphorylation sites: release 2.0.”, Kreegipuu et al. Nucleic Acids Res 27(1):237-239 (1999) see also, WO 02/211 39A2, the disclosure of which is incorporated herein by reference in its entirety.

Exemplary polypeptides having increased stability and/or activity of any polypeptide at low pH or elevated temperature that can be produced according to the methods described herein include but are not limited to, cytokines, inflammatory molecules, growth factors, their receptors, and oncogene products or portions thereof. Examples of cytokines, inflammatory molecules, growth factors, their receptors, and oncogene products include, but are not limited to e.g., alpha-1 antitrypsin, Angiostatin, Antihemolytic factor, antibodies (including an antibody or a functional fragment or derivative thereof selected from: Fab, Fab′, F(ab)₂, Fd, Fv, ScFv, diabody, tribody, tetrabody, dimer, trimer or minibody), angiogenic molecules, angiostatic molecules, Apolipopolypeptide, Apopolypeptide, Asparaginase, Adenosine deaminase, Atrial natriuretic factor, Atrial natriuretic polypeptide, Atrial peptides, Angiotensin family members, Bone Morphogenic Polypeptide (BMP-1, BMP-2, BMP-3, BMP-4, BMP-5, BMP-6, BMP-7, BMP-8a, BMP-8b, BMP-10, BMP-15, etc.); C-X-C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant polypeptide-1, Monocyte chemoattractant polypeptide-2, Monocyte chemoattractant polypeptide-3, Monocyte inflammatory polypeptide-1 alpha, Monocyte inflammatory polypeptide-1 beta, RANTES, 1309, R83915, R91733, HCC1, T58847, D31065, T64262), CD40 ligand, C-kit Ligand, Ciliary Neurotrophic Factor, Collagen, Colony stimulating factor (CSF), Complement factor 5a, Complement inhibitor, Complement receptor 1, cytokines, (e.g., epithelial Neutrophil Activating Peptide-78, GRO alpha/MGSA, GRO beta, GRO gamma, MIP-1 alpha, MIP-1 delta, MCP-1), deoxyribonucleic acids, Epidermal Growth Factor (EGF), Erythropoietin (“EPO”, representing a preferred target for modification by the incorporation of one or more non-natural amino acid), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog polypeptides (e.g., Sonic, Indian, Desert), Hemoglobin, Hepatocyte Growth Factor (HGF), Hepatitis viruses, Hirudin, Human serum albumin, Hyalurin-CD44, Insulin, Insulin-like Growth Factor (IGF-I, IGF-II), interferons (e.g., interferon-alpha, interferon-beta, interferon-gamma, interferon-epsilon, interferon-zeta, interferon-eta, interferon-kappa, interferon-lambda, interferon-T, interferon-zeta, interferon-omega), glucagon-like peptide (GLP-1), GLP-2, GLP receptors, glucagon, other agonists of the GLP-1R, natriuretic peptides (ANP, BNP, and CNP), Fuzeon and other inhibitors of HIV fusion, Hurudin and related anticoagulant peptides, Prokineticins and related agonists including analogs of black mamba snake venom, TRAIL, RANK ligand and its antagonists, calcitonin, amylin and other glucoregulatory peptide hormones, and Fc fragments, exendins (including exendin-4), exendin receptors, interleukins (e.g., IL-1, IL-2, IL-3, IL-4, IL-5, IL-6, IL-7, IL-8, IL-9, IL-10, IL-11, IL-12, etc.), I-CAM-1/LFA-1, Keratinocyte Growth Factor (KGF), Lactoferrin, leukemia inhibitory factor, Luciferase, Neurturin, Neutrophil inhibitory factor (NIF), oncostatin M, Osteogenic polypeptide, Parathyroid hormone, PD-ECSF, PDGF, peptide hormones (e.g., Human Growth Hormone), Oncogene products (Mos, Rel, Ras, Raf, Met, etc.), Pleiotropin, Polypeptide A, Polypeptide G, Pyrogenic exotoxins A, B, and C, Relaxin, Renin, ribonucleic acids, SCF/c-kit, Signal transcriptional activators and suppressors (p53, Tat, Fos, Myc, Jun, Myb, etc.), Soluble complement receptor 1, Soluble I-CAM 1, Soluble interleukin receptors (IL-1, 2, 3, 4, 5, 6, 7, 9, 10, 11, 12, 13, 14, 15), soluble adhesion molecules, Soluble TNF receptor, Somatomedin, Somatostatin, Somatotropin, Streptokinase, Superantigens, i.e., Staphylococcal enterotoxins (SEA, SEB, SECT, SEC2, SEC3, SED, SEE), Steroid hormone receptors (such as those for estrogen, progesterone, testosterone, aldosterone, LDL receptor ligand and corticosterone), Superoxide dismutase (SOD), Toll-like receptors (such as Flagellin), Toxic shock syndrome toxin (TSST-1), Thymosin a 1, Tissue plasminogen activator, transforming growth factor (TGF-alpha, TGF-beta), Tumor necrosis factor beta (TNF beta), Tumor necrosis factor receptor (TNFR), Tumor necrosis factor-alpha (TNF alpha), transcriptional modulators (for example, genes and transcriptional modular polypeptides that regulate cell growth, differentiation and/or cell regulation), Vascular Endothelial Growth Factor (VEGF), virus-like particle, VLA-4NCAM-1, Urokinase, signal transduction molecules, estrogen, progesterone, testosterone, aldosterone, LDL, corticosterone.

Additional polypeptides having increased stability and/or activity of any polypeptide at low pH or elevated temperature that can be produced according to the methods described herein include but are not limited to enzymes (e.g., industrial enzymes) or portions thereof. Examples of enzymes include, but are not limited to amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

Other polypeptides having increased stability and/or activity of any polypeptide at low pH or elevated temperature that can be produced according to the methods described herein include, but are not limited to, agriculturally related polypeptides such as insect resistance polypeptides (e.g., Cry polypeptides), starch and lipid production enzymes, plant and insect toxins, toxin-resistance polypeptides, Mycotoxin detoxification polypeptides, plant growth enzymes (e.g., Ribulose 1,5-Bisphosphate Carboxylase/Oxygenase), lipoxygenase, and Phosphoenolpyruvate carboxylase.

Polypeptides having increased stability and/or activity of any polypeptide at low pH or elevated temperature that can be produced according to the methods described herein include, but are not limited to, antibodies, immunoglobulin domains of antibodies and their fragments. Examples of antibodies include, but are not limited to antibodies, antibody fragments, antibody derivatives, Fab fragments, Fab′ fragments, F(ab)₂ fragments, Fd fragments, Fv fragments, single-chain Fv fragments (scFv), diabodies, tribodies, tetrabodies, dimers, trimers, and minibodies.

In another embodiment, the invention is directed to a composition comprising a recombinant polypeptide having increased stability and/or activity of any polypeptide at low pH or elevated temperature produced according to the methods described herein, and an additional component selected from the group consisting of pharmaceutically acceptable diluents, carriers, excipients and adjuvants.

Polypeptides having increased stability and/or activity of any polypeptide at low pH or elevated temperature that can be produced according to the methods described herein can also further comprise a chemical moiety selected from the group consisting of: cytotoxins, pharmaceutical drugs, dyes or fluorescent labels, a nucleophilic or electrophilic group, a ketone or aldehyde, azide or alkyne compounds, photocaged groups, tags, a peptide, a polypeptide, a polypeptide, an oligosaccharide, polyethylene glycol with any molecular weight and in any geometry, polyvinyl alcohol, metals, metal complexes, polyamines, imidizoles, carbohydrates, lipids, biopolymers, particles, solid supports, a polymer, a targeting agent, an affinity group, any agent to which a complementary reactive chemical group can be attached, biophysical or biochemical probes, isotypically-labeled probes, spin-label amino acids, fluorophores, aryl iodides and bromides.

In some embodiments, the present invention involves mutating nucleotide sequences to add/create or remove/disrupt sequences. Such mutations can me made using any suitable mutagenesis method known in the art, including, but not limited to, site-directed mutagenesis, oligonucleotide-directed mutagenesis, positive antibiotic selection methods, unique restriction site elimination (USE), deoxyuridine incorporation, phosphorothioate incorporation, and PCR-based mutagenesis methods. Details of such methods can be found in, for example, Lewis et al. (1990) Nucl. Acids Res. 18, p3439; Bohnsack et al. (1996) Meth. Mol. Biol. 57, p1; Vavra et al. (1996) Promega Notes 58, 30; Altered SitesII in vitro Mutagenesis Systems Technical Manual #TM001, Promega Corporation; Deng et al. (1992) Anal. Biochem. 200, p81; Kunkel et al. (1985) Proc. Natl. Acad. Sci. USA 82, p488; Kunke et al. (1987) Meth. Enzymol. 154, p367; Taylor et al. (1985) Nucl. Acids Res. 13, p8764; Nakamaye et al. (1986) Nucl. Acids Res. 14, p9679; Higuchi et al. (1988) Nucl. Acids Res. 16, p7351; Shimada et al. (1996) Meth. Mol. Biol. 57, p157; Ho et al. (1989) Gene 77, p51; Horton et al. (1989) Gene 77, p61; and Sarkar et al. (1990) BioTechniques 8, p404. Numerous kits for performing site-directed mutagenesis are commercially available, such as the QuikChange II Site-Directed Mutagenesis Kit and the Altered Sites II in vitro mutagenesis system. Such commercially available kits may also be used to optimize sequences. Other techniques that can be used to generate modified nucleic acid sequences are well known to those of skill in the art. See for example Sambrook et al. (2001) Molecular Cloning: A Laboratory Manual, 3rd Ed., Cold Spring Harbor Laboratory, Cold Spring Harbor, N.Y.

The following examples illustrate the present invention, and are set forth to aid in the understanding of the invention, and should not be construed to limit in any way the scope of the invention as defined in the claims which follow thereafter.

### EXAMPLES

### Example 1

### Paleoenzymology at the Single-Molecule Level: Probing the Chemistry of Resurrected Enzymes

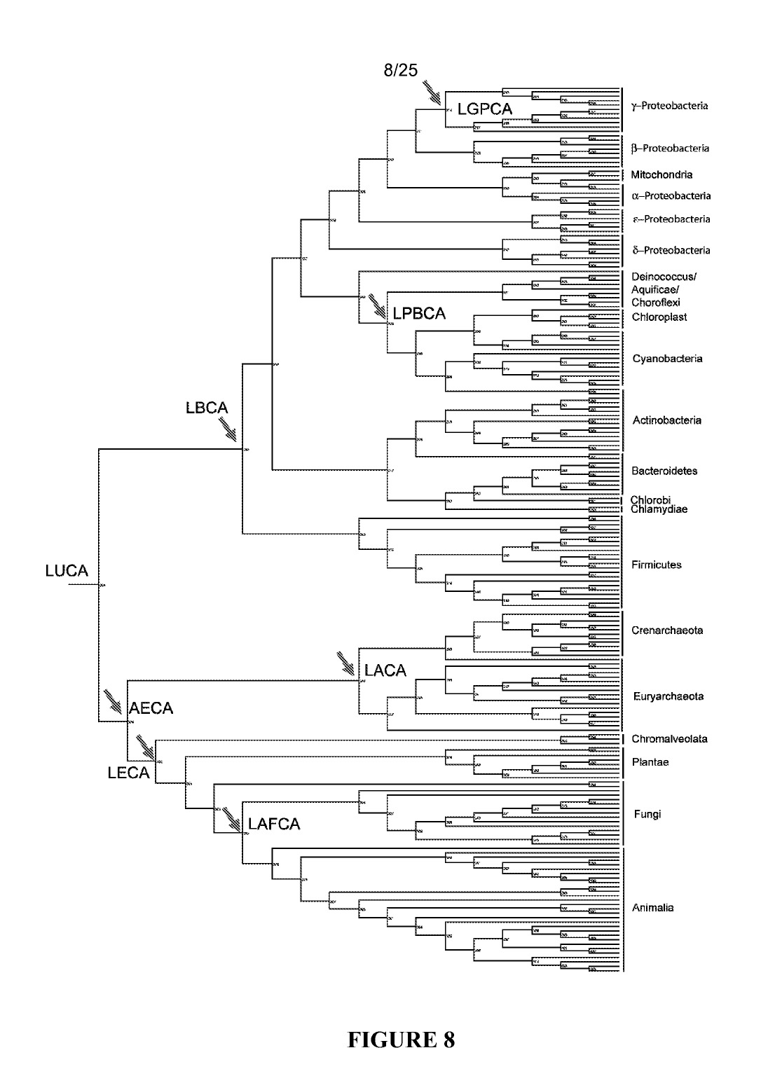

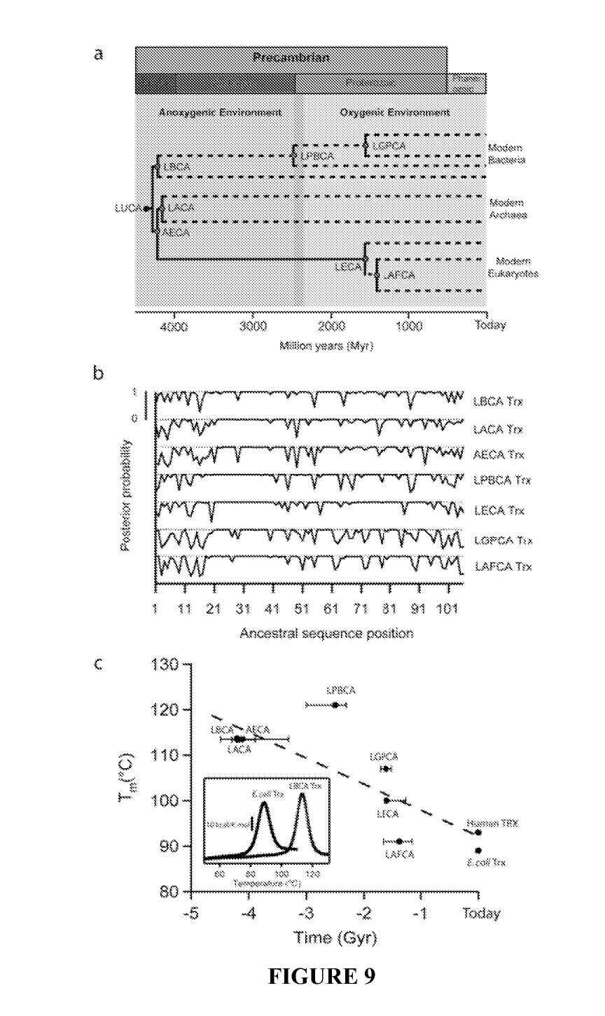

A highly articulated phylogenetic tree encompassing over 200 diverse Trx sequences from the three domains of life was constructed (FIG. 8). Several biologically relevant nodes for sequence reconstruction and laboratory resurrection were sampled from this tree. Divergence dates estimates were applied to nodes in the tree assuming the root of the tree lies between bacteria and the common ancestor of archaea/eukaryotes (Hedges and Kumar, The Timetree of life, xxi, 551 p. (Oxford University Press, Oxford, 2009)). In particular, Trx enzymes belonging to the last bacterial common ancestor (LBCA in FIG. 9), the last archaeal common ancestor (LACA) and the archaeal/eukaryotic common ancestor (AECA) (FIG. 9) were resurrected. These organisms are thought to have inhabited Earth 4.2-3.5 Gyr ago (FIG. 9A) after diverging from the last universal common ancestor (LUCA) (Boussau et al., Nature 456, 942-5 (2008); Hedges and Kumar, The Timetree of life, xxi, 551 p. (Oxford University Press, Oxford, 2009)). A node corresponding to the last eukaryotic common ancestor (LECA) that lived in the Proterozoic, ˜1.60 Gyr ago was also selected. Two other internal nodes in the bacterial lineages were selected; the last common ancestor of cyanobacterial and deinococcus/thermus groups (LPBCA) which existed ˜2.50 Gyr ago and represents the origin of photosynthetic bacteria, and the last common ancestor of γ-proteobacteria, ˜1.61 Gyr old (LGPCA). Finally, the last common ancestor of animals and fungi (LAFCA) that lived ˜1.37 Gyr ago (FIG. 9A) was also chosen.

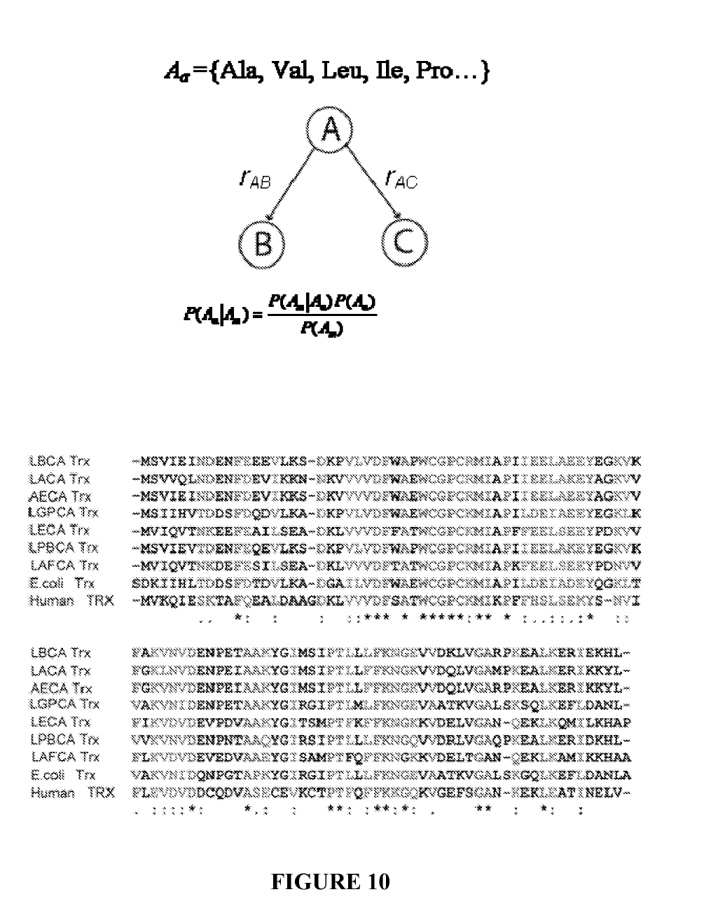

The sequences of the ancestral Trx enzymes were reconstructed using statistical methods based on maximum likelihood (Liberles, Ancestral sequence reconstruction, xiii, 252 p. (Oxford University Press, Oxford; New York, 2007; Gaucher et al., Nature 425, 285-8 (2003)). For a given node in the tree, the posterior probability values for all 20 amino acids were calculated considering each site of the inferred sequence. These values represent the probability that a certain residue occupied a specific position in the sequence at a particular point in the phylogeny. The posterior probabilities were calculated on the basis of an amino acid replacement matrix (Yang et al., Genetics 141, 1641-50 (1995)). The most probabilistic ancestral sequence (M-PAS) at a specific node was then reconstructed by assigning to each site the residue with the highest posterior probability. FIG. 9B shows the posterior probability distribution of the inferred amino acids across 106 sites for the selected sequences. The M-PASs of interest are summarized in FIG. 10. The genes encoding these sequences were synthesized and the proteins were expressed and purified from E. coli cells.

TABLE 1
List of Thioredoxin sequences used for ancestral sequences reconstruction.
The following GI numbers were accessed from GenBank. The names
of the hosting organisms are also provided:

| | | | |
|---|---|---|---|
| 57164261 | 1620905 | 15894825 | 15807833 |
| Ovis | Fagopyrum | Clostridium | Deinococcus |
| 27806783 | 46226985 | 15896334 | 46199687 |
| Bos | Cryptosporidium | Clostridium | Thermus |
| 47523692 | 68350806 | 20807685 | 15805968 |
| Sus | Theileria | Thermoanaerobacter | Deinococcus |
| 126352340 | 148804689 | 76789276 | 147669275 |
| Equus | Plasmodium | Chlamydia | Dehalococcoides |
| 6755911 | 11498883 | 15836191 | 118047160 |
| Mus | Archaeoglobus | Chlamydophila | Chloroflexus |
| 16758644 | 116754023 | 119357517 | 118048687 |
| Rattus | Methanosaeta | Chlorobium | Chloroflexus |
| 146291083 | 91773622 | 119357012 | 118046691 |
| Rabbit | Methanococcoides | Chlorobium | Chloroflexus |
| 135773 | 154149646 | 29345629 | 15606934 |
| Human | Candidatus | Bacteroides | Aquifex |
| 67461921 | 88603734 | 150024368 | 42521808 |
| Ponab | Methanospirillum | Flavobacterium | Bdellovibrio |
| 267126 | 48477193 | 34539910 | 39998535 |
| Macmu | Picrophilus | Porphyromonas | Geobacter |
| 13560979 | 150401020 | 29347639 | 42523902 |
| Callithrix | Methanococcus | Bacteroides | Bdellovibrio |
| 126339826 | 124485138 | 29346087 | 120602368 |
| Monodelphis | Methanocorpusculum | Bacteroides | Desulfovibrio |
| 149412981 | 116754438 | 34540117 | 39998370 |
| Ornithorhynchus | Methanosaeta | Porphyromonas | Geobacter |
| 45382053 | 76802488 | 29346866 | 116619824 |
| Gallus | Natronomonas | Bacteroides | Solibacter |
| 29373131 | 110667588 | 29345628 | 116619449 |
| Melopsittacus | Haloquadratum | Bacteroides | Solibacter |
| 12958636 | 55380304 | 32477354 | 94970094 |
| Ophiophagus | Haloarcula | Rhodopirellula | Acidobacteria |
| 194332745 | 76802694 | 32476401 | 34556879 |
| Xenopus | Natronomonas | Rhodopirellula | Wolinella |
| 47215756 | 16120325 | 15608608 | 15645443 |
| Tetraodon | Halobacterium | Mycobacterium | Helicobacter |
| 9837585 | 11499727 | 57116870 | 57237155 |
| Ictalurus | Archaeoglobus | Mycobacterium | Campylobacter |
| 50539990 | 13541608 | 62391823 | 15646067 |
| Danio | Thermoplasma | Corynebacterium | Helicobacter |
| 194160556 | 119720035 | 72163169 | 34557886 |
| Drosophila | Thermofilum | Thermobifida | Wolinella |
| 17648013 | 159040636 | 21219405 | 34556999 |
| Drosophila | Caldivirga | Streptomyces | Wolinella |
| 194141429 | 70607552 | 72160576 | 159184127 |
| Drosophila | Sulfolobus | Thermobifida | Agrobacterium |
| 48104680 | 15899007 | 15607956 | 150398433 |
| Apis | Sulfolobus | Mycobacterium | Sinorhizobium |
| 91084205 | 15922449 | 21219599 | 17988305 |
| Tribolium | Sulfolobus | Streptomyces | Brucella |
| 148298796 | 124027987 | 62391938 | 15603883 |
| Bombyx | Hyperthermus | Corynebacterium | Rickettsia |
| 90819972 | 118431868 | 21223797 | 108935910 |
| Graphocephala | Aeropyrum | Streptomyces | Bovin Mitochondrio |
| 169639275 | 146304377 | 15611050 | 194226778 |
| Litopenaeus | Metallosphaera | Mycobacterium | Equus |
| 30580603 | 70607229 | 72163508 | 21361403 |
| Geocy | Sulfolobus | Thermobifida | Homo Mitochondrion |
| 115401922 | 15897303 | 21222296 | 16758038 |
| Aspergillus | Sulfolobus | Streptomyces | Rattus Mitochondrio |
| 119479067 | 126465005 | 16329883 | 9903609 |
| Neosartorya | Staphylothermus | Synechocystis | Mus Mitochondrion |
| 40746887 | 118431901 | 17229833 | 74318624 |
| Aspergillus | Aeropyrum | Nostoc | Thiobacillus |
| 115401518 | 15894111 | 17229385 | 121635072 |
| Aspergillus | Clostridium | Nostoc | Neisseria |
| 150951554 | 20808289 | 16331440 | 74316054 |
| Pichia | Thermoanaerobacter | Synechocystis | Thiobacillus |
| 46441186 | 16079205 | 22299829 | 126454139 |
| Candida | Bacillus | Thermosynechococcus | Burkholderia |
| 126213085 | 16077522 | 22297898 | 33602206 |
| Pichia | Bacillus | Thermosynechococcus | Bordetella |
| 50309357 | 15901736 | 16329237 | 74318419 |
| Kluyveromyces | Streptococcus | Synechocystis | Thiobacillus |
| 151943486 | 29377495 | 22299630 | 74316241 |
| Saccharomyces | Enterococcus | Thermosynechococcus | Thiobacillus |
| 50291653 | 153181008 | 17229697 | 33602001 |
| Candida | Listeria | Nostoc | Bordetella |
| 151941211 | 28377165 | 17229859 | 66043570 |
| Saccharomyces | Lactobacillus | Nostoc | Pseudomonas |
| 19114764 | 28379765 | 22298354 | 27364380 |
| Schizosaccharomyces | Lactobacillus | Thermosynechococcus | Vibrio |
| 167537844 | 150393692 | 17229358 | 16124003 |
| Monosiga | Staphylococcus | Nostoc | Yersinia |
| 67479051 | 138896249 | 126696505 | 16767191 |
| Entamoeba | Geobacillus | Prochlorococcus | Salmonella |
| 165988451 | 30264587 | 16331825 | 30064924 |
| Dictyostelium | Bacillus | Synechocystis | Shigella |
| 15236327 | 16079902 | 17227548 | 67005950 |
| Arabidopsis | Bacillus | Nostoc1 | Escherichia |
| 15232567 | 28378864 | 1351239 | 16130507 |
| Arabidopsis | Lactobacillus | Pea Chloroplast | Escherichia |
| 154721452 | 153179313 | 2507458 | 30063983 |
| Limonium | Listeria | Spiol Chloroplast | Shigella |
| 162461510 | 29375972 | 11135474 | 16765969 |
| Zea | Enterococcus | Wheat Chloroplast | Salmonella |
| 157335070 | 15901605 | 15594012 | 16123427 |
| Vitis | Streptococcus | Pisum Chloroplast | Yersinia |
| 145351136 | 110798962 | 11135407 | 27366792 |
| Ostreococcus | Clostridium | Brana Chloroplast | Vibrio |
| 53801490 | 110800418 | 46199419 | |
| Helicosporidium | Clostridium | Thermus | |

Thermal Stability of Ancient Trx Enzymes

As a first step toward investigating the physico-chemical properties of these resurrected enzymes, differential scanning calorimetry (DSC) was used to measure their thermal stabilities. The denaturation temperature (Tₘ) can provide an idea about the temperature range in which the proteins are operative. FIG. 9C shows a plot of the Tₘ of the resurrected enzymes against geological time. A Tₘ of ˜113° C. was measured for LBCA, AECA and LACA Trx. As observed in FIG. 9C (inset), LBCA Trx maintains a highly populated native state up to ˜105° C., where the thermal transition begins. By contrast, a Tₘ for modern E. coli and human Trxs of 88.8 and 93.3° C. respectively, was determined. The ΔTₘ between the oldest and modern Trx is ˜25° C., a similar value than that determined for bacterial EF (Gaucher et al., Nature 451, 704-7 (2008)), which corroborates the hypothesis of the thermophilic nature of LBCA, AECA and LACA (Boussau et al., Nature 456, 942-5 (2008)). In FIG. 9C shows a paleotemperature trend yielding a decrease in the Tₘ of 5.8±1.8 K/Gyr. These results show that, in early life, Trx enzymes functioned in hot environments and that these environments have progressively cooled from 4 to 0.5 Gyr ago (Nisbet and Sleep, Nature 409, 1083-91 (2001); Gaucher et al., Nature 451, 704-7 (2008); Knauth and Lowe, Geol. Soc. Am. Bull. 115, 566-580 (2003); Schulte, M. The Emergence of Life on Earth. Oceanography 20, 42-49 (2007)). Although the thermodynamic denaturation temperatures determined for the ancestral Trxs follow a similar cooling trend that the ancient oceans, the actual values are about 50 degrees higher than the ocean temperatures inferred from maximum δ¹⁸O (Gaucher et al., Nature 451, 704-7 (2008)). Accordingly, Trx evolution may operate primarily on kinetic stability and this could be reflected in thermodynamic stability (Godoy-Ruiz et al., J Mol Biol 362, 966-78 (2006)). However, other than loss of function upon denaturation, the particular way in which the value of Tₘ is related to Trx enzyme fitness is still unknown.

Force-dependent chemical kinetics of disulfide reduction

It is also of great interest to examine the chemical mechanisms of disulfide bond reduction utilized by the resurrected enzymes. Given the ancient origin of the resurrected thioredoxin enzymes, with some of them predating the buildup of atmospheric oxygen, it can be assumed that chemical mechanisms of disulfide bond reduction utilized by the resurrected enzymes are closer to that of simple sulfur based molecules. Simple sulfur based molecules utilize a straightforward collision-driven substitution nucleophilic bimolecular (S_{N}2) mechanism of reduction (Kice et al., Progress in Inorganic Chemistry (ed. Edwards, J. O.) 147-206 (2007)). By contrast, Trx enzymes utilize a complex mixture of chemical mechanisms including a critical substrate binding and rearrangement reaction that accounts for the vast increase in the efficiency of Trx over the simpler sulfur compounds that were available in early geochemistry (Wiita et al., Nature 450, 124-7 (2007); Perez-Jimenez et al., Nat Struct Mol Biol 16, 890-6 (2009)).

A single molecule force-spectroscopy based assay can be used to measure the effect of applying a well-controlled force to a disulfide bonded substrate, on its rate of reduction by a nucleophile. This assay can be used to distinguish the simple S_{N}2 chemistry of nucleophiles (e.g. hydroxide, glutathione and L-Cys), from the more complex reduction chemistry of the Trx enzymes (Wiita et al., Nature 450, 124-7 (2007); Perez-Jimenez et al., Nat Struct Mol Biol 16, 890-6 (2009); Wiita et al., Proc Natl Acad Sci USA 103, 7222-7 (2006); Koti Ainavarapu et al., J Am Chem Soc 130, 6479-87 (2008); Garcia-Manyes et al., Nature Chemistry 1, 236-242 (2009); Liang and Fernandez, Mechanochemistry: One Bond at a Time. ACS Nano (2009)). This feature makes this assay a good system to probe the chemistry of the resurrected enzymes.

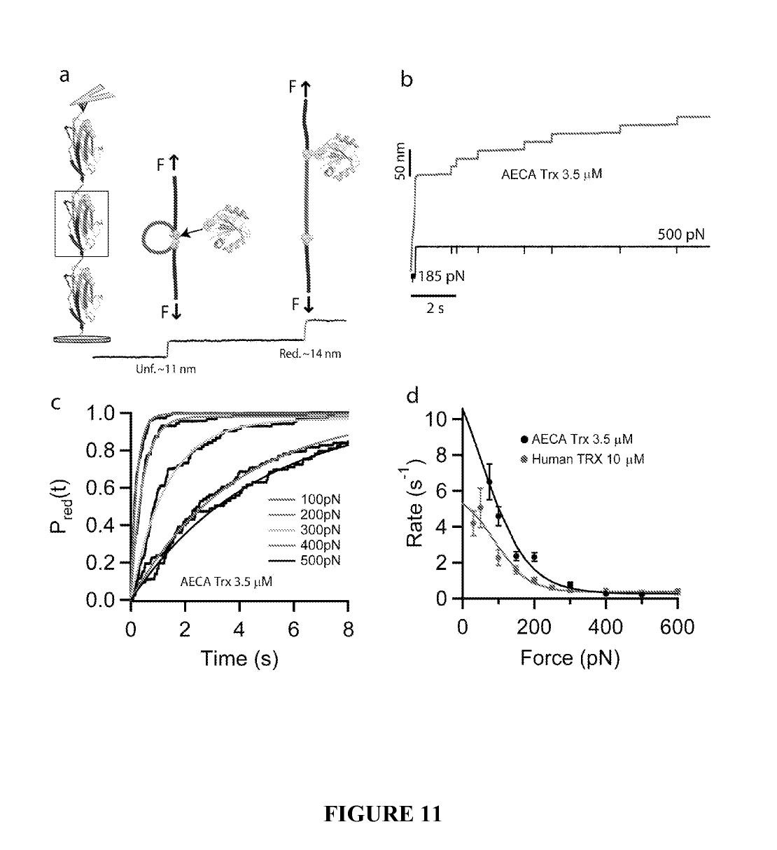

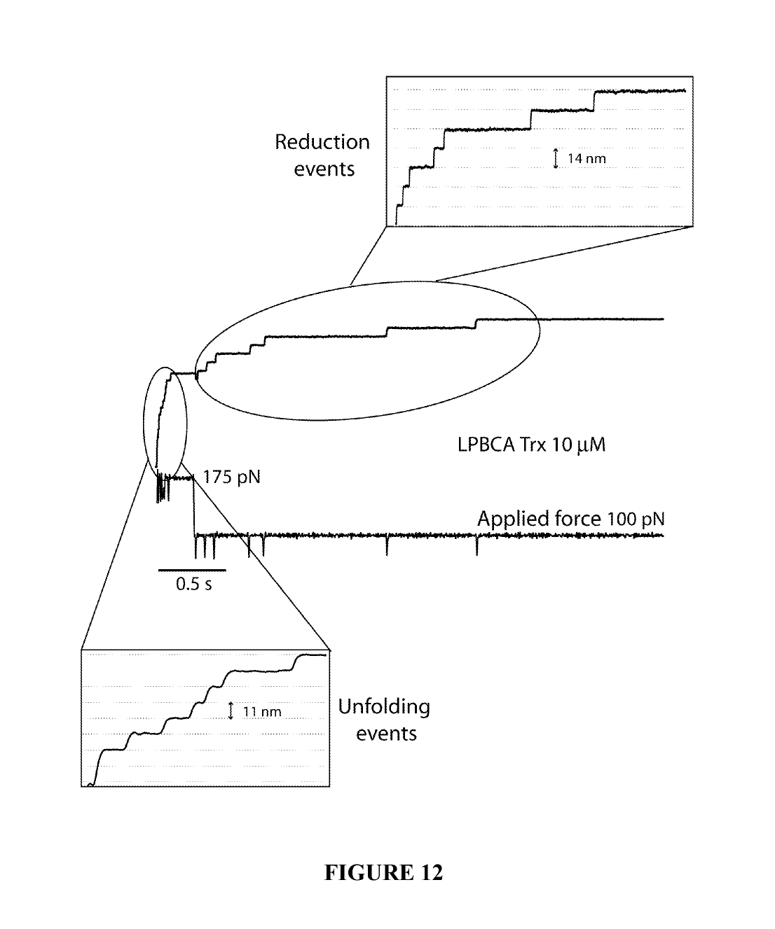

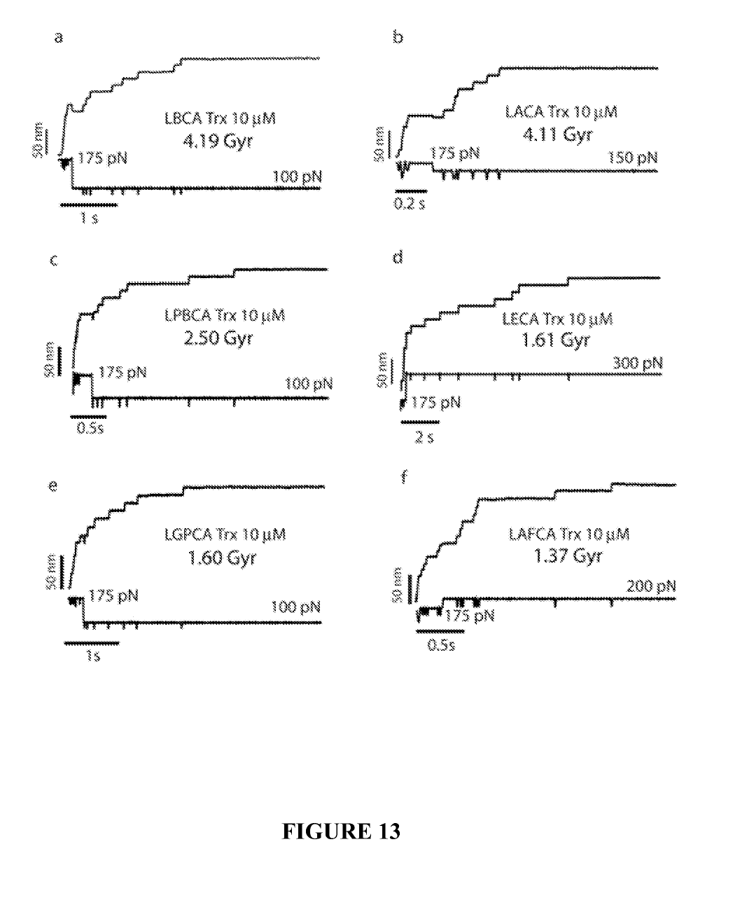

This approach is described in FIG. 11. Although different types of substrates can be used in this approach, in one embodiment, the substrate is an engineered polypeptide made of eight repeats of the I27 immunoglobulin-like protein modified by mutating to Cys positions 32^{nd} and 75^{th}(I27_{G32C-A75C})₈. The cysteines oxidize spontaneously, forming disulfide bonds that are hidden within each folded I27 protein in the chain. Single polypeptides are picked up and stretched in solutions containing the desired nucleophile using an AFM. In a typical experiment, a constant force is applied to the polypeptide (175-185 pN, 0.2-0.3 s). This rapidly unfolds the I27_{G32C-A75C} modules up to the disulfide bond. The unfolding events result in a stepwise increase in the length of the polypeptide where each module contributes with ˜11 nm in length (FIG. 11A, FIG. 12). After unfolding, every disulfide bond becomes exposed to the solvent. If active Trx enzymes are present in the solution, single reduction events of ˜14 nm per module can be observed (FIGS. 11A, B; FIG. 12, FIG. 13). All the ancestral enzymes resurrected using the methods described herein were able to trigger staircases of reduction events (FIG. 11B and FIG. 12, FIG. 13) indicating that they were all active. In order to measure the reduction rate, 15 to 80 reduction staircases similar to the one shown in FIG. 11B can be summed and the resulting average can be fit with a single exponential. This procedure can be fitted for different pulling forces (FIG. 11C). The resulting set of data measures the force-dependency of the rate of reduction of the disulfide bond (FIG. 11D).

The chemical mechanisms of disulfide reduction can be distinguished by their sensitivity to the force applied to the substrate (Perez-Jimenez et al., Nat Struct Mol Biol 16, 890-6 (2009)). Simple thiol reducing agents show a force-dependency where the rate always increased exponentially with the applied force (Wiita et al., Proc Natl Acad Sci USA 103, 7222-7 (2006); Koti Ainavarapu et al., J Am Chem Soc 130, 6479-87 (2008)). By contrast, modern Trx enzymes show a negative force dependency in the range of 30-200 pN (Perez-Jimenez et al., Nat Struct Mol Biol 16, 890-6 (2009)). This mechanism is consistent with a Michaelis-Menten binding reaction followed by a force-inhibited reorientation of the substrate disulfide bond, necessary for an S_{N}2 reaction to occur (Wiita et al., Nature 450, 124-7 (2007)). In a second mechanism, the rate of reduction increases exponentially at forces above 200 pN. This mechanism can be described by a simple S_{N}2 reaction and is found only in Trx enzymes of bacterial origin. Present in all thioredoxin enzymes, there is a force-independent mechanism of reduction that can be ascribed to single electron transfer reaction (Perez-Jimenez et al., Nat Struct Mol Biol 16, 890-6 (2009)).

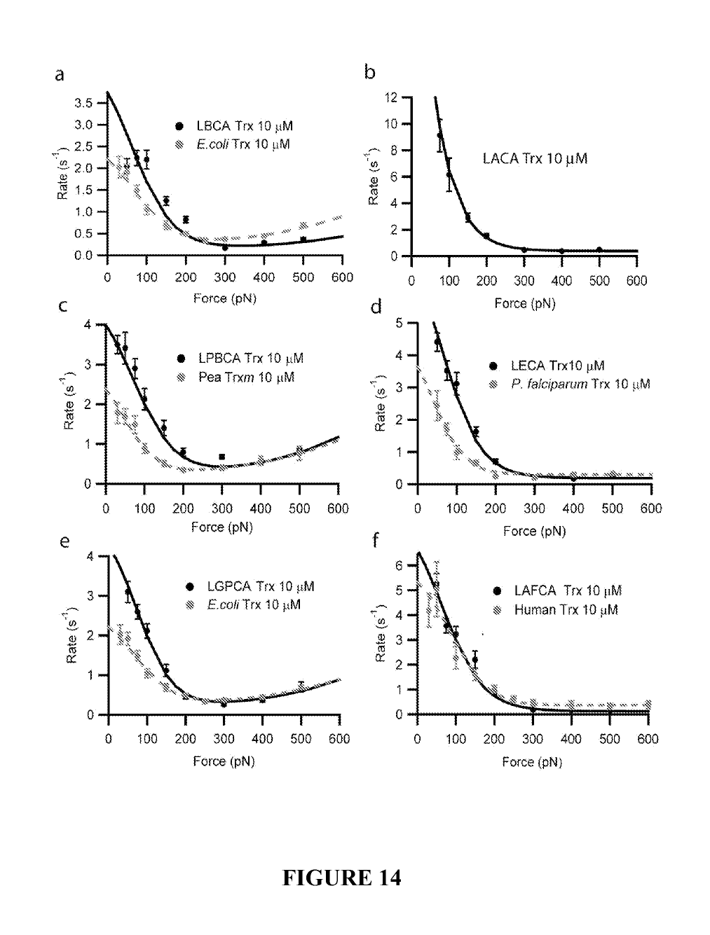

Surprisingly, the same three reduction mechanisms can be observed in the ancient enzymes with similar patterns to those found in extant Trxs (FIG. 11D, FIG. 14). Indeed, the force-dependency of the reduction rate measured from the resurrected enzymes can be fit using the three-state kinetic model used with modern Trxs (Wiita et al., Nature 450, 124-7 (2007); Perez-Jimenez et al., Nat Struct Mol Biol 16, 890-6 (2009)) (Table 2).

TABLE 2
Kinetic parameters for Ancestral Trxs.

| Enzyme | α₀ (μM⁻¹ · s⁻¹) | β₀ (s−¹) | γ₀ (μM⁻¹ · s⁻¹) | k₁₀ (s⁻¹) | Δx₁₂ (Å) | Δx₀₂ (Å) | λ₀ (s⁻¹) |
|---|---|---|---|---|---|---|---|
| LBCA Trx | 0.47 ± 0.08 | 30 ± 2 | 0.004 ± 0.001 | 5.8 ± 0.7 | −0.74 ± 0.06 | 0.19 ± 0.02 | 0.09 ± 0.02 |
| LACA Trx | 8.2 ± 0.2 | 43 ± 3 | — | 3.8 ± 1 | −0.76 ± 0.04 | — | 0.38 ± 0.05 |
| AECA Trx | 4.2 ± 0.3 | 25 ± 2 | 0.019 ± 0.004 | 3.8 ± 0.6 | −0.84 ± 0.05 | 0.19 ± 0.02 | 0.21 ± 0.04 |
| LPBCA Trx | 0.47 ± 0.04 | 30 ± 3 | 0.017 ± 0.002 | 4.9 ± 0.5 | −0.71 ± 0.01 | 0.17 ± 0.02 | 0.19 ± 0.02 |
| LECA Trx | 0.76 ± 0.08 | 38 ± 2 | — | 4.2 ± 0.7 | −0.80 ± 0.03 | — | 0.18 ± 0.01 |
| LGPCA Trx | 0.48 ± 0.02 | 34 ± 2 | 0.012 ± 0.002 | 3.8 ± 0.4 | −0.83 ± 0.02 | 0.17 ± 0.02 | 0.35 ± 0.02 |
| LAFCA Trx | 0.81 ± 0.10 | 37 ± 3 | — | 4.6 ± 0.8 | −0.74 ± 0.03 | — | 0.06 ± 0.02 |
| E. coli Trx1* | 0.25 ± 0.02 | 24 ± 2 | 0.012 ± 0.002 | 4.7 ± 0.5 | −0.74 ± 0.05 | 0.16 ± 0.01 | 0.08 ± 0.02 |
| Human Trx1* | 0.52 ± 0.05 | 33 ± 2 | — | 3.1 ± 0.9 | −0.71 ± 0.05 | — | 0.35 ± 0.02 |

The parameters were obtained using the kinetic model previously described (see methods section and references 14 and 15 in the main text). They are the result of numeric optimization of the global fit using the downhill simplex method. The errors correspond to the standard deviation. E. coli and human Trxs are also included, obtained from refs 14 and 15 (*).

One might expect that Trx enzymes from primitive forms of life should have less-developed chemical mechanisms. For instance, one of the main factors controlling the chemistry of Trx catalysis is the geometry of the binding groove. In the case of modern bacterial-origin Trxs, the binding groove is less pronounced than in eukaryotic Trxs (Perez-Jimenez et al., Nat Struct Mol Biol 16, 890-6 (2009)). This structural difference is responsible for the different chemical behavior observed in eukaryotic versus bacterial Trxs. If ancient enzymes had a less-structured groove, it could make their chemistry more similar to that of simple reducing agents like L-Cys or TCEP (Ainavarapu et al., J Am Chem Soc 130, 436-7 (2008)). However, the chemistry of Trx enzymes seems to have been established very early in evolution, about 4 Gyr ago, in the same manner that it is observed today. This observation shows that the step from simple reducing compounds to well-structured and functional enzymes occurred early in molecular evolution (Nisbet and Sleep, Nature 409, 1083-91 (2001)).

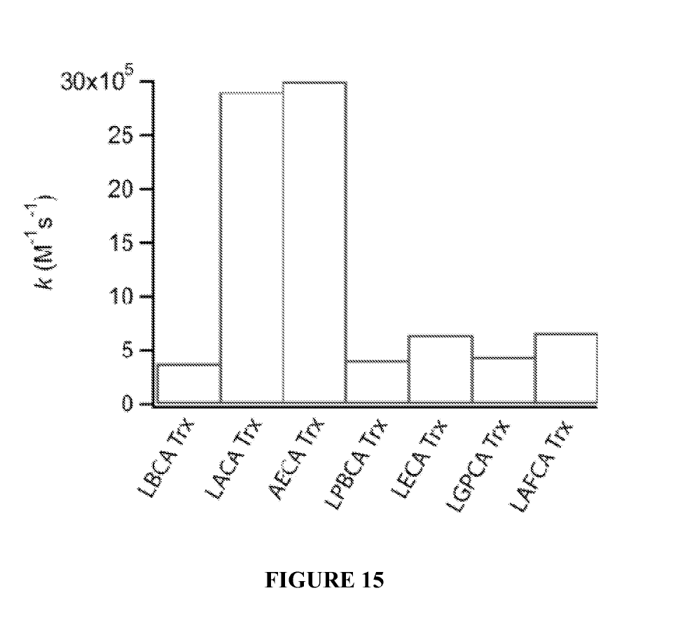

Nevertheless, several aspects of the catalytic mechanisms of some ancestral Trxs are intriguing. For example, high activity is observed for AECA and LACA Trxs when the substrate is pulled at forces below 200 pN (FIGS. 11D and 14B). From the fitting of the reduction rate versus force data to the three-state kinetic model, an extrapolation to zero force yields rate constants of 30×10⁵ M⁻¹ s⁻¹ for AECA Trx and 29×10⁵ M⁻¹ s⁻¹ for LACA Trx. The extrapolation to zero force in the rest of ancestral Trxs predicts rate constants ranging from 3.7×10⁵ M⁻¹ s⁻¹ to 6.6×10⁵ M⁻¹ s¹ (FIG. 15). These latter values are similar to those found in extant Trx enzymes (Perez-Jimenez et al., Nat Struct Mol Biol 16, 890-6 (2009)). Another interesting feature is the small upward slope observed at low forces for LBCA Trx with a maximum at ˜100 pN (FIG. 14A). Although structural information would be needed to fully address this point, it seems possible that the binding between substrate and enzyme is not optimum at zero force. A better conformation can be achieved by applying force to the substrate.

Activity of Ancestral Trxs in Acidic Conditions (pH 5)

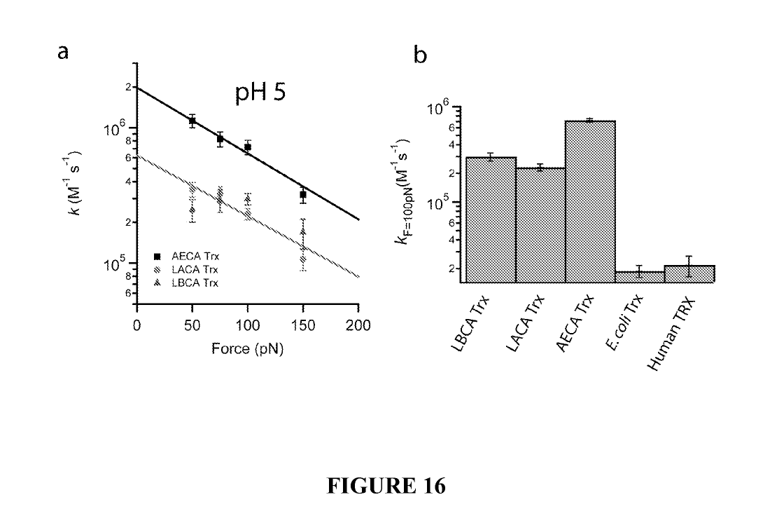

LBCA, AECA and LACA lived in an anoxygenic environment likely rich in sulfur compounds and CO₂ whereas LPBCA, LECA, LGPCA and LAFCA lived in an oxygenic environment (Nisbet and Sleep, Nature 409, 1083-91 (2001)) (FIG. 9A). The high level of CO₂ in the Hadean was partly responsible for the proposed low pH of the ancient oceans (˜5.5) (Walker, Nature 302, 518-520 (1983); Russell and Hall, J Geol Soc Lond 154, 377-402 (1997)). Therefore, following the hypothesis that early life lived in seawater, the natural habitat in which LBCA, AECA and LACA lived was likely to have been acidic in addition to hot. This is especially important given that the reactivity of modern Trx enzymes is due, in part, to the low pKₐ value of the reactive Cys: 6.7 vs. 8.0 for L-Cys (Holmgren, Thioredoxin. Annu Rev Biochem 54, 237-71 (1985)). This low pKₐ is needed to maintain the reactive thiolate anion form of the catalytic cysteine in the active site of the enzyme (Holmgren, Thioredoxin. Annu Rev Biochem 54, 237-71 (1985)) and is a consequence of complex electrostatic interactions between several residues that stabilize the deprotonated form of the reactive cysteine (Dyson, H. J. et al., Biochemistry 36, 2622-36 (1997). Thus, Trx activity is highly sensitive to pH and modern enzymes would not work well at low pH because the catalytic thiol would be protonated and inactive. To examine these considerations the reactivity of LACA, AECA and LBCA enzymes were compared with the extant human and E. coli Trx enzymes at pH 5. This analysis showed that the resurrected enzymes operate in low pH environments. The force dependency of reduction for AECA, LACA and LBCA at pH 5 was measured, over the 50-150 pN force range (FIG. 16A). For AECA Trx, an extrapolation to zero force gives a reduction rate constant of 19×10⁵ M⁻¹ s⁻¹ (FIG. 16A, solid line); similarly for LACA, a rate constant of 6.2×10⁵ M⁻¹ s⁻¹ is estimated, whereas for LBCA Trx the reduction rates observed at pH 5 are strikingly similar to those measured at pH 7.2 (FIG. 16A). These are very high values similar to those measured for some modern Trx enzymes at neutral pH (Perez-Jimenez et al., Nat Struct Mol Biol 16, 890-6 (2009)). FIG. 16B shows a comparison of the rate constants of reduction measured at 100 pN for LBCA, LACA and AECA with modern E. coli and human Trxs also measured at pH 5. It is clear from these data that ancient Trx enzymes were well adapted to function under acidic conditions and that Trx enzymes were able to maintain similar reduction rate constants as they evolved into more alkaline environments.

Methods Summary

Thioredoxin sequences were retrieved from GenBank. Phylogenetic analysis and sequence reconstructions were performed using MrBayes, PAUP and PAML as previously described (Gaucher et al., Nature 451, 704-7 (2008)). The reconstructed sequences were synthesized, cloned into pQE80L vector and expressed in E. coli cells. Protein engineering and purification was carried as described in Wiita et al., Nature 450, 124-7 (2007). Thermal stabilities were measured using a VP-Capillary DSC calorimeter from MicroCal. The heat capacity vs. temperature profiles were analyzed following the two-state thermodynamic model (Ibarra-Molero et al., Biochemistry 38, 8138-49 (1999)). AFM experiments were performed in a custom-made apparatus in its force-clamp mode (Fernandez and Li, Science 303, 1674-8 (2004)). Silicon nitride cantilevers were used with a typical spring constant of 0.02 N/m. The buffer used in the experiments contained 10 mM HEPES, 150 mM NaCl, 1 mM EDTA, 2 mM NADPH, pH 7.2. Individual (I27_{G32C-A75C})₈ proteins are stretched at a constant force of 175-185 pN during 0.2-0.3 s. This pulse unfolds the modules up to the disulfide bond. The test-pulse force is then applied during several seconds to allow capturing all the possible reduction events. Trx reductase 50 nM (eukaryotic or bacterial) or DTE 200 μM was used to keep Trx enzymes in their reduced state. The traces containing reduction events are summated, normalized and fitted with a single exponential obtaining thus the reduction rate (r=1/π). A kinetic model containing two force-dependent rate constants was applied. The kinetic parameters were solved using matrix analysis and the errors were estimated using the bootstrap method. Igor software was used for data collection and analysis.

Phylogenetic Analysis and Ancestral Sequence Reconstruction.

A total of 203 thioredoxin sequences from the three domains of life were retrieved from GenBank (Table 1). Sequences were aligned using MUSCLE (Edgar, Nucleic Acids Res 32, 1792-7 (2004)) and further corrected manually. The phylogenetic analysis was carried out by the minimum evolution distance criterion with 1000 bootstrap replicates using PAUP* 4.0 beta. Ancestral sequences were reconstructed using PAML version 3.14 and incorporated the gamma distribution for variable replacement rates across sites (Yang, Comput Appl Biosci 13, 555-556 (1997)). For each site of the inferred sequences, posterior probabilities were calculated for all 20 amino acids. The amino acid residue with the highest posterior probability was then assigned at each site.

Protein Expression and Purification.

Genes encoding the ancestral Trxs enzymes were synthesized and codon-optimized for expression in E. coli cells. The genes were cloned into pQE80L vector (Qiagen) and transformed in E. coli BL21 (DE3) cells (Invitrogen). Cells were incubated overnight in LB medium at 37° C. and protein expression was induced with 1 mM IPTG. Cell pellets were sonicated and the His 6-tagged proteins were loaded onto His GraviTrap affinity column (GE Healthcare). The purified protein was verified by SDS-PAGE. The proteins were then loaded into PD-10 desalting column (GE Healthcare) and finally dialyzed against 50 mM HEPES, pH 7.0 buffer. The preparation of (I27_{G32C-A75C})₈ was carried out as follows: mutations Gly32Cys and Ala75Cys are introduced into the I27 module using the QuickChange site-directed mutagenesis protocol. Multi-step cloning was performed to produce an N-C-linked eight-domain polypeptide. The gene encoding the polypeptide was cloned into a pQE80L and the protein was expressed at 37° C. for 4 hours in E. coli BLR (DE3) cells. Cell pellet was lysed using a French press. The polypeptide with a His 6-tagged was purified using Talon-Co²⁺ resin. The protein was further purified by size exclusion chromatography on a Superdex 200 HR 10/30 column. The protein was eluted in 10 mM HEPES, 150 mM NaCl, 1 mM EDTA, pH 7.2.

DSC Experiments

Thermal stabilities of ancestral and modern Trx enzymes were measured with a VP-Capillary DSC (MicroCal). Protein solutions were dialyzed into a buffer of 50 mM HEPES, pH 7. The scan speed was set to 1.5 K/min. Several buffer-buffer baselines were first obtained for proper equilibration of the calorimeter. Concentrations were 0.3-0.7 mg/mL and were determined spectrophotometrically at 280 nm using theoretical extinction coefficients and molecular weights. The experimental traces were analyzed following the two-state thermodynamic model (Ibarra-Molero et al., Biochemistry 38, 8138-49 (1999)).

AFM Experiments

The atomic force microscope used is a custom-made design (Fernandez and Li, Science 303, 1674-8 (2004)). Data acquisition is controlled by two PCI cards 6052E and 6703 (National Instruments). Cantilever model MLCT of silicon nitride were used. We calibrate the cantilever using the equipartition theorem (Florin et al., Biosensors & Bioelectronics 10, 895-901 (1995)) giving rise to a typical spring constant of 0.02 N/m. The AFM works in the force-clamp mode with length resolution of 0.5 nm. The feedback response can reach 5 ms. The buffer used in the experiment is 10 mM HEPES, pH 7.2, 150 mM NaCl, 1 mM EDTA, 2 mM NADPH. Trx enzymes are added to a desired concentration. The buffer also contains Trx reductase 50 nM (prokaryotic or eukaryotic) to keep Trx enzymes in their reduced state. E. coli Trx reductase works well with bacterial-origin Trx enzymes whereas eukaryotic Trx reductase works with Archaea/Eukaryote Trx enzymes. Similar results are obtained when using DTE 200 μM to keep Trx enzymes reduced, thus demonstrating that modern Trx reductases maintain fully reduced ancestral Trx enzymes. For the experiments at pH 5, 20 mM sodium acetate buffer and 200 μM DTE was used.

To perform the experiment 3-6 μl of substrate at ˜0.1 mg/mL was deposited on a gold-covered coverslide. A drop of ˜100 μl containing the Trx solution was then added. The force-clamp protocol consists of three pulses of force. In the first pulse the cantilever tip was pressed against the surface at 800 pN for 2 s. In the second pulse the attached (I27_{G32C-A75C})₈ is stretched at 175-185 pN for 0.2-0.3 s. The third pulse is the test force where the reduction events are captured. This pulse is applied at different forces 30-500 pN time enough to capture all the possible reduction events.

The traces were collected and analyzed using custom-written software in Igor Pro 6.03. The traces containing the reduction events at each force were summated, normalized and fitted with a single exponential. From the fitting we can obtain a time constant, π, and thus the reduction rate at a given force (r=1/π). Bootstrapping method was used to obtain the error of the reduction rates. The bootstrapping was run 1000 times for each reduction rate obtaining a distribution from where the s.e.m. can be calculated.

AFM Data Analysis

The data were fitted following a three-state kinetic model previously described (Wiita et al., Nature 450, 124-7 (2007); Perez-Jimenez et al., Nat Struct Mol Biol 16, 890-6 (2009)). In this model three different chemical mechanisms are taken into account. The rate constants used in the kinetic model are:

k₀₁=α₀[Trx]

k₁₂=β₀exp(FΔx₁₂/k_{B}T)+λ₀

k₀₂=γ₀[Trx]exp(FΔx₀₂/k_{B}T)+λ₀

k₁₀=δ₀

Rate constants k₀₁ and kₒ₂ depend on Trx concentration in a linear manner. k₁₂ and k₀₂ exponentially depend on force. The kinetic model is solved using matrix analysis and parameters α₀, β₀, ΔX₁₂, γ₀, Δx₀₂, λ and δ₀ can be obtained for each ancestral enzyme. The optimal kinetic parameters are calculated by numerical optimization using the downhill simplex method (Nelder and Mead, Computer Journal 7, 308-313 (1965) (Table 2).

A brief explanation of the different chemical mechanisms is as follows: when the substrate is stretched at low force (below 200 pN) k₀₁ and k₁₂ dominate. The negative force dependence observed in all Trx enzymes (ancestral and modern) gives rise to a negative value of Δx₁₂. This is consistent with a shortening of the polypeptide chain. This shortening was explained by a force-inhibited rotation of the disulfide bond necessary for the correct alignment of the S—S bond (180°) for an S_{N}2 reaction to occur. This mechanism is similar to a Michaelis-Menten reaction in which the formation of an enzyme-substrate complex is crucial. A second reduction mechanism occurs at forces over 200 pN where k₀₂ dominates. In the case of bacterial-origin Trxs, the rate of reduction is exponentially accelerated. This is consistent with a simple S_{N}2 reaction with an elongation of the disulfide bond at the transition state, Δx₀₂. This elongation, ˜0.18 Å, is only observed in bacterial-origin Trxs. In the case of eukaryotic-origin Trxs the rate of disulfide bond reduction when the substrate is pulled at forces over 200 pN is essentially force-independent. In this case k₀₂=λ₀. This force-independent mechanism is explained by a single-electron transfer reaction accounted for the parameter λ₀ in the kinetic model. This mechanism seems to be ubiquitous to all Trx enzymes but is certainly remarkable in eukaryotic-origin Trxs. The origin of this diversity of chemical mechanisms was explained on the basis of the structural features of the binding groove (Perez-Jimenez et al., Nat Struct Mol Biol 16, 890-6 (2009)).

### Example 2

### Reconstruction of Ancient Thioredoxin Enzymes

Described herein is data demonstrating the feasibility of reconstructing ancient thioredoxin enzymes from predicted nodes. For example, the predicted DNA sequence of a Trx enzyme from the node corresponding to the Last Bacterial Common Ancestor, dated about 4 billion years ago, was selected for gene synthesis and protein expression in our laboratory (FIG. 4).

The resuscitated LBCA Trx showed a 26° C. higher denaturation temperature than that of modern E. coli Trx. Higher denaturation temperatures have also been reported for resuscitated elongation factor proteins (Gaucher et al., Nature, 2008. 451(7179): p. 704-U2; Gaucher et al., Nature, 2003. 425(6955): p. 285-8). The LBCA thioredoxin enzyme also showed a high rate of catalysis at pH 5, where extant enzymes are largely inactive (FIG. 4B). While this ancestral enzyme showed the typical biphasic force-dependent catalysis of the extant enzymes (Wiita, A. P., et al., Nature, 2007. 450(7166): p. 124-7), its peak activity was measured at 100 pN, suggesting a less well developed binding groove (FIG. 4C).

Results with the last bacterial common ancestor Trx enzyme show the feasibility of resurrecting active enzymes that disappeared from Earth millions of years ago. This approach can be used to uncover variations in the chemical mechanisms of thioredoxin catalysis (FIG. 4) and correlate them with the structure of these enzymes. These methods can also be used to generate one or more thioredoxin enzymes with characteristics not present in the extant enzymes (e.g. the absence of a binding groove).

The force-dependent rate of reduction shows that human Trx, which has a much deeper groove than that of E. coli, excludes the force accelerated mechanism of reduction (type III in FIG. 3C). In addition to depth and length, another characteristic of the binding groove that can be examined is the mean hydrophobicity per residue of a Trx enzyme. These parameters can be measured directly from over one hundred Trx structures currently available in PDB. Extreme examples of each groove parameter can be identified. These specific Trxs can be expressed to complete the force-spectroscopy experiments. The relative amplitude of each chemical mechanism of reduction measured using force spectroscopy, and the measured features of the binding groove can be correlated and calculated from the structure.

### Example 3

### Single Molecule Assays to Examine Other Bond Cleaving Enzymes

Any enzyme that cleaves covalent bonds can be investigated using the single molecule force spectroscopy assay described herein. Exemplary molecules that can be examined using the methods described herein include but are not limited to proteases. Proteases are a vast group of proteins with highly important physiological functions (Lopez-Otin and Bond, J Biol Chem, 2008. 283(45): p. 30433-7). The fact that their catalytic mechanisms have been thoroughly studied by traditional techniques facilitates interpretation of the single-molecule results (Frey and Hegeman, Enzymatic reaction mechanisms. 2007, Oxford: Oxford University Press). The high substrate specificity shown by some proteases can be used to design substrates suitable for single-molecule force spectroscopy. The proteolysis of those substrates can be studied under force. The catalytic activity of proteases will a complex force dependency because proteases have substrate-binding grooves that are similar to those found in thioredoxin enzymes and because the chemical mechanism of proteolysis can involve geometric rearrangements at the transition state (Frey and Hegeman, Enzymatic reaction mechanisms. 2007, Oxford: Oxford University Press). As in the case of thioredoxins, the molecular interpretation of the force dependency of proteases will shed light into the sub-Ångström contortions of the substrate atoms as they are cleaved by the protease during catalysis.

To determine the force-dependency of protease catalysis, an appropriate substrate that can detect single protease cleavage events will be constructed. Because simply cleaving the backbone of a mechanically stretched protein would be the end the experiment because the polypeptide would loose its mechanical continuity, a substrate which retains its mechanical integrity upon cleavage and which also extends sufficiently to provide an unmistakable fingerprint will be constructed.

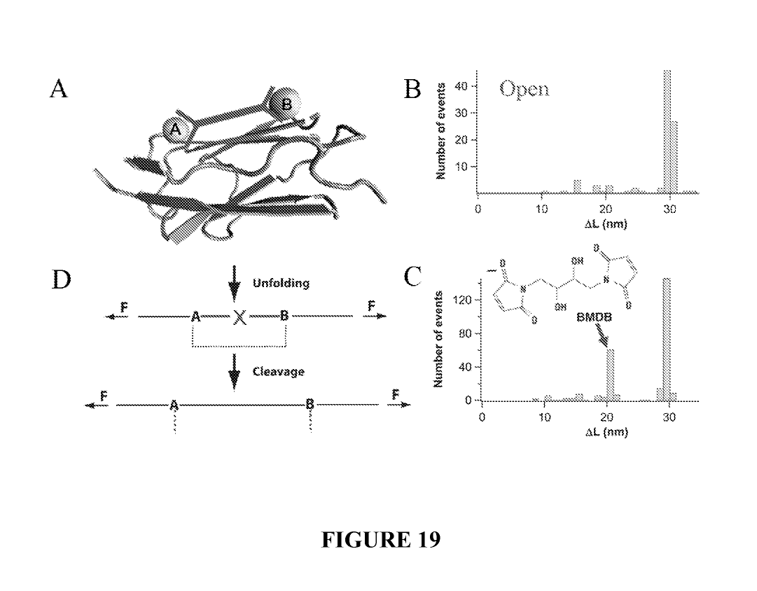

An exemplary substrate, as set forth in FIG. 19, can be designed by introducing two cysteines in a given protein (e.g. the I27 protein). The cysteines can be placed at a distance from one another so that they do not form a disulfide bond (residues A and B, FIG. 19A). The free cysteines can be used as specific conjugation points for a polypeptide containing the protease recognition sequence. The use of cysteines to specifically label proteins is commonplace in modern molecular biology (Wynn et al., Methods Enzymol, 1995. 251: p. 351-6; Crankshaw and Grant, Curr Protoc Protein Sci, 2001. Chapter 15: p. Unit 15 1; Corey, Methods Mol Biol, 2004. 283: p. 197-206). Typically, maleimide (Ji, Methods Enzymol, 1983. 91: p. 580-609) or sulfhydryl reagents (Cecconi et al., Eur Biophys J, 2008. 37(6): p. 729-38)) are employed. Indeed, a variety of bifunctional reagents (Green et al., Protein Sci, 2001. 10(7): p. 1293-304) can be employed to trap proteins in specific conformations (Milanesi et al., Biochemistry, 2008. 47(51): p. 13620-34; Cipriano et al., Proteins, 2008. 73(2): p. 458-67). Cysteine residues were introduced in positions 27 and 55 of the I27 protein (FIG. 19A), and bridged with the bifunctional reagent BMDB to creating a covalent bridge between positions 27 and 55 of the I27 protein. Mechanical unfolding of an I27 protein gives a normal extension of ΔL=29 nm (FIG. 19B). When mutant I27 proteins are reacted with the bifunctional reagent BMDB, the unfolding is now limited to only ΔL=20 nm due to the presence of a covalent bridge formed by the BMDB (FIG. 19C). Bifunctional bridges with short polypeptides that can be cleaved by a protease (e.g. enterokinase) can be created. For example, I27 polypeptides that serve as substrates for the enzyme enterokinase can be generated. This enzyme is readily available and of wide commercial use and specifically cleaves the sequence Asp-Asp-Asp-Asp-Lys-X after the Lys, as long as X is not a proline (Light and H. Janska Trends Biochem Sci, 1989. 14(3): p. 110-2). In such cases, cleavage of the covalent bridge will result into a further extension by ΔL=9 nm which uniquely identifies the cleavage reaction (FIG. 19D). As in the case of thioredoxin activity, the rate of appearance of the 9 nm steps measures the rate of catalysis at different forces.

Although the covalent bridge design works (FIGS. 19A, B, C), the efficiency of the bridging reaction is in the range of 30-40%, leaving open the remaining I27 proteins of a polypeptide. This limits the number of cleavage events that can be detected per polypeptide. A variety of additional bifunctional enterokinase substrates either with thiols or maleimides can be constructed and those that have the highest bridging efficiency can be selected for additional analysis.

Short polypeptides containing a cleavage sequence and terminated by either thiols or maleimides (to covalently link the short polypeptide to the exposed cysteines) can also be generated. Because the intra-molecular conjugation scheme described herein is also dependent on the distance between the reactive groups, the position of the exposed cysteines conjugating bifunctional reagents (recognition sequences) can be varied among different lengths until optimal constructs are identified. The force dependency of the catalytic activity of enterokinase can be studied using these substrates. Given that enterokinase contains a substrate-binding groove (Lu et al., J Mol Biol, 1999. 292(2): p. 361-73), and that the chemistry of proteolysis involves structural rearrangements of the participating atoms (e.g. formation of a tetrahedral intermediate), these substrates can be used to determine whether force exerts a complex effect on enterokinase activity. Once the force-dependency of protease catalysis is measured, kinetic models can be developed to explain the data. In particular, the measured force dependency can be used to formulate activity models as a series of chemical mechanisms that require bond rotations/elongation of the recognition sequence. The effect of width, depth and hydrophobicity of the binding groove can be studies as functions of the measured force dependent mechanisms. This approach can also be extended to study other specific proteases such as factor Xa and thrombin as well as the role of substrate conformations in enzymatic catalysis. This approach can also be important for the development of drug targets given the medical importance of protease inhibitors.

### Example 4

### A Single Molecule Assay for Thioredoxin Catalysis

An octamer of the I27 module can be mutated to incorporate two cysteine residues (G32C, A75C; FIG. 1, gold labeled residues). The two cysteine residues spontaneously form a stable disulfide bond that is buried in the β-sandwich fold of the I27 protein. This is polypeptide (I27_{S-S})₈. The disulfide bond mechanically separates the I27 protein into two parts (FIG. 1A). The unsequestered amino acids that readily unfold and extend under a stretching force are depicted in red. The blue region marks 43 amino acids which are trapped behind the disulfide bond (FIG. 1B) and can be extended if the disulfide bond is reduced by a nucleophile such as the enzyme Trx (FIG. 1C). Force-clamp AFM can be used to extend single (I27_{S-S})₈ polypeptides. The constant force causes individual I27 proteins in the chain to unfold, resulting in stepwise increases in length of the molecule following each unfolding event. After unfolding, the stretching force is directly applied to the now solvent exposed disulfide bond, and if a reducing agent is present in the bathing solution, the bond can be chemically reduced giving rise to a new stepwise increase in length of the polypeptide (FIG. 1D). The size of the step increases in length observed during these force clamp experiments corresponds to the number of amino acids released, serving as a precise fingerprint to identify the reduction events. The rate of disulfide bond reduction can be measured at a given force by fitting a single exponential to an ensemble average of many reduction traces (Wiita, A. P., et al., Nature, 2007. 450(7166): p. 124-7; Perez-Jimenez, et al., Nature Structural & Molecular Biology, 2009. 16(8): p. 890-U120; Ainavarapu et al., Journal of the American Chemical Society, 2008. 130(20): p. 6479-6487). FIG. 1E shows a plot of the rate of reduction as a function of force for experiments done in the presence of human Trx, E. Coli Trx and the simpler nucleophile L-Cysteine. From these data, at least three different types of force-dependencies can be distinguished. These force dependencies may be related to the particular arrangement of the substrate in the binding groove of the enzymes (Perez-Jimenez, et al., Nature Structural & Molecular Biology, 2009. 16(8): p. 890-U120). In the case of L-Cys, the force dependency can arise from the much simpler S_{N}2 arrangement of a simple nucleophile (Ainavarapu et al., Journal of the American Chemical Society, 2008. 130(20): p. 6479-6487; Wiita et al., Proc Natl Acad Sci USA, 2006. 103(19): p. 7222-7). The classical assays for disulfide bond reduction would only show bulk rates at zero force. The increased detail observed in the enzymatic mechanisms can now be interpreted at the molecular/atomic level (Perez-Jimenez, et al., Nature Structural & Molecular Biology, 2009. 16(8): p. 890-U120).

### Example 5

### Simultaneous Measurement of Association/Dissociation and Reduction Reactions in Single Thioredoxin Enzymes

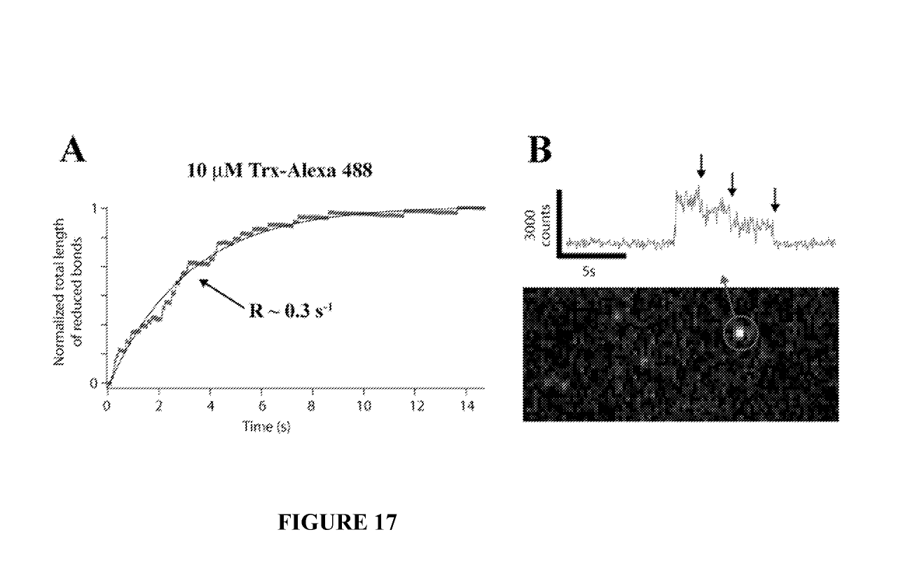

The methods described herein can be used to detect when the enzyme reduces a target disulfide bond. To determine when Trx enzymes bind to a substrate, how long it takes to reduce the substrate after binding and how long the enzyme remains attached to the substrate after the reduction event, force-clamp assays of disulfide bond reduction can be combined with single molecule fluorescence detection of enzyme binding to the exposed substrate using our newly developed AFM/TIRF instrument (FIG. 5). To observe enzymatic binding to a mechanically extended substrate, Trx enzymes can be labeled with a fluorophore (e.g. Alexa Fluor 488 fluorophore). Fluorophores, such as Alexa Fluor 488 dye, can readily be ligated to the exposed primary amines of a protein. A Trx enzyme may contain up to 12 lysine residues with varying degrees of exposure to the solvent. Force-clamp experiments show that labeled E. coli Trx enzymes are bright and reduce the substrate disulfide bonds at a rate of 0.3 s⁻¹, which is only about half of the rate measured with the unlabeled enzyme (FIG. 17A).

The labeled enzymes can be observed in the TIRF microscope. FIG. 17B shows a labeled enzyme visiting the evanescent field of a TIRF microscope driven by Brownian motion. Single enzymes are brightly fluorescent and can be monitored as a function of time using an efficient CCD camera (Andor Technology). These capabilities can be used to follow the binding and dissociation of labeled Trx enzymes interacting with their target disulfide bonds, while at the same time assaying their reduction using force-clamp spectroscopy. Such analysis can be used to measure directly the rates of association and dissociation of single enzymes as they bind and reduce single disulfide bonds in an extended protein. Data sets, such as those shown in FIG. 5B can be collected using the methods described herein. The methods described herein can also be used to determine whether the rates of association and dissociation are force-dependent and to refine simplified models of binding and reduction (Wiita, A. P., et al., Nature, 2007. 450(7166): p. 124-7).

The dissociation dwell time of the enzyme after a disulfide bond has been reduced can be measured from the combined AFM/TIRF experiments (FIG. 5). Since Trx is covalently linked to the substrate immediately after the catalytic reaction (Holmgren, A., Thioredoxin and glutaredoxin systems. J Biol Chem, 1989. 264(24): p. 13963-6), this dwell time depends on both an intermolecular reduction event and the off-rate of the non-covalently bound enzyme. As a control experiment, the WT Trx can be switched for a C35A mutant Trx that is redox active but incapable of detaching from the substrate after reducing it (Wynn et al., Methods Enzymol, 1995. 251: p. 351-6). In this case, the Trx enzyme catalyzing the reaction will remain stationary and visible in the evanescent excitation field until it is photobleached. Such methods can be used to capture the association and dissociation reaction of a single thioredoxin enzyme with its target during disulfide bond reduction. Every step involved in the activity of single thioredoxin enzymes can be separated and measured independently, allowing for the development of detailed kinetic model for this enzyme and the mechanisms by which it finds its target.

### Example 6

### Detecting the Oxidase Activity of Thioredoxin Enzymes

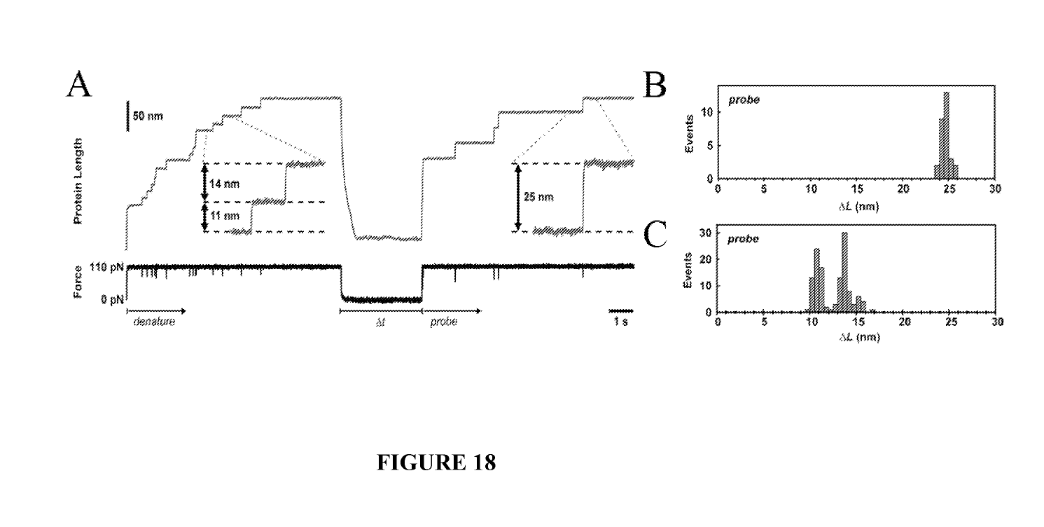

The single molecule assay described herein can also be used to study oxidative folding by thioredoxin enzymes. In vivo, thiol-disulfide exchange reactions are catalyzed by a number of enzymes belonging to the thioredoxin (Trx) superfamily. All of these enzymes share the thioredoxin fold and most feature a CXXC active site motif (Martin, Structure, 1995. 3(3): p. 245-50). In humans and other eukaryotes, thioredoxin catalyzes the cleavage of disulfide bonds whereas PDI enzymes catalyze their oxidation and isomerization. However the function of PDI as an oxidase is not unique given that, in S. cerevisiae, deletion strains lacking the essential gene encoding PDI can be rescued by a gene encoding for a simple thioredoxin C35S mutant (Chivers et al., EMBO J, 1996. 15(11): p. 2659-67). This thioredoxin variant has a CXXS active site, meaning that the conventional pathway for substrate reduction is not possible. In addition, PDI-like enzymes with CXXS active sites have also been shown to complement this yeast deletion strain (Tachibana et al., Mol Cell Biol, 1992. 12 (10): p. 4601-11; LaMantia et al., Cell, 1993. 74(5): p. 899-908). In certain aspects, the new single molecule oxidative folding assay described herein (e.g. FIG. 18) can be used to determine whether (1) the requirements for catalysis of oxidative folding are the same as those for disulfide bond reduction, (2) whether the C-terminal cysteine functions as a switch between these processes, and (3) the binding groove play a key role in oxidative folding.

As shown in FIG. 18, a protein made of eight disulfide bonded repeats (I27_{S-S}) can be picked up and stretched. In one embodiment, the protein is in a buffer containing 10 μM of wild type human Trx enzyme. The polypeptide is then exposed to a pulling force of 110-150 pN (denature), which results into a number of stepwise extensions. As shown in FIGS. 1A-1D, steps of 11 nm correspond to unfolding events where a single domain extends up to the disulfide bond. This exposes the disulfide and enables its reduction by the thioredoxin enzyme. Reduction of the disulfide in turn releases an additional 14 nm of the polypeptide chain. These precise step lengths serve as a fingerprint identifier that unambiguously verify these events. When all domains are unfolded and reduced, the force is switched off and the protein is allowed to refold for some time (Δt=5 s; FIG. 18). The force is then again switched on (probe) triggering again a series of stepwise elongations if any refolding had taken place. As soon as the force is switched on, folding is abruptly stopped and the folded status of each substrate domain can be probed at a time Δt after refolding was initiated. During the probe pulse, the protein extends in steps of 25 nm. This step size corresponds to the sum of unfolding (11 nm) and reduction (14 nm) steps and thus marks the unfolding of a domain without a formed disulfide bond. While not all the domains refolded during the folding period, none of the refolded domains formed a disulfide bond, indicating that the wild type form of thioredoxin does not catalyze reoxidation (FIG. 18B). By contrast if the experiment shown in FIGS. 18A, B is repeated in the presence of the C35S human thioredoxin mutant (hTrx^{C35S}), the step sizes during the probe pulse are now entirely composed of 11 and 14 nm steps, indicating the full reoxidation of all the disulfide bonds (FIG. 18C).

Shown in FIG. 18 is a demonstration of the sensitivity of the oxidative folding assay described herein. As shown, in FIG. 18, the assay enables detection that that the replacement of a single atom in the catalytic site of the enzyme (from sulfur to oxygen) in human thioredoxin is sufficient for hTrx to gain the oxidase function, in addition to keeping intact its reductase activity. These results explain why hTrx^{C35S} can rescue PDI deletion strains of S. cerevisiae (Chivers et al., EMBO J, 1996. 15(11): p. 2659-67).

To study the oxidase mechanisms of thioredoxin, the value of Δt can be varied in order to determine the rate of reoxidation by hTrx^{C35S}. The force dependency of the rate of reoxidation can be measured by quenching the force to different values during the folding/reoxidation period Δt. The methods described herein may also reveal a complex force dependency from substrate-enzyme interactions during oxidative folding. To study the role played by the binding groove in the reoxidation of the substrate, the C35S mutation will be engineered into E. coli thioredoxin enzymes. E. coli thioredoxin enzymes that have a much shallower groove than human Trx and show different mechanisms in its force dependency (FIG. 3). The properties of the binding groove can be an important factor in reoxidation. To determine whether the full folding of the host I27 protein is a necessary condition for reoxidation the number of 11 nm steps (unfolding of a natively folded protein) will be compared with the number of 14 nm steps (reduction of re-oxidized bonds) observed during the probe pulse (FIG. 18A). For example, if folding is not necessary, then there will be more steps of 14 nm than steps of 11 nm, etc. These results can be used to determine how the association and dissociation cycles are affected by the C35S mutation in single thioredoxin enzymes and to correlate the reoxidation events with the binding/unbinding reactions of fluorescently labeled Trx^{C35S} enzymes (FIG. 5). The combined folding/reoxidation assay shown in FIG. 18 together with experiments similar to those highlighted in FIG. 5, can be used to reveal the dynamics of a single thioredoxin enzyme as it oxidizes a target disulfide bond during the folding of the host protein.

The single molecule assays described herein have the ability to identify and separate the different stages of protein folding (Garcia-Manyes et al., PNAS, 2009. 106(26): p. 10534-10539; Garcia-Manyes et al., PNAS, 2009. 106(26): p. 10540-10545), and can thus be used to determine at what stage of folding a thioredoxin enzyme is capable of oxidizing a substrate. Although the finding described herein show that the human thioredoxin mutant hTrx^{C35S} gains oxidase activity, the methods described herein can also be used to determine whether the C35S mutation can have a similar effect on other members of the thioredoxin family with different groove depths.

### Example 7

### Other Activities of Resurrected Enzymes

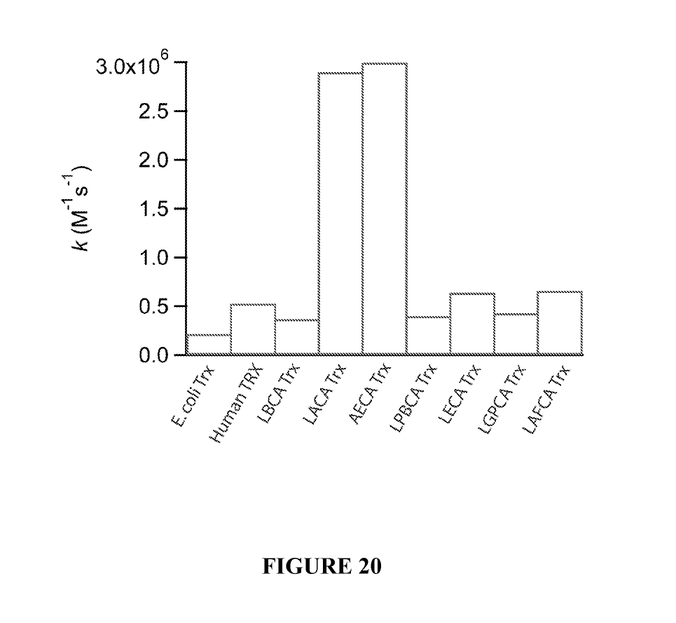

FIG. 20 shows the rate constants for disulfide bond reduction by ancestral and modern Trxs enzymes. Although these latter values are within the same range of those found in extant Trx enzymes using AFM (FIG. 14 and Perez-Jimenez et al., Nat Struct Mol Biol 16, 890-6 (2009)) and bulk experiments (Holmgren et al., J Biol Chem 254, 9113-9 (1979)), there was a trend in the reconstructed enzymes to show higher reduction rates at forces below 200 pN (FIG. 14). It is speculated that this trend may be related to substrate specificity of the enzymes. Ancient enzymes may be less substrate specific than modern ones, and therefore, might be more efficient with generic substrates such as those used herein.

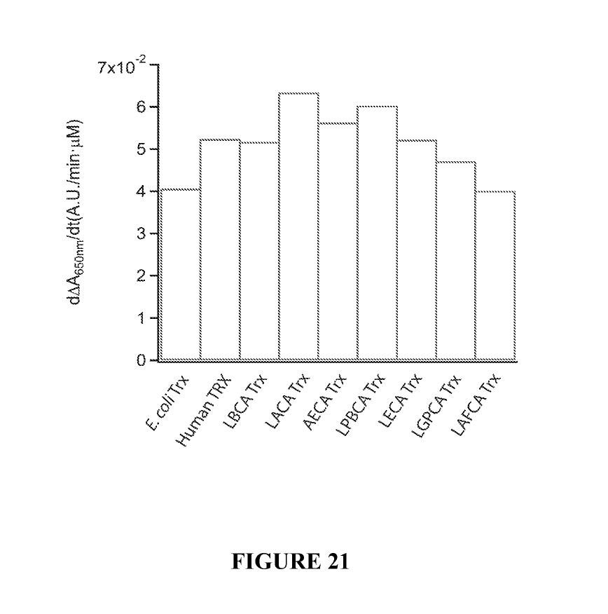

The activity of the ancestral enzymes was measured using the conventional insulin assay (FIG. 21) The values of insulin precipitation rates obtained with this assay are similar to those previously determined for E. coli Trx (Suarez, M. et al., Biophys Chem 147, 13-9 (2010); Holmgren, A., J Biol Chem 254, 9627-32 (1979)).

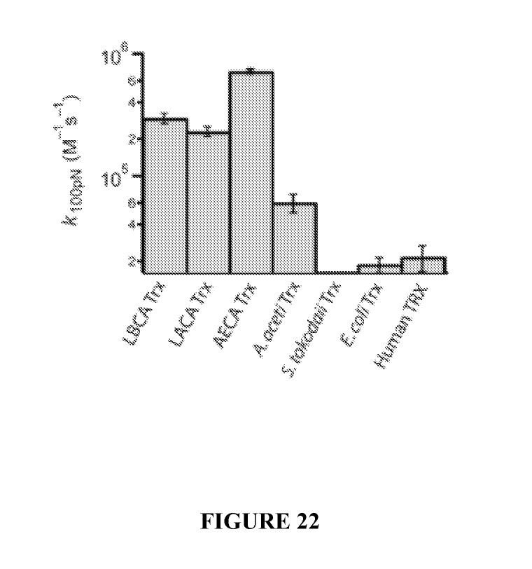

FIG. 22 shows a comparison of the rate of reduction measured at 100 pN for LBCA, LACA and AECA with the rates of some modern Trx enzymes also measured at pH 5.

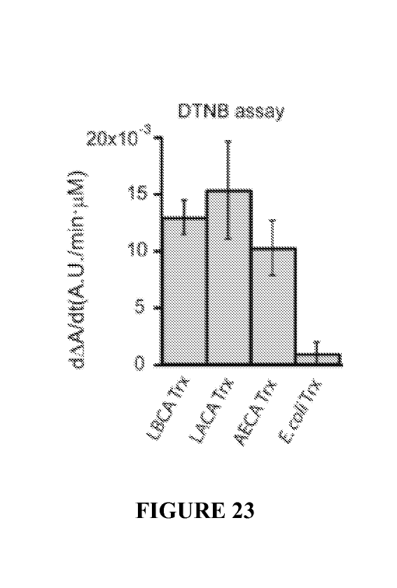

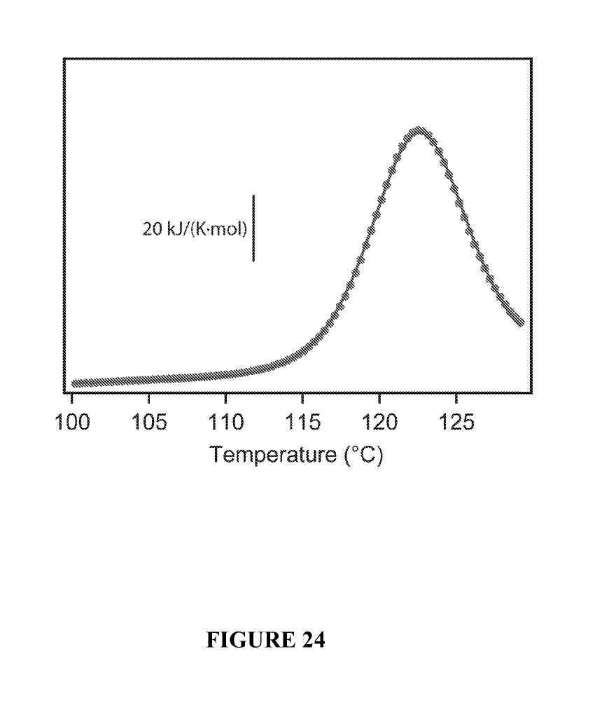

Due to spontaneous precipitation of insulin at pH below 6, DTNB was used as a substrate for disulfide reduction to further verify the ability of the oldest enzymes to work at pH 5 (FIG. 23). This analysis of reconstructed enzymes indicated that ancient Trx enzymes were well adapted to function under acidic conditions and that Trx enzymes could maintain similar reduction rate constants as they evolved in more alkaline environments. A feature of the thioredoxin family of enzymes is that many of them are secreted to the extracellular environment where most disulfide-bonded proteins are found (Xu, S. Z. et al., Nature 451, 69-72 (2008); Windle, H. J., Fox, A., Ni Eidhin, D. & Kelleher, D., J Biol Chem 275, 5081-9 (2000). From this perspective, thioredoxin enzymes are perhaps one of the few types of enzymes for which a correlate can be established between their pH sensitivity and the environmental conditions found outside cells (Xu, S. Z. et al., Nature 451, 69-72 (2008); Windle, H. J., Fox, A., Ni Eidhin, D. & Kelleher, D., J Biol Chem 275, 5081-9 (2000). It is informative to compare the acid tolerance of the resurrected enzymes with enzymes from extant extremophiles. For example, Trx from Sulfolobus tokodaii (thermophilic archaea (Ming, H. et al., Proteins 69, 204-8 (2007)), with a melting temperature of 122° C. (FIG. 24), is active at pH 7 (0.12×10⁵ M⁻¹ s⁻¹ at 50 pN), but does not show detectable activity at pH 5 (FIG. 22) which is not surprising given that Sulfolobus regulates its cytosolic pH (Baker-Austin, C. & Dopson, M., Trends Microbiol 15, 165-71 (2007). By contrast, Trx from Acetobacter aceti (acidophilic bacteria (Starks, C. M., Francois, J. A., MacArthur, K. M., Heard, B. Z. & Kappock, T. J. Protein Sci 16, 92-8 (2007) that grows at pH 4) is active at pH 5 (0.6×10⁵ M⁻¹ s⁻¹ at 100 pN), reflecting its acidic cytosol (Starks, C. M., Francois, J. A., MacArthur, K. M., Heard, B. Z. & Kappock, T. J., Protein Sci 16, 92-8 (2007); Menzel, U. & Gottschalk, G., Archives of Microbiology 143, 47-51 (1985).

Method Summary

Thioredoxin bulk enzymatic measurements. Bulk-solvent oxidoreductase activity for ancestral thioredoxins was determined using the insulin precipitation assay as described (Suarez, M. et al., Biophys Chem 147, 13-9 (2010); Holmgren, A., J Biol Chem 254, 9627-32 (1979); Perez-Jimenez et al., J. Biol. Chem., 283: 27121-27129 (2008)). In order to further verify the activity of ancestral Trxs enzymes at acidic pH, DTNB (5,5′-dithiobis-(2-nitrobenzoic acid)) was used as a substrate at pH 5. In this assay, Trxs enzymes were preactivated by incubation with 1 mM DTT. The reaction was initiated by adding active Trx to a final concentration of 4 μM to the cuvette containing 1 mM DTNB in 20 mM sodium acetate buffer, pH 5. Change in absorbance at 412 nm due to the formation of TNB was followed during 1 min. Activity was determined from the slope dΔA₄₁₂/dt. A control experiment lacking Trx was registered and subtracted as baseline.

### Example 8

### Crystal Structure of Ancestral Enzyme Thioredoxin AECA

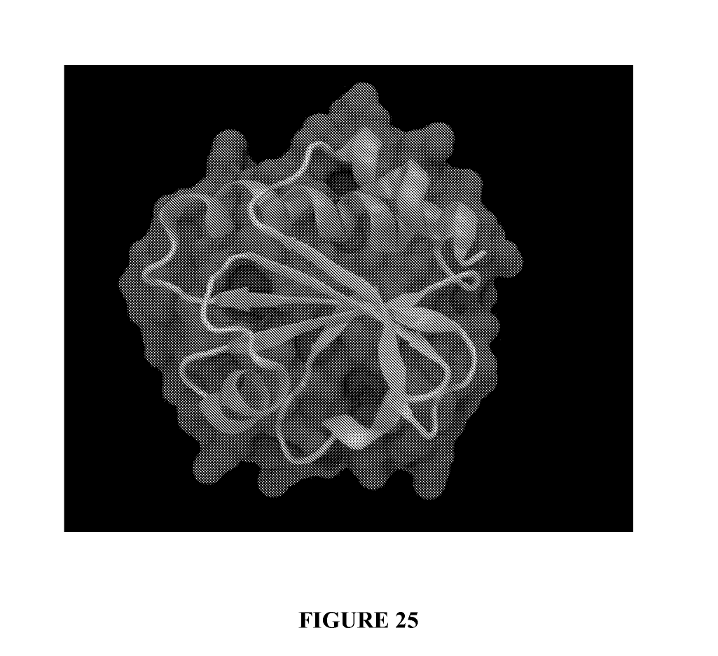

The crystal structure of the ancestral enzyme thioredoxin AECA is depicted in FIG. 25.

TABLE 3
Refinement Summary of Crystal Structure of Ancestral Enzyme
Thioredoxin AECA

| | |
|---|---|
| REMARK | ********************REFINEMENT SUMMARY: QUICK FACTS ******************* |
| REMARK | Start: r_work = 0.3754 r_free = 0.3753 bonds = 0.001 angles = 0.295 |
| REMARK | Final: r_work = 0.2284 r_free = 0.3032 bonds = 0.009 angles = 1.278 |
| REMARK | ************************************************************************ |
| REMARK | |
| REMARK | Rigid body refinement target: auto |
| REMARK | Information about total rigid body shift of selected groups: |

| | | |
|---|---|---|
| REMARK | rotation (deg) | translation |
| (A) | | |

| | | | | |
|---|---|---|---|---|
| REMARK | xyz | total | xyz | total |

| REMARK | group 1: 0.066 −0.035 0.032 0.08 0.02 −2.19 0.01 2.19 | |
|---|---|---|
| REMARK | ****************** REFINEMENT STATISTICS STEP BY STEP ****************** | |
| REMARK | leading digit, like 1_, means number of macro-cycle | |
| REMARK | 0: | statistics at the very beginning when nothing is done yet |
| REMARK | 1_bss: | bulk solvent correction and/or (anisotropic) scaling |
| REMARK | 1_xyz: | refinement of coordinates |
| REMARK | 1_adp: | refinement of ADPs (Atomic Displacement Parameters) |
| REMARK | 1_sar: | simulated annealing refinement of x, y, z |
| REMARK | 1_wat: | ordered solvent update (add/remove) |
| REMARK | 1_rbr: | rigid body refinement |
| REMARK | 1_gbr: | group B-factor refinement |
| REMARK | 1_occ: | refinement of occupancies |

| REMARK | ------------------------------------------------------------------------ | | | | | | | | |
|---|---|---|---|---|---|---|---|---|---|
| REMARK | R-factors, x-ray target values and norm of gradient of x-ray target | | | | | | | | |
| REMARK | stage | r-work | r-free | xray_target_w | | xray_target_t | | | |
| REMARK | 0: | 0.4439 | 0.4580 | 3.996716e+00 | | 4.056539e+00 | | | |
| REMARK | 1_bss: | 0.3754 | 0.3753 | 3.859379e+00 | | 3.906067e+00 | | | |
| REMARK | 1_rbr: | 0.3755 | 0.3716 | 3.857346e+00 | | 3.906505e+00 | | | |
| REMARK | 1_bss: | 0.3754 | 0.3715 | 3.856606e+00 | | 3.901563e+00 | | | |
| REMARK | 1_fit: | 0.3543 | 0.3590 | 3.816897e+00 | | 3.875086e+00 | | | |
| REMARK | 1_xyz: | 0.2973 | 0.3436 | 3.698549e+00 | | 3.866009e+00 | | | |
| REMARK | 1_adp: | 0.2655 | 0.3319 | 3.619059e+00 | | 3.822724e+00 | | | |
| REMARK | 1_occ: | 0.2694 | 0.3249 | 3.623934e+00 | | 3.822419e+00 | | | |
| REMARK | 2_bss: | 0.2644 | 0.3224 | 3.616364e+00 | | 3.822741e+00 | | | |
| REMARK | 2_sar: | 0.2463 | 0.3110 | 3.584169e+00 | | 3.805693e+00 | | | |
| REMARK | 2_fit: | 0.2584 | 0.3092 | 3.610219e+00 | | 3.800907e+00 | | | |
| REMARK | 2_xyz: | 0.2367 | 0.3132 | 3.560169e+00 | | 3.782447e+00 | | | |
| REMARK | 2_adp: | 0.2325 | 0.3113 | 3.548015e+00 | | 3.770248e+00 | | | |
| REMARK | 2_occ: | 0.2325 | 0.3113 | 3.548015e+00 | | 3.770248e+00 | | | |
| REMARK | 3_bss: | 0.2308 | 0.3100 | 3.546772e+00 | | 3.769120e+00 | | | |
| REMARK | 3_fit: | 0.2364 | 0.3034 | 3.546495e+00 | | 3.757393e+00 | | | |
| REMARK | 3_xyz: | 0.2281 | 0.3097 | 3.544193e+00 | | 3.770737e+00 | | | |
| REMARK | 3_adp: | 0.2281 | 0.3080 | 3.542285e+00 | | 3.767369e+00 | | | |
| REMARK | 3_occ: | 0.2281 | 0.3080 | 3.542285e+00 | | 3.767369e+00 | | | |
| REMARK | 3_bss: | 0.2284 | 0.3032 | 3.536917e+00 | | 3.762045e+00 | | | |
| REMARK | ------------------------------------------------------------------------ | | | | | | | | |
| REMARK | stage | k_sol | b_sol | b11 | b22 | b33 | b12 | b13 | b23 |
| REMARK | 0: | 0.000 | 0.000 | 0.000 | 0.000 | 0.000 | 0.000 | 0.000 | 0.000 |
| REMARK | 1_bss: | 0.277 | 22.223 | −8.643 | 18.065 | −9.422 | 0.000 | −7.697 | −0.000 |
| REMARK | 1_rbr: | 0.278 | 22.363 | −8.344 | 18.129 | −9.095 | 0.000 | −7.927 | 0.000 |
| REMARK | 1_bss: | 0.278 | 22.363 | −8.344 | 18.129 | −9.095 | 0.000 | −7.927 | 0.000 |
| REMARK | 1_fit: | 0.278 | 22.363 | −8.344 | 18.129 | −9.095 | 0.000 | −7.927 | 0.000 |
| REMARK | 1_xyz: | 0.278 | 22.363 | −8.344 | 18.129 | −9.095 | 0.000 | −7.927 | 0.000 |
| REMARK | 1_adp: | 0.278 | 22.363 | −8.344 | 18.129 | −9.095 | 0.000 | −7.927 | 0.000 |
| REMARK | 1_occ: | 0.278 | 22.363 | −8.344 | 18.129 | −9.095 | 0.000 | −7.927 | 0.000 |
| REMARK | 2_bss: | 0.288 | 39.551 | −7.348 | 16.799 | −9.450 | 0.000 | −6.425 | 0.000 |
| REMARK | 2_sar: | 0.288 | 39.551 | −7.348 | 16.799 | −9.450 | 0.000 | −6.425 | 0.000 |
| REMARK | 2_fit: | 0.288 | 39.551 | −7.348 | 16.799 | −9.450 | 0.000 | −6.425 | 0.000 |
| REMARK | 2_xyz: | 0.288 | 39.551 | −7.348 | 16.799 | −9.450 | 0.000 | −6.425 | 0.000 |
| REMARK | 2_adp: | 0.288 | 39.551 | −7.348 | 16.799 | −9.450 | 0.000 | −6.425 | 0.000 |
| REMARK | 2_occ: | 0.288 | 39.551 | −7.348 | 16.799 | −9.450 | 0.000 | −6.425 | 0.000 |
| REMARK | 3_bss: | 0.285 | 38.678 | −7.408 | 16.497 | −9.089 | 0.000 | −5.848 | 0.000 |
| REMARK | 3_fit: | 0.285 | 38.678 | −7.408 | 16.497 | −9.089 | 0.000 | −5.848 | 0.000 |
| REMARK | 3_xyz: | 0.285 | 38.678 | −7.408 | 16.497 | −9.089 | 0.000 | −5.848 | 0.000 |
| REMARK | 3_adp: | 0.285 | 38.678 | −7.408 | 16.497 | −9.089 | 0.000 | −5.848 | 0.000 |
| REMARK | 3_occ: | 0.285 | 38.678 | −7.408 | 16.497 | −9.089 | 0.000 | −5.848 | 0.000 |
| REMARK | 3_bss: | 0.288 | 38.710 | −7.489 | 16.446 | −8.957 | 0.000 | −5.560 | 0.000 |

| REMARK | ------------------------------------------------------------------------ | | | | |
|---|---|---|---|---|---|
| REMARK | stage | <pher> | fom | alpha | beta |
| REMARK | 0: | 60.971 | 0.3860 | 0.1586 | 1494.490 |
| REMARK | 1_bss: | 46.439 | 0.5699 | 0.2434 | 752.240 |
| REMARK | 1_rbr: | 47.128 | 0.5609 | 0.2407 | 740.986 |
| REMARK | 1_bss: | 46.772 | 0.5654 | 0.2416 | 735.017 |
| REMARK | 1_fit: | 45.218 | 0.5844 | 0.2462 | 662.267 |
| REMARK | 1_xyz: | 43.306 | 0.6062 | 0.2590 | 574.043 |
| REMARK | 1_adp: | 42.094 | 0.6195 | 0.2520 | 517.705 |
| REMARK | 1_occ: | 41.821 | 0.6230 | 0.2563 | 519.536 |
| REMARK | 2_bss: | 41.744 | 0.6240 | 0.2583 | 520.022 |
| REMARK | 2_sar: | 40.876 | 0.6348 | 0.2599 | 513.188 |
| REMARK | 2_fit: | 41.440 | 0.6281 | 0.2520 | 517.930 |
| REMARK | 2_xyz: | 39.351 | 0.6534 | 0.2654 | 487.068 |
| REMARK | 2_adp: | 38.723 | 0.6608 | 0.2688 | 470.384 |
| REMARK | 2_occ: | 38.723 | 0.6608 | 0.2688 | 470.384 |
| REMARK | 3_bss: | 38.607 | 0.6623 | 0.2642 | 464.464 |
| REMARK | 3_fit: | 37.684 | 0.6736 | 0.2655 | 444.291 |
| REMARK | 3_xyz: | 38.693 | 0.6609 | 0.2638 | 456.285 |
| REMARK | 3_adp: | 38.508 | 0.6631 | 0.2638 | 447.088 |
| REMARK | 3_occ: | 38.508 | 0.6631 | 0.2638 | 447.088 |
| REMARK | 3_bss: | 38.000 | 0.6692 | 0.2653 | 431.262 |

| REMARK | ------------------------------------------------------------------------ | | | | | | | |
|---|---|---|---|---|---|---|---|---|
| REMARK | stage | angl | bond | chir | dihe | plan | repu | geom_target |
| REMARK | 0: | 0.295 | 0.001 | 0.015 | 6.225 | 0.001 | 4.112 | 1.3106e−02 |
| REMARK | 1_bss: | 0.295 | 0.001 | 0.015 | 6.225 | 0.001 | 4.112 | 1.3106e−02 |
| REMARK | 1_rbr: | 0.295 | 0.001 | 0.015 | 6.225 | 0.001 | 4.112 | 1.3115e−02 |
| REMARK | 1_bss: | 0.295 | 0.001 | 0.015 | 6.225 | 0.001 | 4.112 | 1.3115e−02 |
| REMARK | 1_fit: | 1.503 | 0.051 | 0.100 | 13.094 | 0.011 | 4.094 | 9.7914e−01 |
| REMARK | 1_xyz: | 1.386 | 0.012 | 0.087 | 15.419 | 0.006 | 4.124 | 1.4517e−01 |
| REMARK | 1_adp: | 1.386 | 0.012 | 0.087 | 15.419 | 0.006 | 4.124 | 1.4517e−01 |
| REMARK | 1_occ: | 1.386 | 0.012 | 0.087 | 15.419 | 0.006 | 4.124 | 1.4517e−01 |
| REMARK | 2_bss: | 1.386 | 0.012 | 0.087 | 15.419 | 0.006 | 4.124 | 1.4517e−01 |
| REMARK | 2_sar: | 1.599 | 0.017 | 0.103 | 17.177 | 0.007 | 4.104 | 2.0875e−01 |
| REMARK | 2_fit: | 1.663 | 0.026 | 0.115 | 17.316 | 0.008 | 4.100 | 3.0113e−01 |
| REMARK | 2_xyz: | 1.293 | 0.010 | 0.085 | 18.112 | 0.006 | 4.121 | 1.3492e−01 |
| REMARK | 2_adp: | 1.293 | 0.010 | 0.085 | 18.112 | 0.006 | 4.121 | 1.3492e−01 |
| REMARK | 2_occ: | 1.293 | 0.010 | 0.085 | 18.112 | 0.006 | 4.121 | 1.3492e−01 |
| REMARK | 3_bss: | 1.293 | 0.010 | 0.085 | 18.112 | 0.006 | 4.121 | 1.3492e−01 |
| REMARK | 3_fit: | 1.371 | 0.031 | 0.094 | 18.520 | 0.007 | 4.105 | 4.1601e−01 |
| REMARK | 3_xyz: | 1.278 | 0.009 | 0.083 | 18.495 | 0.005 | 4.117 | 1.3388e−01 |
| REMARK | 3_adp: | 1.278 | 0.009 | 0.083 | 18.495 | 0.005 | 4.117 | 1.3388e−01 |
| REMARK | 3_occ: | 1.278 | 0.009 | 0.083 | 18.495 | 0.005 | 4.117 | 1.3388e−01 |
| REMARK | 3_bss: | 1.278 | 0.009 | 0.083 | 18.495 | 0.005 | 4.107 | 1.3469e−01 |

| | |
|---|---|
| REMARK | ------------------------------------------------------------------------ |

| | |
|---|---|
| REMARK | Maximal deviations: |

| | | | | | | | | |
|---|---|---|---|---|---|---|---|---|
| REMARK | stage | angl | bond | chir | dihe | plan | repu | \|grad\| |
| REMARK | 0: | 4.887 | 0.010 | 0.095 | 73.272 | 0.012 | 2.601 | 1.0739e−02 |
| REMARK | 1_bss: | 4.887 | 0.010 | 0.095 | 73.272 | 0.012 | 2.601 | 1.0739e−02 |
| REMARK | 1_rbr: | 4.887 | 0.010 | 0.095 | 73.272 | 0.012 | 2.601 | 1.0756e−02 |
| REMARK | 1_bss: | 4.887 | 0.010 | 0.095 | 73.272 | 0.012 | 2.601 | 1.0756e−02 |
| REMARK | 1_fit: | 26.860 | 1.956 | 0.443 | 89.447 | 0.084 | 0.469 | 7.0512e−01 |
| REMARK | 1_xyz: | 9.306 | 0.066 | 0.366 | 89.866 | 0.061 | 2.069 | 8.9770e−02 |
| REMARK | 1_adp: | 9.306 | 0.066 | 0.366 | 89.866 | 0.061 | 2.069 | 8.9770e−02 |
| REMARK | 1_occ: | 9.306 | 0.066 | 0.366 | 89.866 | 0.061 | 2.069 | 8.9770e−02 |
| REMARK | 2_bss: | 9.306 | 0.066 | 0.366 | 89.866 | 0.061 | 2.069 | 8.9770e−02 |
| REMARK | 2_sar: | 12.521 | 0.195 | 0.728 | 83.560 | 0.052 | 2.138 | 2.7433e−01 |
| REMARK | 2_fit: | 12.521 | 0.578 | 0.728 | 83.560 | 0.052 | 1.860 | 3.1580e−01 |
| REMARK | 2_xyz: | 11.987 | 0.076 | 0.408 | 83.948 | 0.055 | 2.214 | 7.0435e−02 |
| REMARK | 2_adp: | 11.987 | 0.076 | 0.408 | 83.948 | 0.055 | 2.214 | 7.0435e−02 |
| REMARK | 2_occ: | 11.987 | 0.076 | 0.408 | 83.948 | 0.055 | 2.214 | 7.0435e−02 |
| REMARK | 3_bss: | 11.987 | 0.076 | 0.408 | 83.948 | 0.055 | 2.214 | 7.0435e−02 |
| REMARK | 3_fit: | 11.987 | 1.408 | 0.458 | 87.325 | 0.055 | 0.460 | 3.6860e−01 |
| REMARK | 3_xyz: | 12.449 | 0.051 | 0.412 | 85.180 | 0.047 | 2.165 | 6.7907e−02 |
| REMARK | 3_adp: | 12.449 | 0.051 | 0.412 | 85.180 | 0.047 | 2.165 | 6.7907e−02 |
| REMARK | 3_occ: | 12.449 | 0.051 | 0.412 | 85.180 | 0.047 | 2.165 | 6.7907e−02 |
| REMARK | 3_bss: | 12.449 | 0.051 | 0.412 | 85.180 | 0.047 | 2.165 | 6.8308e−02 |

| REMARK | ------------------------------------------------------------------------ | | | | | | | | | |
|---|---|---|---|---|---|---|---|---|---|---|
| REMARK | \|-----overall-----\|---macromolecule----\|------solvent-------\| | | | | | | | | | |
| REMARK | stage | b_max | b_min | b_ave | b_max | b_min | b_ave | b_max | b_min | b_ave |
| REMARK | 0: | 88.25 | 20.00 | 56.09 | 163.22 | 25.82 | 69.41 | 78.07 | 44.85 | 59.47 |
| REMARK | 1_bss: | 99.53 | 31.28 | 67.37 | 163.22 | 25.82 | 69.41 | 78.07 | 44.85 | 59.47 |
| REMARK | 1_rbr: | 99.53 | 31.28 | 67.37 | 163.22 | 25.82 | 69.41 | 78.07 | 44.85 | 59.47 |
| REMARK | 1_bss: | 99.53 | 31.28 | 67.37 | 173.50 | 27.46 | 69.36 | 98.66 | 39.86 | 55.15 |
| REMARK | 1_fit: | 99.53 | 31.28 | 67.37 | 173.50 | 27.46 | 69.36 | 98.66 | 39.86 | 55.15 |
| REMARK | 1_xyz: | 99.53 | 31.28 | 67.37 | 172.62 | 26.59 | 68.49 | 97.79 | 38.99 | 54.28 |
| REMARK | 1_adp: | 163.67 | 26.26 | 69.86 | 172.62 | 26.59 | 68.49 | 66.68 | 35.89 | 52.10 |
| REMARK | 1_occ: | 163.67 | 26.26 | 69.86 | 172.62 | 26.59 | 68.49 | 66.68 | 35.89 | 52.10 |
| REMARK | 2_bss: | 163.22 | 25.82 | 69.41 | 174.53 | 26.64 | 68.49 | 61.07 | 38.80 | 50.33 |
| REMARK | 2_sar: | 163.22 | 25.82 | 69.37 | 174.53 | 26.64 | 68.49 | 61.07 | 38.80 | 50.33 |
| REMARK | 2_fit: | 163.22 | 25.82 | 69.37 | 174.56 | 26.67 | 68.52 | 61.10 | 38.83 | 46.66 |

| REMARK | ------------------------------------------------------------------------ | |
|---|---|---|
| REMARK | stage | Deviation of refined |
| REMARK | | model from start model |

| | | | | |
|---|---|---|---|---|
| REMARK | | max | min | mean |
| REMARK | 0: | 0.000 | 0.000 | 0.000 |
| REMARK | 1_bss: | 0.000 | 0.000 | 0.000 |
| REMARK | 1_rbr: | 2.222 | 2.150 | 2.188 |
| REMARK | 1_bss: | 2.222 | 2.150 | 2.188 |
| REMARK | 1_fit: | 8.290 | 0.420 | 2.307 |
| REMARK | 1_xyz: | 8.288 | 0.507 | 2.324 |
| REMARK | 1_adp: | 8.288 | 0.507 | 2.324 |
| REMARK | 1_occ: | 8.288 | 0.507 | 2.324 |
| REMARK | 2_bss: | 8.288 | 0.507 | 2.324 |
| REMARK | 2_sar: | 9.001 | 0.493 | 2.411 |
| REMARK | 2_fit: | 9.001 | 0.480 | 2.412 |
| REMARK | 2_xyz: | 9.178 | 0.518 | 2.422 |
| REMARK | 2_adp: | 9.178 | 0.518 | 2.422 |
| REMARK | 2_occ: | 9.178 | 0.518 | 2.422 |
| REMARK | 3_bss: | 9.178 | 0.518 | 2.422 |
| REMARK | 3_fit: | 9.178 | 0.518 | 2.423 |
| REMARK | 3_xyz: | 9.175 | 0.593 | 2.433 |
| REMARK | 3_adp: | 9.175 | 0.593 | 2.433 |
| REMARK | 3_occ: | 9.175 | 0.593 | 2.433 |
| REMARK | 3_bss: | 9.175 | 0.593 | 2.433 |

| REMARK | ------------------------------------------------------------------------ | |
|---|---|---|
| REMARK | stage number of ordered solvent | |
| REMARK | 0: | 0 |
| REMARK | 1_bss: | 0 |
| REMARK | 1_rbr: | 0 |
| REMARK | 1_bss: | 0 |
| REMARK | 1_fit: | 0 |
| REMARK | 1_xyz: | 0 |
| REMARK | 1_adp: | 0 |
| REMARK | 1_occ: | 0 |
| REMARK | 2_bss: | 0 |
| REMARK | 2_sar: | 11 |
| REMARK | 2_fit: | 11 |
| REMARK | 2_xyz: | 11 |
| REMARK | 2_adp: | 11 |
| REMARK | 2_occ: | 11 |
| REMARK | 3_bss: | 11 |
| REMARK | 3_fit: | 8 |
| REMARK | 3_xyz: | 8 |
| REMARK | 3_adp: | 8 |
| REMARK | 3_occ: | 8 |
| REMARK | 3_bss: | 5 |

| REMARK | ------------------------------------------------------------------------ | | |
|---|---|---|---|
| REMARK | MODEL CONTENT. | | |
| REMARK | ELEMENT | ATOM RECORD COUNT | OCCUPANCY SUM |
| REMARK | C | 1640 | 1621.00 |
| REMARK | S | 12 | 12.00 |
| REMARK | O | 470 | 464.00 |
| REMARK | N | 402 | 397.00 |
| REMARK | TOTAL | 2524 | 2494.00 |

| REMARK | ------------------------------------------------------------------------ | | |
|---|---|---|---|
| REMARK | r_free_flags.md5.hexdigest 130536f97c5a634b1e93427cc8887f1a | | |
| REMARK | | | |
| REMARK | 3 | REFINEMENT. | |
| REMARK | 3 | PROGRAM | :PHENIX (phenix.refine: 1.6.1_357) |
| REMARK | 3 | AUTHORS | :Adams, Afonine, Chen, Davis, Echols, Gopal, |
| REMARK | 3 | | :Grosse-Kunstleve, Headd, Hung, Immormino, Ioerger, McCoy, |
| REMARK | 3 | | :McKee, Moriarty, Pai, Read, Richardson, Richardson, Romo, |
| REMARK | 3 | | :Sacchettini, Sauter, Smith, Storoni, Terwilliger, Zwart |
| REMARK | 3 | | |

| REMARK | 3 | REFINEMENT TARGET: ML | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS): | 2.485 |
| REMARK | 3 | RESOLUTION RANGE LOW | (ANGSTROMS): | 45.444 |

| REMARK | 3 | MIN(FOBS/SIGMA_FOBS): | | 0.01 |
|---|---|---|---|---|
| REMARK | 3 | COMPLETENESS FOR RANGE | (%): | 91.17 |

| | | | |
|---|---|---|---|
| REMARK | 3 | NUMBER OF REFLECTIONS: | 10755 |
| REMARK | 3 | | |

| REMARK | 3 | FIT TO DATA USED IN REFINEMENT. | | |
|---|---|---|---|---|
| REMARK | 3 | R VALUE | (WORKING + TEST SET): | 0.2322 |
| REMARK | 3 | R VALUE | (WORKING SET): | 0.2284 |

| REMARK | 3 | FREE R VALUE: | | 0.3032 |
|---|---|---|---|---|
| REMARK | 3 | FREE R VALUE TEST SET SIZE | (%): | 4.71 |

| | | | |
|---|---|---|---|
| REMARK | 3 | FREE R VALUE TEST SET COUNT: | 507 |
| REMARK | 3 | | |

| | | |
|---|---|---|
| REMARK | 3 | FIT TO DATA USED IN REFINEMENT (IN BINS). |

| | | | | | | | | |
|---|---|---|---|---|---|---|---|---|
| REMARK | 3 | BIN | RESOLUTION RANGE | COMPL. | NWORK | NFREE | RWORK | RFREE |
| REMARK | 3 | 1 | 45.4520-3.9441 | 0.98 | 2804 | 151 | 0.1900 | 0.2596 |
| REMARK | 3 | 2 | 3.9441-3.1308 | 0.97 | 2713 | 132 | 0.2293 | 0.3307 |
| REMARK | 3 | 3 | 3.1308-2.7351 | 0.89 | 2519 | 110 | 0.2902 | 0.3834 |
| REMARK | 3 | 4 | 2.7351-2.4850 | 0.80 | 2212 | 114 | 0.3442 | 0.4128 |
| REMARK | 3 | | | | | | | |

| REMARK | 3 | BULK SOLVENT MODELLING. | |
|---|---|---|---|
| REMARK | 3 | METHOD USED: | FLAT BULK SOLVENT MODEL |
| REMARK | 3 | SOLVENT RADIUS: | 1.11 |
| REMARK | 3 | SHRINKAGE RADIUS: | 0.90 |
| REMARK | 3 | GRID STEP FACTOR: | 4.00 |
| REMARK | 3 | K_SOL: | 0.288 |
| REMARK | 3 | B_SOL: | 38.710 |
| REMARK | 3 | | |

| REMARK | 3 | ERROR ESTIMATES. | |
|---|---|---|---|
| REMARK | 3 | COORDINATE ERROR (MAXIMUM-LIKELIHOOD BASED): | 0.45 |
| REMARK | 3 | PHASE ERROR (DEGREES, MAXIMUM-LIKELIHOOD BASED): | 38.00 |
| REMARK | 3 | | |

| REMARK | 3 | OVERALL SCALE FACTORS. | |
|---|---|---|---|
| REMARK | 3 | SCALE = SUM(\|F_OBS\|*\|F_MODEL\|)/SUM(\|F_MODEL\|**2): 0.3065 | |
| REMARK | 3 | ANISOTROPIC SCALE MATRIX ELEMENTS (IN CARTESIAN BASIS). | |
| REMARK | 3 | B11: | −7.4891 |
| REMARK | 3 | B22: | 16.4458 |
| REMARK | 3 | B33: | −8.9567 |
| REMARK | 3 | B12: | 0.0000 |
| REMARK | 3 | B13: | −5.5598 |
| REMARK | 3 | B23: | 0.0000 |
| REMARK | 3 | | |

| REMARK | 3 | R FACTOR FORMULA. | | | |
|---|---|---|---|---|---|
| REMARK | 3 | R = SUM(\|\|F_OBS\|−SCALE*\|F_MODEL\|\|)/SUM(\|F_OBS\|) | | | |
| REMARK | 3 | | | | |
| REMARK | 3 | TOTAL MODEL STRUCTURE FACTOR (F_MODEL). | | | |
| REMARK | 3 | F_MODEL = FB_CART * (F_CALC_ATOMS + F_BULK) | | | |
| REMARK | 3 | F_BULK = K_SOL * EXP(−B_SOL * S**2/4) * F_MASK | | | |
| REMARK | 3 | F_CALC_ATOMS = ATOMIC MODEL STRUCTURE FACTORS | | | |
| REMARK | 3 | FB_CART = EXP(−H(t) * A(−1) * B * A(−1t) * H) | | | |
| REMARK | 3 | A = orthogonalization matrix, H = MILLER INDEX | | | |
| REMARK | 3 | (t) = TRANSPOSE, (−1) = INVERSE | | | |
| REMARK | 3 | | | | |
| REMARK | 3 | STRUCTURE FACTORS CALCULATION ALGORITHM: FFT | | | |
| REMARK | 3 | | | | |
| REMARK | 3 | DEVIATIONS FROM IDEAL VALUES. | | | |
| REMARK | 3 | | RMSD | MAX | COUNT |
| REMARK | 3 | BOND: | 0.009 | 0.051 | 2574 |
| REMARK | 3 | ANGLE: | 1.278 | 12.449 | 3478 |
| REMARK | 3 | CHIRALITY: | 0.083 | 0.412 | 388 |
| REMARK | 3 | PLANARITY: | 0.005 | 0.047 | 446 |
| REMARK | 3 | DIHEDRAL: | 18.495 | 85.180 | 982 |

| REMARK | 3 | MIN NONBONDED DISTANCE: 2.165 | |
|---|---|---|---|
| REMARK | 3 | | |
| REMARK | 3 | ATOMIC DISPLACEMENT PARAMETERS. | |
| REMARK | 3 | WILSON B: None | |
| REMARK | 3 | RMS(B_ISO_OR_EQUIVALENT_BONDED): 7.48 | |
| REMARK | 3 | ATOMS | NUMBER OF ATOMS |

| | | | |
|---|---|---|---|
| REMARK | 3 | ISO. | ANISO. |

| | | | | |
|---|---|---|---|---|
| REMARK | 3 | ALL: | 2524 | 0 |
| REMARK | 3 | ALL (NO H): | 2524 | 0 |
| REMARK | 3 | SOLVENT: | 5 | 0 |
| REMARK | 3 | NON-SOLVENT: | 2519 | 0 |
| REMARK | 3 | HYDROGENS: | 0 | 0 |
| REMARK | 3 | | | |

TABLE 4
Atomic Coordinates for Residues of a Crystal Structure of
Ancestral Enzyme Thioredoxin AECA

| | | | | | | | |
|---|---|---|---|---|---|---|---|
| CRYST1 | 37.573 | 48.783 | 91.033 | 90.00 | 93.22 | 90.00 | P 1 21 1 |

| | | | | |
|---|---|---|---|---|
| SCALE1 | 0.026615 | 0.000000 | 0.001500 | 0.00000 |
| SCALE2 | 0.000000 | 0.020499 | 0.000000 | 0.00000 |
| SCALE3 | 0.000000 | 0.000000 | 0.011002 | 0.00000 |

| | | | | | | | | | | | |
|---|---|---|---|---|---|---|---|---|---|---|---|
| ATOM | 1 | N | SER | A | 1 | 18.325 | 18.563 | 30.461 | 1.00 | 93.03 | N |
| ATOM | 2 | CA | SER | A | 1 | 17.742 | 17.660 | 31.452 | 1.00 | 95.56 | C |
| ATOM | 3 | CB | SER | A | 1 | 16.427 | 17.069 | 30.946 | 1.00 | 89.37 | C |
| ATOM | 4 | OG | SER | A | 1 | 15.755 | 16.394 | 31.990 | 1.00 | 98.90 | O |
| ATOM | 5 | C | SER | A | 1 | 18.726 | 16.551 | 31.847 | 1.00 | 93.26 | C |
| ATOM | 6 | O | SER | A | 1 | 19.647 | 16.804 | 32.621 | 1.00 | 94.14 | O |
| ATOM | 7 | N | VAL | A | 2 | 18.536 | 15.335 | 31.331 | 1.00 | 63.44 | N |
| ATOM | 8 | CA | VAL | A | 2 | 19.472 | 14.237 | 31.601 | 1.00 | 64.27 | C |
| ATOM | 9 | CB | VAL | A | 2 | 19.418 | 13.136 | 30.509 | 1.00 | 54.51 | C |
| ATOM | 10 | CG1 | VAL | A | 2 | 20.456 | 12.086 | 30.797 | 1.00 | 54.85 | C |
| ATOM | 11 | CG2 | VAL | A | 2 | 18.056 | 12.486 | 30.449 | 1.00 | 57.38 | C |
| ATOM | 12 | C | VAL | A | 2 | 20.923 | 14.719 | 31.729 | 1.00 | 60.52 | C |
| ATOM | 13 | O | VAL | A | 2 | 21.687 | 14.680 | 30.769 | 1.00 | 61.25 | O |
| ATOM | 14 | N | ILE | A | 3 | 21.297 | 15.182 | 32.914 | 1.00 | 76.52 | N |
| ATOM | 15 | CA | ILE | A | 3 | 22.631 | 15.739 | 33.111 | 1.00 | 82.49 | C |
| ATOM | 16 | CB | ILE | A | 3 | 22.777 | 16.448 | 34.488 | 1.00 | 75.29 | C |
| ATOM | 17 | CG1 | ILE | A | 3 | 24.249 | 16.775 | 34.797 | 1.00 | 81.94 | C |
| ATOM | 18 | CD1 | ILE | A | 3 | 24.866 | 17.886 | 33.926 | 1.00 | 82.01 | C |
| ATOM | 19 | CG2 | ILE | A | 3 | 22.182 | 15.603 | 35.588 | 1.00 | 74.23 | C |
| ATOM | 20 | C | ILE | A | 3 | 23.693 | 14.664 | 32.953 | 1.00 | 77.76 | C |
| ATOM | 21 | O | ILE | A | 3 | 23.445 | 13.493 | 33.221 | 1.00 | 79.68 | O |
| ATOM | 22 | N | GLU | A | 4 | 24.869 | 15.072 | 32.497 | 1.00 | 68.06 | N |
| ATOM | 23 | CA | GLU | A | 4 | 25.980 | 14.160 | 32.314 | 1.00 | 67.25 | C |
| ATOM | 24 | CB | GLU | A | 4 | 26.657 | 14.414 | 30.970 | 1.00 | 78.88 | C |
| ATOM | 25 | CG | GLU | A | 4 | 26.798 | 15.892 | 30.611 | 1.00 | 88.69 | C |
| ATOM | 26 | CD | GLU | A | 4 | 25.528 | 16.489 | 30.011 | 1.00 | 87.15 | C |
| ATOM | 27 | OE1 | GLU | A | 4 | 25.146 | 16.067 | 28.896 | 1.00 | 82.96 | O |
| ATOM | 28 | OE2 | GLU | A | 4 | 24.915 | 17.378 | 30.651 | 1.00 | 79.79 | O |
| ATOM | 29 | C | GLU | A | 4 | 26.950 | 14.411 | 33.433 | 1.00 | 78.75 | C |
| ATOM | 30 | O | GLU | A | 4 | 27.749 | 15.343 | 33.380 | 1.00 | 89.72 | O |
| ATOM | 31 | N | ILE | A | 5 | 26.876 | 13.589 | 34.465 | 1.00 | 61.04 | N |
| ATOM | 32 | CA | ILE | A | 5 | 27.680 | 13.846 | 35.641 | 1.00 | 58.02 | C |
| ATOM | 33 | CB | ILE | A | 5 | 26.984 | 13.325 | 36.915 | 1.00 | 59.29 | C |
| ATOM | 34 | CG1 | ILE | A | 5 | 27.476 | 11.924 | 37.269 | 1.00 | 61.82 | C |
| ATOM | 35 | CD1 | ILE | A | 5 | 28.652 | 11.900 | 38.232 | 1.00 | 65.42 | C |
| ATOM | 36 | CG2 | ILE | A | 5 | 25.474 | 13.315 | 36.751 | 1.00 | 54.50 | C |
| ATOM | 37 | C | ILE | A | 5 | 29.083 | 13.236 | 35.512 | 1.00 | 61.60 | C |
| ATOM | 38 | O | ILE | A | 5 | 29.248 | 12.142 | 34.960 | 1.00 | 49.59 | O |
| ATOM | 39 | N | ASN | A | 6 | 30.079 | 13.960 | 36.029 | 1.00 | 73.16 | N |
| ATOM | 40 | CA | ASN | A | 6 | 31.471 | 13.506 | 36.097 | 1.00 | 71.38 | C |
| ATOM | 41 | CB | ASN | A | 6 | 32.326 | 14.337 | 35.156 | 1.00 | 72.01 | C |
| ATOM | 42 | CG | ASN | A | 6 | 32.189 | 15.818 | 35.425 | 1.00 | 77.59 | C |
| ATOM | 43 | OD1 | ASN | A | 6 | 32.898 | 16.370 | 36.270 | 1.00 | 81.58 | O |
| ATOM | 44 | ND2 | ASN | A | 6 | 31.259 | 16.470 | 34.725 | 1.00 | 69.66 | N |
| ATOM | 45 | C | ASN | A | 6 | 32.026 | 13.649 | 37.510 | 1.00 | 74.67 | C |
| ATOM | 46 | O | ASN | A | 6 | 31.438 | 14.338 | 38.349 | 1.00 | 72.89 | O |
| ATOM | 47 | N | ASP | A | 7 | 33.175 | 13.022 | 37.758 | 1.00 | 96.40 | N |
| ATOM | 48 | CA | ASP | A | 7 | 33.815 | 12.995 | 39.090 | 1.00 | 102.57 | C |
| ATOM | 49 | CB | ASP | A | 7 | 35.270 | 12.508 | 38.978 | 1.00 | 90.00 | C |
| ATOM | 50 | CG | ASP | A | 7 | 35.411 | 11.225 | 38.150 | 1.00 | 96.84 | C |
| ATOM | 51 | OD1 | ASP | A | 7 | 35.570 | 11.312 | 36.908 | 1.00 | 93.02 | O |
| ATOM | 52 | OD2 | ASP | A | 7 | 35.387 | 10.125 | 38.744 | 1.00 | 96.83 | O |
| ATOM | 53 | C | ASP | A | 7 | 33.788 | 14.302 | 39.910 | 1.00 | 98.35 | C |
| ATOM | 54 | O | ASP | A | 7 | 33.919 | 14.266 | 41.132 | 1.00 | 99.35 | O |
| ATOM | 55 | N | GLU | A | 8 | 33.624 | 15.439 | 39.240 | 1.00 | 94.34 | N |
| ATOM | 56 | CA | GLU | A | 8 | 33.701 | 16.747 | 39.892 | 1.00 | 93.93 | C |
| ATOM | 57 | CB | GLU | A | 8 | 34.539 | 17.708 | 39.045 | 1.00 | 104.22 | C |
| ATOM | 58 | CG | GLU | A | 8 | 35.984 | 17.849 | 39.531 | 1.00 | 109.88 | C |
| ATOM | 59 | CD | GLU | A | 8 | 36.678 | 16.502 | 39.713 | 1.00 | 112.71 | C |
| ATOM | 60 | OE1 | GLU | A | 8 | 36.910 | 16.108 | 40.878 | 1.00 | 110.85 | O |
| ATOM | 61 | OE2 | GLU | A | 8 | 36.992 | 15.846 | 38.691 | 1.00 | 115.09 | O |
| ATOM | 62 | C | GLU | A | 8 | 32.355 | 17.374 | 40.243 | 1.00 | 89.65 | C |
| ATOM | 63 | O | GLU | A | 8 | 32.095 | 17.682 | 41.410 | 1.00 | 84.85 | O |
| ATOM | 64 | N | ASN | A | 9 | 31.504 | 17.569 | 39.238 | 1.00 | 70.09 | N |
| ATOM | 65 | CA | ASN | A | 9 | 30.125 | 17.996 | 39.496 | 1.00 | 68.14 | C |
| ATOM | 66 | CB | ASN | A | 9 | 29.423 | 18.422 | 38.203 | 1.00 | 69.98 | C |
| ATOM | 67 | CG | ASN | A | 9 | 28.974 | 17.231 | 37.340 | 1.00 | 70.02 | C |
| ATOM | 68 | OD1 | ASN | A | 9 | 28.025 | 17.342 | 36.557 | 1.00 | 65.59 | O |
| ATOM | 69 | ND2 | ASN | A | 9 | 29.652 | 16.097 | 37.482 | 1.00 | 65.99 | N |
| ATOM | 70 | C | ASN | A | 9 | 29.277 | 16.953 | 40.248 | 1.00 | 62.48 | C |
| ATOM | 71 | O | ASN | A | 9 | 28.146 | 17.229 | 40.618 | 1.00 | 62.41 | O |
| ATOM | 72 | N | PHE | A | 10 | 29.834 | 15.770 | 40.498 | 1.00 | 73.47 | N |
| ATOM | 73 | CA | PHE | A | 10 | 29.079 | 14.673 | 41.116 | 1.00 | 71.62 | C |
| ATOM | 74 | CB | PHE | A | 10 | 29.983 | 13.519 | 41.562 | 1.00 | 70.83 | C |
| ATOM | 75 | CG | PHE | A | 10 | 29.223 | 12.402 | 42.240 | 1.00 | 74.73 | C |
| ATOM | 76 | CD2 | PHE | A | 10 | 28.534 | 11.456 | 41.484 | 1.00 | 75.00 | C |
| ATOM | 77 | CE2 | PHE | A | 10 | 27.816 | 10.435 | 42.086 | 1.00 | 66.17 | C |
| ATOM | 78 | CZ | PHE | A | 10 | 27.767 | 10.352 | 43.473 | 1.00 | 72.89 | C |
| ATOM | 79 | CE1 | PHE | A | 10 | 28.434 | 11.289 | 44.243 | 1.00 | 68.64 | C |
| ATOM | 80 | CD1 | PHE | A | 10 | 29.154 | 12.319 | 43.623 | 1.00 | 71.79 | C |
| ATOM | 81 | C | PHE | A | 10 | 28.210 | 15.053 | 42.301 | 1.00 | 76.07 | C |
| ATOM | 82 | O | PHE | A | 10 | 27.230 | 14.376 | 42.602 | 1.00 | 81.66 | O |
| ATOM | 83 | N | ASP | A | 11 | 28.577 | 16.113 | 43.001 | 1.00 | 93.76 | N |
| ATOM | 84 | CA | ASP | A | 11 | 27.897 | 16.415 | 44.250 | 1.00 | 86.23 | C |
| ATOM | 85 | CB | ASP | A | 11 | 28.820 | 17.152 | 45.203 | 1.00 | 90.97 | C |
| ATOM | 86 | CG | ASP | A | 11 | 30.026 | 16.311 | 45.582 | 1.00 | 96.54 | C |
| ATOM | 87 | OD1 | ASP | A | 11 | 30.474 | 15.501 | 44.738 | 1.00 | 95.75 | O |
| ATOM | 88 | OD2 | ASP | A | 11 | 30.523 | 16.442 | 46.718 | 1.00 | 103.21 | O |
| ATOM | 89 | C | ASP | A | 11 | 26.579 | 17.129 | 44.034 | 1.00 | 89.66 | C |
| ATOM | 90 | O | ASP | A | 11 | 26.232 | 18.057 | 44.755 | 1.00 | 87.34 | O |
| ATOM | 91 | N | AGLU | A | 12 | 25.859 | 16.671 | 43.011 | 1.00 | 95.44 | N |
| ATOM | 92 | CA | AGLU | A | 12 | 24.486 | 17.080 | 42.746 | 1.00 | 96.77 | C |
| ATOM | 93 | CB | AGLU | A | 12 | 24.349 | 17.496 | 41.292 | 1.00 | 93.86 | C |
| ATOM | 94 | CG | AGLU | A | 12 | 25.441 | 18.483 | 40.919 | 1.00 | 99.84 | C |
| ATOM | 95 | CD | AGLU | A | 12 | 25.920 | 19.307 | 42.128 | 1.00 | 102.33 | C |
| ATOM | 96 | OE1 | AGLU | A | 12 | 27.153 | 19.363 | 42.381 | 1.00 | 96.27 | O |
| ATOM | 97 | OE2 | AGLU | A | 12 | 25.056 | 19.889 | 42.830 | 1.00 | 98.47 | O |
| ATOM | 98 | C | AGLU | A | 12 | 23.618 | 15.891 | 43.081 | 1.00 | 92.75 | C |
| ATOM | 99 | O | AGLU | A | 12 | 22.412 | 15.878 | 42.872 | 1.00 | 87.89 | O |
| ATOM | 100 | N | BGLU | A | 12 | 25.846 | 16.698 | 43.014 | 0.00 | 95.31 | N |
| ATOM | 101 | CA | BGLU | A | 12 | 24.475 | 17.142 | 42.827 | 0.00 | 96.51 | C |
| ATOM | 102 | CB | BGLU | A | 12 | 24.205 | 17.518 | 41.368 | 0.00 | 93.89 | C |
| ATOM | 103 | CG | BGLU | A | 12 | 23.983 | 16.344 | 40.436 | 0.00 | 90.85 | C |
| ATOM | 104 | CD | BGLU | A | 12 | 25.275 | 15.800 | 39.866 | 0.00 | 91.65 | C |
| ATOM | 105 | OE1 | BGLU | A | 12 | 25.838 | 16.450 | 38.962 | 0.00 | 92.17 | O |
| ATOM | 106 | OE2 | BGLU | A | 12 | 25.728 | 14.727 | 40.317 | 0.00 | 92.30 | O |
| ATOM | 107 | C | BGLU | A | 12 | 23.609 | 15.975 | 43.272 | 0.00 | 92.78 | C |
| ATOM | 108 | O | BGLU | A | 12 | 22.383 | 16.053 | 43.301 | 0.00 | 89.36 | O |
| ATOM | 109 | N | VAL | A | 13 | 24.291 | 14.889 | 43.624 | 1.00 | 82.34 | N |
| ATOM | 110 | CA | VAL | A | 13 | 23.678 | 13.726 | 44.207 | 1.00 | 82.59 | C |
| ATOM | 111 | CB | VAL | A | 13 | 24.518 | 12.487 | 43.847 | 1.00 | 78.66 | C |
| ATOM | 112 | CG1 | VAL | A | 13 | 24.043 | 11.276 | 44.614 | 1.00 | 74.13 | C |
| ATOM | 113 | CG2 | VAL | A | 13 | 24.488 | 12.238 | 42.346 | 1.00 | 77.77 | C |
| ATOM | 114 | C | VAL | A | 13 | 23.709 | 13.934 | 45.729 | 1.00 | 85.18 | C |
| ATOM | 115 | O | VAL | A | 13 | 22.894 | 13.369 | 46.474 | 1.00 | 77.97 | O |
| ATOM | 116 | N | ILE | A | 14 | 24.654 | 14.768 | 46.171 | 1.00 | 80.06 | N |
| ATOM | 117 | CA | ILE | A | 14 | 24.880 | 15.035 | 47.597 | 1.00 | 84.88 | C |
| ATOM | 118 | CB | ILE | A | 14 | 26.403 | 14.894 | 48.015 | 1.00 | 80.37 | C |
| ATOM | 119 | CG1 | ILE | A | 14 | 27.000 | 13.556 | 47.575 | 1.00 | 69.92 | C |
| ATOM | 120 | CD1 | ILE | A | 14 | 28.332 | 13.228 | 48.239 | 1.00 | 64.35 | C |
| ATOM | 121 | CG2 | ILE | A | 14 | 26.571 | 14.985 | 49.514 | 1.00 | 91.17 | C |
| ATOM | 122 | C | ILE | A | 14 | 24.339 | 16.409 | 48.045 | 1.00 | 88.80 | C |
| ATOM | 123 | O | ILE | A | 14 | 24.547 | 16.825 | 49.189 | 1.00 | 93.22 | O |
| ATOM | 124 | N | LYS | A | 15 | 23.641 | 17.115 | 47.159 | 1.00 | 84.90 | N |
| ATOM | 125 | CA | LYS | A | 15 | 23.075 | 18.413 | 47.537 | 1.00 | 84.96 | C |
| ATOM | 126 | CB | LYS | A | 15 | 24.014 | 19.548 | 47.117 | 1.00 | 92.62 | C |
| ATOM | 127 | CG | LYS | A | 15 | 25.493 | 19.251 | 47.346 | 1.00 | 91.34 | C |
| ATOM | 128 | CD | LYS | A | 15 | 26.381 | 20.084 | 46.441 | 1.00 | 83.09 | C |
| ATOM | 129 | CE | LYS | A | 15 | 26.775 | 21.400 | 47.069 | 1.00 | 87.11 | C |
| ATOM | 130 | NZ | LYS | A | 15 | 27.702 | 22.143 | 46.153 | 1.00 | 74.02 | N |
| ATOM | 131 | C | LYS | A | 15 | 21.667 | 18.645 | 46.967 | 1.00 | 82.89 | C |
| ATOM | 132 | O | LYS | A | 15 | 21.323 | 19.759 | 46.552 | 1.00 | 77.76 | O |
| ATOM | 133 | N | LYS | A | 16 | 20.852 | 17.596 | 46.956 | 1.00 | 80.48 | N |
| ATOM | 134 | CA | LYS | A | 16 | 19.491 | 17.713 | 46.456 | 1.00 | 81.00 | C |
| ATOM | 135 | CB | LYS | A | 16 | 19.392 | 17.217 | 45.007 | 1.00 | 79.63 | C |
| ATOM | 136 | CG | LYS | A | 16 | 18.033 | 17.485 | 44.339 | 1.00 | 76.94 | C |
| ATOM | 137 | CD | LYS | A | 16 | 17.866 | 18.938 | 43.819 | 1.00 | 78.71 | C |
| ATOM | 138 | CE | LYS | A | 16 | 17.916 | 20.013 | 44.931 | 1.00 | 80.92 | C |
| ATOM | 139 | NZ | LYS | A | 16 | 16.731 | 20.030 | 45.865 | 1.00 | 78.45 | N |
| ATOM | 140 | C | LYS | A | 16 | 18.509 | 16.944 | 47.317 | 1.00 | 78.57 | C |
| ATOM | 141 | O | LYS | A | 16 | 18.801 | 15.837 | 47.778 | 1.00 | 73.23 | O |
| ATOM | 142 | N | ASP | A | 17 | 17.336 | 17.533 | 47.518 | 1.00 | 68.38 | N |
| ATOM | 143 | CA | ASP | A | 17 | 16.276 | 16.859 | 48.248 | 1.00 | 62.15 | C |
| ATOM | 144 | CB | ASP | A | 17 | 15.275 | 17.883 | 48.797 | 1.00 | 68.54 | C |
| ATOM | 145 | CG | ASP | A | 17 | 15.684 | 18.464 | 50.148 | 1.00 | 67.94 | C |
| ATOM | 146 | OD1 | ASP | A | 17 | 14.774 | 18.876 | 50.905 | 1.00 | 58.95 | O |
| ATOM | 147 | OD2 | ASP | A | 17 | 16.898 | 18.513 | 50.447 | 1.00 | 76.04 | O |
| ATOM | 148 | C | ASP | A | 17 | 15.557 | 15.875 | 47.323 | 1.00 | 73.48 | C |
| ATOM | 149 | O | ASP | A | 17 | 14.738 | 15.073 | 47.779 | 1.00 | 72.90 | O |
| ATOM | 150 | N | LYS | A | 18 | 15.877 | 15.947 | 46.025 | 1.00 | 72.08 | N |
| ATOM | 151 | CA | LYS | A | 18 | 15.144 | 15.237 | 44.963 | 1.00 | 64.54 | C |
| ATOM | 152 | CB | LYS | A | 18 | 14.957 | 16.153 | 43.739 | 1.00 | 57.20 | C |
| ATOM | 153 | CG | LYS | A | 18 | 13.611 | 16.856 | 43.685 | 1.00 | 59.65 | C |
| ATOM | 154 | CD | LYS | A | 18 | 13.261 | 17.513 | 45.023 | 1.00 | 76.14 | C |
| ATOM | 155 | CE | LYS | A | 18 | 11.780 | 17.941 | 45.115 | 1.00 | 68.61 | C |
| ATOM | 156 | NZ | LYS | A | 18 | 11.439 | 18.690 | 46.391 | 1.00 | 46.87 | N |
| ATOM | 157 | C | LYS | A | 18 | 15.819 | 13.936 | 44.531 | 1.00 | 58.93 | C |
| ATOM | 158 | O | LYS | A | 18 | 17.034 | 13.892 | 44.352 | 1.00 | 64.80 | O |
| ATOM | 159 | N | VAL | A | 19 | 15.030 | 12.881 | 44.361 | 1.00 | 46.98 | N |
| ATOM | 160 | CA | VAL | A | 19 | 15.556 | 11.609 | 43.882 | 1.00 | 48.15 | C |
| ATOM | 161 | CB | VAL | A | 19 | 14.424 | 10.613 | 43.613 | 1.00 | 51.03 | C |
| ATOM | 162 | CG1 | VAL | A | 19 | 14.988 | 9.218 | 43.324 | 1.00 | 44.51 | C |
| ATOM | 163 | CG2 | VAL | A | 19 | 13.493 | 10.565 | 44.788 | 1.00 | 54.70 | C |
| ATOM | 164 | C | VAL | A | 19 | 16.390 | 11.760 | 42.607 | 1.00 | 47.76 | C |
| ATOM | 165 | O | VAL | A | 19 | 15.907 | 12.203 | 41.568 | 1.00 | 52.65 | O |
| ATOM | 166 | N | VAL | A | 20 | 17.649 | 11.372 | 42.692 | 1.00 | 42.79 | N |
| ATOM | 167 | CA | VAL | A | 20 | 18.532 | 11.390 | 41.542 | 1.00 | 38.05 | C |
| ATOM | 168 | CB | VAL | A | 20 | 19.954 | 11.887 | 41.911 | 1.00 | 38.95 | C |
| ATOM | 169 | CG1 | VAL | A | 20 | 20.823 | 11.924 | 40.690 | 1.00 | 43.85 | C |
| ATOM | 170 | CG2 | VAL | A | 20 | 19.901 | 13.273 | 42.532 | 1.00 | 34.16 | C |
| ATOM | 171 | C | VAL | A | 20 | 18.617 | 9.974 | 40.995 | 1.00 | 32.43 | C |
| ATOM | 172 | O | VAL | A | 20 | 18.954 | 9.016 | 41.704 | 1.00 | 29.51 | O |
| ATOM | 173 | N | VAL | A | 21 | 18.272 | 9.830 | 39.730 | 1.00 | 45.32 | N |
| ATOM | 174 | CA | VAL | A | 21 | 18.428 | 8.552 | 39.080 | 1.00 | 46.21 | C |
| ATOM | 175 | CB | VAL | A | 21 | 17.229 | 8.203 | 38.223 | 1.00 | 43.66 | C |
| ATOM | 176 | CG1 | VAL | A | 21 | 17.588 | 7.049 | 37.299 | 1.00 | 36.41 | C |
| ATOM | 177 | CG2 | VAL | A | 21 | 16.049 | 7.872 | 39.114 | 1.00 | 43.66 | C |
| ATOM | 178 | C | VAL | A | 21 | 19.634 | 8.658 | 38.193 | 1.00 | 40.45 | C |
| ATOM | 179 | O | VAL | A | 21 | 19.712 | 9.548 | 37.349 | 1.00 | 43.66 | O |
| ATOM | 180 | N | VAL | A | 22 | 20.584 | 7.760 | 38.390 | 1.00 | 34.03 | N |
| ATOM | 181 | CA | VAL | A | 22 | 21.810 | 7.815 | 37.612 | 1.00 | 42.77 | C |
| ATOM | 182 | CB | VAL | A | 22 | 23.049 | 8.363 | 38.417 | 1.00 | 36.79 | C |
| ATOM | 183 | CG1 | VAL | A | 22 | 23.152 | 7.715 | 39.732 | 1.00 | 37.85 | C |
| ATOM | 184 | CG2 | VAL | A | 22 | 24.357 | 8.161 | 37.636 | 1.00 | 38.48 | C |
| ATOM | 185 | C | VAL | A | 22 | 22.088 | 6.501 | 36.894 | 1.00 | 34.67 | C |
| ATOM | 186 | O | VAL | A | 22 | 21.996 | 5.416 | 37.462 | 1.00 | 36.22 | O |
| ATOM | 187 | N | ASP | A | 23 | 22.437 | 6.646 | 35.626 | 1.00 | 27.78 | N |
| ATOM | 188 | CA | ASP | A | 23 | 22.554 | 5.555 | 34.701 | 1.00 | 29.29 | C |
| ATOM | 189 | CB | ASP | A | 23 | 21.702 | 5.905 | 33.474 | 1.00 | 40.54 | C |
| ATOM | 190 | CG | ASP | A | 23 | 21.840 | 4.910 | 32.346 | 1.00 | 46.95 | C |
| ATOM | 191 | OD1 | ASP | A | 23 | 22.114 | 3.709 | 32.595 | 1.00 | 50.35 | O |
| ATOM | 192 | OD2 | ASP | A | 23 | 21.648 | 5.332 | 31.187 | 1.00 | 59.45 | O |
| ATOM | 193 | C | ASP | A | 23 | 24.020 | 5.400 | 34.336 | 1.00 | 32.65 | C |
| ATOM | 194 | O | ASP | A | 23 | 24.677 | 6.354 | 33.930 | 1.00 | 34.36 | O |
| ATOM | 195 | N | PHE | A | 24 | 24.543 | 4.195 | 34.495 | 1.00 | 40.40 | N |
| ATOM | 196 | CA | PHE | A | 24 | 25.948 | 3.955 | 34.225 | 1.00 | 41.68 | C |
| ATOM | 197 | CB | PHE | A | 24 | 26.553 | 3.093 | 35.335 | 1.00 | 47.07 | C |
| ATOM | 198 | CG | PHE | A | 24 | 26.608 | 3.780 | 36.668 | 1.00 | 48.84 | C |
| ATOM | 199 | CD2 | PHE | A | 24 | 27.632 | 4.678 | 36.968 | 1.00 | 54.62 | C |
| ATOM | 200 | CE2 | PHE | A | 24 | 27.674 | 5.329 | 38.205 | 1.00 | 52.58 | C |
| ATOM | 201 | CZ | PHE | A | 24 | 26.694 | 5.084 | 39.144 | 1.00 | 50.65 | C |
| ATOM | 202 | CE1 | PHE | A | 24 | 25.669 | 4.191 | 38.859 | 1.00 | 51.01 | C |
| ATOM | 203 | CD1 | PHE | A | 24 | 25.630 | 3.544 | 37.622 | 1.00 | 48.65 | C |
| ATOM | 204 | C | PHE | A | 24 | 26.043 | 3.226 | 32.913 | 1.00 | 41.92 | C |
| ATOM | 205 | O | PHE | A | 24 | 25.516 | 2.130 | 32.786 | 1.00 | 42.21 | O |
| ATOM | 206 | N | TRP | A | 25 | 26.719 | 3.818 | 31.935 | 1.00 | 40.66 | N |
| ATOM | 207 | CA | TRP | A | 25 | 26.711 | 3.270 | 30.577 | 1.00 | 43.71 | C |
| ATOM | 208 | CB | TRP | A | 25 | 25.732 | 4.069 | 29.712 | 1.00 | 39.40 | C |
| ATOM | 209 | CG | TRP | A | 25 | 26.231 | 5.477 | 29.562 | 1.00 | 38.20 | C |
| ATOM | 210 | CD1 | TRP | A | 25 | 26.342 | 6.413 | 30.557 | 1.00 | 43.71 | C |
| ATOM | 211 | NE1 | TRP | A | 25 | 26.876 | 7.571 | 30.055 | 1.00 | 44.68 | N |
| ATOM | 212 | CE2 | TRP | A | 25 | 27.139 | 7.400 | 28.721 | 1.00 | 38.78 | C |
| ATOM | 213 | CD2 | TRP | A | 25 | 26.751 | 6.086 | 28.378 | 1.00 | 33.04 | C |
| ATOM | 214 | CE3 | TRP | A | 25 | 26.917 | 5.657 | 27.060 | 1.00 | 35.02 | C |
| ATOM | 215 | CZ3 | TRP | A | 25 | 27.449 | 6.540 | 26.136 | 1.00 | 37.50 | C |
| ATOM | 216 | CH2 | TRP | A | 25 | 27.829 | 7.845 | 26.511 | 1.00 | 43.16 | C |
| ATOM | 217 | CZ2 | TRP | A | 25 | 27.683 | 8.287 | 27.799 | 1.00 | 43.79 | C |
| ATOM | 218 | C | TRP | A | 25 | 28.104 | 3.388 | 29.961 | 1.00 | 46.18 | C |
| ATOM | 219 | O | TRP | A | 25 | 29.066 | 3.811 | 30.625 | 1.00 | 40.29 | O |
| ATOM | 220 | N | ALA | A | 26 | 28.191 | 3.045 | 28.675 | 1.00 | 33.67 | N |
| ATOM | 221 | CA | ALA | A | 26 | 29.414 | 3.205 | 27.929 | 1.00 | 32.26 | C |
| ATOM | 222 | CB | ALA | A | 26 | 30.515 | 2.283 | 28.497 | 1.00 | 41.42 | C |
| ATOM | 223 | C | ALA | A | 26 | 29.198 | 2.935 | 26.465 | 1.00 | 31.60 | C |
| ATOM | 224 | O | ALA | A | 26 | 28.469 | 2.018 | 26.087 | 1.00 | 38.72 | O |
| ATOM | 225 | N | GLU | A | 27 | 29.866 | 3.723 | 25.638 | 1.00 | 34.85 | N |
| ATOM | 226 | CA | GLU | A | 27 | 29.734 | 3.614 | 24.205 | 1.00 | 36.73 | C |
| ATOM | 227 | CB | GLU | A | 27 | 30.776 | 4.481 | 23.520 | 1.00 | 38.76 | C |
| ATOM | 228 | CG | GLU | A | 27 | 30.297 | 5.037 | 22.179 | 1.00 | 52.02 | C |
| ATOM | 229 | CD | GLU | A | 27 | 29.128 | 5.996 | 22.328 | 1.00 | 59.51 | C |
| ATOM | 230 | OE1 | GLU | A | 27 | 29.331 | 7.122 | 22.838 | 1.00 | 58.26 | O |
| ATOM | 231 | OE2 | GLU | A | 27 | 27.995 | 5.608 | 21.957 | 1.00 | 66.32 | O |
| ATOM | 232 | C | GLU | A | 27 | 29.732 | 2.186 | 23.640 | 1.00 | 43.36 | C |
| ATOM | 233 | O | GLU | A | 27 | 29.006 | 1.891 | 22.674 | 1.00 | 47.24 | O |
| ATOM | 234 | N | TRP | A | 28 | 30.531 | 1.298 | 24.215 | 1.00 | 38.70 | N |
| ATOM | 235 | CA | TRP | A | 28 | 30.633 | −0.061 | 23.665 | 1.00 | 40.05 | C |
| ATOM | 236 | CB | TRP | A | 28 | 31.995 | −0.685 | 24.006 | 1.00 | 39.35 | C |
| ATOM | 237 | CG | TRP | A | 28 | 32.297 | −0.554 | 25.466 | 1.00 | 36.23 | C |
| ATOM | 238 | CD1 | TRP | A | 28 | 33.007 | 0.453 | 26.074 | 1.00 | 36.47 | C |
| ATOM | 239 | NE1 | TRP | A | 28 | 33.045 | 0.237 | 27.440 | 1.00 | 43.64 | N |
| ATOM | 240 | CE2 | TRP | A | 28 | 32.344 | −0.905 | 27.734 | 1.00 | 38.86 | C |
| ATOM | 241 | CD2 | TRP | A | 28 | 31.862 | −1.435 | 26.515 | 1.00 | 30.95 | C |
| ATOM | 242 | CE3 | TRP | A | 28 | 31.098 | −2.608 | 26.543 | 1.00 | 42.18 | C |
| ATOM | 243 | CZ3 | TRP | A | 28 | 30.851 | −3.219 | 27.778 | 1.00 | 41.80 | C |
| ATOM | 244 | CH2 | TRP | A | 28 | 31.348 | −2.665 | 28.967 | 1.00 | 38.31 | C |
| ATOM | 245 | CZ2 | TRP | A | 28 | 32.089 | −1.509 | 28.966 | 1.00 | 33.73 | C |
| ATOM | 246 | C | TRP | A | 28 | 29.516 | −0.971 | 24.171 | 1.00 | 35.51 | C |
| ATOM | 247 | O | TRP | A | 28 | 29.500 | −2.157 | 23.883 | 1.00 | 43.56 | O |
| ATOM | 248 | N | CYS | A | 29 | 28.575 | −0.439 | 24.932 | 1.00 | 38.50 | N |
| ATOM | 249 | CA | CYS | A | 29 | 27.582 | −1.316 | 25.552 | 1.00 | 44.43 | C |
| ATOM | 250 | CB | CYS | A | 29 | 27.392 | −0.947 | 27.024 | 1.00 | 44.93 | C |
| ATOM | 251 | SG | CYS | A | 29 | 26.007 | −1.798 | 27.803 | 1.00 | 50.55 | S |
| ATOM | 252 | C | CYS | A | 29 | 26.240 | −1.364 | 24.812 | 1.00 | 44.88 | C |
| ATOM | 253 | O | CYS | A | 29 | 25.497 | −0.370 | 24.753 | 1.00 | 43.32 | O |
| ATOM | 254 | N | GLY | A | 30 | 25.943 | −2.532 | 24.258 | 1.00 | 52.78 | N |
| ATOM | 255 | CA | GLY | A | 30 | 24.747 | −2.721 | 23.468 | 1.00 | 55.23 | C |
| ATOM | 256 | C | GLY | A | 30 | 23.499 | −2.497 | 24.284 | 1.00 | 56.80 | C |
| ATOM | 257 | O | GLY | A | 30 | 22.715 | −1.588 | 23.986 | 1.00 | 54.84 | O |
| ATOM | 258 | N | PRO | A | 31 | 23.305 | −3.336 | 25.310 | 1.00 | 48.40 | N |
| ATOM | 259 | CA | PRO | A | 31 | 22.186 | −3.256 | 26.253 | 1.00 | 48.15 | C |
| ATOM | 260 | CB | PRO | A | 31 | 22.612 | −4.201 | 27.370 | 1.00 | 43.17 | C |
| ATOM | 261 | CG | PRO | A | 31 | 23.353 | −5.277 | 26.658 | 1.00 | 42.21 | C |
| ATOM | 262 | CD | PRO | A | 31 | 24.057 | −4.594 | 25.466 | 1.00 | 45.02 | C |
| ATOM | 263 | C | PRO | A | 31 | 21.960 | −1.860 | 26.798 | 1.00 | 50.22 | C |
| ATOM | 264 | O | PRO | A | 31 | 20.826 | −1.514 | 27.131 | 1.00 | 52.28 | O |
| ATOM | 265 | N | CYS | A | 32 | 23.008 | −1.056 | 26.868 | 1.00 | 38.97 | N |
| ATOM | 266 | CA | CYS | A | 32 | 22.851 | 0.298 | 27.377 | 1.00 | 43.30 | C |
| ATOM | 267 | CB | CYS | A | 32 | 24.219 | 0.949 | 27.599 | 1.00 | 50.17 | C |
| ATOM | 268 | SG | CYS | A | 32 | 25.334 | −0.040 | 28.648 | 1.00 | 58.61 | S |
| ATOM | 269 | C | CYS | A | 32 | 22.047 | 1.136 | 26.401 | 1.00 | 47.60 | C |
| ATOM | 270 | O | CYS | A | 32 | 21.446 | 2.146 | 26.771 | 1.00 | 49.30 | O |
| ATOM | 271 | N | ARG | A | 33 | 22.057 | 0.718 | 25.139 | 1.00 | 51.00 | N |
| ATOM | 272 | CA | ARG | A | 33 | 21.318 | 1.417 | 24.100 | 1.00 | 46.52 | C |
| ATOM | 273 | C | ARG | A | 33 | 19.815 | 1.196 | 24.257 | 1.00 | 45.22 | C |
| ATOM | 274 | O | ARG | A | 33 | 19.016 | 1.997 | 23.783 | 1.00 | 48.76 | O |
| ATOM | 275 | CB | ARG | A | 33 | 21.782 | 0.965 | 22.723 | 1.00 | 45.56 | C |
| ATOM | 276 | CG | ARG | A | 33 | 23.195 | 1.373 | 22.411 | 1.00 | 46.47 | C |
| ATOM | 277 | CD | ARG | A | 33 | 23.806 | 0.573 | 21.258 | 1.00 | 39.31 | C |
| ATOM | 278 | NE | ARG | A | 33 | 25.171 | 1.023 | 21.055 | 1.00 | 47.75 | N |
| ATOM | 279 | CZ | ARG | A | 33 | 26.187 | 0.249 | 20.707 | 1.00 | 49.20 | C |
| ATOM | 280 | NH1 | ARG | A | 33 | 26.008 | −1.041 | 20.467 | 1.00 | 47.25 | N |
| ATOM | 281 | NH2 | ARG | A | 33 | 27.384 | 0.790 | 20.590 | 1.00 | 50.60 | N |
| ATOM | 282 | N | MET | A | 34 | 19.422 | 0.117 | 24.921 | 1.00 | 44.44 | N |
| ATOM | 283 | CA | MET | A | 34 | 18.007 | −0.054 | 25.222 | 1.00 | 55.68 | C |
| ATOM | 284 | C | MET | A | 34 | 17.455 | 1.069 | 26.113 | 1.00 | 53.24 | C |
| ATOM | 285 | O | MET | A | 34 | 16.485 | 1.723 | 25.746 | 1.00 | 53.05 | O |
| ATOM | 286 | CB | MET | A | 34 | 17.712 | −1.456 | 25.767 | 1.00 | 51.44 | C |
| ATOM | 287 | CG | MET | A | 34 | 17.687 | −2.508 | 24.663 | 1.00 | 43.70 | C |
| ATOM | 288 | SD | MET | A | 34 | 18.086 | −4.175 | 25.212 | 1.00 | 62.13 | S |
| ATOM | 289 | CE | MET | A | 34 | 16.486 | −4.717 | 25.777 | 1.00 | 57.11 | C |
| ATOM | 290 | N | ILE | A | 35 | 18.080 | 1.338 | 27.252 | 1.00 | 41.35 | N |
| ATOM | 291 | CA | ILE | A | 35 | 17.477 | 2.311 | 28.161 | 1.00 | 45.25 | C |
| ATOM | 292 | CB | ILE | A | 35 | 17.625 | 1.905 | 29.617 | 1.00 | 57.49 | C |
| ATOM | 293 | CG1 | ILE | A | 35 | 19.096 | 1.858 | 30.020 | 1.00 | 48.32 | C |
| ATOM | 294 | CD1 | ILE | A | 35 | 19.306 | 0.990 | 31.288 | 1.00 | 40.60 | C |
| ATOM | 295 | CG2 | ILE | A | 35 | 16.915 | 0.559 | 29.867 | 1.00 | 60.05 | C |
| ATOM | 296 | C | ILE | A | 35 | 17.837 | 3.784 | 28.000 | 1.00 | 47.14 | C |
| ATOM | 297 | O | ILE | A | 35 | 17.153 | 4.661 | 28.552 | 1.00 | 40.82 | O |
| ATOM | 298 | N | ALA | A | 36 | 18.905 | 4.054 | 27.257 | 1.00 | 47.27 | N |
| ATOM | 299 | CA | ALA | A | 36 | 19.294 | 5.432 | 26.958 | 1.00 | 43.95 | C |
| ATOM | 300 | CB | ALA | A | 36 | 20.377 | 5.456 | 25.916 | 1.00 | 43.77 | C |
| ATOM | 301 | C | ALA | A | 36 | 18.104 | 6.294 | 26.505 | 1.00 | 44.39 | C |
| ATOM | 302 | O | ALA | A | 36 | 17.889 | 7.382 | 27.032 | 1.00 | 44.41 | O |
| ATOM | 303 | N | PRO | A | 37 | 17.326 | 5.806 | 25.523 | 1.00 | 39.47 | N |
| ATOM | 304 | CA | PRO | A | 37 | 16.149 | 6.543 | 25.044 | 1.00 | 44.55 | C |
| ATOM | 305 | CB | PRO | A | 37 | 15.601 | 5.632 | 23.948 | 1.00 | 40.58 | C |
| ATOM | 306 | CG | PRO | A | 37 | 16.147 | 4.254 | 24.284 | 1.00 | 42.38 | C |
| ATOM | 307 | CD | PRO | A | 37 | 17.499 | 4.524 | 24.812 | 1.00 | 38.01 | C |
| ATOM | 308 | C | PRO | A | 37 | 15.072 | 6.723 | 26.121 | 1.00 | 42.99 | C |
| ATOM | 309 | O | PRO | A | 37 | 14.384 | 7.756 | 26.107 | 1.00 | 35.37 | O |
| ATOM | 310 | N | ILE | A | 38 | 14.938 | 5.726 | 27.005 | 1.00 | 37.72 | N |
| ATOM | 311 | CA | ILE | A | 38 | 13.970 | 5.709 | 28.111 | 1.00 | 43.02 | C |
| ATOM | 312 | CB | ILE | A | 38 | 13.783 | 4.289 | 28.656 | 1.00 | 43.66 | C |
| ATOM | 313 | CG1 | ILE | A | 38 | 13.147 | 3.382 | 27.605 | 1.00 | 42.38 | C |
| ATOM | 314 | CD1 | ILE | A | 38 | 12.943 | 1.953 | 28.095 | 1.00 | 42.09 | C |
| ATOM | 315 | CG2 | ILE | A | 38 | 12.971 | 4.307 | 29.947 | 1.00 | 35.62 | C |
| ATOM | 316 | C | ILE | A | 38 | 14.332 | 6.607 | 29.307 | 1.00 | 45.32 | C |
| ATOM | 317 | O | ILE | A | 38 | 13.458 | 7.199 | 29.965 | 1.00 | 38.85 | O |
| ATOM | 318 | N | ILE | A | 39 | 15.614 | 6.686 | 29.636 | 1.00 | 55.36 | N |
| ATOM | 319 | CA | ILE | A | 39 | 15.987 | 7.617 | 30.684 | 1.00 | 54.74 | C |
| ATOM | 320 | CB | ILE | A | 39 | 17.488 | 7.528 | 31.081 | 1.00 | 50.42 | C |
| ATOM | 321 | CG1 | ILE | A | 39 | 17.707 | 6.418 | 32.111 | 1.00 | 47.26 | C |
| ATOM | 322 | CD1 | ILE | A | 39 | 17.695 | 5.035 | 31.522 | 1.00 | 55.04 | C |
| ATOM | 323 | CG2 | ILE | A | 39 | 17.948 | 8.819 | 31.697 | 1.00 | 47.52 | C |
| ATOM | 324 | C | ILE | A | 39 | 15.602 | 8.991 | 30.163 | 1.00 | 51.78 | C |
| ATOM | 325 | O | ILE | A | 39 | 15.035 | 9.792 | 30.889 | 1.00 | 55.74 | O |
| ATOM | 326 | N | GLU | A | 40 | 15.867 | 9.231 | 28.881 | 1.00 | 51.45 | N |
| ATOM | 327 | CA | GLU | A | 40 | 15.653 | 10.546 | 28.271 | 1.00 | 57.83 | C |
| ATOM | 328 | CB | GLU | A | 40 | 16.241 | 10.588 | 26.854 | 1.00 | 58.08 | C |
| ATOM | 329 | CG | GLU | A | 40 | 17.765 | 10.442 | 26.786 | 1.00 | 68.61 | C |
| ATOM | 330 | CD | GLU | A | 40 | 18.542 | 11.730 | 27.122 | 1.00 | 80.83 | C |
| ATOM | 331 | OE1 | GLU | A | 40 | 17.926 | 12.829 | 27.167 | 1.00 | 78.79 | O |
| ATOM | 332 | OE2 | GLU | A | 40 | 19.781 | 11.630 | 27.329 | 1.00 | 71.59 | O |
| ATOM | 333 | C | GLU | A | 40 | 14.191 | 11.047 | 28.282 | 1.00 | 55.84 | C |
| ATOM | 334 | O | GLU | A | 40 | 13.945 | 12.214 | 28.577 | 1.00 | 50.91 | O |
| ATOM | 335 | N | GLU | A | 41 | 13.227 | 10.181 | 27.976 | 1.00 | 59.10 | N |
| ATOM | 336 | CA | GLU | A | 41 | 11.818 | 10.598 | 27.996 | 1.00 | 62.42 | C |
| ATOM | 337 | CB | GLU | A | 41 | 10.979 | 9.844 | 26.947 | 1.00 | 70.65 | C |
| ATOM | 338 | CG | GLU | A | 41 | 10.362 | 8.593 | 27.480 | 1.00 | 73.03 | C |
| ATOM | 339 | CD | GLU | A | 41 | 11.367 | 7.824 | 28.277 | 1.00 | 74.65 | C |
| ATOM | 340 | OE1 | GLU | A | 41 | 12.529 | 7.813 | 27.828 | 1.00 | 76.53 | O |
| ATOM | 341 | OE2 | GLU | A | 41 | 11.024 | 7.279 | 29.352 | 1.00 | 75.48 | O |
| ATOM | 342 | C | GLU | A | 41 | 11.166 | 10.520 | 29.382 | 1.00 | 68.27 | C |
| ATOM | 343 | O | GLU | A | 41 | 10.053 | 10.997 | 29.567 | 1.00 | 67.91 | O |
| ATOM | 344 | N | LEU | A | 42 | 11.856 | 9.917 | 30.350 | 1.00 | 60.11 | N |
| ATOM | 345 | CA | LEU | A | 42 | 11.400 | 9.973 | 31.734 | 1.00 | 50.89 | C |
| ATOM | 346 | CB | LEU | A | 42 | 11.890 | 8.773 | 32.546 | 1.00 | 50.48 | C |
| ATOM | 347 | CG | LEU | A | 42 | 11.289 | 7.365 | 32.359 | 1.00 | 51.90 | C |
| ATOM | 348 | CD1 | LEU | A | 42 | 12.166 | 6.317 | 33.041 | 1.00 | 51.41 | C |
| ATOM | 349 | CD2 | LEU | A | 42 | 9.882 | 7.256 | 32.891 | 1.00 | 47.33 | C |
| ATOM | 350 | C | LEU | A | 42 | 11.847 | 11.292 | 32.361 | 1.00 | 56.20 | C |
| ATOM | 351 | O | LEU | A | 42 | 11.141 | 11.866 | 33.183 | 1.00 | 61.90 | O |
| ATOM | 352 | N | ALA | A | 43 | 13.013 | 11.785 | 31.953 | 1.00 | 61.33 | N |
| ATOM | 353 | CA | ALA | A | 43 | 13.491 | 13.095 | 32.402 | 1.00 | 58.95 | C |
| ATOM | 354 | CB | ALA | A | 43 | 14.847 | 13.405 | 31.801 | 1.00 | 58.44 | C |
| ATOM | 355 | C | ALA | A | 43 | 12.489 | 14.185 | 32.037 | 1.00 | 66.39 | C |
| ATOM | 356 | O | ALA | A | 43 | 12.115 | 15.007 | 32.872 | 1.00 | 71.98 | O |
| ATOM | 357 | N | GLU | A | 44 | 12.041 | 14.169 | 30.787 | 1.00 | 102.79 | N |
| ATOM | 358 | CA | GLU | A | 44 | 11.005 | 15.087 | 30.316 | 1.00 | 104.45 | C |
| ATOM | 359 | CB | GLU | A | 44 | 10.829 | 14.949 | 28.802 | 1.00 | 101.94 | C |
| ATOM | 360 | CG | GLU | A | 44 | 9.495 | 15.455 | 28.284 | 1.00 | 104.58 | C |
| ATOM | 361 | CD | GLU | A | 44 | 9.264 | 16.932 | 28.555 | 1.00 | 114.25 | C |
| ATOM | 362 | OE1 | GLU | A | 44 | 9.492 | 17.384 | 29.701 | 1.00 | 107.52 | O |
| ATOM | 363 | OE2 | GLU | A | 44 | 8.845 | 17.640 | 27.612 | 1.00 | 113.75 | O |
| ATOM | 364 | C | GLU | A | 44 | 9.665 | 14.856 | 31.015 | 1.00 | 100.58 | C |
| ATOM | 365 | O | GLU | A | 44 | 9.031 | 15.790 | 31.496 | 1.00 | 108.77 | O |
| ATOM | 366 | N | GLU | A | 45 | 9.245 | 13.600 | 31.052 | 1.00 | 61.49 | N |
| ATOM | 367 | CA | GLU | A | 45 | 8.012 | 13.182 | 31.703 | 1.00 | 58.65 | C |
| ATOM | 368 | CB | GLU | A | 45 | 7.876 | 11.665 | 31.552 | 1.00 | 60.06 | C |
| ATOM | 369 | CG | GLU | A | 45 | 6.478 | 11.111 | 31.635 | 1.00 | 68.80 | C |
| ATOM | 370 | CD | GLU | A | 45 | 5.992 | 10.957 | 33.060 | 1.00 | 79.02 | C |
| ATOM | 371 | OE1 | GLU | A | 45 | 6.760 | 11.299 | 33.992 | 1.00 | 84.33 | O |
| ATOM | 372 | OE2 | GLU | A | 45 | 4.842 | 10.492 | 33.252 | 1.00 | 72.78 | O |
| ATOM | 373 | C | GLU | A | 45 | 7.935 | 13.582 | 33.190 | 1.00 | 68.45 | C |
| ATOM | 374 | O | GLU | A | 45 | 6.852 | 13.804 | 33.712 | 1.00 | 70.96 | O |
| ATOM | 375 | N | TYR | A | 46 | 9.074 | 13.662 | 33.874 | 1.00 | 76.24 | N |
| ATOM | 376 | CA | TYR | A | 46 | 9.091 | 13.983 | 35.304 | 1.00 | 76.56 | C |
| ATOM | 377 | CB | TYR | A | 46 | 10.020 | 13.038 | 36.071 | 1.00 | 72.55 | C |
| ATOM | 378 | CG | TYR | A | 46 | 9.428 | 11.708 | 36.466 | 1.00 | 64.06 | C |
| ATOM | 379 | CD1 | TYR | A | 46 | 8.379 | 11.633 | 37.357 | 1.00 | 73.18 | C |
| ATOM | 380 | CE1 | TYR | A | 46 | 7.849 | 10.411 | 37.734 | 1.00 | 72.99 | C |
| ATOM | 381 | CZ | TYR | A | 46 | 8.377 | 9.253 | 37.223 | 1.00 | 67.54 | C |
| ATOM | 382 | OH | TYR | A | 46 | 7.857 | 8.032 | 37.608 | 1.00 | 72.25 | O |
| ATOM | 383 | CE2 | TYR | A | 46 | 9.424 | 9.309 | 36.341 | 1.00 | 62.50 | C |
| ATOM | 384 | CD2 | TYR | A | 46 | 9.951 | 10.526 | 35.979 | 1.00 | 64.36 | C |
| ATOM | 385 | C | TYR | A | 46 | 9.569 | 15.400 | 35.565 | 1.00 | 79.28 | C |
| ATOM | 386 | O | TYR | A | 46 | 9.575 | 15.852 | 36.709 | 1.00 | 79.41 | O |
| ATOM | 387 | N | ALA | A | 47 | 10.008 | 16.079 | 34.513 | 1.00 | 90.53 | N |
| ATOM | 388 | CA | ALA | A | 47 | 10.575 | 17.424 | 34.632 | 1.00 | 94.35 | C |
| ATOM | 389 | CB | ALA | A | 47 | 10.109 | 18.305 | 33.477 | 1.00 | 94.31 | C |
| ATOM | 390 | C | ALA | A | 47 | 10.293 | 18.101 | 35.978 | 1.00 | 90.88 | C |
| ATOM | 391 | O | ALA | A | 47 | 9.147 | 18.440 | 36.284 | 1.00 | 84.10 | O |
| ATOM | 392 | N | GLY | A | 48 | 11.345 | 18.282 | 36.777 | 1.00 | 97.59 | N |
| ATOM | 393 | CA | GLY | A | 48 | 11.248 | 18.998 | 38.036 | 1.00 | 99.33 | C |
| ATOM | 394 | C | GLY | A | 48 | 10.733 | 18.158 | 39.190 | 1.00 | 104.25 | C |
| ATOM | 395 | O | GLY | A | 48 | 10.209 | 18.690 | 40.172 | 1.00 | 108.99 | O |
| ATOM | 396 | N | LYS | A | 49 | 10.867 | 16.842 | 39.068 | 1.00 | 99.69 | N |
| ATOM | 397 | CA | LYS | A | 49 | 10.507 | 15.933 | 40.148 | 1.00 | 94.06 | C |
| ATOM | 398 | CB | LYS | A | 49 | 9.346 | 15.036 | 39.742 | 1.00 | 94.57 | C |
| ATOM | 399 | CG | LYS | A | 49 | 8.019 | 15.743 | 39.599 | 1.00 | 96.63 | C |
| ATOM | 400 | CD | LYS | A | 49 | 6.983 | 14.780 | 39.051 | 1.00 | 94.95 | C |
| ATOM | 401 | CE | LYS | A | 49 | 5.582 | 15.373 | 39.080 | 1.00 | 100.99 | C |
| ATOM | 402 | NZ | LYS | A | 49 | 4.567 | 14.369 | 38.640 | 1.00 | 96.64 | N |
| ATOM | 403 | C | LYS | A | 49 | 11.711 | 15.067 | 40.444 | 1.00 | 92.39 | C |
| ATOM | 404 | O | LYS | A | 49 | 12.290 | 15.138 | 41.527 | 1.00 | 99.57 | O |
| ATOM | 405 | N | VAL | A | 50 | 12.078 | 14.247 | 39.467 | 1.00 | 62.39 | N |
| ATOM | 406 | CA | VAL | A | 50 | 13.270 | 13.433 | 39.561 | 1.00 | 56.12 | C |
| ATOM | 407 | CB | VAL | A | 50 | 12.991 | 11.990 | 39.117 | 1.00 | 52.89 | C |
| ATOM | 408 | CG1 | VAL | A | 50 | 14.238 | 11.134 | 39.280 | 1.00 | 47.70 | C |
| ATOM | 409 | CG2 | VAL | A | 50 | 11.826 | 11.408 | 39.910 | 1.00 | 55.45 | C |
| ATOM | 410 | C | VAL | A | 50 | 14.327 | 14.026 | 38.660 | 1.00 | 58.14 | C |
| ATOM | 411 | O | VAL | A | 50 | 14.033 | 14.509 | 37.560 | 1.00 | 54.59 | O |
| ATOM | 412 | N | VAL | A | 51 | 15.556 | 14.001 | 39.151 | 1.00 | 56.06 | N |
| ATOM | 413 | CA | VAL | A | 51 | 16.710 | 14.437 | 38.391 | 1.00 | 54.13 | C |
| ATOM | 414 | CB | VAL | A | 51 | 17.748 | 15.076 | 39.334 | 1.00 | 54.20 | C |
| ATOM | 415 | CG1 | VAL | A | 51 | 18.970 | 15.568 | 38.569 | 1.00 | 44.70 | C |
| ATOM | 416 | CG2 | VAL | A | 51 | 17.108 | 16.202 | 40.131 | 1.00 | 55.34 | C |
| ATOM | 417 | C | VAL | A | 51 | 17.314 | 13.196 | 37.751 | 1.00 | 52.68 | C |
| ATOM | 418 | O | VAL | A | 51 | 17.424 | 12.151 | 38.400 | 1.00 | 49.82 | O |
| ATOM | 419 | N | PHE | A | 52 | 17.709 | 13.298 | 36.487 | 1.00 | 44.22 | N |
| ATOM | 420 | CA | PHE | A | 52 | 18.283 | 12.141 | 35.808 | 1.00 | 45.68 | C |
| ATOM | 421 | CB | PHE | A | 52 | 17.374 | 11.692 | 34.655 | 1.00 | 41.91 | C |
| ATOM | 422 | CG | PHE | A | 52 | 16.031 | 11.212 | 35.101 | 1.00 | 39.09 | C |
| ATOM | 423 | CD2 | PHE | A | 52 | 15.776 | 9.853 | 35.233 | 1.00 | 42.63 | C |
| ATOM | 424 | CE2 | PHE | A | 52 | 14.535 | 9.392 | 35.650 | 1.00 | 38.82 | C |
| ATOM | 425 | CZ | PHE | A | 52 | 13.517 | 10.296 | 35.939 | 1.00 | 46.06 | C |
| ATOM | 426 | CE1 | PHE | A | 52 | 13.762 | 11.666 | 35.818 | 1.00 | 52.73 | C |
| ATOM | 427 | CD1 | PHE | A | 52 | 15.023 | 12.112 | 35.405 | 1.00 | 47.52 | C |
| ATOM | 428 | C | PHE | A | 52 | 19.696 | 12.424 | 35.302 | 1.00 | 48.32 | C |
| ATOM | 429 | O | PHE | A | 52 | 19.924 | 13.415 | 34.613 | 1.00 | 53.51 | O |
| ATOM | 430 | N | GLY | A | 53 | 20.636 | 11.542 | 35.630 | 1.00 | 44.13 | N |
| ATOM | 431 | CA | GLY | A | 53 | 22.032 | 11.738 | 35.266 | 1.00 | 46.50 | C |
| ATOM | 432 | C | GLY | A | 53 | 22.720 | 10.505 | 34.698 | 1.00 | 47.50 | C |
| ATOM | 433 | O | GLY | A | 53 | 22.436 | 9.374 | 35.099 | 1.00 | 46.48 | O |
| ATOM | 434 | N | LYS | A | 54 | 23.637 | 10.725 | 33.762 | 1.00 | 43.69 | N |
| ATOM | 435 | CA | LYS | A | 54 | 24.380 | 9.641 | 33.146 | 1.00 | 46.23 | C |
| ATOM | 436 | CB | LYS | A | 54 | 24.305 | 9.750 | 31.619 | 1.00 | 48.25 | C |
| ATOM | 437 | CG | LYS | A | 54 | 22.898 | 9.819 | 31.064 | 1.00 | 59.49 | C |
| ATOM | 438 | CD | LYS | A | 54 | 22.904 | 9.871 | 29.535 | 1.00 | 62.01 | C |
| ATOM | 439 | CE | LYS | A | 54 | 23.287 | 8.522 | 28.941 | 1.00 | 63.95 | C |
| ATOM | 440 | NZ | LYS | A | 54 | 22.511 | 7.412 | 29.558 | 1.00 | 62.92 | N |
| ATOM | 441 | C | LYS | A | 54 | 25.836 | 9.681 | 33.579 | 1.00 | 45.36 | C |
| ATOM | 442 | O | LYS | A | 54 | 26.397 | 10.755 | 33.774 | 1.00 | 47.64 | O |
| ATOM | 443 | N | VAL | A | 55 | 26.446 | 8.506 | 33.692 | 1.00 | 38.19 | N |
| ATOM | 444 | CA | VAL | A | 55 | 27.850 | 8.386 | 34.061 | 1.00 | 33.44 | C |
| ATOM | 445 | CB | VAL | A | 55 | 28.018 | 7.946 | 35.528 | 1.00 | 44.45 | C |
| ATOM | 446 | CG1 | VAL | A | 55 | 29.493 | 7.646 | 35.858 | 1.00 | 34.00 | C |
| ATOM | 447 | CG2 | VAL | A | 55 | 27.458 | 9.007 | 36.475 | 1.00 | 39.10 | C |
| ATOM | 448 | C | VAL | A | 55 | 28.598 | 7.373 | 33.201 | 1.00 | 36.90 | C |
| ATOM | 449 | O | VAL | A | 55 | 28.381 | 6.167 | 33.320 | 1.00 | 39.80 | O |
| ATOM | 450 | N | ASN | A | 56 | 29.510 | 7.871 | 32.369 | 1.00 | 41.37 | N |
| ATOM | 451 | CA | ASN | A | 56 | 30.322 | 7.039 | 31.488 | 1.00 | 40.98 | C |
| ATOM | 452 | CB | ASN | A | 56 | 30.999 | 7.922 | 30.432 | 1.00 | 43.39 | C |
| ATOM | 453 | CG | ASN | A | 56 | 31.533 | 7.129 | 29.248 | 1.00 | 56.82 | C |
| ATOM | 454 | OD1 | ASN | A | 56 | 32.252 | 6.147 | 29.422 | 1.00 | 58.12 | O |
| ATOM | 455 | ND2 | ASN | A | 56 | 31.168 | 7.552 | 28.025 | 1.00 | 55.17 | N |
| ATOM | 456 | C | ASN | A | 56 | 31.360 | 6.209 | 32.250 | 1.00 | 47.59 | C |
| ATOM | 457 | O | ASN | A | 56 | 32.392 | 6.699 | 32.702 | 1.00 | 56.82 | O |
| ATOM | 458 | N | VAL | A | 57 | 31.088 | 4.931 | 32.380 | 1.00 | 35.23 | N |
| ATOM | 459 | CA | VAL | A | 57 | 31.937 | 4.065 | 33.165 | 1.00 | 38.26 | C |
| ATOM | 460 | CB | VAL | A | 57 | 31.213 | 2.751 | 33.344 | 1.00 | 35.80 | C |
| ATOM | 461 | CG1 | VAL | A | 57 | 32.169 | 1.599 | 33.536 | 1.00 | 49.63 | C |
| ATOM | 462 | CG2 | VAL | A | 57 | 30.221 | 2.897 | 34.503 | 1.00 | 32.54 | C |
| ATOM | 463 | C | VAL | A | 57 | 33.392 | 3.935 | 32.645 | 1.00 | 56.19 | C |
| ATOM | 464 | O | VAL | A | 57 | 34.306 | 3.509 | 33.373 | 1.00 | 54.69 | O |
| ATOM | 465 | N | ASP | A | 58 | 33.610 | 4.342 | 31.401 | 1.00 | 56.01 | N |
| ATOM | 466 | CA | ASP | A | 58 | 34.962 | 4.470 | 30.874 | 1.00 | 61.21 | C |
| ATOM | 467 | CB | ASP | A | 58 | 34.964 | 4.525 | 29.341 | 1.00 | 59.67 | C |
| ATOM | 468 | CG | ASP | A | 58 | 34.598 | 3.193 | 28.710 | 1.00 | 66.87 | C |
| ATOM | 469 | OD1 | ASP | A | 58 | 34.975 | 2.138 | 29.275 | 1.00 | 62.78 | O |
| ATOM | 470 | OD2 | ASP | A | 58 | 33.919 | 3.200 | 27.655 | 1.00 | 78.46 | O |
| ATOM | 471 | C | ASP | A | 58 | 35.614 | 5.722 | 31.433 | 1.00 | 67.99 | C |
| ATOM | 472 | O | ASP | A | 58 | 36.644 | 5.649 | 32.091 | 1.00 | 78.17 | O |
| ATOM | 473 | N | GLU | A | 59 | 34.994 | 6.870 | 31.196 | 1.00 | 65.81 | N |
| ATOM | 474 | CA | GLU | A | 59 | 35.532 | 8.137 | 31.676 | 1.00 | 67.13 | C |
| ATOM | 475 | CB | GLU | A | 59 | 34.695 | 9.301 | 31.124 | 1.00 | 68.72 | C |
| ATOM | 476 | CG | GLU | A | 59 | 34.163 | 9.059 | 29.691 | 1.00 | 71.52 | C |
| ATOM | 477 | CD | GLU | A | 59 | 33.552 | 10.309 | 29.021 | 1.00 | 79.95 | C |
| ATOM | 478 | OE1 | GLU | A | 59 | 32.885 | 11.108 | 29.718 | 1.00 | 79.67 | O |
| ATOM | 479 | OE2 | GLU | A | 59 | 33.736 | 10.488 | 27.790 | 1.00 | 72.91 | O |
| ATOM | 480 | C | GLU | A | 59 | 35.648 | 8.218 | 33.216 | 1.00 | 77.98 | C |
| ATOM | 481 | O | GLU | A | 59 | 36.376 | 9.062 | 33.736 | 1.00 | 76.37 | O |
| ATOM | 482 | N | ASN | A | 60 | 34.950 | 7.337 | 33.942 | 1.00 | 67.37 | N |
| ATOM | 483 | CA | ASN | A | 60 | 34.921 | 7.407 | 35.412 | 1.00 | 60.51 | C |
| ATOM | 484 | CB | ASN | A | 60 | 33.711 | 8.214 | 35.890 | 1.00 | 46.14 | C |
| ATOM | 485 | CG | ASN | A | 60 | 33.282 | 9.282 | 34.886 | 1.00 | 60.40 | C |
| ATOM | 486 | OD1 | ASN | A | 60 | 33.563 | 10.473 | 35.052 | 1.00 | 64.54 | O |
| ATOM | 487 | ND2 | ASN | A | 60 | 32.590 | 8.854 | 33.838 | 1.00 | 53.67 | N |
| ATOM | 488 | C | ASN | A | 60 | 34.913 | 6.042 | 36.103 | 1.00 | 59.53 | C |
| ATOM | 489 | O | ASN | A | 60 | 33.963 | 5.710 | 36.813 | 1.00 | 52.80 | O |
| ATOM | 490 | N | PRO | A | 61 | 35.996 | 5.265 | 35.930 | 1.00 | 72.66 | N |
| ATOM | 491 | CA | PRO | A | 61 | 36.143 | 3.863 | 36.375 | 1.00 | 69.61 | C |
| ATOM | 492 | CB | PRO | A | 61 | 37.554 | 3.477 | 35.898 | 1.00 | 63.03 | C |
| ATOM | 493 | CG | PRO | A | 61 | 37.995 | 4.586 | 34.989 | 1.00 | 69.75 | C |
| ATOM | 494 | CD | PRO | A | 61 | 37.249 | 5.808 | 35.383 | 1.00 | 69.43 | C |
| ATOM | 495 | C | PRO | A | 61 | 36.075 | 3.648 | 37.891 | 1.00 | 73.17 | C |
| ATOM | 496 | O | PRO | A | 61 | 35.811 | 2.521 | 38.340 | 1.00 | 64.06 | O |
| ATOM | 497 | N | GLU | A | 62 | 36.331 | 4.708 | 38.656 | 1.00 | 84.34 | N |
| ATOM | 498 | CA | GLU | A | 62 | 36.444 | 4.615 | 40.110 | 1.00 | 93.89 | C |
| ATOM | 499 | CB | GLU | A | 62 | 37.537 | 5.555 | 40.636 | 1.00 | 84.86 | C |
| ATOM | 500 | CG | GLU | A | 62 | 37.250 | 7.031 | 40.410 | 1.00 | 92.10 | C |
| ATOM | 501 | CD | GLU | A | 62 | 36.924 | 7.346 | 38.949 | 1.00 | 93.85 | C |
| ATOM | 502 | OE1 | GLU | A | 62 | 37.863 | 7.557 | 38.153 | 1.00 | 94.51 | O |
| ATOM | 503 | OE2 | GLU | A | 62 | 35.728 | 7.377 | 38.590 | 1.00 | 93.66 | O |
| ATOM | 504 | C | GLU | A | 62 | 35.104 | 4.874 | 40.808 | 1.00 | 88.68 | C |
| ATOM | 505 | O | GLU | A | 62 | 34.675 | 4.068 | 41.626 | 1.00 | 78.72 | O |
| ATOM | 506 | N | ILE | A | 63 | 34.451 | 5.992 | 40.486 | 1.00 | 74.77 | N |
| ATOM | 507 | CA | ILE | A | 63 | 33.091 | 6.252 | 40.969 | 1.00 | 77.65 | C |
| ATOM | 508 | CB | ILE | A | 63 | 32.531 | 7.531 | 40.355 | 1.00 | 65.68 | C |
| ATOM | 509 | CG1 | ILE | A | 63 | 31.035 | 7.633 | 40.587 | 1.00 | 65.15 | C |
| ATOM | 510 | CD1 | ILE | A | 63 | 30.475 | 8.968 | 40.147 | 1.00 | 68.07 | C |
| ATOM | 511 | CG2 | ILE | A | 63 | 32.758 | 7.515 | 38.893 | 1.00 | 68.04 | C |
| ATOM | 512 | C | ILE | A | 63 | 32.241 | 5.045 | 40.595 | 1.00 | 66.12 | C |
| ATOM | 513 | O | ILE | A | 63 | 31.450 | 4.529 | 41.386 | 1.00 | 62.36 | O |
| ATOM | 514 | N | ALA | A | 64 | 32.454 | 4.570 | 39.383 | 1.00 | 48.98 | N |
| ATOM | 515 | CA | ALA | A | 64 | 31.961 | 3.258 | 39.018 | 1.00 | 52.44 | C |
| ATOM | 516 | CB | ALA | A | 64 | 32.567 | 2.814 | 37.683 | 1.00 | 49.52 | C |
| ATOM | 517 | C | ALA | A | 64 | 32.291 | 2.259 | 40.134 | 1.00 | 47.56 | C |
| ATOM | 518 | O | ALA | A | 64 | 31.393 | 1.709 | 40.772 | 1.00 | 47.94 | O |
| ATOM | 519 | N | ALA | A | 65 | 33.582 | 2.057 | 40.390 | 1.00 | 76.11 | N |
| ATOM | 520 | CA | ALA | A | 65 | 34.043 | 1.109 | 41.414 | 1.00 | 77.00 | C |
| ATOM | 521 | CB | ALA | A | 65 | 35.537 | 0.858 | 41.267 | 1.00 | 75.70 | C |
| ATOM | 522 | C | ALA | A | 65 | 33.713 | 1.532 | 42.850 | 1.00 | 66.64 | C |
| ATOM | 523 | O | ALA | A | 65 | 33.484 | 0.678 | 43.701 | 1.00 | 61.04 | O |
| ATOM | 524 | N | LYS | A | 66 | 33.696 | 2.837 | 43.114 | 1.00 | 44.96 | N |
| ATOM | 525 | CA | LYS | A | 66 | 33.328 | 3.336 | 44.433 | 1.00 | 51.42 | C |
| ATOM | 526 | CB | LYS | A | 66 | 33.148 | 4.852 | 44.413 | 1.00 | 53.94 | C |
| ATOM | 527 | CG | LYS | A | 66 | 32.494 | 5.390 | 45.701 | 1.00 | 59.24 | C |
| ATOM | 528 | CD | LYS | A | 66 | 32.471 | 6.934 | 45.774 | 1.00 | 62.72 | C |
| ATOM | 529 | CE | LYS | A | 66 | 33.814 | 7.565 | 45.381 | 1.00 | 70.11 | C |
| ATOM | 530 | NZ | LYS | A | 66 | 34.074 | 7.548 | 43.891 | 1.00 | 73.37 | N |
| ATOM | 531 | C | LYS | A | 66 | 32.017 | 2.710 | 44.854 | 1.00 | 57.94 | C |
| ATOM | 532 | O | LYS | A | 66 | 31.956 | 1.915 | 45.797 | 1.00 | 59.93 | O |
| ATOM | 533 | N | TYR | A | 67 | 30.972 | 3.081 | 44.122 | 1.00 | 60.30 | N |
| ATOM | 534 | CA | TYR | A | 67 | 29.635 | 2.589 | 44.346 | 1.00 | 51.39 | C |
| ATOM | 535 | CB | TYR | A | 67 | 28.639 | 3.549 | 43.719 | 1.00 | 48.16 | C |
| ATOM | 536 | CG | TYR | A | 67 | 28.561 | 4.916 | 44.374 | 1.00 | 50.71 | C |
| ATOM | 537 | CD1 | TYR | A | 67 | 27.942 | 5.085 | 45.611 | 1.00 | 52.64 | C |
| ATOM | 538 | CE1 | TYR | A | 67 | 27.848 | 6.349 | 46.214 | 1.00 | 50.96 | C |
| ATOM | 539 | CZ | TYR | A | 67 | 28.372 | 7.462 | 45.570 | 1.00 | 54.02 | C |
| ATOM | 540 | OH | TYR | A | 67 | 28.277 | 8.721 | 46.157 | 1.00 | 48.49 | O |
| ATOM | 541 | CE2 | TYR | A | 67 | 28.987 | 7.313 | 44.331 | 1.00 | 49.25 | C |
| ATOM | 542 | CD2 | TYR | A | 67 | 29.078 | 6.044 | 43.746 | 1.00 | 48.30 | C |
| ATOM | 543 | C | TYR | A | 67 | 29.437 | 1.184 | 43.776 | 1.00 | 54.82 | C |
| ATOM | 544 | O | TYR | A | 67 | 28.310 | 0.766 | 43.515 | 1.00 | 56.39 | O |
| ATOM | 545 | N | GLY | A | 68 | 30.536 | 0.466 | 43.574 | 1.00 | 56.40 | N |
| ATOM | 546 | CA | GLY | A | 68 | 30.482 | −0.950 | 43.254 | 1.00 | 55.05 | C |
| ATOM | 547 | C | GLY | A | 68 | 29.645 | −1.348 | 42.056 | 1.00 | 67.16 | C |
| ATOM | 548 | O | GLY | A | 68 | 28.865 | −2.312 | 42.113 | 1.00 | 64.48 | O |
| ATOM | 549 | N | ILE | A | 69 | 29.816 | −0.617 | 40.959 | 1.00 | 60.21 | N |
| ATOM | 550 | CA | ILE | A | 69 | 29.095 | −0.923 | 39.733 | 1.00 | 64.12 | C |
| ATOM | 551 | CB | ILE | A | 69 | 29.036 | 0.286 | 38.769 | 1.00 | 65.56 | C |
| ATOM | 552 | CG1 | ILE | A | 69 | 28.344 | 1.460 | 39.460 | 1.00 | 60.89 | C |
| ATOM | 553 | CD1 | ILE | A | 69 | 26.968 | 1.108 | 39.974 | 1.00 | 52.49 | C |
| ATOM | 554 | CG2 | ILE | A | 69 | 28.291 | −0.088 | 37.491 | 1.00 | 53.30 | C |
| ATOM | 555 | C | ILE | A | 69 | 29.735 | −2.120 | 39.040 | 1.00 | 68.87 | C |
| ATOM | 556 | O | ILE | A | 69 | 30.838 | −2.029 | 38.498 | 1.00 | 56.10 | O |
| ATOM | 557 | N | MET | A | 70 | 29.025 | −3.240 | 39.062 | 1.00 | 96.40 | N |
| ATOM | 558 | CA | MET | A | 70 | 29.536 | −4.486 | 38.519 | 1.00 | 91.03 | C |
| ATOM | 559 | CB | MET | A | 70 | 28.636 | −5.641 | 38.952 | 1.00 | 92.70 | C |
| ATOM | 560 | CG | MET | A | 70 | 28.511 | −5.796 | 40.462 | 1.00 | 110.83 | C |
| ATOM | 561 | SD | MET | A | 70 | 30.035 | −6.383 | 41.252 | 1.00 | 131.58 | S |
| ATOM | 562 | CE | MET | A | 70 | 30.629 | −4.890 | 42.043 | 1.00 | 107.40 | C |
| ATOM | 563 | C | MET | A | 70 | 29.578 | −4.434 | 37.005 | 1.00 | 94.80 | C |
| ATOM | 564 | O | MET | A | 70 | 30.609 | −4.117 | 36.413 | 1.00 | 94.51 | O |
| ATOM | 565 | N | ASER | A | 71 | 28.444 | −4.755 | 36.403 | 1.00 | 66.34 | N |
| ATOM | 566 | CA | ASER | A | 71 | 28.257 | −4.705 | 34.972 | 1.00 | 60.37 | C |
| ATOM | 567 | CB | ASER | A | 71 | 27.592 | −5.995 | 34.517 | 1.00 | 62.84 | C |
| ATOM | 568 | OG | ASER | A | 71 | 26.418 | −6.218 | 35.276 | 1.00 | 60.95 | O |
| ATOM | 569 | C | ASER | A | 71 | 27.327 | −3.561 | 34.670 | 1.00 | 56.56 | C |
| ATOM | 570 | O | ASER | A | 71 | 26.519 | −3.190 | 35.496 | 1.00 | 60.71 | O |
| ATOM | 571 | N | BSER | A | 71 | 28.455 | −4.756 | 36.382 | 0.00 | 66.65 | N |
| ATOM | 572 | CA | BSER | A | 71 | 28.339 | −4.711 | 34.932 | 0.00 | 59.78 | C |
| ATOM | 573 | CB | BSER | A | 71 | 28.094 | −6.109 | 34.360 | 0.00 | 60.66 | C |
| ATOM | 574 | OG | BSER | A | 71 | 29.157 | −6.993 | 34.676 | 0.00 | 61.36 | O |
| ATOM | 575 | C | BSER | A | 71 | 27.208 | −3.773 | 34.530 | 0.00 | 57.10 | C |
| ATOM | 576 | O | BSER | A | 71 | 26.156 | −3.743 | 35.160 | 0.00 | 56.96 | O |
| ATOM | 577 | N | ILE | A | 72 | 27.437 | −3.003 | 33.476 | 1.00 | 46.79 | N |
| ATOM | 578 | CA | ILE | A | 72 | 26.457 | −2.046 | 32.998 | 1.00 | 44.17 | C |
| ATOM | 579 | CB | ILE | A | 72 | 27.123 | −0.807 | 32.385 | 1.00 | 39.94 | C |
| ATOM | 580 | CG1 | ILE | A | 72 | 28.158 | −1.201 | 31.325 | 1.00 | 30.91 | C |
| ATOM | 581 | CD1 | ILE | A | 72 | 28.853 | 0.020 | 30.721 | 1.00 | 35.85 | C |
| ATOM | 582 | CG2 | ILE | A | 72 | 27.713 | 0.064 | 33.487 | 1.00 | 33.09 | C |
| ATOM | 583 | C | ILE | A | 72 | 25.540 | −2.738 | 31.980 | 1.00 | 42.80 | C |
| ATOM | 584 | O | ILE | A | 72 | 25.865 | −3.833 | 31.516 | 1.00 | 45.02 | O |
| ATOM | 585 | N | PRO | A | 73 | 24.364 | −2.139 | 31.681 | 1.00 | 45.08 | N |
| ATOM | 586 | CA | PRO | A | 73 | 23.842 | −0.898 | 32.275 | 1.00 | 42.17 | C |
| ATOM | 587 | CB | PRO | A | 73 | 22.685 | −0.538 | 31.352 | 1.00 | 40.79 | C |
| ATOM | 588 | CG | PRO | A | 73 | 22.118 | −1.894 | 30.988 | 1.00 | 44.40 | C |
| ATOM | 589 | CD | PRO | A | 73 | 23.324 | −2.840 | 30.894 | 1.00 | 48.45 | C |
| ATOM | 590 | C | PRO | A | 73 | 23.287 | −1.187 | 33.661 | 1.00 | 41.24 | C |
| ATOM | 591 | O | PRO | A | 73 | 22.857 | −2.311 | 33.931 | 1.00 | 41.06 | O |
| ATOM | 592 | N | THR | A | 74 | 23.272 | −0.168 | 34.506 | 1.00 | 34.88 | N |
| ATOM | 593 | CA | THR | A | 74 | 22.797 | −0.297 | 35.859 | 1.00 | 36.45 | C |
| ATOM | 594 | CB | THR | A | 74 | 23.935 | −0.797 | 36.765 | 1.00 | 46.44 | C |
| ATOM | 595 | OG1 | THR | A | 74 | 23.949 | −2.232 | 36.738 | 1.00 | 43.67 | O |
| ATOM | 596 | CG2 | THR | A | 74 | 23.781 | −0.283 | 38.210 | 1.00 | 44.52 | C |
| ATOM | 597 | C | THR | A | 74 | 22.294 | 1.059 | 36.300 | 1.00 | 33.11 | C |
| ATOM | 598 | O | THR | A | 74 | 22.888 | 2.086 | 35.977 | 1.00 | 34.58 | O |
| ATOM | 599 | N | LEU | A | 75 | 21.178 | 1.076 | 37.011 | 1.00 | 36.36 | N |
| ATOM | 600 | CA | LEU | A | 75 | 20.620 | 2.336 | 37.476 | 1.00 | 38.78 | C |
| ATOM | 601 | C | LEU | A | 75 | 20.703 | 2.435 | 38.988 | 1.00 | 38.71 | C |
| ATOM | 602 | O | LEU | A | 75 | 20.427 | 1.463 | 39.678 | 1.00 | 40.16 | O |
| ATOM | 603 | CB | LEU | A | 75 | 19.170 | 2.431 | 37.056 | 1.00 | 33.84 | C |
| ATOM | 604 | CG | LEU | A | 75 | 18.827 | 3.192 | 35.790 | 1.00 | 36.18 | C |
| ATOM | 605 | CD1 | LEU | A | 75 | 19.805 | 2.879 | 34.700 | 1.00 | 44.96 | C |
| ATOM | 606 | CD2 | LEU | A | 75 | 17.427 | 2.779 | 35.397 | 1.00 | 34.75 | C |
| ATOM | 607 | N | LEU | A | 76 | 21.048 | 3.610 | 39.502 | 1.00 | 35.54 | N |
| ATOM | 608 | CA | LEU | A | 76 | 21.203 | 3.805 | 40.942 | 1.00 | 35.11 | C |
| ATOM | 609 | CB | LEU | A | 76 | 22.633 | 4.274 | 41.254 | 1.00 | 43.47 | C |
| ATOM | 610 | CG | LEU | A | 76 | 23.510 | 3.459 | 42.201 | 1.00 | 39.70 | C |
| ATOM | 611 | CD1 | LEU | A | 76 | 23.430 | 1.980 | 41.853 | 1.00 | 37.85 | C |
| ATOM | 612 | CD2 | LEU | A | 76 | 24.945 | 3.951 | 42.112 | 1.00 | 45.25 | C |
| ATOM | 613 | C | LEU | A | 76 | 20.256 | 4.888 | 41.377 | 1.00 | 35.01 | C |
| ATOM | 614 | O | LEU | A | 76 | 20.291 | 5.982 | 40.830 | 1.00 | 37.73 | O |
| ATOM | 615 | N | PHE | A | 77 | 19.399 | 4.611 | 42.353 | 1.00 | 48.58 | N |
| ATOM | 616 | CA | PHE | A | 77 | 18.599 | 5.696 | 42.910 | 1.00 | 48.57 | C |
| ATOM | 617 | CB | PHE | A | 77 | 17.170 | 5.259 | 43.240 | 1.00 | 48.66 | C |
| ATOM | 618 | CG | PHE | A | 77 | 16.444 | 4.633 | 42.079 | 1.00 | 51.12 | C |
| ATOM | 619 | CD1 | PHE | A | 77 | 17.073 | 4.469 | 40.857 | 1.00 | 57.06 | C |
| ATOM | 620 | CE1 | PHE | A | 77 | 16.428 | 3.885 | 39.789 | 1.00 | 48.27 | C |
| ATOM | 621 | CZ | PHE | A | 77 | 15.131 | 3.466 | 39.921 | 1.00 | 55.24 | C |
| ATOM | 622 | CE2 | PHE | A | 77 | 14.479 | 3.633 | 41.125 | 1.00 | 64.99 | C |
| ATOM | 623 | CD2 | PHE | A | 77 | 15.139 | 4.216 | 42.201 | 1.00 | 59.20 | C |
| ATOM | 624 | C | PHE | A | 77 | 19.307 | 6.247 | 44.139 | 1.00 | 50.97 | C |
| ATOM | 625 | O | PHE | A | 77 | 19.667 | 5.506 | 45.060 | 1.00 | 46.84 | O |
| ATOM | 626 | N | PHE | A | 78 | 19.521 | 7.558 | 44.122 | 1.00 | 50.78 | N |
| ATOM | 627 | CA | PHE | A | 78 | 20.211 | 8.260 | 45.182 | 1.00 | 49.34 | C |
| ATOM | 628 | CB | PHE | A | 78 | 21.293 | 9.164 | 44.597 | 1.00 | 49.00 | C |
| ATOM | 629 | CG | PHE | A | 78 | 22.602 | 8.474 | 44.329 | 1.00 | 55.83 | C |
| ATOM | 630 | CD2 | PHE | A | 78 | 22.878 | 7.942 | 43.083 | 1.00 | 57.57 | C |
| ATOM | 631 | CE2 | PHE | A | 78 | 24.099 | 7.320 | 42.834 | 1.00 | 56.85 | C |
| ATOM | 632 | CZ | PHE | A | 78 | 25.059 | 7.241 | 43.825 | 1.00 | 54.28 | C |
| ATOM | 633 | CE1 | PHE | A | 78 | 24.799 | 7.772 | 45.065 | 1.00 | 51.56 | C |
| ATOM | 634 | CD1 | PHE | A | 78 | 23.580 | 8.391 | 45.312 | 1.00 | 59.69 | C |
| ATOM | 635 | C | PHE | A | 78 | 19.208 | 9.138 | 45.893 | 1.00 | 54.17 | C |
| ATOM | 636 | O | PHE | A | 78 | 18.461 | 9.863 | 45.249 | 1.00 | 51.45 | O |
| ATOM | 637 | N | LYS | A | 79 | 19.204 | 9.097 | 47.217 | 1.00 | 63.44 | N |
| ATOM | 638 | CA | LYS | A | 79 | 18.310 | 9.947 | 47.986 | 1.00 | 64.82 | C |
| ATOM | 639 | CB | LYS | A | 79 | 17.132 | 9.125 | 48.525 | 1.00 | 62.83 | C |
| ATOM | 640 | CG | LYS | A | 79 | 16.005 | 9.963 | 49.076 | 1.00 | 57.94 | C |
| ATOM | 641 | CD | LYS | A | 79 | 15.606 | 11.011 | 48.078 | 1.00 | 64.26 | C |
| ATOM | 642 | CE | LYS | A | 79 | 14.563 | 11.945 | 48.646 | 1.00 | 64.13 | C |
| ATOM | 643 | NZ | LYS | A | 79 | 15.199 | 13.174 | 49.167 | 1.00 | 67.89 | N |
| ATOM | 644 | C | LYS | A | 79 | 19.094 | 10.592 | 49.127 | 1.00 | 60.03 | C |
| ATOM | 645 | O | LYS | A | 79 | 19.835 | 9.913 | 49.844 | 1.00 | 62.59 | O |
| ATOM | 646 | N | ASN | A | 80 | 18.941 | 11.902 | 49.284 | 1.00 | 46.36 | N |
| ATOM | 647 | CA | ASN | A | 80 | 19.639 | 12.617 | 50.347 | 1.00 | 57.29 | C |
| ATOM | 648 | CB | ASN | A | 80 | 19.017 | 12.305 | 51.721 | 1.00 | 54.52 | C |
| ATOM | 649 | CG | ASN | A | 80 | 17.672 | 12.980 | 51.922 | 1.00 | 56.75 | C |
| ATOM | 650 | OD1 | ASN | A | 80 | 17.488 | 14.149 | 51.562 | 1.00 | 55.42 | O |
| ATOM | 651 | ND2 | ASN | A | 80 | 16.720 | 12.246 | 52.498 | 1.00 | 52.91 | N |
| ATOM | 652 | C | ASN | A | 80 | 21.128 | 12.286 | 50.375 | 1.00 | 54.15 | C |
| ATOM | 653 | O | ASN | A | 80 | 21.720 | 12.210 | 51.442 | 1.00 | 59.64 | O |
| ATOM | 654 | N | GLY | A | 81 | 21.722 | 12.070 | 49.204 | 1.00 | 46.36 | N |
| ATOM | 655 | CA | GLY | A | 81 | 23.153 | 11.855 | 49.089 | 1.00 | 37.03 | C |
| ATOM | 656 | C | GLY | A | 81 | 23.558 | 10.402 | 49.131 | 1.00 | 40.71 | C |
| ATOM | 657 | O | GLY | A | 81 | 24.663 | 10.053 | 48.713 | 1.00 | 46.00 | O |
| ATOM | 658 | N | LYS | A | 82 | 22.678 | 9.543 | 49.631 | 1.00 | 40.79 | N |
| ATOM | 659 | CA | LYS | A | 82 | 23.013 | 8.121 | 49.717 | 1.00 | 45.83 | C |
| ATOM | 660 | CB | LYS | A | 82 | 22.887 | 7.605 | 51.161 | 1.00 | 51.57 | C |
| ATOM | 661 | CG | LYS | A | 82 | 21.467 | 7.575 | 51.741 | 1.00 | 51.54 | C |
| ATOM | 662 | CD | LYS | A | 82 | 21.505 | 7.293 | 53.262 | 1.00 | 47.42 | C |
| ATOM | 663 | CE | LYS | A | 82 | 20.107 | 7.116 | 53.849 | 1.00 | 58.79 | C |
| ATOM | 664 | NZ | LYS | A | 82 | 19.113 | 8.117 | 53.333 | 1.00 | 57.60 | N |
| ATOM | 665 | C | LYS | A | 82 | 22.218 | 7.239 | 48.742 | 1.00 | 53.14 | C |
| ATOM | 666 | O | LYS | A | 82 | 21.035 | 7.495 | 48.453 | 1.00 | 45.11 | O |
| ATOM | 667 | N | VAL | A | 83 | 22.882 | 6.201 | 48.238 | 1.00 | 57.57 | N |
| ATOM | 668 | CA | VAL | A | 83 | 22.258 | 5.242 | 47.325 | 1.00 | 47.88 | C |
| ATOM | 669 | CB | VAL | A | 83 | 23.292 | 4.241 | 46.806 | 1.00 | 42.21 | C |
| ATOM | 670 | CG1 | VAL | A | 83 | 24.342 | 4.002 | 47.879 | 1.00 | 72.41 | C |
| ATOM | 671 | CG2 | VAL | A | 83 | 22.618 | 2.935 | 46.380 | 1.00 | 46.97 | C |
| ATOM | 672 | C | VAL | A | 83 | 21.072 | 4.512 | 47.983 | 1.00 | 57.40 | C |
| ATOM | 673 | O | VAL | A | 83 | 21.165 | 4.056 | 49.126 | 1.00 | 52.23 | O |
| ATOM | 674 | N | VAL | A | 84 | 19.955 | 4.401 | 47.258 | 1.00 | 55.17 | N |
| ATOM | 675 | CA | VAL | A | 84 | 18.708 | 3.932 | 47.866 | 1.00 | 50.27 | C |
| ATOM | 676 | CB | VAL | A | 84 | 17.729 | 5.132 | 48.079 | 1.00 | 55.25 | C |
| ATOM | 677 | CG1 | VAL | A | 84 | 16.433 | 4.967 | 47.298 | 1.00 | 51.31 | C |
| ATOM | 678 | CG2 | VAL | A | 84 | 17.470 | 5.338 | 49.559 | 1.00 | 48.68 | C |
| ATOM | 679 | C | VAL | A | 84 | 18.047 | 2.739 | 47.157 | 1.00 | 45.69 | C |
| ATOM | 680 | O | VAL | A | 84 | 17.122 | 2.142 | 47.679 | 1.00 | 47.56 | O |
| ATOM | 681 | N | ASP | A | 85 | 18.557 | 2.381 | 45.986 | 1.00 | 48.47 | N |
| ATOM | 682 | CA | ASP | A | 85 | 18.022 | 1.284 | 45.181 | 1.00 | 49.86 | C |
| ATOM | 683 | CB | ASP | A | 85 | 16.589 | 1.590 | 44.721 | 1.00 | 49.28 | C |
| ATOM | 684 | CG | ASP | A | 85 | 15.822 | 0.339 | 44.322 | 1.00 | 59.16 | C |
| ATOM | 685 | OD1 | ASP | A | 85 | 16.341 | −0.779 | 44.565 | 1.00 | 58.58 | O |
| ATOM | 686 | OD2 | ASP | A | 85 | 14.696 | 0.471 | 43.781 | 1.00 | 62.86 | O |
| ATOM | 687 | C | ASP | A | 85 | 18.933 | 1.083 | 43.968 | 1.00 | 50.64 | C |
| ATOM | 688 | O | ASP | A | 85 | 19.682 | 1.985 | 43.598 | 1.00 | 41.86 | O |
| ATOM | 689 | N | GLN | A | 86 | 18.856 | −0.085 | 43.340 | 1.00 | 46.27 | N |
| ATOM | 690 | CA | GLN | A | 86 | 19.793 | −0.421 | 42.284 | 1.00 | 49.48 | C |
| ATOM | 691 | CB | GLN | A | 86 | 21.074 | −1.028 | 42.875 | 1.00 | 49.78 | C |
| ATOM | 692 | CG | GLN | A | 86 | 22.111 | −1.422 | 41.853 | 1.00 | 51.92 | C |
| ATOM | 693 | CD | GLN | A | 86 | 23.285 | −2.173 | 42.453 | 1.00 | 59.84 | C |
| ATOM | 694 | OE1 | GLN | A | 86 | 24.215 | −1.567 | 42.995 | 1.00 | 58.16 | O |
| ATOM | 695 | NE2 | GLN | A | 86 | 23.248 | −3.506 | 42.360 | 1.00 | 52.42 | N |
| ATOM | 696 | C | GLN | A | 86 | 19.158 | −1.397 | 41.328 | 1.00 | 52.56 | C |
| ATOM | 697 | O | GLN | A | 86 | 18.871 | −2.534 | 41.700 | 1.00 | 52.85 | O |
| ATOM | 698 | N | LEU | A | 87 | 18.928 | −0.943 | 40.097 | 1.00 | 51.65 | N |
| ATOM | 699 | CA | LEU | A | 87 | 18.352 | −1.793 | 39.063 | 1.00 | 43.49 | C |
| ATOM | 700 | CB | LEU | A | 87 | 17.211 | −1.095 | 38.310 | 1.00 | 50.83 | C |
| ATOM | 701 | CG | LEU | A | 87 | 16.283 | −0.033 | 38.925 | 1.00 | 58.10 | C |
| ATOM | 702 | CD1 | LEU | A | 87 | 14.878 | −0.165 | 38.333 | 1.00 | 55.28 | C |
| ATOM | 703 | CD2 | LEU | A | 87 | 16.215 | −0.073 | 40.446 | 1.00 | 56.98 | C |
| ATOM | 704 | C | LEU | A | 87 | 19.456 | −2.191 | 38.094 | 1.00 | 49.58 | C |
| ATOM | 705 | O | LEU | A | 87 | 20.049 | −1.337 | 37.428 | 1.00 | 49.69 | O |
| ATOM | 706 | N | VAL | A | 88 | 19.748 | −3.488 | 38.021 | 1.00 | 46.19 | N |
| ATOM | 707 | CA | VAL | A | 88 | 20.809 | −3.938 | 37.129 | 1.00 | 47.56 | C |
| ATOM | 708 | CB | VAL | A | 88 | 21.862 | −4.821 | 37.826 | 1.00 | 50.30 | C |
| ATOM | 709 | CG1 | VAL | A | 88 | 22.759 | −5.486 | 36.782 | 1.00 | 40.02 | C |
| ATOM | 710 | CG2 | VAL | A | 88 | 22.689 | −3.981 | 38.767 | 1.00 | 37.73 | C |
| ATOM | 711 | C | VAL | A | 88 | 20.274 | −4.678 | 35.930 | 1.00 | 44.65 | C |
| ATOM | 712 | O | VAL | A | 88 | 19.534 | −5.636 | 36.067 | 1.00 | 46.30 | O |
| ATOM | 713 | N | GLY | A | 89 | 20.685 | −4.230 | 34.746 | 1.00 | 47.50 | N |
| ATOM | 714 | CA | GLY | A | 89 | 20.172 | −4.749 | 33.494 | 1.00 | 38.50 | C |
| ATOM | 715 | C | GLY | A | 89 | 19.080 | −3.855 | 32.922 | 1.00 | 35.81 | C |
| ATOM | 716 | O | GLY | A | 89 | 18.469 | −3.057 | 33.636 | 1.00 | 36.36 | O |
| ATOM | 717 | N | ALA | A | 90 | 18.841 | −3.987 | 31.621 | 1.00 | 58.32 | N |
| ATOM | 718 | CA | ALA | A | 90 | 17.763 | −3.255 | 30.952 | 1.00 | 62.38 | C |
| ATOM | 719 | CB | ALA | A | 90 | 17.927 | −3.352 | 29.441 | 1.00 | 48.77 | C |
| ATOM | 720 | C | ALA | A | 90 | 16.378 | −3.765 | 31.378 | 1.00 | 51.30 | C |
| ATOM | 721 | O | ALA | A | 90 | 16.197 | −4.964 | 31.602 | 1.00 | 56.70 | O |
| ATOM | 722 | N | ARG | A | 91 | 15.417 | −2.850 | 31.508 | 1.00 | 47.07 | N |
| ATOM | 723 | CA | ARG | A | 91 | 14.019 | −3.212 | 31.763 | 1.00 | 52.53 | C |
| ATOM | 724 | CB | ARG | A | 91 | 13.800 | −3.542 | 33.244 | 1.00 | 62.63 | C |
| ATOM | 725 | CG | ARG | A | 91 | 14.097 | −2.384 | 34.191 | 1.00 | 62.42 | C |
| ATOM | 726 | CD | ARG | A | 91 | 14.445 | −2.868 | 35.591 | 1.00 | 70.13 | C |
| ATOM | 727 | NE | ARG | A | 91 | 15.411 | −3.967 | 35.569 | 1.00 | 68.83 | N |
| ATOM | 728 | CZ | ARG | A | 91 | 15.690 | −4.740 | 36.616 | 1.00 | 67.30 | C |
| ATOM | 729 | NH1 | ARG | A | 91 | 15.079 | −4.531 | 37.782 | 1.00 | 46.68 | N |
| ATOM | 730 | NH2 | ARG | A | 91 | 16.578 | −5.725 | 36.488 | 1.00 | 65.15 | N |
| ATOM | 731 | C | ARG | A | 91 | 13.066 | −2.093 | 31.326 | 1.00 | 57.03 | C |
| ATOM | 732 | O | ARG | A | 91 | 13.449 | −0.918 | 31.345 | 1.00 | 55.46 | O |
| ATOM | 733 | N | PRO | A | 92 | 11.823 | −2.461 | 30.925 | 1.00 | 57.44 | N |
| ATOM | 734 | CA | PRO | A | 92 | 10.780 | −1.584 | 30.364 | 1.00 | 53.47 | C |
| ATOM | 735 | CB | PRO | A | 92 | 9.520 | −2.441 | 30.476 | 1.00 | 44.62 | C |
| ATOM | 736 | CG | PRO | A | 92 | 10.018 | −3.806 | 30.212 | 1.00 | 49.42 | C |
| ATOM | 737 | CD | PRO | A | 92 | 11.387 | −3.871 | 30.892 | 1.00 | 56.99 | C |
| ATOM | 738 | C | PRO | A | 92 | 10.563 | −0.267 | 31.084 | 1.00 | 51.47 | C |
| ATOM | 739 | O | PRO | A | 92 | 10.686 | −0.212 | 32.299 | 1.00 | 58.71 | O |
| ATOM | 740 | N | LYS | A | 93 | 10.219 | 0.773 | 30.326 | 1.00 | 44.84 | N |
| ATOM | 741 | CA | LYS | A | 93 | 9.884 | 2.080 | 30.891 | 1.00 | 40.64 | C |
| ATOM | 742 | CB | LYS | A | 93 | 9.434 | 3.040 | 29.787 | 1.00 | 34.36 | C |
| ATOM | 743 | CG | LYS | A | 93 | 8.944 | 4.408 | 30.224 | 1.00 | 33.81 | C |
| ATOM | 744 | CD | LYS | A | 93 | 8.691 | 5.313 | 28.991 | 1.00 | 33.35 | C |
| ATOM | 745 | CE | LYS | A | 93 | 8.075 | 6.643 | 29.434 | 1.00 | 49.53 | C |
| ATOM | 746 | NZ | LYS | A | 93 | 7.792 | 7.616 | 28.346 | 1.00 | 58.07 | N |
| ATOM | 747 | C | LYS | A | 93 | 8.846 | 2.000 | 32.023 | 1.00 | 52.11 | C |
| ATOM | 748 | O | LYS | A | 93 | 9.012 | 2.645 | 33.058 | 1.00 | 53.14 | O |
| ATOM | 749 | N | GLU | A | 94 | 7.795 | 1.202 | 31.851 | 1.00 | 59.73 | N |
| ATOM | 750 | CA | GLU | A | 94 | 6.747 | 1.121 | 32.878 | 1.00 | 61.21 | C |
| ATOM | 751 | CB | GLU | A | 94 | 5.506 | 0.370 | 32.374 | 1.00 | 58.21 | C |
| ATOM | 752 | CG | GLU | A | 94 | 5.787 | −0.599 | 31.226 | 1.00 | 69.25 | C |
| ATOM | 753 | CD | GLU | A | 94 | 5.912 | 0.080 | 29.856 | 1.00 | 64.98 | C |
| ATOM | 754 | OE1 | GLU | A | 94 | 4.948 | 0.767 | 29.453 | 1.00 | 58.73 | O |
| ATOM | 755 | OE2 | GLU | A | 94 | 6.973 | −0.068 | 29.193 | 1.00 | 56.11 | O |
| ATOM | 756 | C | GLU | A | 94 | 7.250 | 0.521 | 34.191 | 1.00 | 62.23 | C |
| ATOM | 757 | O | GLU | A | 94 | 6.772 | 0.881 | 35.267 | 1.00 | 66.11 | O |
| ATOM | 758 | N | ALA | A | 95 | 8.223 | −0.381 | 34.101 | 1.00 | 46.59 | N |
| ATOM | 759 | CA | ALA | A | 95 | 8.789 | −0.997 | 35.291 | 1.00 | 49.78 | C |
| ATOM | 760 | CB | ALA | A | 95 | 9.497 | −2.281 | 34.939 | 1.00 | 48.29 | C |
| ATOM | 761 | C | ALA | A | 95 | 9.730 | −0.039 | 36.034 | 1.00 | 54.56 | C |
| ATOM | 762 | O | ALA | A | 95 | 9.744 | −0.005 | 37.262 | 1.00 | 49.49 | O |
| ATOM | 763 | N | LEU | A | 96 | 10.512 | 0.742 | 35.296 | 1.00 | 56.16 | N |
| ATOM | 764 | CA | LEU | A | 96 | 11.303 | 1.802 | 35.916 | 1.00 | 58.31 | C |
| ATOM | 765 | CB | LEU | A | 96 | 12.123 | 2.562 | 34.870 | 1.00 | 60.94 | C |
| ATOM | 766 | CG | LEU | A | 96 | 13.495 | 1.992 | 34.509 | 1.00 | 61.66 | C |
| ATOM | 767 | CD1 | LEU | A | 96 | 13.378 | 0.533 | 34.148 | 1.00 | 64.27 | C |
| ATOM | 768 | CD2 | LEU | A | 96 | 14.138 | 2.764 | 33.364 | 1.00 | 60.70 | C |
| ATOM | 769 | C | LEU | A | 96 | 10.394 | 2.772 | 36.653 | 1.00 | 57.57 | C |
| ATOM | 770 | O | LEU | A | 96 | 10.743 | 3.269 | 37.717 | 1.00 | 56.98 | O |
| ATOM | 771 | N | LYS | A | 97 | 9.227 | 3.042 | 36.074 | 1.00 | 67.97 | N |
| ATOM | 772 | CA | LYS | A | 97 | 8.277 | 3.981 | 36.656 | 1.00 | 65.41 | C |
| ATOM | 773 | CB | LYS | A | 97 | 7.199 | 4.351 | 35.646 | 1.00 | 73.77 | C |
| ATOM | 774 | CG | LYS | A | 97 | 7.665 | 5.274 | 34.556 | 1.00 | 74.08 | C |
| ATOM | 775 | CD | LYS | A | 97 | 7.110 | 6.667 | 34.758 | 1.00 | 71.53 | C |
| ATOM | 776 | CE | LYS | A | 97 | 5.611 | 6.691 | 34.580 | 1.00 | 80.59 | C |
| ATOM | 777 | NZ | LYS | A | 97 | 5.069 | 8.053 | 34.847 | 1.00 | 91.82 | N |
| ATOM | 778 | C | LYS | A | 97 | 7.615 | 3.407 | 37.888 | 1.00 | 71.95 | C |
| ATOM | 779 | O | LYS | A | 97 | 7.095 | 4.149 | 38.712 | 1.00 | 81.45 | O |
| ATOM | 780 | N | GLU | A | 98 | 7.614 | 2.084 | 37.999 | 1.00 | 65.39 | N |
| ATOM | 781 | CA | GLU | A | 98 | 7.033 | 1.424 | 39.159 | 1.00 | 73.85 | C |
| ATOM | 782 | CB | GLU | A | 98 | 6.702 | −0.041 | 38.843 | 1.00 | 69.72 | C |
| ATOM | 783 | CG | GLU | A | 98 | 5.233 | −0.406 | 39.107 | 1.00 | 72.59 | C |
| ATOM | 784 | CD | GLU | A | 98 | 4.771 | −1.638 | 38.336 | 1.00 | 81.27 | C |
| ATOM | 785 | OE1 | GLU | A | 98 | 3.776 | −1.519 | 37.583 | 1.00 | 76.85 | O |
| ATOM | 786 | OE2 | GLU | A | 98 | 5.391 | −2.719 | 38.485 | 1.00 | 80.46 | O |
| ATOM | 787 | C | GLU | A | 98 | 8.010 | 1.522 | 40.329 | 1.00 | 71.10 | C |
| ATOM | 788 | O | GLU | A | 98 | 7.617 | 1.557 | 41.498 | 1.00 | 69.60 | O |
| ATOM | 789 | N | ARG | A | 99 | 9.290 | 1.607 | 39.987 | 1.00 | 66.73 | N |
| ATOM | 790 | CA | ARG | A | 99 | 10.376 | 1.529 | 40.956 | 1.00 | 58.23 | C |
| ATOM | 791 | CB | ARG | A | 99 | 11.594 | 0.897 | 40.287 | 1.00 | 52.06 | C |
| ATOM | 792 | CG | ARG | A | 99 | 12.685 | 0.471 | 41.226 | 1.00 | 73.40 | C |
| ATOM | 793 | CD | ARG | A | 99 | 12.531 | −0.973 | 41.685 | 1.00 | 73.80 | C |
| ATOM | 794 | NE | ARG | A | 99 | 13.804 | −1.481 | 42.202 | 1.00 | 71.07 | N |
| ATOM | 795 | CZ | ARG | A | 99 | 14.251 | −2.718 | 42.017 | 1.00 | 72.57 | C |
| ATOM | 796 | NH1 | ARG | A | 99 | 13.521 | −3.600 | 41.342 | 1.00 | 74.25 | N |
| ATOM | 797 | NH2 | ARG | A | 99 | 15.428 | −3.072 | 42.511 | 1.00 | 72.33 | N |
| ATOM | 798 | C | ARG | A | 99 | 10.702 | 2.919 | 41.504 | 1.00 | 62.05 | C |
| ATOM | 799 | O | ARG | A | 99 | 11.247 | 3.058 | 42.595 | 1.00 | 63.69 | O |
| ATOM | 800 | N | ILE | A | 100 | 10.319 | 3.946 | 40.752 | 1.00 | 56.12 | N |
| ATOM | 801 | CA | ILE | A | 100 | 10.591 | 5.336 | 41.118 | 1.00 | 52.23 | C |
| ATOM | 802 | CB | ILE | A | 100 | 10.609 | 6.250 | 39.870 | 1.00 | 47.29 | C |
| ATOM | 803 | CG1 | ILE | A | 100 | 11.885 | 6.013 | 39.075 | 1.00 | 45.49 | C |
| ATOM | 804 | CD1 | ILE | A | 100 | 11.943 | 6.842 | 37.826 | 1.00 | 47.64 | C |
| ATOM | 805 | CG2 | ILE | A | 100 | 10.570 | 7.710 | 40.244 | 1.00 | 43.48 | C |
| ATOM | 806 | C | ILE | A | 100 | 9.607 | 5.915 | 42.129 | 1.00 | 59.82 | C |
| ATOM | 807 | O | ILE | A | 100 | 9.983 | 6.741 | 42.959 | 1.00 | 59.68 | O |
| ATOM | 808 | N | LYS | A | 101 | 8.344 | 5.509 | 42.046 | 1.00 | 79.70 | N |
| ATOM | 809 | CA | LYS | A | 101 | 7.317 | 6.043 | 42.940 | 1.00 | 76.96 | C |
| ATOM | 810 | CB | LYS | A | 101 | 5.938 | 5.598 | 42.465 | 1.00 | 82.89 | C |
| ATOM | 811 | CG | LYS | A | 101 | 5.803 | 4.091 | 42.343 | 1.00 | 80.32 | C |
| ATOM | 812 | CD | LYS | A | 101 | 4.356 | 3.683 | 42.139 | 1.00 | 86.67 | C |
| ATOM | 813 | CE | LYS | A | 101 | 4.218 | 2.172 | 42.011 | 1.00 | 87.56 | C |
| ATOM | 814 | NZ | LYS | A | 101 | 4.712 | 1.456 | 43.229 | 1.00 | 87.65 | N |
| ATOM | 815 | C | LYS | A | 101 | 7.537 | 5.607 | 44.388 | 1.00 | 74.25 | C |
| ATOM | 816 | O | LYS | A | 101 | 7.328 | 6.377 | 45.325 | 1.00 | 71.03 | O |
| ATOM | 817 | N | LYS | A | 102 | 7.959 | 4.361 | 44.562 | 1.00 | 77.52 | N |
| ATOM | 818 | CA | LYS | A | 102 | 8.247 | 3.833 | 45.884 | 1.00 | 77.12 | C |
| ATOM | 819 | C | LYS | A | 102 | 9.395 | 4.604 | 46.531 | 1.00 | 80.30 | C |
| ATOM | 820 | O | LYS | A | 102 | 9.802 | 4.300 | 47.650 | 1.00 | 85.37 | O |
| ATOM | 821 | CB | LYS | A | 102 | 8.572 | 2.338 | 45.808 | 1.00 | 86.43 | C |
| ATOM | 822 | CG | LYS | A | 102 | 9.894 | 1.982 | 45.119 | 1.00 | 88.05 | C |
| ATOM | 823 | CD | LYS | A | 102 | 10.096 | 0.464 | 45.091 | 1.00 | 89.96 | C |
| ATOM | 824 | CE | LYS | A | 102 | 11.524 | 0.062 | 45.477 | 1.00 | 92.22 | C |
| ATOM | 825 | NZ | LYS | A | 102 | 11.553 | −1.174 | 46.334 | 1.00 | 94.05 | N |
| ATOM | 826 | N | TYR | A | 103 | 9.911 | 5.591 | 45.801 | 1.00 | 79.35 | N |
| ATOM | 827 | CA | TYR | A | 103 | 10.954 | 6.497 | 46.270 | 1.00 | 85.72 | C |
| ATOM | 828 | CB | TYR | A | 103 | 12.336 | 6.078 | 45.758 | 1.00 | 82.75 | C |
| ATOM | 829 | CG | TYR | A | 103 | 12.917 | 4.897 | 46.481 | 1.00 | 76.37 | C |
| ATOM | 830 | CD1 | TYR | A | 103 | 13.259 | 4.988 | 47.817 | 1.00 | 76.14 | C |
| ATOM | 831 | CE1 | TYR | A | 103 | 13.787 | 3.901 | 48.496 | 1.00 | 81.23 | C |
| ATOM | 832 | CZ | TYR | A | 103 | 13.978 | 2.709 | 47.826 | 1.00 | 85.28 | C |
| ATOM | 833 | OH | TYR | A | 103 | 14.502 | 1.620 | 48.494 | 1.00 | 77.89 | O |
| ATOM | 834 | CE2 | TYR | A | 103 | 13.637 | 2.602 | 46.489 | 1.00 | 80.80 | C |
| ATOM | 835 | CD2 | TYR | A | 103 | 13.115 | 3.690 | 45.830 | 1.00 | 73.41 | C |
| ATOM | 836 | C | TYR | A | 103 | 10.652 | 7.864 | 45.717 | 1.00 | 87.35 | C |
| ATOM | 837 | O | TYR | A | 103 | 11.529 | 8.518 | 45.163 | 1.00 | 91.19 | O |
| ATOM | 838 | N | LEU | A | 104 | 9.402 | 8.287 | 45.834 | 1.00 | 99.12 | N |
| ATOM | 839 | CA | LEU | A | 104 | 8.986 | 9.539 | 45.220 | 1.00 | 104.27 | C |
| ATOM | 840 | CB | LEU | A | 104 | 7.620 | 9.379 | 44.541 | 1.00 | 95.39 | C |
| ATOM | 841 | CG | LEU | A | 104 | 7.483 | 10.111 | 43.204 | 1.00 | 101.70 | C |
| ATOM | 842 | CD1 | LEU | A | 104 | 8.861 | 10.305 | 42.566 | 1.00 | 92.92 | C |
| ATOM | 843 | CD2 | LEU | A | 104 | 6.530 | 9.362 | 42.268 | 1.00 | 87.83 | C |
| ATOM | 844 | C | LEU | A | 104 | 8.972 | 10.683 | 46.234 | 1.00 | 114.63 | C |
| ATOM | 845 | O | LEU | A | 104 | 9.117 | 11.851 | 45.868 | 1.00 | 109.80 | O |
| ATOM | 846 | OXT | LEU | A | 104 | 8.828 | 10.466 | 47.442 | 1.00 | 117.02 | O |
| TER | | | | | | | | | | | |
| ATOM | 847 | N | SER | B | 1 | −0.577 | −26.412 | 26.761 | 1.00 | 105.17 | N |
| ATOM | 848 | CA | SER | B | 1 | −0.553 | −25.853 | 25.415 | 1.00 | 101.88 | C |
| ATOM | 849 | CB | SER | B | 1 | −1.921 | −25.273 | 25.050 | 1.00 | 98.08 | C |
| ATOM | 850 | OG | SER | B | 1 | −2.578 | −26.089 | 24.089 | 1.00 | 102.28 | O |
| ATOM | 851 | C | SER | B | 1 | 0.521 | −24.784 | 25.291 | 1.00 | 97.18 | C |
| ATOM | 852 | O | SER | B | 1 | 1.470 | −24.920 | 24.518 | 1.00 | 97.53 | O |
| ATOM | 853 | N | VAL | B | 2 | 0.363 | −23.721 | 26.068 | 1.00 | 95.24 | N |
| ATOM | 854 | CA | VAL | B | 2 | 1.298 | −22.609 | 26.053 | 1.00 | 90.10 | C |
| ATOM | 855 | CB | VAL | B | 2 | 0.544 | −21.265 | 25.957 | 1.00 | 91.49 | C |
| ATOM | 856 | CG1 | VAL | B | 2 | −0.568 | −21.208 | 27.007 | 1.00 | 93.18 | C |
| ATOM | 857 | CG2 | VAL | B | 2 | 1.507 | −20.099 | 26.091 | 1.00 | 88.83 | C |
| ATOM | 858 | C | VAL | B | 2 | 2.196 | −22.664 | 27.285 | 1.00 | 91.82 | C |
| ATOM | 859 | O | VAL | B | 2 | 1.712 | −22.725 | 28.410 | 1.00 | 95.00 | O |
| ATOM | 860 | N | ILE | B | 3 | 3.504 | −22.619 | 27.050 | 1.00 | 75.97 | N |
| ATOM | 861 | CA | ILE | B | 3 | 4.518 | −23.056 | 28.012 | 1.00 | 69.74 | C |
| ATOM | 862 | CB | ILE | B | 3 | 5.629 | −23.779 | 27.249 | 1.00 | 69.49 | C |
| ATOM | 863 | CG1 | ILE | B | 3 | 5.104 | −25.083 | 26.668 | 1.00 | 67.00 | C |
| ATOM | 864 | CD1 | ILE | B | 3 | 6.057 | −25.728 | 25.717 | 1.00 | 64.77 | C |
| ATOM | 865 | CG2 | ILE | B | 3 | 6.829 | −24.023 | 28.142 | 1.00 | 77.43 | C |
| ATOM | 866 | C | ILE | B | 3 | 5.229 | −21.946 | 28.786 | 1.00 | 71.35 | C |
| ATOM | 867 | O | ILE | B | 3 | 5.940 | −21.134 | 28.187 | 1.00 | 70.42 | O |
| ATOM | 868 | N | GLU | B | 4 | 5.096 | −21.928 | 30.112 | 1.00 | 74.48 | N |
| ATOM | 869 | CA | GLU | B | 4 | 5.899 | −20.991 | 30.881 | 1.00 | 77.86 | C |
| ATOM | 870 | CB | GLU | B | 4 | 5.808 | −21.218 | 32.389 | 1.00 | 81.96 | C |
| ATOM | 871 | CG | GLU | B | 4 | 6.914 | −20.455 | 33.138 | 1.00 | 85.95 | C |
| ATOM | 872 | CD | GLU | B | 4 | 6.794 | −20.518 | 34.664 | 1.00 | 97.96 | C |
| ATOM | 873 | OE1 | GLU | B | 4 | 5.676 | −20.806 | 35.157 | 1.00 | 90.44 | O |
| ATOM | 874 | OE2 | GLU | B | 4 | 7.815 | −20.264 | 35.363 | 1.00 | 74.71 | O |
| ATOM | 875 | C | GLU | B | 4 | 7.334 | −21.178 | 30.424 | 1.00 | 79.85 | C |
| ATOM | 876 | O | GLU | B | 4 | 7.807 | −22.304 | 30.274 | 1.00 | 86.78 | O |
| ATOM | 877 | N | ILE | B | 5 | 8.020 | −20.072 | 30.176 | 1.00 | 59.31 | N |
| ATOM | 878 | CA | ILE | B | 5 | 9.414 | −20.119 | 29.768 | 1.00 | 60.43 | C |
| ATOM | 879 | CB | ILE | B | 5 | 9.588 | −19.668 | 28.303 | 1.00 | 59.16 | C |
| ATOM | 880 | CG1 | ILE | B | 5 | 9.065 | −20.730 | 27.335 | 1.00 | 53.06 | C |
| ATOM | 881 | CD1 | ILE | B | 5 | 9.234 | −20.316 | 25.872 | 1.00 | 41.17 | C |
| ATOM | 882 | CG2 | ILE | B | 5 | 11.043 | −19.349 | 27.988 | 1.00 | 55.00 | C |
| ATOM | 883 | C | ILE | B | 5 | 10.230 | −19.216 | 30.691 | 1.00 | 57.22 | C |
| ATOM | 884 | O | ILE | B | 5 | 9.790 | −18.123 | 31.051 | 1.00 | 43.81 | O |
| ATOM | 885 | N | ASN | B | 6 | 11.420 | −19.680 | 31.066 | 1.00 | 69.05 | N |
| ATOM | 886 | CA | ASN | B | 6 | 12.307 | −18.951 | 31.976 | 1.00 | 66.62 | C |
| ATOM | 887 | CB | ASN | B | 6 | 12.015 | −19.335 | 33.432 | 1.00 | 63.10 | C |
| ATOM | 888 | CG | ASN | B | 6 | 11.629 | −20.793 | 33.583 | 1.00 | 66.21 | C |
| ATOM | 889 | OD1 | ASN | B | 6 | 12.439 | −21.687 | 33.348 | 1.00 | 73.63 | O |
| ATOM | 890 | ND2 | ASN | B | 6 | 10.389 | −21.039 | 33.989 | 1.00 | 72.15 | N |
| ATOM | 891 | C | ASN | B | 6 | 13.773 | −19.199 | 31.645 | 1.00 | 65.85 | C |
| ATOM | 892 | O | ASN | B | 6 | 14.100 | −20.171 | 30.962 | 1.00 | 64.29 | O |
| ATOM | 893 | N | ASP | B | 7 | 14.645 | −18.322 | 32.136 | 1.00 | 86.42 | N |
| ATOM | 894 | CA | ASP | B | 7 | 16.083 | −18.444 | 31.902 | 1.00 | 98.58 | C |
| ATOM | 895 | CB | ASP | B | 7 | 16.890 | −17.596 | 32.900 | 1.00 | 87.77 | C |
| ATOM | 896 | CG | ASP | B | 7 | 16.026 | −16.648 | 33.719 | 1.00 | 91.48 | C |
| ATOM | 897 | OD1 | ASP | B | 7 | 15.779 | −15.512 | 33.245 | 1.00 | 86.27 | O |
| ATOM | 898 | OD2 | ASP | B | 7 | 15.617 | −17.035 | 34.845 | 1.00 | 86.15 | O |
| ATOM | 899 | C | ASP | B | 7 | 16.564 | −19.900 | 31.961 | 1.00 | 102.54 | C |
| ATOM | 900 | O | ASP | B | 7 | 17.521 | −20.282 | 31.279 | 1.00 | 95.68 | O |
| ATOM | 901 | N | GLU | B | 8 | 15.887 | −20.703 | 32.775 | 1.00 | 93.37 | N |
| ATOM | 902 | CA | GLU | B | 8 | 16.221 | −22.109 | 32.938 | 1.00 | 90.14 | C |
| ATOM | 903 | CB | GLU | B | 8 | 15.522 | −22.661 | 34.173 | 1.00 | 98.10 | C |
| ATOM | 904 | CG | GLU | B | 8 | 16.004 | −22.044 | 35.468 | 1.00 | 112.15 | C |
| ATOM | 905 | CD | GLU | B | 8 | 15.239 | −22.557 | 36.667 | 1.00 | 124.79 | C |
| ATOM | 906 | OE1 | GLU | B | 8 | 14.001 | −22.707 | 36.557 | 1.00 | 114.62 | O |
| ATOM | 907 | OE2 | GLU | B | 8 | 15.879 | −22.815 | 37.712 | 1.00 | 137.10 | O |
| ATOM | 908 | C | GLU | B | 8 | 15.860 | −22.959 | 31.730 | 1.00 | 92.49 | C |
| ATOM | 909 | O | GLU | B | 8 | 16.725 | −23.590 | 31.133 | 1.00 | 96.60 | O |
| ATOM | 910 | N | ASN | B | 9 | 14.581 | −22.978 | 31.372 | 1.00 | 91.20 | N |
| ATOM | 911 | CA | ASN | B | 9 | 14.114 | −23.859 | 30.303 | 1.00 | 93.39 | C |
| ATOM | 912 | CB | ASN | B | 9 | 12.707 | −24.396 | 30.609 | 1.00 | 89.12 | C |
| ATOM | 913 | CG | ASN | B | 9 | 11.639 | −23.308 | 30.575 | 1.00 | 94.54 | C |
| ATOM | 914 | OD1 | ASN | B | 9 | 11.928 | −22.130 | 30.805 | 1.00 | 91.22 | O |
| ATOM | 915 | ND2 | ASN | B | 9 | 10.397 | −23.701 | 30.286 | 1.00 | 88.21 | N |
| ATOM | 916 | C | ASN | B | 9 | 14.146 | −23.222 | 28.917 | 1.00 | 95.07 | C |
| ATOM | 917 | O | ASN | B | 9 | 13.634 | −23.792 | 27.957 | 1.00 | 99.72 | O |
| ATOM | 918 | N | PHE | B | 10 | 14.765 | −22.051 | 28.805 | 1.00 | 81.25 | N |
| ATOM | 919 | CA | PHE | B | 10 | 14.684 | −21.284 | 27.560 | 1.00 | 78.84 | C |
| ATOM | 920 | CB | PHE | B | 10 | 15.344 | −19.918 | 27.685 | 1.00 | 71.60 | C |
| ATOM | 921 | CG | PHE | B | 10 | 15.207 | −19.089 | 26.452 | 1.00 | 68.18 | C |
| ATOM | 922 | CD1 | PHE | B | 10 | 13.967 | −18.577 | 26.093 | 1.00 | 76.57 | C |
| ATOM | 923 | CE1 | PHE | B | 10 | 13.808 | −17.809 | 24.944 | 1.00 | 67.39 | C |
| ATOM | 924 | CZ | PHE | B | 10 | 14.898 | −17.548 | 24.129 | 1.00 | 66.06 | C |
| ATOM | 925 | CE2 | PHE | B | 10 | 16.152 | −18.058 | 24.472 | 1.00 | 81.01 | C |
| ATOM | 926 | CD2 | PHE | B | 10 | 16.299 | −18.830 | 25.637 | 1.00 | 79.21 | C |
| ATOM | 927 | C | PHE | B | 10 | 15.265 | −21.989 | 26.352 | 1.00 | 86.33 | C |
| ATOM | 928 | O | PHE | B | 10 | 14.567 | −22.192 | 25.364 | 1.00 | 81.53 | O |
| ATOM | 929 | N | ASP | B | 11 | 16.548 | −22.336 | 26.436 | 1.00 | 100.81 | N |
| ATOM | 930 | CA | ASP | B | 11 | 17.284 | −22.965 | 25.337 | 1.00 | 99.39 | C |
| ATOM | 931 | CB | ASP | B | 11 | 18.611 | −23.503 | 25.848 | 1.00 | 101.34 | C |
| ATOM | 932 | CG | ASP | B | 11 | 18.432 | −24.567 | 26.912 | 1.00 | 110.25 | C |
| ATOM | 933 | OD2 | ASP | B | 11 | 19.357 | −25.396 | 27.070 | 1.00 | 111.74 | O |
| ATOM | 934 | OD1 | ASP | B | 11 | 17.372 | −24.574 | 27.588 | 1.00 | 100.69 | O |
| ATOM | 935 | C | ASP | B | 11 | 16.533 | −24.074 | 24.588 | 1.00 | 106.68 | C |
| ATOM | 936 | O | ASP | B | 11 | 17.053 | −24.623 | 23.616 | 1.00 | 108.36 | O |
| ATOM | 937 | N | GLU | B | 12 | 15.328 | −24.407 | 25.052 | 1.00 | 96.91 | N |
| ATOM | 938 | CA | GLU | B | 12 | 14.396 | −25.249 | 24.301 | 1.00 | 99.82 | C |
| ATOM | 939 | CB | GLU | B | 12 | 13.186 | −25.596 | 25.157 | 1.00 | 94.57 | C |
| ATOM | 940 | CG | GLU | B | 12 | 12.264 | −24.406 | 25.382 | 1.00 | 94.67 | C |
| ATOM | 941 | CD | GLU | B | 12 | 10.992 | −24.774 | 26.123 | 1.00 | 91.32 | C |
| ATOM | 942 | OE1 | GLU | B | 12 | 10.058 | −25.299 | 25.476 | 1.00 | 90.27 | O |
| ATOM | 943 | OE2 | GLU | B | 12 | 10.924 | −24.524 | 27.349 | 1.00 | 85.22 | O |
| ATOM | 944 | C | GLU | B | 12 | 13.909 | −24.532 | 23.035 | 1.00 | 99.22 | C |
| ATOM | 945 | O | GLU | B | 12 | 12.918 | −24.922 | 22.423 | 1.00 | 88.70 | O |
| ATOM | 946 | N | VAL | B | 13 | 14.598 | −23.460 | 22.667 | 1.00 | 108.98 | N |
| ATOM | 947 | CA | VAL | B | 13 | 14.391 | −22.831 | 21.373 | 1.00 | 112.79 | C |
| ATOM | 948 | CB | VAL | B | 13 | 14.660 | −21.308 | 21.414 | 1.00 | 99.59 | C |
| ATOM | 949 | CG1 | VAL | B | 13 | 13.493 | −20.565 | 22.055 | 1.00 | 81.14 | C |
| ATOM | 950 | CG2 | VAL | B | 13 | 15.967 | −21.015 | 22.148 | 1.00 | 98.96 | C |
| ATOM | 951 | C | VAL | B | 13 | 15.346 | −23.481 | 20.370 | 1.00 | 124.68 | C |
| ATOM | 952 | O | VAL | B | 13 | 14.929 | −23.972 | 19.314 | 1.00 | 119.14 | O |
| ATOM | 953 | N | ILE | B | 14 | 16.627 | −23.500 | 20.731 | 1.00 | 169.10 | N |
| ATOM | 954 | CA | ILE | B | 14 | 17.688 | −23.980 | 19.851 | 1.00 | 172.43 | C |
| ATOM | 955 | CB | ILE | B | 14 | 19.076 | −23.486 | 20.335 | 1.00 | 174.56 | C |
| ATOM | 956 | CG1 | ILE | B | 14 | 20.126 | −23.628 | 19.224 | 1.00 | 174.32 | C |
| ATOM | 957 | CD1 | ILE | B | 14 | 20.735 | −22.301 | 18.771 | 1.00 | 162.43 | C |
| ATOM | 958 | CG2 | ILE | B | 14 | 19.476 | −24.191 | 21.631 | 1.00 | 170.25 | C |
| ATOM | 959 | C | ILE | B | 14 | 17.693 | −25.501 | 19.785 | 1.00 | 171.95 | C |
| ATOM | 960 | O | ILE | B | 14 | 18.152 | −26.097 | 18.808 | 1.00 | 172.20 | O |
| ATOM | 961 | N | LYS | B | 15 | 17.176 | −26.123 | 20.836 | 1.00 | 117.60 | N |
| ATOM | 962 | CA | LYS | B | 15 | 17.145 | −27.569 | 20.913 | 1.00 | 118.01 | C |
| ATOM | 963 | CB | LYS | B | 15 | 17.407 | −28.011 | 22.346 | 1.00 | 114.54 | C |
| ATOM | 964 | CG | LYS | B | 15 | 18.072 | −29.358 | 22.442 | 1.00 | 107.96 | C |
| ATOM | 965 | CD | LYS | B | 15 | 18.619 | −29.593 | 23.833 | 1.00 | 100.90 | C |
| ATOM | 966 | CE | LYS | B | 15 | 18.017 | −30.839 | 24.449 | 1.00 | 91.85 | C |
| ATOM | 967 | NZ | LYS | B | 15 | 18.863 | −31.357 | 25.549 | 1.00 | 85.25 | N |
| ATOM | 968 | C | LYS | B | 15 | 15.800 | −28.087 | 20.431 | 1.00 | 110.32 | C |
| ATOM | 969 | O | LYS | B | 15 | 15.615 | −29.289 | 20.245 | 1.00 | 111.28 | O |
| ATOM | 970 | N | LYS | B | 16 | 14.863 | −27.169 | 20.230 | 1.00 | 139.88 | N |
| ATOM | 971 | CA | LYS | B | 16 | 13.550 | −27.529 | 19.723 | 1.00 | 140.00 | C |
| ATOM | 972 | CB | LYS | B | 16 | 12.636 | −26.304 | 19.654 | 1.00 | 138.11 | C |
| ATOM | 973 | CG | LYS | B | 16 | 11.186 | −26.586 | 20.016 | 1.00 | 125.86 | C |
| ATOM | 974 | CD | LYS | B | 16 | 11.081 | −27.192 | 21.405 | 1.00 | 130.26 | C |
| ATOM | 975 | CE | LYS | B | 16 | 9.635 | −27.294 | 21.865 | 1.00 | 126.28 | C |
| ATOM | 976 | NZ | LYS | B | 16 | 9.514 | −27.922 | 23.213 | 1.00 | 119.19 | N |
| ATOM | 977 | C | LYS | B | 16 | 13.700 | −28.155 | 18.346 | 1.00 | 136.26 | C |
| ATOM | 978 | O | LYS | B | 16 | 14.334 | −29.195 | 18.205 | 1.00 | 141.73 | O |
| ATOM | 979 | N | ASP | B | 17 | 13.122 | −27.515 | 17.333 | 1.00 | 109.86 | N |
| ATOM | 980 | CA | ASP | B | 17 | 13.114 | −28.057 | 15.980 | 1.00 | 105.60 | C |
| ATOM | 981 | CB | ASP | B | 17 | 12.647 | −29.517 | 16.006 | 1.00 | 109.33 | C |
| ATOM | 982 | CG | ASP | B | 17 | 11.488 | −29.756 | 16.983 | 1.00 | 114.87 | C |
| ATOM | 983 | OD1 | ASP | B | 17 | 11.755 | −30.089 | 18.160 | 1.00 | 122.12 | O |
| ATOM | 984 | OD2 | ASP | B | 17 | 10.310 | −29.633 | 16.573 | 1.00 | 108.52 | O |
| ATOM | 985 | C | ASP | B | 17 | 12.184 | −27.249 | 15.092 | 1.00 | 108.34 | C |
| ATOM | 986 | O | ASP | B | 17 | 12.600 | −26.585 | 14.140 | 1.00 | 106.04 | O |
| ATOM | 987 | N | LYS | B | 18 | 10.906 | −27.332 | 15.427 | 1.00 | 111.78 | N |
| ATOM | 988 | CA | LYS | B | 18 | 9.853 | −26.655 | 14.696 | 1.00 | 99.36 | C |
| ATOM | 989 | CB | LYS | B | 18 | 8.592 | −27.510 | 14.707 | 1.00 | 80.93 | C |
| ATOM | 990 | CG | LYS | B | 18 | 7.321 | −26.758 | 14.777 | 1.00 | 84.98 | C |
| ATOM | 991 | CD | LYS | B | 18 | 6.200 | −27.725 | 14.499 | 1.00 | 95.91 | C |
| ATOM | 992 | CE | LYS | B | 18 | 5.179 | −27.154 | 13.532 | 1.00 | 96.56 | C |
| ATOM | 993 | NZ | LYS | B | 18 | 5.667 | −26.386 | 12.308 | 1.00 | 83.38 | N |
| ATOM | 994 | C | LYS | B | 18 | 9.623 | −25.278 | 15.314 | 1.00 | 95.51 | C |
| ATOM | 995 | O | LYS | B | 18 | 10.154 | −24.972 | 16.389 | 1.00 | 93.84 | O |
| ATOM | 996 | N | VAL | B | 19 | 8.857 | −24.447 | 14.616 | 1.00 | 81.27 | N |
| ATOM | 997 | CA | VAL | B | 19 | 8.782 | −23.024 | 14.917 | 1.00 | 79.25 | C |
| ATOM | 998 | CB | VAL | B | 19 | 8.163 | −22.241 | 13.743 | 1.00 | 80.30 | C |
| ATOM | 999 | CG1 | VAL | B | 19 | 6.661 | −22.296 | 13.803 | 1.00 | 75.87 | C |
| ATOM | 1000 | CG2 | VAL | B | 19 | 8.635 | −20.801 | 13.766 | 1.00 | 82.15 | C |
| ATOM | 1001 | C | VAL | B | 19 | 8.077 | −22.673 | 16.232 | 1.00 | 80.13 | C |
| ATOM | 1002 | O | VAL | B | 19 | 6.993 | −23.192 | 16.560 | 1.00 | 72.36 | O |
| ATOM | 1003 | N | VAL | B | 20 | 8.712 | −21.756 | 16.959 | 1.00 | 74.87 | N |
| ATOM | 1004 | CA | VAL | B | 20 | 8.283 | −21.384 | 18.294 | 1.00 | 66.27 | C |
| ATOM | 1005 | CB | VAL | B | 20 | 9.371 | −21.662 | 19.314 | 1.00 | 66.59 | C |
| ATOM | 1006 | CG1 | VAL | B | 20 | 8.972 | −21.117 | 20.666 | 1.00 | 64.65 | C |
| ATOM | 1007 | CG2 | VAL | B | 20 | 9.625 | −23.143 | 19.398 | 1.00 | 75.54 | C |
| ATOM | 1008 | C | VAL | B | 20 | 7.877 | −19.920 | 18.384 | 1.00 | 62.49 | C |
| ATOM | 1009 | O | VAL | B | 20 | 8.608 | −19.012 | 17.966 | 1.00 | 55.20 | O |
| ATOM | 1010 | N | VAL | B | 21 | 6.695 | −19.715 | 18.953 | 1.00 | 53.78 | N |
| ATOM | 1011 | CA | VAL | B | 21 | 6.098 | −18.401 | 19.096 | 1.00 | 49.56 | C |
| ATOM | 1012 | CB | VAL | B | 21 | 4.649 | −18.468 | 18.615 | 1.00 | 46.98 | C |
| ATOM | 1013 | CG1 | VAL | B | 21 | 3.974 | −17.100 | 18.697 | 1.00 | 48.40 | C |
| ATOM | 1014 | CG2 | VAL | B | 21 | 4.626 | −18.988 | 17.203 | 1.00 | 49.53 | C |
| ATOM | 1015 | C | VAL | B | 21 | 6.151 | −17.962 | 20.561 | 1.00 | 46.23 | C |
| ATOM | 1016 | O | VAL | B | 21 | 5.605 | −18.642 | 21.426 | 1.00 | 48.59 | O |
| ATOM | 1017 | N | VAL | B | 22 | 6.796 | −16.831 | 20.843 | 1.00 | 50.79 | N |
| ATOM | 1018 | CA | VAL | B | 22 | 7.003 | −16.405 | 22.234 | 1.00 | 55.45 | C |
| ATOM | 1019 | CB | VAL | B | 22 | 8.513 | −16.390 | 22.612 | 1.00 | 51.44 | C |
| ATOM | 1020 | CG1 | VAL | B | 22 | 8.699 | −15.963 | 24.051 | 1.00 | 37.20 | C |
| ATOM | 1021 | CG2 | VAL | B | 22 | 9.143 | −17.749 | 22.383 | 1.00 | 51.37 | C |
| ATOM | 1022 | C | VAL | B | 22 | 6.395 | −15.047 | 22.605 | 1.00 | 49.52 | C |
| ATOM | 1023 | O | VAL | B | 22 | 6.863 | −14.001 | 22.146 | 1.00 | 49.62 | O |
| ATOM | 1024 | N | ASP | B | 23 | 5.387 | −15.076 | 23.474 | 1.00 | 46.70 | N |
| ATOM | 1025 | CA | ASP | B | 23 | 4.746 | −13.867 | 23.988 | 1.00 | 46.13 | C |
| ATOM | 1026 | CB | ASP | B | 23 | 3.273 | −14.163 | 24.328 | 1.00 | 50.01 | C |
| ATOM | 1027 | CG | ASP | B | 23 | 2.434 | −12.894 | 24.565 | 1.00 | 66.21 | C |
| ATOM | 1028 | OD1 | ASP | B | 23 | 2.999 | −11.786 | 24.740 | 1.00 | 68.81 | O |
| ATOM | 1029 | OD2 | ASP | B | 23 | 1.186 | −13.015 | 24.579 | 1.00 | 73.34 | O |
| ATOM | 1030 | C | ASP | B | 23 | 5.456 | −13.328 | 25.232 | 1.00 | 47.10 | C |
| ATOM | 1031 | O | ASP | B | 23 | 5.420 | −13.933 | 26.314 | 1.00 | 43.31 | O |
| ATOM | 1032 | N | PHE | B | 24 | 6.078 | −12.170 | 25.083 | 1.00 | 47.25 | N |
| ATOM | 1033 | CA | PHE | B | 24 | 6.665 | −11.475 | 26.217 | 1.00 | 44.68 | C |
| ATOM | 1034 | CB | PHE | B | 24 | 7.865 | −10.651 | 25.748 | 1.00 | 49.34 | C |
| ATOM | 1035 | CG | PHE | B | 24 | 9.039 | −11.485 | 25.331 | 1.00 | 48.06 | C |
| ATOM | 1036 | CD2 | PHE | B | 24 | 10.181 | −11.526 | 26.104 | 1.00 | 51.49 | C |
| ATOM | 1037 | CE2 | PHE | B | 24 | 11.264 | −12.311 | 25.730 | 1.00 | 48.77 | C |
| ATOM | 1038 | CZ | PHE | B | 24 | 11.210 | −13.052 | 24.581 | 1.00 | 51.16 | C |
| ATOM | 1039 | CE1 | PHE | B | 24 | 10.075 | −13.022 | 23.795 | 1.00 | 53.41 | C |
| ATOM | 1040 | CD1 | PHE | B | 24 | 8.995 | −12.242 | 24.171 | 1.00 | 51.72 | C |
| ATOM | 1041 | C | PHE | B | 24 | 5.643 | −10.581 | 26.914 | 1.00 | 46.41 | C |
| ATOM | 1042 | O | PHE | B | 24 | 5.231 | −9.551 | 26.385 | 1.00 | 52.44 | O |
| ATOM | 1043 | N | TRP | B | 25 | 5.251 | −10.958 | 28.122 | 1.00 | 49.55 | N |
| ATOM | 1044 | CA | TRP | B | 25 | 4.166 | −10.248 | 28.810 | 1.00 | 50.21 | C |
| ATOM | 1045 | CB | TRP | B | 25 | 2.917 | −11.126 | 28.853 | 1.00 | 38.06 | C |
| ATOM | 1046 | CG | TRP | B | 25 | 3.125 | −12.338 | 29.694 | 1.00 | 36.57 | C |
| ATOM | 1047 | CD1 | TRP | B | 25 | 3.812 | −13.467 | 29.352 | 1.00 | 43.51 | C |
| ATOM | 1048 | NE1 | TRP | B | 25 | 3.794 | −14.368 | 30.385 | 1.00 | 47.39 | N |
| ATOM | 1049 | CE2 | TRP | B | 25 | 3.104 | −13.823 | 31.434 | 1.00 | 53.97 | C |
| ATOM | 1050 | CD2 | TRP | B | 25 | 2.659 | −12.541 | 31.032 | 1.00 | 49.97 | C |
| ATOM | 1051 | CE3 | TRP | B | 25 | 1.918 | −11.762 | 31.933 | 1.00 | 41.36 | C |
| ATOM | 1052 | CZ3 | TRP | B | 25 | 1.644 | −12.284 | 33.178 | 1.00 | 41.49 | C |
| ATOM | 1053 | CH2 | TRP | B | 25 | 2.102 | −13.568 | 33.551 | 1.00 | 33.48 | C |
| ATOM | 1054 | CZ2 | TRP | B | 25 | 2.824 | −14.345 | 32.699 | 1.00 | 36.37 | C |
| ATOM | 1055 | C | TRP | B | 25 | 4.520 | −9.803 | 30.230 | 1.00 | 47.79 | C |
| ATOM | 1056 | O | TRP | B | 25 | 5.602 | −10.078 | 30.722 | 1.00 | 42.92 | O |
| ATOM | 1057 | N | ALA | B | 26 | 3.581 | −9.119 | 30.875 | 1.00 | 46.23 | N |
| ATOM | 1058 | CA | ALA | B | 26 | 3.747 | −8.649 | 32.243 | 1.00 | 46.32 | C |
| ATOM | 1059 | CB | ALA | B | 26 | 4.668 | −7.423 | 32.285 | 1.00 | 46.20 | C |
| ATOM | 1060 | C | ALA | B | 26 | 2.402 | −8.328 | 32.878 | 1.00 | 49.68 | C |
| ATOM | 1061 | O | ALA | B | 26 | 1.490 | −7.832 | 32.202 | 1.00 | 49.52 | O |
| ATOM | 1062 | N | GLU | B | 27 | 2.284 | −8.601 | 34.176 | 1.00 | 55.10 | N |
| ATOM | 1063 | CA | GLU | B | 27 | 1.053 | −8.320 | 34.910 | 1.00 | 47.35 | C |
| ATOM | 1064 | CB | GLU | B | 27 | 1.115 | −8.847 | 36.360 | 1.00 | 47.60 | C |
| ATOM | 1065 | CG | GLU | B | 27 | 1.030 | −10.402 | 36.512 | 1.00 | 50.82 | C |
| ATOM | 1066 | CD | GLU | B | 27 | 2.314 | −11.069 | 37.075 | 1.00 | 69.56 | C |
| ATOM | 1067 | OE1 | GLU | B | 27 | 3.443 | −10.633 | 36.735 | 1.00 | 73.38 | O |
| ATOM | 1068 | OE2 | GLU | B | 27 | 2.195 | −12.043 | 37.861 | 1.00 | 59.42 | O |
| ATOM | 1069 | C | GLU | B | 27 | 0.650 | −6.837 | 34.837 | 1.00 | 48.74 | C |
| ATOM | 1070 | O | GLU | B | 27 | −0.537 | −6.525 | 34.872 | 1.00 | 57.81 | O |
| ATOM | 1071 | N | TRP | B | 28 | 1.613 | −5.926 | 34.696 | 1.00 | 42.43 | N |
| ATOM | 1072 | CA | TRP | B | 28 | 1.281 | −4.495 | 34.609 | 1.00 | 46.07 | C |
| ATOM | 1073 | CB | TRP | B | 28 | 2.445 | −3.603 | 35.044 | 1.00 | 47.45 | C |
| ATOM | 1074 | CG | TRP | B | 28 | 3.780 | −4.015 | 34.526 | 1.00 | 53.22 | C |
| ATOM | 1075 | CD1 | TRP | B | 28 | 4.767 | −4.644 | 35.225 | 1.00 | 58.40 | C |
| ATOM | 1076 | NE1 | TRP | B | 28 | 5.862 | −4.846 | 34.422 | 1.00 | 57.59 | N |
| ATOM | 1077 | CE2 | TRP | B | 28 | 5.594 | −4.343 | 33.180 | 1.00 | 54.43 | C |
| ATOM | 1078 | CD2 | TRP | B | 28 | 4.296 | −3.801 | 33.208 | 1.00 | 52.87 | C |
| ATOM | 1079 | CE3 | TRP | B | 28 | 3.781 | −3.223 | 32.047 | 1.00 | 58.66 | C |
| ATOM | 1080 | CZ3 | TRP | B | 28 | 4.569 | −3.205 | 30.910 | 1.00 | 56.83 | C |
| ATOM | 1081 | CH2 | TRP | B | 28 | 5.859 | −3.740 | 30.921 | 1.00 | 54.72 | C |
| ATOM | 1082 | CZ2 | TRP | B | 28 | 6.382 | −4.312 | 32.040 | 1.00 | 56.31 | C |
| ATOM | 1083 | C | TRP | B | 28 | 0.801 | −4.050 | 33.230 | 1.00 | 44.67 | C |
| ATOM | 1084 | O | TRP | B | 28 | 0.530 | −2.878 | 33.000 | 1.00 | 40.25 | O |
| ATOM | 1085 | N | CYS | B | 29 | 0.683 | −4.994 | 32.318 | 1.00 | 41.27 | N |
| ATOM | 1086 | CA | CYS | B | 29 | 0.408 | −4.660 | 30.942 | 1.00 | 47.77 | C |
| ATOM | 1087 | CB | CYS | B | 29 | 1.359 | −5.461 | 30.050 | 1.00 | 49.44 | C |
| ATOM | 1088 | SG | CYS | B | 29 | 0.923 | −5.573 | 28.315 | 1.00 | 54.33 | S |
| ATOM | 1089 | C | CYS | B | 29 | −1.060 | −4.929 | 30.579 | 1.00 | 46.98 | C |
| ATOM | 1090 | O | CYS | B | 29 | −1.463 | −6.083 | 30.396 | 1.00 | 47.42 | O |
| ATOM | 1091 | N | GLY | B | 30 | −1.853 | −3.862 | 30.478 | 1.00 | 32.22 | N |
| ATOM | 1092 | CA | GLY | B | 30 | −3.271 | −3.982 | 30.144 | 1.00 | 39.33 | C |
| ATOM | 1093 | C | GLY | B | 30 | −3.574 | −4.867 | 28.940 | 1.00 | 41.54 | C |
| ATOM | 1094 | O | GLY | B | 30 | −4.165 | −5.936 | 29.115 | 1.00 | 39.21 | O |
| ATOM | 1095 | N | PRO | B | 31 | −3.150 | −4.445 | 27.721 | 1.00 | 39.76 | N |
| ATOM | 1096 | CA | PRO | B | 31 | −3.418 | −5.185 | 26.472 | 1.00 | 35.11 | C |
| ATOM | 1097 | CB | PRO | B | 31 | −2.670 | −4.393 | 25.384 | 1.00 | 30.68 | C |
| ATOM | 1098 | CG | PRO | B | 31 | −1.772 | −3.406 | 26.103 | 1.00 | 43.17 | C |
| ATOM | 1099 | CD | PRO | B | 31 | −2.306 | −3.248 | 27.513 | 1.00 | 40.78 | C |
| ATOM | 1100 | C | PRO | B | 31 | −2.882 | −6.598 | 26.518 | 1.00 | 41.34 | C |
| ATOM | 1101 | O | PRO | B | 31 | −3.345 | −7.476 | 25.782 | 1.00 | 44.92 | O |
| ATOM | 1102 | N | CYS | B | 32 | −1.892 | −6.827 | 27.368 | 1.00 | 43.72 | N |
| ATOM | 1103 | CA | CYS | B | 32 | −1.439 | −8.194 | 27.587 | 1.00 | 46.16 | C |
| ATOM | 1104 | CB | CYS | B | 32 | −0.264 | −8.224 | 28.572 | 1.00 | 41.98 | C |
| ATOM | 1105 | SG | CYS | B | 32 | 1.277 | −7.564 | 27.865 | 1.00 | 44.15 | S |
| ATOM | 1106 | C | CYS | B | 32 | −2.579 | −9.148 | 28.004 | 1.00 | 47.57 | C |
| ATOM | 1107 | O | CYS | B | 32 | −2.584 | −10.320 | 27.628 | 1.00 | 43.74 | O |
| ATOM | 1108 | N | ARG | B | 33 | −3.552 | −8.631 | 28.752 | 1.00 | 61.53 | N |
| ATOM | 1109 | CA | ARG | B | 33 | −4.694 | −9.434 | 29.205 | 1.00 | 71.74 | C |
| ATOM | 1110 | CB | ARG | B | 33 | −5.498 | −8.694 | 30.285 | 1.00 | 73.19 | C |
| ATOM | 1111 | CG | ARG | B | 33 | −4.725 | −8.433 | 31.573 | 1.00 | 59.71 | C |
| ATOM | 1112 | CD | ARG | B | 33 | −5.361 | −7.276 | 32.360 | 1.00 | 73.59 | C |
| ATOM | 1113 | NE | ARG | B | 33 | −4.756 | −6.965 | 33.667 | 1.00 | 88.65 | N |
| ATOM | 1114 | CZ | ARG | B | 33 | −3.522 | −7.286 | 34.070 | 1.00 | 78.95 | C |
| ATOM | 1115 | NH1 | ARG | B | 33 | −2.674 | −7.949 | 33.280 | 1.00 | 74.46 | N |
| ATOM | 1116 | NH2 | ARG | B | 33 | −3.129 | −6.924 | 35.285 | 1.00 | 71.42 | N |
| ATOM | 1117 | C | ARG | B | 33 | −5.627 | −9.914 | 28.071 | 1.00 | 68.14 | C |
| ATOM | 1118 | O | ARG | B | 33 | −6.235 | −10.976 | 28.174 | 1.00 | 73.53 | O |
| ATOM | 1119 | N | MET | B | 34 | −5.726 | −9.158 | 26.982 | 1.00 | 59.03 | N |
| ATOM | 1120 | CA | MET | B | 34 | −6.558 | −9.590 | 25.852 | 1.00 | 65.04 | C |
| ATOM | 1121 | CB | MET | B | 34 | −6.949 | −8.396 | 24.991 | 1.00 | 59.79 | C |
| ATOM | 1122 | CG | MET | B | 34 | −6.418 | −7.075 | 25.521 | 1.00 | 63.46 | C |
| ATOM | 1123 | SD | MET | B | 34 | −6.419 | −5.810 | 24.228 | 1.00 | 85.43 | S |
| ATOM | 1124 | CE | MET | B | 34 | −8.103 | −5.956 | 23.641 | 1.00 | 51.55 | C |
| ATOM | 1125 | C | MET | B | 34 | −5.866 | −10.628 | 24.986 | 1.00 | 59.71 | C |
| ATOM | 1126 | O | MET | B | 34 | −6.519 | −11.439 | 24.339 | 1.00 | 59.98 | O |
| ATOM | 1127 | N | ILE | B | 35 | −4.538 | −10.585 | 24.973 | 1.00 | 49.33 | N |
| ATOM | 1128 | CA | ILE | B | 35 | −3.744 | −11.481 | 24.134 | 1.00 | 53.69 | C |
| ATOM | 1129 | CB | ILE | B | 35 | −2.370 | −10.866 | 23.776 | 1.00 | 49.08 | C |
| ATOM | 1130 | CG1 | ILE | B | 35 | −2.531 | −9.542 | 23.043 | 1.00 | 51.33 | C |
| ATOM | 1131 | CD1 | ILE | B | 35 | −1.306 | −8.598 | 23.213 | 1.00 | 51.09 | C |
| ATOM | 1132 | CG2 | ILE | B | 35 | −1.566 | −11.812 | 22.933 | 1.00 | 49.18 | C |
| ATOM | 1133 | C | ILE | B | 35 | −3.498 | −12.835 | 24.802 | 1.00 | 56.23 | C |
| ATOM | 1134 | O | ILE | B | 35 | −3.266 | −13.842 | 24.132 | 1.00 | 57.30 | O |
| ATOM | 1135 | N | ALA | B | 36 | −3.524 | −12.859 | 26.126 | 1.00 | 53.64 | N |
| ATOM | 1136 | CA | ALA | B | 36 | −3.303 | −14.117 | 26.840 | 1.00 | 55.57 | C |
| ATOM | 1137 | CB | ALA | B | 36 | −3.463 | −13.936 | 28.361 | 1.00 | 47.65 | C |
| ATOM | 1138 | C | ALA | B | 36 | −4.226 | −15.221 | 26.321 | 1.00 | 56.96 | C |
| ATOM | 1139 | O | ALA | B | 36 | −3.747 | −16.286 | 25.916 | 1.00 | 56.63 | O |
| ATOM | 1140 | N | PRO | B | 37 | −5.554 | −14.975 | 26.345 | 1.00 | 64.12 | N |
| ATOM | 1141 | CA | PRO | B | 37 | −6.522 | −15.985 | 25.884 | 1.00 | 62.85 | C |
| ATOM | 1142 | C | PRO | B | 37 | −6.298 | −16.368 | 24.438 | 1.00 | 58.27 | C |
| ATOM | 1143 | O | PRO | B | 37 | −6.281 | −17.546 | 24.089 | 1.00 | 64.00 | O |
| ATOM | 1144 | CB | PRO | B | 37 | −7.874 | −15.275 | 26.017 | 1.00 | 56.53 | C |
| ATOM | 1145 | CG | PRO | B | 37 | −7.541 | −13.803 | 26.125 | 1.00 | 64.70 | C |
| ATOM | 1146 | CD | PRO | B | 37 | −6.231 | −13.772 | 26.862 | 1.00 | 58.90 | C |
| ATOM | 1147 | N | ILE | B | 38 | −6.122 | −15.368 | 23.594 | 1.00 | 49.38 | N |
| ATOM | 1148 | CA | ILE | B | 38 | −5.946 | −15.628 | 22.172 | 1.00 | 50.32 | C |
| ATOM | 1149 | CB | ILE | B | 38 | −5.780 | −14.307 | 21.404 | 1.00 | 50.26 | C |
| ATOM | 1150 | CG1 | ILE | B | 38 | −7.080 | −13.502 | 21.547 | 1.00 | 48.34 | C |
| ATOM | 1151 | CD1 | ILE | B | 38 | −7.072 | −12.139 | 20.896 | 1.00 | 46.09 | C |
| ATOM | 1152 | CG2 | ILE | B | 38 | −5.445 | −14.558 | 19.960 | 1.00 | 47.52 | C |
| ATOM | 1153 | C | ILE | B | 38 | −4.817 | −16.614 | 21.888 | 1.00 | 55.58 | C |
| ATOM | 1154 | O | ILE | B | 38 | −5.057 | −17.657 | 21.290 | 1.00 | 60.38 | O |
| ATOM | 1155 | N | ILE | B | 39 | −3.597 | −16.297 | 22.327 | 1.00 | 60.95 | N |
| ATOM | 1156 | CA | ILE | B | 39 | −2.441 | −17.192 | 22.145 | 1.00 | 54.32 | C |
| ATOM | 1157 | CB | ILE | B | 39 | −1.226 | −16.736 | 22.973 | 1.00 | 60.90 | C |
| ATOM | 1158 | CG1 | ILE | B | 39 | −0.316 | −15.853 | 22.126 | 1.00 | 58.32 | C |
| ATOM | 1159 | CD1 | ILE | B | 39 | −1.041 | −14.921 | 21.228 | 1.00 | 57.66 | C |
| ATOM | 1160 | CG2 | ILE | B | 39 | −0.414 | −17.936 | 23.474 | 1.00 | 55.24 | C |
| ATOM | 1161 | C | ILE | B | 39 | −2.772 | −18.614 | 22.544 | 1.00 | 55.84 | C |
| ATOM | 1162 | O | ILE | B | 39 | −2.341 | −19.556 | 21.891 | 1.00 | 56.53 | O |
| ATOM | 1163 | N | GLU | B | 40 | −3.524 | −18.755 | 23.633 | 1.00 | 67.89 | N |
| ATOM | 1164 | CA | GLU | B | 40 | −4.045 | −20.047 | 24.069 | 1.00 | 71.72 | C |
| ATOM | 1165 | CB | GLU | B | 40 | −4.896 | −19.884 | 25.323 | 1.00 | 69.49 | C |
| ATOM | 1166 | CG | GLU | B | 40 | −4.114 | −19.907 | 26.602 | 1.00 | 76.37 | C |
| ATOM | 1167 | CD | GLU | B | 40 | −4.923 | −19.363 | 27.744 | 1.00 | 85.01 | C |
| ATOM | 1168 | OE2 | GLU | B | 40 | −4.316 | −19.017 | 28.783 | 1.00 | 97.30 | O |
| ATOM | 1169 | OE1 | GLU | B | 40 | −6.164 | −19.269 | 27.588 | 1.00 | 80.90 | O |
| ATOM | 1170 | C | GLU | B | 40 | −4.891 | −20.711 | 22.998 | 1.00 | 73.56 | C |
| ATOM | 1171 | O | GLU | B | 40 | −4.647 | −21.867 | 22.657 | 1.00 | 69.17 | O |
| ATOM | 1172 | N | GLU | B | 41 | −5.901 | −19.986 | 22.503 | 1.00 | 77.03 | N |
| ATOM | 1173 | CA | GLU | B | 41 | −6.799 | −20.497 | 21.468 | 1.00 | 74.03 | C |
| ATOM | 1174 | CB | GLU | B | 41 | −7.797 | −19.418 | 21.003 | 1.00 | 67.29 | C |
| ATOM | 1175 | CG | GLU | B | 41 | −8.783 | −18.942 | 22.095 | 1.00 | 74.98 | C |
| ATOM | 1176 | CD | GLU | B | 41 | −9.725 | −17.840 | 21.604 | 1.00 | 77.61 | C |
| ATOM | 1177 | OE1 | GLU | B | 41 | −10.750 | −17.566 | 22.275 | 1.00 | 72.53 | O |
| ATOM | 1178 | OE2 | GLU | B | 41 | −9.430 | −17.239 | 20.544 | 1.00 | 68.23 | O |
| ATOM | 1179 | C | GLU | B | 41 | −5.963 | −21.019 | 20.306 | 1.00 | 80.18 | C |
| ATOM | 1180 | O | GLU | B | 41 | −6.243 | −22.091 | 19.762 | 1.00 | 86.25 | O |
| ATOM | 1181 | N | LEU | B | 42 | −4.916 | −20.273 | 19.954 | 1.00 | 58.10 | N |
| ATOM | 1182 | CA | LEU | B | 42 | −4.032 | −20.665 | 18.865 | 1.00 | 61.89 | C |
| ATOM | 1183 | CB | LEU | B | 42 | −3.186 | −19.481 | 18.397 | 1.00 | 65.15 | C |
| ATOM | 1184 | CG | LEU | B | 42 | −3.908 | −18.392 | 17.605 | 1.00 | 70.91 | C |
| ATOM | 1185 | CD1 | LEU | B | 42 | −2.932 | −17.298 | 17.166 | 1.00 | 67.89 | C |
| ATOM | 1186 | CD2 | LEU | B | 42 | −4.583 | −19.007 | 16.408 | 1.00 | 69.23 | C |
| ATOM | 1187 | C | LEU | B | 42 | −3.131 | −21.839 | 19.247 | 1.00 | 65.88 | C |
| ATOM | 1188 | O | LEU | B | 42 | −2.709 | −22.608 | 18.392 | 1.00 | 69.34 | O |
| ATOM | 1189 | N | ALA | B | 43 | −2.830 | −21.977 | 20.531 | 1.00 | 92.93 | N |
| ATOM | 1190 | CA | ALA | B | 43 | −2.002 | −23.090 | 20.977 | 1.00 | 96.49 | C |
| ATOM | 1191 | CB | ALA | B | 43 | −1.586 | −22.917 | 22.437 | 1.00 | 87.63 | C |
| ATOM | 1192 | C | ALA | B | 43 | −2.769 | −24.388 | 20.783 | 1.00 | 98.90 | C |
| ATOM | 1193 | O | ALA | B | 43 | −2.179 | −25.451 | 20.589 | 1.00 | 99.34 | O |
| ATOM | 1194 | N | GLU | B | 44 | −4.093 | −24.287 | 20.830 | 1.00 | 94.24 | N |
| ATOM | 1195 | CA | GLU | B | 44 | −4.959 | −25.441 | 20.640 | 1.00 | 94.43 | C |
| ATOM | 1196 | CB | GLU | B | 44 | −6.255 | −25.275 | 21.441 | 1.00 | 91.68 | C |
| ATOM | 1197 | CG | GLU | B | 44 | −6.018 | −25.073 | 22.942 | 1.00 | 102.06 | C |
| ATOM | 1198 | CD | GLU | B | 44 | −7.241 | −25.394 | 23.793 | 1.00 | 113.53 | C |
| ATOM | 1199 | OE1 | GLU | B | 44 | −8.382 | −25.204 | 23.313 | 1.00 | 111.24 | O |
| ATOM | 1200 | OE2 | GLU | B | 44 | −7.056 | −25.849 | 24.943 | 1.00 | 111.95 | O |
| ATOM | 1201 | C | GLU | B | 44 | −5.234 | −25.642 | 19.155 | 1.00 | 95.11 | C |
| ATOM | 1202 | O | GLU | B | 44 | −5.150 | −26.756 | 18.637 | 1.00 | 88.62 | O |
| ATOM | 1203 | N | GLU | B | 45 | −5.542 | −24.549 | 18.469 | 1.00 | 94.20 | N |
| ATOM | 1204 | CA | GLU | B | 45 | −5.684 | −24.583 | 17.023 | 1.00 | 91.51 | C |
| ATOM | 1205 | CB | GLU | B | 45 | −5.832 | −23.165 | 16.472 | 1.00 | 88.51 | C |
| ATOM | 1206 | CG | GLU | B | 45 | −6.013 | −23.099 | 14.968 | 1.00 | 91.15 | C |
| ATOM | 1207 | CD | GLU | B | 45 | −6.631 | −21.788 | 14.502 | 1.00 | 92.61 | C |
| ATOM | 1208 | OE1 | GLU | B | 45 | −7.329 | −21.127 | 15.303 | 1.00 | 86.51 | O |
| ATOM | 1209 | OE2 | GLU | B | 45 | −6.420 | −21.420 | 13.326 | 1.00 | 102.52 | O |
| ATOM | 1210 | C | GLU | B | 45 | −4.480 | −25.294 | 16.393 | 1.00 | 92.91 | C |
| ATOM | 1211 | O | GLU | B | 45 | −4.578 | −26.455 | 15.995 | 1.00 | 94.92 | O |
| ATOM | 1212 | N | TYR | B | 46 | −3.341 | −24.610 | 16.336 | 1.00 | 99.92 | N |
| ATOM | 1213 | CA | TYR | B | 46 | −2.123 | −25.177 | 15.758 | 1.00 | 102.82 | C |
| ATOM | 1214 | CB | TYR | B | 46 | −1.180 | −24.062 | 15.334 | 1.00 | 104.93 | C |
| ATOM | 1215 | CG | TYR | B | 46 | −1.800 | −23.002 | 14.476 | 1.00 | 97.94 | C |
| ATOM | 1216 | CD2 | TYR | B | 46 | −1.707 | −23.070 | 13.103 | 1.00 | 102.67 | C |
| ATOM | 1217 | CE2 | TYR | B | 46 | −2.261 | −22.102 | 12.305 | 1.00 | 109.54 | C |
| ATOM | 1218 | CZ | TYR | B | 46 | −2.914 | −21.034 | 12.878 | 1.00 | 104.52 | C |
| ATOM | 1219 | OH | TYR | B | 46 | −3.465 | −20.069 | 12.065 | 1.00 | 103.24 | O |
| ATOM | 1220 | CE1 | TYR | B | 46 | −3.013 | −20.938 | 14.252 | 1.00 | 98.32 | C |
| ATOM | 1221 | CD1 | TYR | B | 46 | −2.454 | −21.921 | 15.039 | 1.00 | 96.54 | C |
| ATOM | 1222 | C | TYR | B | 46 | −1.333 | −26.075 | 16.700 | 1.00 | 107.17 | C |
| ATOM | 1223 | O | TYR | B | 46 | −0.105 | −25.973 | 16.753 | 1.00 | 103.47 | O |
| ATOM | 1224 | N | ALA | B | 47 | −2.016 | −26.950 | 17.432 | 1.00 | 111.29 | N |
| ATOM | 1225 | CA | ALA | B | 47 | −1.338 | −27.823 | 18.390 | 1.00 | 113.07 | C |
| ATOM | 1226 | CB | ALA | B | 47 | −2.307 | −28.307 | 19.459 | 1.00 | 108.38 | C |
| ATOM | 1227 | C | ALA | B | 47 | −0.653 | −29.011 | 17.706 | 1.00 | 110.68 | C |
| ATOM | 1228 | O | ALA | B | 47 | −1.286 | −29.773 | 16.974 | 1.00 | 103.96 | O |
| ATOM | 1229 | N | GLY | B | 48 | 0.646 | −29.159 | 17.952 | 1.00 | 86.03 | N |
| ATOM | 1230 | CA | GLY | B | 48 | 1.429 | −30.205 | 17.319 | 1.00 | 83.47 | C |
| ATOM | 1231 | C | GLY | B | 48 | 2.157 | −29.693 | 16.095 | 1.00 | 81.29 | C |
| ATOM | 1232 | O | GLY | B | 48 | 3.154 | −30.275 | 15.670 | 1.00 | 77.77 | O |
| ATOM | 1233 | N | LYS | B | 49 | 1.658 | −28.597 | 15.529 | 1.00 | 90.65 | N |
| ATOM | 1234 | CA | LYS | B | 49 | 2.282 | −27.995 | 14.354 | 1.00 | 94.47 | C |
| ATOM | 1235 | CB | LYS | B | 49 | 1.284 | −27.871 | 13.197 | 1.00 | 95.48 | C |
| ATOM | 1236 | CG | LYS | B | 49 | 0.179 | −28.885 | 13.222 | 1.00 | 95.02 | C |
| ATOM | 1237 | CD | LYS | B | 49 | −1.025 | −28.370 | 12.483 | 1.00 | 95.05 | C |
| ATOM | 1238 | CE | LYS | B | 49 | −2.260 | −28.636 | 13.311 | 1.00 | 99.74 | C |
| ATOM | 1239 | NZ | LYS | B | 49 | −1.981 | −28.402 | 14.764 | 1.00 | 94.62 | N |
| ATOM | 1240 | C | LYS | B | 49 | 2.871 | −26.608 | 14.611 | 1.00 | 88.60 | C |
| ATOM | 1241 | O | LYS | B | 49 | 3.048 | −25.841 | 13.673 | 1.00 | 91.68 | O |
| ATOM | 1242 | N | VAL | B | 50 | 3.180 | −26.282 | 15.858 | 1.00 | 77.93 | N |
| ATOM | 1243 | CA | VAL | B | 50 | 3.825 | −25.013 | 16.198 | 1.00 | 77.88 | C |
| ATOM | 1244 | CB | VAL | B | 50 | 3.130 | −23.762 | 15.600 | 1.00 | 82.64 | C |
| ATOM | 1245 | CG1 | VAL | B | 50 | 3.147 | −22.611 | 16.607 | 1.00 | 72.07 | C |
| ATOM | 1246 | CG2 | VAL | B | 50 | 3.797 | −23.327 | 14.302 | 1.00 | 77.07 | C |
| ATOM | 1247 | C | VAL | B | 50 | 3.723 | −24.895 | 17.681 | 1.00 | 72.44 | C |
| ATOM | 1248 | O | VAL | B | 50 | 2.640 | −25.095 | 18.240 | 1.00 | 68.36 | O |
| ATOM | 1249 | N | VAL | B | 51 | 4.848 | −24.560 | 18.308 | 1.00 | 69.10 | N |
| ATOM | 1250 | CA | VAL | B | 51 | 4.933 | −24.515 | 19.762 | 1.00 | 71.66 | C |
| ATOM | 1251 | CB | VAL | B | 51 | 6.173 | −25.296 | 20.270 | 1.00 | 74.70 | C |
| ATOM | 1252 | CG1 | VAL | B | 51 | 7.223 | −25.401 | 19.167 | 1.00 | 72.04 | C |
| ATOM | 1253 | CG2 | VAL | B | 51 | 6.750 | −24.662 | 21.525 | 1.00 | 66.82 | C |
| ATOM | 1254 | C | VAL | B | 51 | 4.899 | −23.081 | 20.293 | 1.00 | 68.23 | C |
| ATOM | 1255 | O | VAL | B | 51 | 5.461 | −22.158 | 19.682 | 1.00 | 62.46 | O |
| ATOM | 1256 | N | PHE | B | 52 | 4.222 | −22.912 | 21.427 | 1.00 | 84.28 | N |
| ATOM | 1257 | CA | PHE | B | 52 | 3.961 | −21.597 | 21.998 | 1.00 | 80.51 | C |
| ATOM | 1258 | CB | PHE | B | 52 | 2.461 | −21.344 | 22.068 | 1.00 | 74.73 | C |
| ATOM | 1259 | CG | PHE | B | 52 | 1.790 | −21.304 | 20.750 | 1.00 | 75.32 | C |
| ATOM | 1260 | CD2 | PHE | B | 52 | 1.763 | −20.133 | 20.017 | 1.00 | 80.85 | C |
| ATOM | 1261 | CE2 | PHE | B | 52 | 1.126 | −20.077 | 18.785 | 1.00 | 83.29 | C |
| ATOM | 1262 | CZ | PHE | B | 52 | 0.502 | −21.209 | 18.281 | 1.00 | 86.94 | C |
| ATOM | 1263 | CE1 | PHE | B | 52 | 0.522 | −22.385 | 19.013 | 1.00 | 91.21 | C |
| ATOM | 1264 | CD1 | PHE | B | 52 | 1.164 | −22.428 | 20.243 | 1.00 | 85.14 | C |
| ATOM | 1265 | C | PHE | B | 52 | 4.470 | −21.462 | 23.416 | 1.00 | 75.60 | C |
| ATOM | 1266 | O | PHE | B | 52 | 4.026 | −22.182 | 24.312 | 1.00 | 77.16 | O |
| ATOM | 1267 | N | GLY | B | 53 | 5.363 | −20.507 | 23.634 | 1.00 | 71.47 | N |
| ATOM | 1268 | CA | GLY | B | 53 | 5.771 | −20.169 | 24.986 | 1.00 | 70.87 | C |
| ATOM | 1269 | C | GLY | B | 53 | 5.553 | −18.705 | 25.325 | 1.00 | 67.68 | C |
| ATOM | 1270 | O | GLY | B | 53 | 5.697 | −17.835 | 24.464 | 1.00 | 71.11 | O |
| ATOM | 1271 | N | LYS | B | 54 | 5.187 | −18.434 | 26.575 | 1.00 | 57.51 | N |
| ATOM | 1272 | CA | LYS | B | 54 | 5.186 | −17.068 | 27.103 | 1.00 | 50.75 | C |
| ATOM | 1273 | CB | LYS | B | 54 | 3.859 | −16.751 | 27.790 | 1.00 | 47.07 | C |
| ATOM | 1274 | CG | LYS | B | 54 | 3.507 | −17.711 | 28.918 | 1.00 | 57.82 | C |
| ATOM | 1275 | CD | LYS | B | 54 | 2.023 | −17.635 | 29.279 | 1.00 | 62.01 | C |
| ATOM | 1276 | CE | LYS | B | 54 | 1.623 | −18.735 | 30.251 | 1.00 | 68.17 | C |
| ATOM | 1277 | NZ | LYS | B | 54 | 0.148 | −18.757 | 30.487 | 1.00 | 76.46 | N |
| ATOM | 1278 | C | LYS | B | 54 | 6.313 | −16.904 | 28.103 | 1.00 | 43.13 | C |
| ATOM | 1279 | O | LYS | B | 54 | 6.665 | −17.846 | 28.820 | 1.00 | 44.19 | O |
| ATOM | 1280 | N | VAL | B | 55 | 6.871 | −15.703 | 28.161 | 1.00 | 35.64 | N |
| ATOM | 1281 | CA | VAL | B | 55 | 7.829 | −15.380 | 29.214 | 1.00 | 33.56 | C |
| ATOM | 1282 | CB | VAL | B | 55 | 9.323 | −15.269 | 28.710 | 1.00 | 35.61 | C |
| ATOM | 1283 | CG1 | VAL | B | 55 | 9.461 | −15.659 | 27.268 | 1.00 | 38.04 | C |
| ATOM | 1284 | CG2 | VAL | B | 55 | 9.911 | −13.879 | 28.938 | 1.00 | 30.88 | C |
| ATOM | 1285 | C | VAL | B | 55 | 7.412 | −14.131 | 29.980 | 1.00 | 35.37 | C |
| ATOM | 1286 | O | VAL | B | 55 | 7.279 | −13.061 | 29.399 | 1.00 | 39.78 | O |
| ATOM | 1287 | N | ASN | B | 56 | 7.157 | −14.270 | 31.277 | 1.00 | 43.17 | N |
| ATOM | 1288 | CA | ASN | B | 56 | 6.908 | −13.095 | 32.096 | 1.00 | 43.41 | C |
| ATOM | 1289 | CB | ASN | B | 56 | 6.518 | −13.464 | 33.528 | 1.00 | 40.96 | C |
| ATOM | 1290 | CG | ASN | B | 56 | 5.988 | −12.281 | 34.331 | 1.00 | 44.32 | C |
| ATOM | 1291 | OD1 | ASN | B | 56 | 6.453 | −11.149 | 34.200 | 1.00 | 46.25 | O |
| ATOM | 1292 | ND2 | ASN | B | 56 | 5.012 | −12.552 | 35.184 | 1.00 | 46.57 | N |
| ATOM | 1293 | C | ASN | B | 56 | 8.198 | −12.315 | 32.071 | 1.00 | 39.09 | C |
| ATOM | 1294 | O | ASN | B | 56 | 9.255 | −12.852 | 32.338 | 1.00 | 46.22 | O |
| ATOM | 1295 | N | VAL | B | 57 | 8.102 | −11.050 | 31.707 | 1.00 | 40.11 | N |
| ATOM | 1296 | CA | VAL | B | 57 | 9.252 | −10.197 | 31.483 | 1.00 | 38.23 | C |
| ATOM | 1297 | CB | VAL | B | 57 | 8.809 | −8.957 | 30.656 | 1.00 | 41.82 | C |
| ATOM | 1298 | CG1 | VAL | B | 57 | 8.891 | −7.670 | 31.477 | 1.00 | 48.41 | C |
| ATOM | 1299 | CG2 | VAL | B | 57 | 9.587 | −8.842 | 29.386 | 1.00 | 37.48 | C |
| ATOM | 1300 | C | VAL | B | 57 | 9.869 | −9.768 | 32.813 | 1.00 | 43.99 | C |
| ATOM | 1301 | O | VAL | B | 57 | 11.073 | −9.584 | 32.908 | 1.00 | 43.97 | O |
| ATOM | 1302 | N | ASP | B | 58 | 9.032 | −9.624 | 33.836 | 1.00 | 39.39 | N |
| ATOM | 1303 | CA | ASP | B | 58 | 9.470 | −9.129 | 35.136 | 1.00 | 44.62 | C |
| ATOM | 1304 | CB | ASP | B | 58 | 8.272 | −8.663 | 35.962 | 1.00 | 44.16 | C |
| ATOM | 1305 | CG | ASP | B | 58 | 7.940 | −7.199 | 35.728 | 1.00 | 55.90 | C |
| ATOM | 1306 | OD1 | ASP | B | 58 | 8.867 | −6.431 | 35.388 | 1.00 | 60.79 | O |
| ATOM | 1307 | OD2 | ASP | B | 58 | 6.760 | −6.819 | 35.888 | 1.00 | 58.41 | O |
| ATOM | 1308 | C | ASP | B | 58 | 10.259 | −10.174 | 35.917 | 1.00 | 50.25 | C |
| ATOM | 1309 | O | ASP | B | 58 | 11.089 | −9.827 | 36.753 | 1.00 | 41.44 | O |
| ATOM | 1310 | N | GLU | B | 59 | 9.988 | −11.441 | 35.611 | 1.00 | 60.17 | N |
| ATOM | 1311 | CA | GLU | B | 59 | 10.555 | −12.592 | 36.296 | 1.00 | 56.54 | C |
| ATOM | 1312 | CB | GLU | B | 59 | 9.434 | −13.592 | 36.618 | 1.00 | 58.67 | C |
| ATOM | 1313 | CG | GLU | B | 59 | 8.463 | −13.102 | 37.702 | 1.00 | 59.69 | C |
| ATOM | 1314 | CD | GLU | B | 59 | 7.190 | −13.951 | 37.829 | 1.00 | 74.62 | C |
| ATOM | 1315 | OE1 | GLU | B | 59 | 7.067 | −14.972 | 37.100 | 1.00 | 59.26 | O |
| ATOM | 1316 | OE2 | GLU | B | 59 | 6.310 | −13.581 | 38.664 | 1.00 | 77.74 | O |
| ATOM | 1317 | C | GLU | B | 59 | 11.647 | −13.251 | 35.458 | 1.00 | 63.18 | C |
| ATOM | 1318 | O | GLU | B | 59 | 12.380 | −14.121 | 35.928 | 1.00 | 67.55 | O |
| ATOM | 1319 | N | ASN | B | 60 | 11.744 | −12.826 | 34.204 | 1.00 | 46.77 | N |
| ATOM | 1320 | CA | ASN | B | 60 | 12.788 | −13.294 | 33.309 | 1.00 | 44.58 | C |
| ATOM | 1321 | CB | ASN | B | 60 | 12.245 | −14.349 | 32.351 | 1.00 | 42.72 | C |
| ATOM | 1322 | CG | ASN | B | 60 | 11.783 | −15.592 | 33.069 | 1.00 | 50.38 | C |
| ATOM | 1323 | OD1 | ASN | B | 60 | 12.597 | −16.333 | 33.604 | 1.00 | 53.96 | O |
| ATOM | 1324 | ND2 | ASN | B | 60 | 10.472 | −15.832 | 33.084 | 1.00 | 45.59 | N |
| ATOM | 1325 | C | ASN | B | 60 | 13.399 | −12.115 | 32.548 | 1.00 | 53.55 | C |
| ATOM | 1326 | O | ASN | B | 60 | 13.521 | −12.131 | 31.307 | 1.00 | 53.82 | O |
| ATOM | 1327 | N | PRO | B | 61 | 13.822 | −11.098 | 33.304 | 1.00 | 53.19 | N |
| ATOM | 1328 | CA | PRO | B | 61 | 14.252 | −9.815 | 32.752 | 1.00 | 54.72 | C |
| ATOM | 1329 | CB | PRO | B | 61 | 14.613 | −9.009 | 34.010 | 1.00 | 44.21 | C |
| ATOM | 1330 | CG | PRO | B | 61 | 15.069 | −10.042 | 34.976 | 1.00 | 51.64 | C |
| ATOM | 1331 | CD | PRO | B | 61 | 14.228 | −11.261 | 34.713 | 1.00 | 57.31 | C |
| ATOM | 1332 | C | PRO | B | 61 | 15.472 | −9.976 | 31.848 | 1.00 | 63.18 | C |
| ATOM | 1333 | O | PRO | B | 61 | 15.664 | −9.162 | 30.939 | 1.00 | 65.32 | O |
| ATOM | 1334 | N | GLU | B | 62 | 16.275 | −11.011 | 32.091 | 1.00 | 58.07 | N |
| ATOM | 1335 | CA | GLU | B | 62 | 17.520 | −11.184 | 31.364 | 1.00 | 53.43 | C |
| ATOM | 1336 | CB | GLU | B | 62 | 18.441 | −12.206 | 32.040 | 1.00 | 60.80 | C |
| ATOM | 1337 | CG | GLU | B | 62 | 19.451 | −11.591 | 33.014 | 1.00 | 64.64 | C |
| ATOM | 1338 | CD | GLU | B | 62 | 19.140 | −11.933 | 34.463 | 1.00 | 76.08 | C |
| ATOM | 1339 | OE1 | GLU | B | 62 | 18.060 | −12.522 | 34.716 | 1.00 | 85.98 | O |
| ATOM | 1340 | OE2 | GLU | B | 62 | 19.970 | −11.625 | 35.345 | 1.00 | 70.48 | O |
| ATOM | 1341 | C | GLU | B | 62 | 17.234 | −11.593 | 29.943 | 1.00 | 55.62 | C |
| ATOM | 1342 | O | GLU | B | 62 | 17.930 | −11.174 | 29.022 | 1.00 | 56.09 | O |
| ATOM | 1343 | N | ILE | B | 63 | 16.204 | −12.409 | 29.759 | 1.00 | 42.81 | N |
| ATOM | 1344 | CA | ILE | B | 63 | 15.777 | −12.736 | 28.402 | 1.00 | 43.39 | C |
| ATOM | 1345 | CB | ILE | B | 63 | 14.720 | −13.819 | 28.382 | 1.00 | 42.88 | C |
| ATOM | 1346 | CG1 | ILE | B | 63 | 15.267 | −15.100 | 29.009 | 1.00 | 46.88 | C |
| ATOM | 1347 | CD1 | ILE | B | 63 | 14.198 | −16.106 | 29.375 | 1.00 | 42.53 | C |
| ATOM | 1348 | CG2 | ILE | B | 63 | 14.260 | −14.074 | 26.948 | 1.00 | 49.11 | C |
| ATOM | 1349 | C | ILE | B | 63 | 15.259 | −11.509 | 27.638 | 1.00 | 42.65 | C |
| ATOM | 1350 | O | ILE | B | 63 | 15.710 | −11.234 | 26.528 | 1.00 | 43.67 | O |
| ATOM | 1351 | N | ALA | B | 64 | 14.324 | −10.766 | 28.219 | 1.00 | 65.39 | N |
| ATOM | 1352 | CA | ALA | B | 64 | 13.843 | −9.552 | 27.563 | 1.00 | 69.64 | C |
| ATOM | 1353 | CB | ALA | B | 64 | 12.933 | −8.756 | 28.503 | 1.00 | 60.36 | C |
| ATOM | 1354 | C | ALA | B | 64 | 15.032 | −8.701 | 27.107 | 1.00 | 66.52 | C |
| ATOM | 1355 | O | ALA | B | 64 | 15.119 | −8.296 | 25.936 | 1.00 | 61.59 | O |
| ATOM | 1356 | N | ALA | B | 65 | 15.949 | −8.475 | 28.048 | 1.00 | 67.05 | N |
| ATOM | 1357 | CA | ALA | B | 65 | 17.155 | −7.667 | 27.848 | 1.00 | 68.07 | C |
| ATOM | 1358 | CB | ALA | B | 65 | 17.865 | −7.438 | 29.164 | 1.00 | 57.43 | C |
| ATOM | 1359 | C | ALA | B | 65 | 18.129 | −8.252 | 26.834 | 1.00 | 73.63 | C |
| ATOM | 1360 | O | ALA | B | 65 | 18.877 | −7.512 | 26.195 | 1.00 | 76.90 | O |
| ATOM | 1361 | N | LYS | B | 66 | 18.132 | −9.575 | 26.697 | 1.00 | 67.41 | N |
| ATOM | 1362 | CA | LYS | B | 66 | 19.007 | −10.231 | 25.729 | 1.00 | 63.61 | C |
| ATOM | 1363 | CB | LYS | B | 66 | 19.112 | −11.731 | 26.007 | 1.00 | 62.32 | C |
| ATOM | 1364 | CG | LYS | B | 66 | 19.873 | −12.483 | 24.941 | 1.00 | 66.41 | C |
| ATOM | 1365 | CD | LYS | B | 66 | 19.660 | −13.985 | 25.044 | 1.00 | 68.88 | C |
| ATOM | 1366 | CE | LYS | B | 66 | 20.333 | −14.712 | 23.878 | 1.00 | 74.39 | C |
| ATOM | 1367 | NZ | LYS | B | 66 | 21.815 | −14.444 | 23.763 | 1.00 | 69.21 | N |
| ATOM | 1368 | C | LYS | B | 66 | 18.528 | −10.014 | 24.298 | 1.00 | 62.69 | C |
| ATOM | 1369 | O | LYS | B | 66 | 19.335 | −9.996 | 23.372 | 1.00 | 66.63 | O |
| ATOM | 1370 | N | TYR | B | 67 | 17.218 | −9.842 | 24.121 | 1.00 | 57.10 | N |
| ATOM | 1371 | CA | TYR | B | 67 | 16.618 | −9.795 | 22.783 | 1.00 | 43.01 | C |
| ATOM | 1372 | CB | TYR | B | 67 | 15.536 | −10.869 | 22.661 | 1.00 | 46.50 | C |
| ATOM | 1373 | CG | TYR | B | 67 | 16.097 | −12.259 | 22.482 | 1.00 | 54.72 | C |
| ATOM | 1374 | CD1 | TYR | B | 67 | 16.707 | −12.620 | 21.288 | 1.00 | 60.24 | C |
| ATOM | 1375 | CE1 | TYR | B | 67 | 17.244 | −13.883 | 21.104 | 1.00 | 59.07 | C |
| ATOM | 1376 | CZ | TYR | B | 67 | 17.162 | −14.809 | 22.119 | 1.00 | 59.32 | C |
| ATOM | 1377 | OH | TYR | B | 67 | 17.693 | −16.055 | 21.902 | 1.00 | 58.97 | O |
| ATOM | 1378 | CE2 | TYR | B | 67 | 16.546 | −14.485 | 23.317 | 1.00 | 54.78 | C |
| ATOM | 1379 | CD2 | TYR | B | 67 | 16.021 | −13.210 | 23.494 | 1.00 | 46.66 | C |
| ATOM | 1380 | C | TYR | B | 67 | 16.077 | −8.425 | 22.366 | 1.00 | 41.54 | C |
| ATOM | 1381 | O | TYR | B | 67 | 15.415 | −8.305 | 21.339 | 1.00 | 51.20 | O |
| ATOM | 1382 | N | GLY | B | 68 | 16.348 | −7.391 | 23.158 | 1.00 | 49.99 | N |
| ATOM | 1383 | CA | GLY | B | 68 | 15.949 | −6.042 | 22.772 | 1.00 | 50.67 | C |
| ATOM | 1384 | C | GLY | B | 68 | 14.503 | −5.683 | 23.084 | 1.00 | 46.34 | C |
| ATOM | 1385 | O | GLY | B | 68 | 14.074 | −4.548 | 22.886 | 1.00 | 51.01 | O |
| ATOM | 1386 | N | ILE | B | 69 | 13.770 | −6.666 | 23.594 | 1.00 | 39.72 | N |
| ATOM | 1387 | CA | ILE | B | 69 | 12.362 | −6.545 | 23.930 | 1.00 | 44.17 | C |
| ATOM | 1388 | CB | ILE | B | 69 | 11.764 | −7.927 | 24.174 | 1.00 | 39.48 | C |
| ATOM | 1389 | CG1 | ILE | B | 69 | 12.041 | −8.809 | 22.960 | 1.00 | 33.21 | C |
| ATOM | 1390 | CD1 | ILE | B | 69 | 11.710 | −10.224 | 23.186 | 1.00 | 42.31 | C |
| ATOM | 1391 | CG2 | ILE | B | 69 | 10.267 | −7.814 | 24.485 | 1.00 | 36.65 | C |
| ATOM | 1392 | C | ILE | B | 69 | 12.103 | −5.670 | 25.152 | 1.00 | 47.36 | C |
| ATOM | 1393 | O | ILE | B | 69 | 11.912 | −6.166 | 26.272 | 1.00 | 48.29 | O |
| ATOM | 1394 | N | MET | B | 70 | 12.082 | −4.364 | 24.917 | 1.00 | 36.20 | N |
| ATOM | 1395 | CA | MET | B | 70 | 11.853 | −3.391 | 25.965 | 1.00 | 37.27 | C |
| ATOM | 1396 | CB | MET | B | 70 | 12.869 | −2.263 | 25.829 | 1.00 | 40.10 | C |
| ATOM | 1397 | CG | MET | B | 70 | 14.223 | −2.611 | 26.409 | 1.00 | 45.93 | C |
| ATOM | 1398 | SD | MET | B | 70 | 14.030 | −3.287 | 28.078 | 1.00 | 50.06 | S |
| ATOM | 1399 | CE | MET | B | 70 | 14.320 | −5.052 | 27.827 | 1.00 | 43.96 | C |
| ATOM | 1400 | C | MET | B | 70 | 10.445 | −2.818 | 25.891 | 1.00 | 47.22 | C |
| ATOM | 1401 | O | MET | B | 70 | 10.148 | −1.800 | 26.496 | 1.00 | 45.24 | O |
| ATOM | 1402 | N | SER | B | 71 | 9.580 | −3.486 | 25.145 | 1.00 | 35.71 | N |
| ATOM | 1403 | CA | SER | B | 71 | 8.271 | −2.959 | 24.869 | 1.00 | 38.89 | C |
| ATOM | 1404 | CB | SER | B | 71 | 8.330 | −2.079 | 23.614 | 1.00 | 50.71 | C |
| ATOM | 1405 | OG | SER | B | 71 | 7.230 | −2.312 | 22.747 | 1.00 | 40.16 | O |
| ATOM | 1406 | C | SER | B | 71 | 7.307 | −4.118 | 24.694 | 1.00 | 37.69 | C |
| ATOM | 1407 | O | SER | B | 71 | 7.499 | −4.977 | 23.843 | 1.00 | 33.26 | O |
| ATOM | 1408 | N | ILE | B | 72 | 6.281 | −4.161 | 25.530 | 1.00 | 39.92 | N |
| ATOM | 1409 | CA | ILE | B | 72 | 5.363 | −5.284 | 25.484 | 1.00 | 42.55 | C |
| ATOM | 1410 | CB | ILE | B | 72 | 5.526 | −6.245 | 26.700 | 1.00 | 43.71 | C |
| ATOM | 1411 | CG1 | ILE | B | 72 | 5.012 | −5.585 | 27.971 | 1.00 | 47.17 | C |
| ATOM | 1412 | CD1 | ILE | B | 72 | 4.395 | −6.576 | 28.971 | 1.00 | 48.94 | C |
| ATOM | 1413 | CG2 | ILE | B | 72 | 6.985 | −6.712 | 26.853 | 1.00 | 35.49 | C |
| ATOM | 1414 | C | ILE | B | 72 | 3.919 | −4.825 | 25.369 | 1.00 | 39.41 | C |
| ATOM | 1415 | O | ILE | B | 72 | 3.569 | −3.721 | 25.792 | 1.00 | 31.99 | O |
| ATOM | 1416 | N | PRO | B | 73 | 3.073 | −5.677 | 24.783 | 1.00 | 41.55 | N |
| ATOM | 1417 | CA | PRO | B | 73 | 3.464 | −7.010 | 24.299 | 1.00 | 46.92 | C |
| ATOM | 1418 | CB | PRO | B | 73 | 2.130 | −7.628 | 23.886 | 1.00 | 39.32 | C |
| ATOM | 1419 | CG | PRO | B | 73 | 1.323 | −6.446 | 23.478 | 1.00 | 43.11 | C |
| ATOM | 1420 | CD | PRO | B | 73 | 1.669 | −5.379 | 24.482 | 1.00 | 32.04 | C |
| ATOM | 1421 | C | PRO | B | 73 | 4.405 | −7.025 | 23.089 | 1.00 | 47.78 | C |
| ATOM | 1422 | O | PRO | B | 73 | 4.523 | −6.055 | 22.333 | 1.00 | 43.58 | O |
| ATOM | 1423 | N | THR | B | 74 | 5.087 | −8.150 | 22.927 | 1.00 | 47.82 | N |
| ATOM | 1424 | CA | THR | B | 74 | 5.666 | −8.476 | 21.654 | 1.00 | 48.72 | C |
| ATOM | 1425 | CB | THR | B | 74 | 7.144 | −8.121 | 21.564 | 1.00 | 54.30 | C |
| ATOM | 1426 | OG1 | THR | B | 74 | 7.354 | −6.788 | 22.040 | 1.00 | 48.35 | O |
| ATOM | 1427 | CG2 | THR | B | 74 | 7.618 | −8.243 | 20.106 | 1.00 | 53.29 | C |
| ATOM | 1428 | C | THR | B | 74 | 5.521 | −9.963 | 21.460 | 1.00 | 54.24 | C |
| ATOM | 1429 | O | THR | B | 74 | 5.196 | −10.689 | 22.397 | 1.00 | 59.23 | O |
| ATOM | 1430 | N | LEU | B | 75 | 5.734 | −10.397 | 20.224 | 1.00 | 45.57 | N |
| ATOM | 1431 | CA | LEU | B | 75 | 5.930 | −11.803 | 19.917 | 1.00 | 50.43 | C |
| ATOM | 1432 | C | LEU | B | 75 | 7.283 | −11.981 | 19.270 | 1.00 | 44.61 | C |
| ATOM | 1433 | O | LEU | B | 75 | 7.724 | −11.120 | 18.510 | 1.00 | 45.35 | O |
| ATOM | 1434 | CB | LEU | B | 75 | 4.888 | −12.267 | 18.928 | 1.00 | 46.56 | C |
| ATOM | 1435 | CG | LEU | B | 75 | 3.470 | −12.336 | 19.431 | 1.00 | 46.27 | C |
| ATOM | 1436 | CD1 | LEU | B | 75 | 2.586 | −12.744 | 18.240 | 1.00 | 36.57 | C |
| ATOM | 1437 | CD2 | LEU | B | 75 | 3.459 | −13.363 | 20.549 | 1.00 | 42.31 | C |
| ATOM | 1438 | N | LEU | B | 76 | 7.938 | −13.096 | 19.555 | 1.00 | 46.08 | N |
| ATOM | 1439 | CA | LEU | B | 76 | 9.137 | −13.451 | 18.811 | 1.00 | 45.22 | C |
| ATOM | 1440 | CB | LEU | B | 76 | 10.377 | −13.493 | 19.700 | 1.00 | 43.70 | C |
| ATOM | 1441 | CG | LEU | B | 76 | 10.947 | −12.105 | 19.986 | 1.00 | 50.29 | C |
| ATOM | 1442 | CD1 | LEU | B | 76 | 12.311 | −12.227 | 20.664 | 1.00 | 51.08 | C |
| ATOM | 1443 | CD2 | LEU | B | 76 | 11.026 | −11.285 | 18.700 | 1.00 | 38.02 | C |
| ATOM | 1444 | C | LEU | B | 76 | 8.935 | −14.785 | 18.156 | 1.00 | 45.16 | C |
| ATOM | 1445 | O | LEU | B | 76 | 8.422 | −15.723 | 18.760 | 1.00 | 51.28 | O |
| ATOM | 1446 | N | PHE | B | 77 | 9.349 | −14.873 | 16.908 | 1.00 | 51.28 | N |
| ATOM | 1447 | CA | PHE | B | 77 | 9.280 | −16.137 | 16.214 | 1.00 | 58.35 | C |
| ATOM | 1448 | CB | PHE | B | 77 | 8.692 | −15.964 | 14.813 | 1.00 | 63.75 | C |
| ATOM | 1449 | CG | PHE | B | 77 | 7.233 | −15.617 | 14.819 | 1.00 | 57.56 | C |
| ATOM | 1450 | CD2 | PHE | B | 77 | 6.792 | −14.433 | 15.396 | 1.00 | 58.07 | C |
| ATOM | 1451 | CE2 | PHE | B | 77 | 5.441 | −14.108 | 15.424 | 1.00 | 60.94 | C |
| ATOM | 1452 | CZ | PHE | B | 77 | 4.514 | −14.973 | 14.867 | 1.00 | 63.33 | C |
| ATOM | 1453 | CE1 | PHE | B | 77 | 4.945 | −16.158 | 14.293 | 1.00 | 72.32 | C |
| ATOM | 1454 | CD1 | PHE | B | 77 | 6.302 | −16.479 | 14.281 | 1.00 | 66.36 | C |
| ATOM | 1455 | C | PHE | B | 77 | 10.664 | −16.711 | 16.167 | 1.00 | 53.90 | C |
| ATOM | 1456 | O | PHE | B | 77 | 11.620 | −16.027 | 15.815 | 1.00 | 53.13 | O |
| ATOM | 1457 | N | PHE | B | 78 | 10.752 | −17.968 | 16.571 | 1.00 | 61.37 | N |
| ATOM | 1458 | CA | PHE | B | 78 | 11.994 | −18.715 | 16.550 | 1.00 | 64.37 | C |
| ATOM | 1459 | CB | PHE | B | 78 | 12.331 | −19.201 | 17.957 | 1.00 | 58.40 | C |
| ATOM | 1460 | CG | PHE | B | 78 | 12.809 | −18.108 | 18.870 | 1.00 | 64.52 | C |
| ATOM | 1461 | CD1 | PHE | B | 78 | 14.169 | −17.846 | 19.011 | 1.00 | 68.90 | C |
| ATOM | 1462 | CE1 | PHE | B | 78 | 14.628 | −16.824 | 19.846 | 1.00 | 43.26 | C |
| ATOM | 1463 | CZ | PHE | B | 78 | 13.725 | −16.053 | 20.550 | 1.00 | 45.14 | C |
| ATOM | 1464 | CE2 | PHE | B | 78 | 12.356 | −16.296 | 20.414 | 1.00 | 50.61 | C |
| ATOM | 1465 | CD2 | PHE | B | 78 | 11.906 | −17.321 | 19.574 | 1.00 | 52.92 | C |
| ATOM | 1466 | C | PHE | B | 78 | 11.843 | −19.897 | 15.599 | 1.00 | 74.30 | C |
| ATOM | 1467 | O | PHE | B | 78 | 10.903 | −20.690 | 15.729 | 1.00 | 72.40 | O |
| ATOM | 1468 | N | LYS | B | 79 | 12.759 | −19.981 | 14.631 | 1.00 | 79.99 | N |
| ATOM | 1469 | CA | LYS | B | 79 | 12.861 | −21.108 | 13.705 | 1.00 | 81.26 | C |
| ATOM | 1470 | CB | LYS | B | 79 | 12.570 | −20.658 | 12.264 | 1.00 | 77.17 | C |
| ATOM | 1471 | CG | LYS | B | 79 | 12.658 | −21.748 | 11.197 | 1.00 | 82.29 | C |
| ATOM | 1472 | CD | LYS | B | 79 | 11.640 | −22.858 | 11.430 | 1.00 | 85.56 | C |
| ATOM | 1473 | CE | LYS | B | 79 | 11.744 | −23.951 | 10.380 | 1.00 | 73.31 | C |
| ATOM | 1474 | NZ | LYS | B | 79 | 11.107 | −25.184 | 10.896 | 1.00 | 70.08 | N |
| ATOM | 1475 | C | LYS | B | 79 | 14.274 | −21.667 | 13.814 | 1.00 | 87.80 | C |
| ATOM | 1476 | O | LYS | B | 79 | 15.226 | −21.041 | 13.348 | 1.00 | 89.09 | O |
| ATOM | 1477 | N | ASN | B | 80 | 14.403 | −22.822 | 14.464 | 1.00 | 92.98 | N |
| ATOM | 1478 | CA | ASN | B | 80 | 15.688 | −23.519 | 14.617 | 1.00 | 92.12 | C |
| ATOM | 1479 | CB | ASN | B | 80 | 16.262 | −23.918 | 13.254 | 1.00 | 91.38 | C |
| ATOM | 1480 | CG | ASN | B | 80 | 15.234 | −24.597 | 12.367 | 1.00 | 89.52 | C |
| ATOM | 1481 | OD1 | ASN | B | 80 | 14.184 | −25.043 | 12.838 | 1.00 | 91.37 | O |
| ATOM | 1482 | ND2 | ASN | B | 80 | 15.529 | −24.673 | 11.072 | 1.00 | 92.24 | N |
| ATOM | 1483 | C | ASN | B | 80 | 16.750 | −22.781 | 15.426 | 1.00 | 89.02 | C |
| ATOM | 1484 | O | ASN | B | 80 | 17.902 | −22.701 | 15.014 | 1.00 | 84.72 | O |
| ATOM | 1485 | N | GLY | B | 81 | 16.357 | −22.253 | 16.579 | 1.00 | 82.82 | N |
| ATOM | 1486 | CA | GLY | B | 81 | 17.293 | −21.642 | 17.505 | 1.00 | 85.38 | C |
| ATOM | 1487 | C | GLY | B | 81 | 17.628 | −20.189 | 17.201 | 1.00 | 78.57 | C |
| ATOM | 1488 | O | GLY | B | 81 | 18.407 | −19.565 | 17.921 | 1.00 | 70.97 | O |
| ATOM | 1489 | N | ALYS | B | 82 | 17.026 | −19.650 | 16.144 | 1.00 | 96.32 | N |
| ATOM | 1490 | CA | ALYS | B | 82 | 17.306 | −18.286 | 15.724 | 1.00 | 96.56 | C |
| ATOM | 1491 | CB | ALYS | B | 82 | 18.232 | −18.294 | 14.505 | 1.00 | 100.32 | C |
| ATOM | 1492 | CG | ALYS | B | 82 | 19.627 | −18.860 | 14.794 | 1.00 | 101.24 | C |
| ATOM | 1493 | CD | ALYS | B | 82 | 20.232 | −19.551 | 13.572 | 1.00 | 107.58 | C |
| ATOM | 1494 | CE | ALYS | B | 82 | 19.888 | −21.037 | 13.538 | 1.00 | 107.81 | C |
| ATOM | 1495 | NZ | ALYS | B | 82 | 20.635 | −21.806 | 14.591 | 1.00 | 108.32 | N |
| ATOM | 1496 | C | ALYS | B | 82 | 16.026 | −17.510 | 15.434 | 1.00 | 89.39 | C |
| ATOM | 1497 | O | ALYS | B | 82 | 15.122 | −18.010 | 14.778 | 1.00 | 88.12 | O |
| ATOM | 1498 | N | BLYS | B | 82 | 17.046 | −19.647 | 16.138 | 0.00 | 96.38 | N |
| ATOM | 1499 | CA | BLYS | B | 82 | 17.317 | −18.269 | 15.756 | 0.00 | 96.47 | C |
| ATOM | 1500 | CB | BLYS | B | 82 | 18.274 | −18.216 | 14.562 | 0.00 | 100.17 | C |
| ATOM | 1501 | CG | BLYS | B | 82 | 19.566 | −18.985 | 14.772 | 0.00 | 101.64 | C |
| ATOM | 1502 | CD | BLYS | B | 82 | 20.476 | −18.274 | 15.762 | 0.00 | 102.96 | C |
| ATOM | 1503 | CE | BLYS | B | 82 | 21.117 | −17.046 | 15.130 | 0.00 | 105.77 | C |
| ATOM | 1504 | NZ | BLYS | B | 82 | 22.212 | −16.489 | 15.975 | 0.00 | 112.72 | N |
| ATOM | 1505 | C | BLYS | B | 82 | 16.030 | −17.512 | 15.450 | 0.00 | 89.32 | C |
| ATOM | 1506 | O | BLYS | B | 82 | 15.128 | −18.022 | 14.796 | 0.00 | 88.08 | O |
| ATOM | 1507 | N | VAL | B | 83 | 15.962 | −16.281 | 15.940 | 1.00 | 70.50 | N |
| ATOM | 1508 | CA | VAL | B | 83 | 14.815 | −15.406 | 15.716 | 1.00 | 68.94 | C |
| ATOM | 1509 | CB | VAL | B | 83 | 14.990 | −14.073 | 16.466 | 1.00 | 65.36 | C |
| ATOM | 1510 | CG1 | VAL | B | 83 | 14.127 | −12.994 | 15.850 | 1.00 | 61.63 | C |
| ATOM | 1511 | CG2 | VAL | B | 83 | 14.644 | −14.262 | 17.926 | 1.00 | 63.19 | C |
| ATOM | 1512 | C | VAL | B | 83 | 14.555 | −15.090 | 14.240 | 1.00 | 66.41 | C |
| ATOM | 1513 | O | VAL | B | 83 | 15.390 | −14.471 | 13.568 | 1.00 | 56.18 | O |
| ATOM | 1514 | N | VAL | B | 84 | 13.381 | −15.487 | 13.753 | 1.00 | 79.05 | N |
| ATOM | 1515 | CA | VAL | B | 84 | 13.001 | −15.233 | 12.364 | 1.00 | 86.32 | C |
| ATOM | 1516 | CB | VAL | B | 84 | 12.349 | −16.475 | 11.709 | 1.00 | 85.25 | C |
| ATOM | 1517 | CG1 | VAL | B | 84 | 13.157 | −17.703 | 12.043 | 1.00 | 89.52 | C |
| ATOM | 1518 | CG2 | VAL | B | 84 | 10.910 | −16.647 | 12.167 | 1.00 | 79.83 | C |
| ATOM | 1519 | C | VAL | B | 84 | 12.080 | −14.017 | 12.190 | 1.00 | 86.11 | C |
| ATOM | 1520 | O | VAL | B | 84 | 12.040 | −13.410 | 11.119 | 1.00 | 85.53 | O |
| ATOM | 1521 | N | ASP | B | 85 | 11.351 | −13.655 | 13.240 | 1.00 | 78.29 | N |
| ATOM | 1522 | CA | ASP | B | 85 | 10.361 | −12.587 | 13.124 | 1.00 | 78.64 | C |
| ATOM | 1523 | CB | ASP | B | 85 | 9.117 | −13.117 | 12.383 | 1.00 | 66.82 | C |
| ATOM | 1524 | CG | ASP | B | 85 | 8.340 | −12.017 | 11.679 | 1.00 | 74.43 | C |
| ATOM | 1525 | OD1 | ASP | B | 85 | 8.954 | −11.001 | 11.273 | 1.00 | 83.29 | O |
| ATOM | 1526 | OD2 | ASP | B | 85 | 7.108 | −12.172 | 11.518 | 1.00 | 82.00 | O |
| ATOM | 1527 | C | ASP | B | 85 | 9.986 | −11.986 | 14.492 | 1.00 | 70.23 | C |
| ATOM | 1528 | O | ASP | B | 85 | 9.995 | −12.691 | 15.499 | 1.00 | 65.42 | O |
| ATOM | 1529 | N | GLN | B | 86 | 9.642 | −10.696 | 14.504 | 1.00 | 57.50 | N |
| ATOM | 1530 | CA | GLN | B | 86 | 9.254 | −9.969 | 15.723 | 1.00 | 56.17 | C |
| ATOM | 1531 | CB | GLN | B | 86 | 10.466 | −9.149 | 16.249 | 1.00 | 45.97 | C |
| ATOM | 1532 | CG | GLN | B | 86 | 10.167 | −7.937 | 17.144 | 1.00 | 48.87 | C |
| ATOM | 1533 | CD | GLN | B | 86 | 11.376 | −7.540 | 18.002 | 1.00 | 68.16 | C |
| ATOM | 1534 | OE1 | GLN | B | 86 | 12.137 | −8.402 | 18.441 | 1.00 | 76.49 | O |
| ATOM | 1535 | NE2 | GLN | B | 86 | 11.556 | −6.239 | 18.240 | 1.00 | 64.10 | N |
| ATOM | 1536 | C | GLN | B | 86 | 7.992 | −9.092 | 15.467 | 1.00 | 51.41 | C |
| ATOM | 1537 | O | GLN | B | 86 | 7.943 | −8.326 | 14.497 | 1.00 | 42.63 | O |
| ATOM | 1538 | N | LEU | B | 87 | 6.959 | −9.226 | 16.306 | 1.00 | 51.41 | N |
| ATOM | 1539 | CA | LEU | B | 87 | 5.706 | −8.461 | 16.110 | 1.00 | 48.41 | C |
| ATOM | 1540 | CB | LEU | B | 87 | 4.523 | −9.363 | 15.714 | 1.00 | 41.88 | C |
| ATOM | 1541 | CG | LEU | B | 87 | 4.505 | −10.087 | 14.366 | 1.00 | 49.21 | C |
| ATOM | 1542 | CD1 | LEU | B | 87 | 5.682 | −11.018 | 14.257 | 1.00 | 55.08 | C |
| ATOM | 1543 | CD2 | LEU | B | 87 | 3.184 | −10.883 | 14.172 | 1.00 | 47.17 | C |
| ATOM | 1544 | C | LEU | B | 87 | 5.345 | −7.659 | 17.355 | 1.00 | 46.76 | C |
| ATOM | 1545 | O | LEU | B | 87 | 4.829 | −8.205 | 18.338 | 1.00 | 46.15 | O |
| ATOM | 1546 | N | VAL | B | 88 | 5.630 | −6.362 | 17.296 | 1.00 | 51.16 | N |
| ATOM | 1547 | CA | VAL | B | 88 | 5.438 | −5.443 | 18.416 | 1.00 | 49.53 | C |
| ATOM | 1548 | CB | VAL | B | 88 | 6.321 | −4.184 | 18.232 | 1.00 | 44.56 | C |
| ATOM | 1549 | CG1 | VAL | B | 88 | 6.031 | −3.144 | 19.314 | 1.00 | 44.38 | C |
| ATOM | 1550 | CG2 | VAL | B | 88 | 7.774 | −4.576 | 18.232 | 1.00 | 43.01 | C |
| ATOM | 1551 | C | VAL | B | 88 | 3.981 | −5.007 | 18.557 | 1.00 | 41.29 | C |
| ATOM | 1552 | O | VAL | B | 88 | 3.337 | −4.642 | 17.577 | 1.00 | 54.63 | O |
| ATOM | 1553 | N | GLY | B | 89 | 3.467 | −5.043 | 19.779 | 1.00 | 35.82 | N |
| ATOM | 1554 | CA | GLY | B | 89 | 2.135 | −4.538 | 20.068 | 1.00 | 36.29 | C |
| ATOM | 1555 | C | GLY | B | 89 | 1.015 | −5.494 | 19.681 | 1.00 | 39.63 | C |
| ATOM | 1556 | O | GLY | B | 89 | 1.171 | −6.319 | 18.767 | 1.00 | 36.00 | O |
| ATOM | 1557 | N | ALA | B | 90 | −0.120 | −5.392 | 20.380 | 1.00 | 35.32 | N |
| ATOM | 1558 | CA | ALA | B | 90 | −1.260 | −6.303 | 20.148 | 1.00 | 41.64 | C |
| ATOM | 1559 | CB | ALA | B | 90 | −2.455 | −5.918 | 21.030 | 1.00 | 34.39 | C |
| ATOM | 1560 | C | ALA | B | 90 | −1.678 | −6.358 | 18.678 | 1.00 | 34.93 | C |
| ATOM | 1561 | O | ALA | B | 90 | −1.876 | −5.318 | 18.054 | 1.00 | 26.67 | O |
| ATOM | 1562 | N | ARG | B | 91 | −1.785 | −7.570 | 18.137 | 1.00 | 49.75 | N |
| ATOM | 1563 | CA | ARG | B | 91 | −2.217 | −7.799 | 16.749 | 1.00 | 55.13 | C |
| ATOM | 1564 | CB | ARG | B | 91 | −1.115 | −8.505 | 15.943 | 1.00 | 55.02 | C |
| ATOM | 1565 | CG | ARG | B | 91 | 0.259 | −7.871 | 16.020 | 1.00 | 51.47 | C |
| ATOM | 1566 | CD | ARG | B | 91 | 0.237 | −6.436 | 15.522 | 1.00 | 57.35 | C |
| ATOM | 1567 | NE | ARG | B | 91 | 1.565 | −5.832 | 15.564 | 1.00 | 56.80 | N |
| ATOM | 1568 | CZ | ARG | B | 91 | 2.509 | −6.021 | 14.646 | 1.00 | 59.60 | C |
| ATOM | 1569 | NH1 | ARG | B | 91 | 2.261 | −6.792 | 13.597 | 1.00 | 61.33 | N |
| ATOM | 1570 | NH2 | ARG | B | 91 | 3.701 | −5.436 | 14.774 | 1.00 | 47.41 | N |
| ATOM | 1571 | C | ARG | B | 91 | −3.461 | −8.688 | 16.726 | 1.00 | 53.06 | C |
| ATOM | 1572 | O | ARG | B | 91 | −3.599 | −9.590 | 17.550 | 1.00 | 61.54 | O |
| ATOM | 1573 | N | PRO | B | 92 | −4.366 | −8.461 | 15.767 | 1.00 | 55.10 | N |
| ATOM | 1574 | CA | PRO | B | 92 | −5.576 | −9.306 | 15.674 | 1.00 | 57.39 | C |
| ATOM | 1575 | CB | PRO | B | 92 | −6.317 | −8.735 | 14.465 | 1.00 | 55.36 | C |
| ATOM | 1576 | CG | PRO | B | 92 | −5.243 | −7.993 | 13.669 | 1.00 | 52.14 | C |
| ATOM | 1577 | CD | PRO | B | 92 | −4.338 | −7.417 | 14.729 | 1.00 | 52.97 | C |
| ATOM | 1578 | C | PRO | B | 92 | −5.273 | −10.779 | 15.438 | 1.00 | 51.84 | C |
| ATOM | 1579 | O | PRO | B | 92 | −4.349 | −11.107 | 14.718 | 1.00 | 56.79 | O |
| ATOM | 1580 | N | LYS | B | 93 | −6.057 | −11.661 | 16.035 | 1.00 | 61.95 | N |
| ATOM | 1581 | CA | LYS | B | 93 | −5.827 | −13.094 | 15.890 | 1.00 | 66.27 | C |
| ATOM | 1582 | CB | LYS | B | 93 | −6.964 | −13.877 | 16.547 | 1.00 | 69.50 | C |
| ATOM | 1583 | CG | LYS | B | 93 | −6.745 | −15.383 | 16.631 | 1.00 | 69.49 | C |
| ATOM | 1584 | CD | LYS | B | 93 | −7.968 | −16.096 | 17.229 | 1.00 | 73.18 | C |
| ATOM | 1585 | CE | LYS | B | 93 | −7.735 | −17.608 | 17.337 | 1.00 | 79.39 | C |
| ATOM | 1586 | NZ | LYS | B | 93 | −8.948 | −18.393 | 17.739 | 1.00 | 71.25 | N |
| ATOM | 1587 | C | LYS | B | 93 | −5.647 | −13.546 | 14.435 | 1.00 | 67.82 | C |
| ATOM | 1588 | O | LYS | B | 93 | −4.878 | −14.459 | 14.160 | 1.00 | 72.13 | O |
| ATOM | 1589 | N | GLU | B | 94 | −6.348 | −12.916 | 13.501 | 1.00 | 60.92 | N |
| ATOM | 1590 | CA | GLU | B | 94 | −6.267 | −13.338 | 12.098 | 1.00 | 61.01 | C |
| ATOM | 1591 | CB | GLU | B | 94 | −7.475 | −12.841 | 11.281 | 1.00 | 63.11 | C |
| ATOM | 1592 | CG | GLU | B | 94 | −8.238 | −11.668 | 11.911 | 1.00 | 64.32 | C |
| ATOM | 1593 | CD | GLU | B | 94 | −9.408 | −12.111 | 12.758 | 1.00 | 64.58 | C |
| ATOM | 1594 | OE1 | GLU | B | 94 | −10.372 | −12.632 | 12.168 | 1.00 | 58.04 | O |
| ATOM | 1595 | OE2 | GLU | B | 94 | −9.358 | −11.951 | 14.003 | 1.00 | 70.15 | O |
| ATOM | 1596 | C | GLU | B | 94 | −4.951 | −12.941 | 11.416 | 1.00 | 56.16 | C |
| ATOM | 1597 | O | GLU | B | 94 | −4.429 | −13.680 | 10.567 | 1.00 | 50.41 | O |
| ATOM | 1598 | N | ALA | B | 95 | −4.416 | −11.779 | 11.781 | 1.00 | 52.48 | N |
| ATOM | 1599 | CA | ALA | B | 95 | −3.101 | −11.362 | 11.275 | 1.00 | 52.05 | C |
| ATOM | 1600 | CB | ALA | B | 95 | −2.791 | −9.928 | 11.684 | 1.00 | 45.22 | C |
| ATOM | 1601 | C | ALA | B | 95 | −1.959 | −12.308 | 11.676 | 1.00 | 53.20 | C |
| ATOM | 1602 | O | ALA | B | 95 | −1.036 | −12.540 | 10.889 | 1.00 | 55.60 | O |
| ATOM | 1603 | N | LEU | B | 96 | −2.005 | −12.850 | 12.891 | 1.00 | 49.28 | N |
| ATOM | 1604 | CA | LEU | B | 96 | −1.002 | −13.850 | 13.251 | 1.00 | 62.57 | C |
| ATOM | 1605 | CB | LEU | B | 96 | −0.451 | −13.711 | 14.690 | 1.00 | 63.49 | C |
| ATOM | 1606 | CG | LEU | B | 96 | −1.244 | −13.315 | 15.936 | 1.00 | 60.05 | C |
| ATOM | 1607 | CD1 | LEU | B | 96 | −1.449 | −11.799 | 16.044 | 1.00 | 55.99 | C |
| ATOM | 1608 | CD2 | LEU | B | 96 | −2.559 | −14.075 | 16.000 | 1.00 | 66.28 | C |
| ATOM | 1609 | C | LEU | B | 96 | −1.362 | −15.300 | 12.887 | 1.00 | 61.46 | C |
| ATOM | 1610 | O | LEU | B | 96 | −0.651 | −16.235 | 13.234 | 1.00 | 64.83 | O |
| ATOM | 1611 | N | LYS | B | 97 | −2.451 | −15.489 | 12.164 | 1.00 | 69.90 | N |
| ATOM | 1612 | CA | LYS | B | 97 | −2.617 | −16.744 | 11.436 | 1.00 | 73.60 | C |
| ATOM | 1613 | CB | LYS | B | 97 | −4.095 | −17.100 | 11.251 | 1.00 | 77.04 | C |
| ATOM | 1614 | CG | LYS | B | 97 | −4.855 | −17.368 | 12.544 | 1.00 | 73.67 | C |
| ATOM | 1615 | CD | LYS | B | 97 | −6.207 | −18.029 | 12.241 | 1.00 | 82.49 | C |
| ATOM | 1616 | CE | LYS | B | 97 | −7.336 | −17.457 | 13.108 | 1.00 | 87.82 | C |
| ATOM | 1617 | NZ | LYS | B | 97 | −7.424 | −15.958 | 13.027 | 1.00 | 74.66 | N |
| ATOM | 1618 | C | LYS | B | 97 | −1.872 | −16.682 | 10.076 | 1.00 | 67.25 | C |
| ATOM | 1619 | O | LYS | B | 97 | −1.305 | −17.680 | 9.635 | 1.00 | 71.22 | O |
| ATOM | 1620 | N | GLU | B | 98 | −1.853 | −15.510 | 9.432 | 1.00 | 57.34 | N |
| ATOM | 1621 | CA | GLU | B | 98 | −1.103 | −15.317 | 8.182 | 1.00 | 59.76 | C |
| ATOM | 1622 | CB | GLU | B | 98 | −1.276 | −13.900 | 7.627 | 1.00 | 53.73 | C |
| ATOM | 1623 | CG | GLU | B | 98 | −2.497 | −13.687 | 6.759 | 1.00 | 72.23 | C |
| ATOM | 1624 | CD | GLU | B | 98 | −2.493 | −14.478 | 5.440 | 1.00 | 71.43 | C |
| ATOM | 1625 | OE1 | GLU | B | 98 | −1.579 | −14.273 | 4.587 | 1.00 | 54.72 | O |
| ATOM | 1626 | OE2 | GLU | B | 98 | −3.439 | −15.282 | 5.255 | 1.00 | 67.60 | O |
| ATOM | 1627 | C | GLU | B | 98 | 0.380 | −15.588 | 8.365 | 1.00 | 70.75 | C |
| ATOM | 1628 | O | GLU | B | 98 | 0.967 | −16.361 | 7.612 | 1.00 | 72.12 | O |
| ATOM | 1629 | N | ARG | B | 99 | 0.998 | −14.925 | 9.339 | 1.00 | 62.62 | N |
| ATOM | 1630 | CA | ARG | B | 99 | 2.368 | −15.264 | 9.675 | 1.00 | 59.67 | C |
| ATOM | 1631 | CB | ARG | B | 99 | 2.970 | −14.245 | 10.631 | 1.00 | 58.28 | C |
| ATOM | 1632 | CG | ARG | B | 99 | 3.313 | −12.925 | 9.977 | 1.00 | 59.63 | C |
| ATOM | 1633 | CD | ARG | B | 99 | 2.693 | −11.770 | 10.732 | 1.00 | 60.38 | C |
| ATOM | 1634 | NE | ARG | B | 99 | 2.944 | −10.481 | 10.095 | 1.00 | 60.28 | N |
| ATOM | 1635 | CZ | ARG | B | 99 | 4.158 | −10.011 | 9.826 | 1.00 | 65.01 | C |
| ATOM | 1636 | NH1 | ARG | B | 99 | 5.232 | −10.732 | 10.129 | 1.00 | 60.06 | N |
| ATOM | 1637 | NH2 | ARG | B | 99 | 4.297 | −8.822 | 9.250 | 1.00 | 77.41 | N |
| ATOM | 1638 | C | ARG | B | 99 | 2.270 | −16.624 | 10.322 | 1.00 | 63.68 | C |
| ATOM | 1639 | O | ARG | B | 99 | 1.264 | −16.924 | 10.947 | 1.00 | 61.27 | O |
| ATOM | 1640 | N | ILE | B | 100 | 3.312 | −17.430 | 10.163 | 1.00 | 75.54 | N |
| ATOM | 1641 | CA | ILE | B | 100 | 3.314 | −18.854 | 10.526 | 1.00 | 84.64 | C |
| ATOM | 1642 | CB | ILE | B | 100 | 2.458 | −19.234 | 11.786 | 1.00 | 86.09 | C |
| ATOM | 1643 | CG1 | ILE | B | 100 | 0.982 | −19.437 | 11.437 | 1.00 | 82.58 | C |
| ATOM | 1644 | CD1 | ILE | B | 100 | 0.107 | −19.504 | 12.665 | 1.00 | 82.56 | C |
| ATOM | 1645 | CG2 | ILE | B | 100 | 2.668 | −18.254 | 12.948 | 1.00 | 77.23 | C |
| ATOM | 1646 | C | ILE | B | 100 | 2.930 | −19.736 | 9.340 | 1.00 | 89.48 | C |
| ATOM | 1647 | O | ILE | B | 100 | 3.314 | −20.899 | 9.286 | 1.00 | 93.34 | O |
| ATOM | 1648 | N | LYS | B | 101 | 2.175 | −19.190 | 8.392 | 1.00 | 76.24 | N |
| ATOM | 1649 | CA | LYS | B | 101 | 2.011 | −19.864 | 7.108 | 1.00 | 71.99 | C |
| ATOM | 1650 | CB | LYS | B | 101 | 0.917 | −19.204 | 6.277 | 1.00 | 75.08 | C |
| ATOM | 1651 | CG | LYS | B | 101 | −0.443 | −19.751 | 6.599 | 1.00 | 76.84 | C |
| ATOM | 1652 | CD | LYS | B | 101 | −0.503 | −20.028 | 8.091 | 1.00 | 81.30 | C |
| ATOM | 1653 | CE | LYS | B | 101 | −1.750 | −20.793 | 8.483 | 1.00 | 87.45 | C |
| ATOM | 1654 | NZ | LYS | B | 101 | −1.690 | −21.131 | 9.929 | 1.00 | 80.07 | N |
| ATOM | 1655 | C | LYS | B | 101 | 3.335 | −19.807 | 6.373 | 1.00 | 67.99 | C |
| ATOM | 1656 | O | LYS | B | 101 | 3.847 | −20.826 | 5.901 | 1.00 | 65.13 | O |
| ATOM | 1657 | N | LYS | B | 102 | 3.884 | −18.600 | 6.289 | 1.00 | 70.37 | N |
| ATOM | 1658 | CA | LYS | B | 102 | 5.244 | −18.413 | 5.819 | 1.00 | 75.89 | C |
| ATOM | 1659 | CB | LYS | B | 102 | 5.739 | −17.007 | 6.167 | 1.00 | 73.74 | C |
| ATOM | 1660 | CG | LYS | B | 102 | 7.236 | −16.782 | 5.935 | 1.00 | 81.11 | C |
| ATOM | 1661 | CD | LYS | B | 102 | 7.551 | −16.428 | 4.482 | 1.00 | 79.05 | C |
| ATOM | 1662 | C | LYS | B | 102 | 6.143 | −19.451 | 6.479 | 1.00 | 80.78 | C |
| ATOM | 1663 | O | LYS | B | 102 | 7.019 | −20.026 | 5.828 | 1.00 | 80.87 | O |
| ATOM | 1664 | N | TYR | B | 103 | 5.890 | −19.705 | 7.765 | 1.00 | 84.47 | N |
| ATOM | 1665 | CA | TYR | B | 103 | 6.774 | −20.519 | 8.607 | 1.00 | 87.95 | C |
| ATOM | 1666 | CB | TYR | B | 103 | 7.049 | −19.803 | 9.944 | 1.00 | 84.27 | C |
| ATOM | 1667 | CG | TYR | B | 103 | 7.702 | −18.456 | 9.750 | 1.00 | 84.36 | C |
| ATOM | 1668 | CD2 | TYR | B | 103 | 9.056 | −18.361 | 9.471 | 1.00 | 86.66 | C |
| ATOM | 1669 | CE2 | TYR | B | 103 | 9.661 | −17.135 | 9.261 | 1.00 | 93.19 | C |
| ATOM | 1670 | CZ | TYR | B | 103 | 8.907 | −15.982 | 9.326 | 1.00 | 88.00 | C |
| ATOM | 1671 | OH | TYR | B | 103 | 9.517 | −14.765 | 9.123 | 1.00 | 87.49 | O |
| ATOM | 1672 | CE1 | TYR | B | 103 | 7.556 | −16.048 | 9.596 | 1.00 | 78.13 | C |
| ATOM | 1673 | CD1 | TYR | B | 103 | 6.960 | −17.284 | 9.801 | 1.00 | 83.04 | C |
| ATOM | 1674 | C | TYR | B | 103 | 6.282 | −21.946 | 8.857 | 1.00 | 87.16 | C |
| ATOM | 1675 | O | TYR | B | 103 | 6.984 | −22.756 | 9.458 | 1.00 | 88.52 | O |
| ATOM | 1676 | N | LEU | B | 104 | 5.078 | −22.262 | 8.403 | 1.00 | 87.34 | N |
| ATOM | 1677 | CA | LEU | B | 104 | 4.589 | −23.622 | 8.560 | 1.00 | 93.65 | C |
| ATOM | 1678 | CB | LEU | B | 104 | 3.057 | −23.683 | 8.499 | 1.00 | 93.72 | C |
| ATOM | 1679 | CG | LEU | B | 104 | 2.354 | −24.161 | 9.780 | 1.00 | 93.51 | C |
| ATOM | 1680 | CD1 | LEU | B | 104 | 2.397 | −23.100 | 10.870 | 1.00 | 90.69 | C |
| ATOM | 1681 | CD2 | LEU | B | 104 | 0.912 | −24.574 | 9.513 | 1.00 | 88.83 | C |
| ATOM | 1682 | C | LEU | B | 104 | 5.222 | −24.504 | 7.487 | 1.00 | 100.89 | C |
| ATOM | 1683 | O | LEU | B | 104 | 5.289 | −25.729 | 7.616 | 1.00 | 98.15 | O |
| ATOM | 1684 | OXT | LEU | B | 104 | 5.696 | −24.000 | 6.464 | 1.00 | 98.63 | O |
| TER | | | | | | | | | | | |
| ATOM | 1685 | N | SER | C | 1 | 21.966 | 8.837 | −1.633 | 1.00 | 64.26 | N |
| ATOM | 1686 | CA | SER | C | 1 | 21.381 | 7.576 | −1.190 | 1.00 | 81.80 | C |
| ATOM | 1687 | CB | SER | C | 1 | 21.451 | 6.533 | −2.312 | 1.00 | 80.09 | C |
| ATOM | 1688 | OG | SER | C | 1 | 20.604 | 5.427 | −2.053 | 1.00 | 85.01 | O |
| ATOM | 1689 | C | SER | C | 1 | 22.071 | 7.057 | 0.085 | 1.00 | 90.63 | C |
| ATOM | 1690 | O | SER | C | 1 | 21.822 | 7.561 | 1.185 | 1.00 | 85.12 | O |
| ATOM | 1691 | N | VAL | C | 2 | 22.937 | 6.057 | −0.063 | 1.00 | 74.46 | N |
| ATOM | 1692 | CA | VAL | C | 2 | 23.580 | 5.430 | 1.090 | 1.00 | 65.71 | C |
| ATOM | 1693 | CB | VAL | C | 2 | 23.432 | 3.898 | 1.073 | 1.00 | 67.40 | C |
| ATOM | 1694 | CG1 | VAL | C | 2 | 24.017 | 3.311 | 2.338 | 1.00 | 67.52 | C |
| ATOM | 1695 | CG2 | VAL | C | 2 | 21.980 | 3.511 | 0.956 | 1.00 | 75.97 | C |
| ATOM | 1696 | C | VAL | C | 2 | 25.059 | 5.802 | 1.217 | 1.00 | 70.44 | C |
| ATOM | 1697 | O | VAL | C | 2 | 25.943 | 5.130 | 0.672 | 1.00 | 68.67 | O |
| ATOM | 1698 | N | ILE | C | 3 | 25.314 | 6.883 | 1.942 | 1.00 | 73.24 | N |
| ATOM | 1699 | CA | ILE | C | 3 | 26.670 | 7.308 | 2.242 | 1.00 | 73.46 | C |
| ATOM | 1700 | CB | ILE | C | 3 | 26.652 | 8.357 | 3.351 | 1.00 | 82.71 | C |
| ATOM | 1701 | CG1 | ILE | C | 3 | 25.635 | 9.455 | 3.015 | 1.00 | 79.03 | C |
| ATOM | 1702 | CD1 | ILE | C | 3 | 25.171 | 10.266 | 4.227 | 1.00 | 79.41 | C |
| ATOM | 1703 | CG2 | ILE | C | 3 | 28.061 | 8.899 | 3.599 | 1.00 | 75.14 | C |
| ATOM | 1704 | C | ILE | C | 3 | 27.548 | 6.131 | 2.694 | 1.00 | 71.31 | C |
| ATOM | 1705 | O | ILE | C | 3 | 27.119 | 5.266 | 3.454 | 1.00 | 68.26 | O |
| ATOM | 1706 | N | GLU | C | 4 | 28.779 | 6.080 | 2.211 | 1.00 | 84.63 | N |
| ATOM | 1707 | CA | GLU | C | 4 | 29.697 | 5.069 | 2.707 | 1.00 | 85.17 | C |
| ATOM | 1708 | CB | GLU | C | 4 | 30.568 | 4.517 | 1.590 | 1.00 | 77.58 | C |
| ATOM | 1709 | CG | GLU | C | 4 | 31.487 | 3.393 | 2.057 | 1.00 | 92.86 | C |
| ATOM | 1710 | CD | GLU | C | 4 | 30.829 | 2.020 | 2.021 | 1.00 | 99.67 | C |
| ATOM | 1711 | OE1 | GLU | C | 4 | 30.276 | 1.657 | 0.958 | 1.00 | 110.67 | O |
| ATOM | 1712 | OE2 | GLU | C | 4 | 30.877 | 1.305 | 3.053 | 1.00 | 85.60 | O |
| ATOM | 1713 | C | GLU | C | 4 | 30.554 | 5.717 | 3.787 | 1.00 | 79.62 | C |
| ATOM | 1714 | O | GLU | C | 4 | 31.275 | 6.681 | 3.517 | 1.00 | 71.18 | O |
| ATOM | 1715 | N | ILE | C | 5 | 30.458 | 5.206 | 5.012 | 1.00 | 60.08 | N |
| ATOM | 1716 | CA | ILE | C | 5 | 31.066 | 5.895 | 6.147 | 1.00 | 65.03 | C |
| ATOM | 1717 | CB | ILE | C | 5 | 30.122 | 5.993 | 7.369 | 1.00 | 58.57 | C |
| ATOM | 1718 | CG1 | ILE | C | 5 | 28.756 | 6.525 | 6.964 | 1.00 | 63.17 | C |
| ATOM | 1719 | CD1 | ILE | C | 5 | 27.832 | 6.822 | 8.140 | 1.00 | 57.48 | C |
| ATOM | 1720 | CG2 | ILE | C | 5 | 30.710 | 6.922 | 8.410 | 1.00 | 66.63 | C |
| ATOM | 1721 | C | ILE | C | 5 | 32.391 | 5.273 | 6.588 | 1.00 | 66.19 | C |
| ATOM | 1722 | O | ILE | C | 5 | 32.621 | 4.059 | 6.451 | 1.00 | 63.25 | O |
| ATOM | 1723 | N | ASN | C | 6 | 33.251 | 6.129 | 7.127 | 1.00 | 58.23 | N |
| ATOM | 1724 | CA | ASN | C | 6 | 34.556 | 5.723 | 7.620 | 1.00 | 60.85 | C |
| ATOM | 1725 | CB | ASN | C | 6 | 35.507 | 5.411 | 6.455 | 1.00 | 65.81 | C |
| ATOM | 1726 | CG | ASN | C | 6 | 36.063 | 6.665 | 5.776 | 1.00 | 67.62 | C |
| ATOM | 1727 | OD1 | ASN | C | 6 | 35.831 | 7.795 | 6.218 | 1.00 | 68.37 | O |
| ATOM | 1728 | ND2 | ASN | C | 6 | 36.826 | 6.458 | 4.697 | 1.00 | 59.28 | N |
| ATOM | 1729 | C | ASN | C | 6 | 35.180 | 6.750 | 8.569 | 1.00 | 64.42 | C |
| ATOM | 1730 | O | ASN | C | 6 | 34.545 | 7.757 | 8.923 | 1.00 | 57.88 | O |
| ATOM | 1731 | N | ASP | C | 7 | 36.425 | 6.481 | 8.967 | 1.00 | 85.22 | N |
| ATOM | 1732 | CA | ASP | C | 7 | 37.148 | 7.292 | 9.945 | 1.00 | 81.70 | C |
| ATOM | 1733 | CB | ASP | C | 7 | 38.371 | 6.530 | 10.465 | 1.00 | 84.40 | C |
| ATOM | 1734 | CG | ASP | C | 7 | 37.996 | 5.325 | 11.328 | 1.00 | 93.67 | C |
| ATOM | 1735 | OD1 | ASP | C | 7 | 37.969 | 5.452 | 12.583 | 1.00 | 87.03 | O |
| ATOM | 1736 | OD2 | ASP | C | 7 | 37.733 | 4.247 | 10.744 | 1.00 | 87.15 | O |
| ATOM | 1737 | C | ASP | C | 7 | 37.599 | 8.631 | 9.382 | 1.00 | 88.94 | C |
| ATOM | 1738 | O | ASP | C | 7 | 38.773 | 8.816 | 9.074 | 1.00 | 100.71 | O |
| ATOM | 1739 | N | GLU | C | 8 | 36.667 | 9.566 | 9.268 | 1.00 | 88.93 | N |
| ATOM | 1740 | CA | GLU | C | 8 | 36.950 | 10.896 | 8.739 | 1.00 | 95.99 | C |
| ATOM | 1741 | CB | GLU | C | 8 | 37.713 | 10.828 | 7.414 | 1.00 | 91.81 | C |
| ATOM | 1742 | CG | GLU | C | 8 | 37.948 | 12.208 | 6.798 | 1.00 | 96.51 | C |
| ATOM | 1743 | CD | GLU | C | 8 | 38.331 | 12.160 | 5.330 | 1.00 | 113.79 | C |
| ATOM | 1744 | OE1 | GLU | C | 8 | 37.847 | 11.253 | 4.614 | 1.00 | 111.15 | O |
| ATOM | 1745 | OE2 | GLU | C | 8 | 39.115 | 13.037 | 4.897 | 1.00 | 116.45 | O |
| ATOM | 1746 | C | GLU | C | 8 | 35.628 | 11.598 | 8.506 | 1.00 | 86.39 | C |
| ATOM | 1747 | O | GLU | C | 8 | 35.493 | 12.804 | 8.713 | 1.00 | 85.44 | O |
| ATOM | 1748 | N | ASN | C | 9 | 34.647 | 10.829 | 8.061 | 1.00 | 64.70 | N |
| ATOM | 1749 | CA | ASN | C | 9 | 33.329 | 11.385 | 7.842 | 1.00 | 70.05 | C |
| ATOM | 1750 | CB | ASN | C | 9 | 32.788 | 11.019 | 6.447 | 1.00 | 63.47 | C |
| ATOM | 1751 | CG | ASN | C | 9 | 32.600 | 9.517 | 6.248 | 1.00 | 69.21 | C |
| ATOM | 1752 | OD1 | ASN | C | 9 | 33.166 | 8.709 | 6.980 | 1.00 | 80.45 | O |
| ATOM | 1753 | ND2 | ASN | C | 9 | 31.803 | 9.143 | 5.251 | 1.00 | 61.36 | N |
| ATOM | 1754 | C | ASN | C | 9 | 32.373 | 10.977 | 8.955 | 1.00 | 78.58 | C |
| ATOM | 1755 | O | ASN | C | 9 | 31.351 | 11.623 | 9.177 | 1.00 | 81.76 | O |
| ATOM | 1756 | N | PHE | C | 10 | 32.719 | 9.916 | 9.676 | 1.00 | 79.97 | N |
| ATOM | 1757 | CA | PHE | C | 10 | 31.831 | 9.413 | 10.721 | 1.00 | 82.16 | C |
| ATOM | 1758 | CB | PHE | C | 10 | 32.482 | 8.266 | 11.501 | 1.00 | 76.49 | C |
| ATOM | 1759 | CG | PHE | C | 10 | 31.534 | 7.556 | 12.432 | 1.00 | 59.53 | C |
| ATOM | 1760 | CD2 | PHE | C | 10 | 31.114 | 6.266 | 12.156 | 1.00 | 64.35 | C |
| ATOM | 1761 | CE2 | PHE | C | 10 | 30.232 | 5.608 | 13.000 | 1.00 | 57.56 | C |
| ATOM | 1762 | CZ | PHE | C | 10 | 29.768 | 6.239 | 14.138 | 1.00 | 59.92 | C |
| ATOM | 1763 | CE1 | PHE | C | 10 | 30.184 | 7.529 | 14.433 | 1.00 | 62.62 | C |
| ATOM | 1764 | CD1 | PHE | C | 10 | 31.061 | 8.180 | 13.576 | 1.00 | 67.80 | C |
| ATOM | 1765 | C | PHE | C | 10 | 31.400 | 10.517 | 11.691 | 1.00 | 83.27 | C |
| ATOM | 1766 | O | PHE | C | 10 | 30.318 | 10.456 | 12.268 | 1.00 | 75.76 | O |
| ATOM | 1767 | N | ASP | C | 11 | 32.243 | 11.524 | 11.886 | 1.00 | 91.76 | N |
| ATOM | 1768 | CA | ASP | C | 11 | 31.886 | 12.570 | 12.833 | 1.00 | 98.24 | C |
| ATOM | 1769 | CB | ASP | C | 11 | 33.019 | 13.579 | 13.003 | 1.00 | 101.70 | C |
| ATOM | 1770 | CG | ASP | C | 11 | 32.930 | 14.322 | 14.327 | 1.00 | 107.93 | C |
| ATOM | 1771 | OD1 | ASP | C | 11 | 32.228 | 13.814 | 15.236 | 1.00 | 98.14 | O |
| ATOM | 1772 | OD2 | ASP | C | 11 | 33.558 | 15.400 | 14.460 | 1.00 | 106.54 | O |
| ATOM | 1773 | C | ASP | C | 11 | 30.579 | 13.263 | 12.441 | 1.00 | 96.84 | C |
| ATOM | 1774 | O | ASP | C | 11 | 29.764 | 13.612 | 13.305 | 1.00 | 89.24 | O |
| ATOM | 1775 | N | GLU | C | 12 | 30.395 | 13.461 | 11.137 | 1.00 | 94.36 | N |
| ATOM | 1776 | CA | GLU | C | 12 | 29.145 | 13.979 | 10.585 | 1.00 | 95.64 | C |
| ATOM | 1777 | CB | GLU | C | 12 | 29.106 | 13.718 | 9.084 | 1.00 | 85.23 | C |
| ATOM | 1778 | CG | GLU | C | 12 | 27.724 | 13.374 | 8.563 | 1.00 | 94.75 | C |
| ATOM | 1779 | CD | GLU | C | 12 | 27.229 | 12.014 | 9.029 | 1.00 | 94.48 | C |
| ATOM | 1780 | OE1 | GLU | C | 12 | 27.980 | 11.024 | 8.882 | 1.00 | 87.80 | O |
| ATOM | 1781 | OE2 | GLU | C | 12 | 26.089 | 11.939 | 9.544 | 1.00 | 96.99 | O |
| ATOM | 1782 | C | GLU | C | 12 | 27.897 | 13.362 | 11.239 | 1.00 | 97.26 | C |
| ATOM | 1783 | O | GLU | C | 12 | 26.827 | 13.974 | 11.250 | 1.00 | 94.53 | O |
| ATOM | 1784 | N | VAL | C | 13 | 28.047 | 12.143 | 11.762 | 1.00 | 107.14 | N |
| ATOM | 1785 | CA | VAL | C | 13 | 26.959 | 11.400 | 12.392 | 1.00 | 97.21 | C |
| ATOM | 1786 | CB | VAL | C | 13 | 27.451 | 10.068 | 12.988 | 1.00 | 87.18 | C |
| ATOM | 1787 | CG1 | VAL | C | 13 | 26.384 | 9.462 | 13.857 | 1.00 | 88.32 | C |
| ATOM | 1788 | CG2 | VAL | C | 13 | 27.823 | 9.101 | 11.892 | 1.00 | 87.20 | C |
| ATOM | 1789 | C | VAL | C | 13 | 26.270 | 12.200 | 13.486 | 1.00 | 100.00 | C |
| ATOM | 1790 | O | VAL | C | 13 | 25.054 | 12.382 | 13.457 | 1.00 | 107.71 | O |
| ATOM | 1791 | N | ILE | C | 14 | 27.044 | 12.663 | 14.460 | 1.00 | 132.86 | N |
| ATOM | 1792 | CA | ILE | C | 14 | 26.496 | 13.520 | 15.499 | 1.00 | 138.62 | C |
| ATOM | 1793 | CB | ILE | C | 14 | 27.522 | 13.822 | 16.620 | 1.00 | 144.97 | C |
| ATOM | 1794 | CG1 | ILE | C | 14 | 28.830 | 14.337 | 16.016 | 1.00 | 142.59 | C |
| ATOM | 1795 | CD1 | ILE | C | 14 | 29.745 | 15.006 | 17.011 | 1.00 | 140.56 | C |
| ATOM | 1796 | CG2 | ILE | C | 14 | 27.766 | 12.584 | 17.492 | 1.00 | 136.12 | C |
| ATOM | 1797 | C | ILE | C | 14 | 26.048 | 14.826 | 14.857 | 1.00 | 139.06 | C |
| ATOM | 1798 | O | ILE | C | 14 | 24.910 | 15.263 | 15.039 | 1.00 | 132.27 | O |
| ATOM | 1799 | N | LYS | C | 15 | 26.943 | 15.423 | 14.074 | 1.00 | 97.81 | N |
| ATOM | 1800 | CA | LYS | C | 15 | 26.711 | 16.745 | 13.494 | 1.00 | 103.23 | C |
| ATOM | 1801 | CB | LYS | C | 15 | 28.042 | 17.378 | 13.092 | 1.00 | 97.70 | C |
| ATOM | 1802 | CG | LYS | C | 15 | 28.726 | 18.091 | 14.228 | 1.00 | 88.92 | C |
| ATOM | 1803 | CD | LYS | C | 15 | 27.880 | 19.264 | 14.702 | 1.00 | 93.45 | C |
| ATOM | 1804 | CE | LYS | C | 15 | 27.496 | 20.173 | 13.544 | 1.00 | 90.28 | C |
| ATOM | 1805 | NZ | LYS | C | 15 | 26.459 | 21.167 | 13.936 | 1.00 | 77.54 | N |
| ATOM | 1806 | C | LYS | C | 15 | 25.725 | 16.810 | 12.318 | 1.00 | 100.88 | C |
| ATOM | 1807 | O | LYS | C | 15 | 26.009 | 17.447 | 11.300 | 1.00 | 98.28 | O |
| ATOM | 1808 | N | LYS | C | 16 | 24.570 | 16.167 | 12.474 | 1.00 | 122.08 | N |
| ATOM | 1809 | CA | LYS | C | 16 | 23.475 | 16.292 | 11.514 | 1.00 | 124.81 | C |
| ATOM | 1810 | CB | LYS | C | 16 | 23.221 | 14.967 | 10.788 | 1.00 | 120.60 | C |
| ATOM | 1811 | CG | LYS | C | 16 | 24.247 | 14.684 | 9.693 | 1.00 | 118.99 | C |
| ATOM | 1812 | CD | LYS | C | 16 | 24.728 | 16.007 | 9.078 | 1.00 | 123.36 | C |
| ATOM | 1813 | CE | LYS | C | 16 | 25.646 | 15.823 | 7.871 | 1.00 | 114.55 | C |
| ATOM | 1814 | NZ | LYS | C | 16 | 26.325 | 17.101 | 7.487 | 1.00 | 103.42 | N |
| ATOM | 1815 | C | LYS | C | 16 | 22.202 | 16.837 | 12.163 | 1.00 | 124.26 | C |
| ATOM | 1816 | O | LYS | C | 16 | 22.176 | 17.988 | 12.608 | 1.00 | 122.97 | O |
| ATOM | 1817 | N | ASP | C | 17 | 21.158 | 16.014 | 12.224 | 1.00 | 125.26 | N |
| ATOM | 1818 | CA | ASP | C | 17 | 19.882 | 16.444 | 12.796 | 1.00 | 126.53 | C |
| ATOM | 1819 | CB | ASP | C | 17 | 19.398 | 17.726 | 12.120 | 1.00 | 126.59 | C |
| ATOM | 1820 | CG | ASP | C | 17 | 19.161 | 17.542 | 10.636 | 1.00 | 127.49 | C |
| ATOM | 1821 | OD1 | ASP | C | 17 | 19.875 | 16.727 | 10.016 | 1.00 | 127.15 | O |
| ATOM | 1822 | OD2 | ASP | C | 17 | 18.259 | 18.207 | 10.091 | 1.00 | 129.53 | O |
| ATOM | 1823 | C | ASP | C | 17 | 18.810 | 15.373 | 12.635 | 1.00 | 126.23 | C |
| ATOM | 1824 | O | ASP | C | 17 | 18.091 | 15.041 | 13.580 | 1.00 | 118.87 | O |
| ATOM | 1825 | N | LYS | C | 18 | 18.705 | 14.844 | 11.422 | 1.00 | 127.53 | N |
| ATOM | 1826 | CA | LYS | C | 18 | 17.726 | 13.818 | 11.115 | 1.00 | 118.25 | C |
| ATOM | 1827 | CB | LYS | C | 18 | 17.571 | 13.694 | 9.604 | 1.00 | 116.09 | C |
| ATOM | 1828 | CG | LYS | C | 18 | 16.161 | 13.927 | 9.103 | 1.00 | 111.92 | C |
| ATOM | 1829 | CD | LYS | C | 18 | 16.214 | 14.379 | 7.670 | 1.00 | 109.70 | C |
| ATOM | 1830 | CE | LYS | C | 18 | 17.228 | 13.543 | 6.909 | 1.00 | 105.74 | C |
| ATOM | 1831 | NZ | LYS | C | 18 | 17.451 | 14.052 | 5.526 | 1.00 | 110.32 | N |
| ATOM | 1832 | C | LYS | C | 18 | 18.152 | 12.479 | 11.698 | 1.00 | 115.56 | C |
| ATOM | 1833 | O | LYS | C | 18 | 19.312 | 12.284 | 12.069 | 1.00 | 112.51 | O |
| ATOM | 1834 | N | VAL | C | 19 | 17.208 | 11.554 | 11.778 | 1.00 | 86.34 | N |
| ATOM | 1835 | CA | VAL | C | 19 | 17.535 | 10.209 | 12.211 | 1.00 | 81.95 | C |
| ATOM | 1836 | CB | VAL | C | 19 | 16.287 | 9.337 | 12.355 | 1.00 | 78.41 | C |
| ATOM | 1837 | CG1 | VAL | C | 19 | 16.644 | 8.005 | 13.008 | 1.00 | 67.56 | C |
| ATOM | 1838 | CG2 | VAL | C | 19 | 15.240 | 10.067 | 13.164 | 1.00 | 86.51 | C |
| ATOM | 1839 | C | VAL | C | 19 | 18.482 | 9.548 | 11.214 | 1.00 | 74.85 | C |
| ATOM | 1840 | O | VAL | C | 19 | 18.073 | 9.121 | 10.132 | 1.00 | 69.37 | O |
| ATOM | 1841 | N | VAL | C | 20 | 19.756 | 9.472 | 11.584 | 1.00 | 77.40 | N |
| ATOM | 1842 | CA | VAL | C | 20 | 20.704 | 8.687 | 10.811 | 1.00 | 64.56 | C |
| ATOM | 1843 | CB | VAL | C | 20 | 22.115 | 9.250 | 10.884 | 1.00 | 53.98 | C |
| ATOM | 1844 | CG1 | VAL | C | 20 | 22.361 | 9.920 | 12.233 | 1.00 | 66.30 | C |
| ATOM | 1845 | CG2 | VAL | C | 20 | 23.103 | 8.149 | 10.599 | 1.00 | 53.87 | C |
| ATOM | 1846 | C | VAL | C | 20 | 20.717 | 7.233 | 11.255 | 1.00 | 59.30 | C |
| ATOM | 1847 | O | VAL | C | 20 | 20.784 | 6.934 | 12.452 | 1.00 | 62.03 | O |
| ATOM | 1848 | N | VAL | C | 21 | 20.622 | 6.336 | 10.278 | 1.00 | 53.04 | N |
| ATOM | 1849 | CA | VAL | C | 21 | 20.697 | 4.902 | 10.519 | 1.00 | 48.71 | C |
| ATOM | 1850 | CB | VAL | C | 21 | 19.419 | 4.158 | 10.045 | 1.00 | 44.74 | C |
| ATOM | 1851 | CG1 | VAL | C | 21 | 19.078 | 4.518 | 8.620 | 1.00 | 53.87 | C |
| ATOM | 1852 | CG2 | VAL | C | 21 | 19.592 | 2.658 | 10.168 | 1.00 | 48.21 | C |
| ATOM | 1853 | C | VAL | C | 21 | 21.955 | 4.341 | 9.838 | 1.00 | 55.50 | C |
| ATOM | 1854 | O | VAL | C | 21 | 22.075 | 4.333 | 8.606 | 1.00 | 54.24 | O |
| ATOM | 1855 | N | VAL | C | 22 | 22.902 | 3.897 | 10.661 | 1.00 | 59.83 | N |
| ATOM | 1856 | CA | VAL | C | 22 | 24.165 | 3.357 | 10.196 | 1.00 | 48.17 | C |
| ATOM | 1857 | CB | VAL | C | 22 | 25.306 | 3.844 | 11.064 | 1.00 | 48.09 | C |
| ATOM | 1858 | CG1 | VAL | C | 22 | 26.587 | 3.876 | 10.246 | 1.00 | 51.91 | C |
| ATOM | 1859 | CG2 | VAL | C | 22 | 24.985 | 5.227 | 11.608 | 1.00 | 55.30 | C |
| ATOM | 1860 | C | VAL | C | 22 | 24.148 | 1.833 | 10.232 | 1.00 | 49.83 | C |
| ATOM | 1861 | O | VAL | C | 22 | 23.703 | 1.218 | 11.202 | 1.00 | 56.58 | O |
| ATOM | 1862 | N | ASP | C | 23 | 24.649 | 1.224 | 9.172 | 1.00 | 50.94 | N |
| ATOM | 1863 | CA | ASP | C | 23 | 24.622 | −0.222 | 9.038 | 1.00 | 55.85 | C |
| ATOM | 1864 | CB | ASP | C | 23 | 23.868 | −0.595 | 7.748 | 1.00 | 59.63 | C |
| ATOM | 1865 | CG | ASP | C | 23 | 24.357 | −1.891 | 7.111 | 1.00 | 70.01 | C |
| ATOM | 1866 | OD1 | ASP | C | 23 | 24.894 | −1.818 | 5.984 | 1.00 | 78.00 | O |
| ATOM | 1867 | OD2 | ASP | C | 23 | 24.186 | −2.980 | 7.703 | 1.00 | 62.63 | O |
| ATOM | 1868 | C | ASP | C | 23 | 26.063 | −0.739 | 9.078 | 1.00 | 55.02 | C |
| ATOM | 1869 | O | ASP | C | 23 | 26.978 | −0.058 | 8.619 | 1.00 | 53.05 | O |
| ATOM | 1870 | N | PHE | C | 24 | 26.265 | −1.918 | 9.657 | 1.00 | 49.12 | N |
| ATOM | 1871 | CA | PHE | C | 24 | 27.611 | −2.449 | 9.860 | 1.00 | 54.08 | C |
| ATOM | 1872 | CB | PHE | C | 24 | 27.910 | −2.630 | 11.352 | 1.00 | 47.86 | C |
| ATOM | 1873 | CG | PHE | C | 24 | 28.182 | −1.331 | 12.082 | 1.00 | 49.85 | C |
| ATOM | 1874 | CD2 | PHE | C | 24 | 27.133 | −0.548 | 12.556 | 1.00 | 43.22 | C |
| ATOM | 1875 | CE2 | PHE | C | 24 | 27.369 | 0.643 | 13.214 | 1.00 | 45.41 | C |
| ATOM | 1876 | CZ | PHE | C | 24 | 28.689 | 1.077 | 13.418 | 1.00 | 48.74 | C |
| ATOM | 1877 | CE1 | PHE | C | 24 | 29.744 | 0.306 | 12.949 | 1.00 | 37.27 | C |
| ATOM | 1878 | CD1 | PHE | C | 24 | 29.486 | −0.894 | 12.282 | 1.00 | 42.49 | C |
| ATOM | 1879 | C | PHE | C | 24 | 27.741 | −3.773 | 9.140 | 1.00 | 56.04 | C |
| ATOM | 1880 | O | PHE | C | 24 | 27.046 | −4.727 | 9.473 | 1.00 | 54.54 | O |
| ATOM | 1881 | N | TRP | C | 25 | 28.642 | −3.837 | 8.165 | 1.00 | 55.66 | N |
| ATOM | 1882 | CA | TRP | C | 25 | 28.610 | −4.928 | 7.198 | 1.00 | 55.80 | C |
| ATOM | 1883 | CB | TRP | C | 25 | 27.767 | −4.505 | 6.003 | 1.00 | 58.74 | C |
| ATOM | 1884 | CG | TRP | C | 25 | 28.465 | −3.451 | 5.175 | 1.00 | 60.49 | C |
| ATOM | 1885 | CD1 | TRP | C | 25 | 28.636 | −2.129 | 5.496 | 1.00 | 64.26 | C |
| ATOM | 1886 | NE1 | TRP | C | 25 | 29.338 | −1.488 | 4.496 | 1.00 | 61.51 | N |
| ATOM | 1887 | CE2 | TRP | C | 25 | 29.630 | −2.387 | 3.513 | 1.00 | 53.51 | C |
| ATOM | 1888 | CD2 | TRP | C | 25 | 29.107 | −3.640 | 3.902 | 1.00 | 52.48 | C |
| ATOM | 1889 | CE3 | TRP | C | 25 | 29.280 | −4.742 | 3.061 | 1.00 | 59.06 | C |
| ATOM | 1890 | CZ3 | TRP | C | 25 | 29.966 | −4.566 | 1.866 | 1.00 | 61.55 | C |
| ATOM | 1891 | CH2 | TRP | C | 25 | 30.476 | −3.311 | 1.505 | 1.00 | 62.77 | C |
| ATOM | 1892 | CZ2 | TRP | C | 25 | 30.320 | −2.214 | 2.312 | 1.00 | 59.11 | C |
| ATOM | 1893 | C | TRP | C | 25 | 29.972 | −5.310 | 6.675 | 1.00 | 57.07 | C |
| ATOM | 1894 | O | TRP | C | 25 | 30.980 | −4.660 | 6.967 | 1.00 | 52.56 | O |
| ATOM | 1895 | N | ALA | C | 26 | 29.969 | −6.353 | 5.855 | 1.00 | 55.09 | N |
| ATOM | 1896 | CA | ALA | C | 26 | 31.176 | −6.844 | 5.211 | 1.00 | 63.91 | C |
| ATOM | 1897 | CB | ALA | C | 26 | 31.991 | −7.686 | 6.183 | 1.00 | 58.13 | C |
| ATOM | 1898 | C | ALA | C | 26 | 30.818 | −7.656 | 3.974 | 1.00 | 65.29 | C |
| ATOM | 1899 | O | ALA | C | 26 | 29.841 | −8.406 | 3.982 | 1.00 | 59.41 | O |
| ATOM | 1900 | N | GLU | C | 27 | 31.629 | −7.508 | 2.928 | 1.00 | 71.48 | N |
| ATOM | 1901 | CA | GLU | C | 27 | 31.403 | −8.157 | 1.644 | 1.00 | 61.11 | C |
| ATOM | 1902 | CB | GLU | C | 27 | 32.582 | −7.872 | 0.711 | 1.00 | 70.23 | C |
| ATOM | 1903 | CG | GLU | C | 27 | 32.220 | −7.821 | −0.772 | 1.00 | 85.45 | C |
| ATOM | 1904 | CD | GLU | C | 27 | 31.488 | −6.541 | −1.176 | 1.00 | 88.32 | C |
| ATOM | 1905 | OE1 | GLU | C | 27 | 31.859 | −5.447 | −0.697 | 1.00 | 83.38 | O |
| ATOM | 1906 | OE2 | GLU | C | 27 | 30.540 | −6.633 | −1.988 | 1.00 | 95.63 | O |
| ATOM | 1907 | C | GLU | C | 27 | 31.162 | −9.668 | 1.736 | 1.00 | 70.29 | C |
| ATOM | 1908 | O | GLU | C | 27 | 30.540 | −10.251 | 0.849 | 1.00 | 85.86 | O |
| ATOM | 1909 | N | TRP | C | 28 | 31.634 | −10.308 | 2.799 | 1.00 | 79.45 | N |
| ATOM | 1910 | CA | TRP | C | 28 | 31.536 | −11.765 | 2.891 | 1.00 | 82.42 | C |
| ATOM | 1911 | CB | TRP | C | 28 | 32.756 | −12.333 | 3.621 | 1.00 | 82.22 | C |
| ATOM | 1912 | CG | TRP | C | 28 | 32.769 | −12.008 | 5.086 | 1.00 | 82.11 | C |
| ATOM | 1913 | CD1 | TRP | C | 28 | 31.969 | −12.547 | 6.063 | 1.00 | 82.39 | C |
| ATOM | 1914 | NE1 | TRP | C | 28 | 32.276 | −11.993 | 7.284 | 1.00 | 81.81 | N |
| ATOM | 1915 | CE2 | TRP | C | 28 | 33.280 | −11.096 | 7.118 | 1.00 | 73.76 | C |
| ATOM | 1916 | CD2 | TRP | C | 28 | 33.627 | −11.068 | 5.744 | 1.00 | 79.24 | C |
| ATOM | 1917 | CE3 | TRP | C | 28 | 34.640 | −10.224 | 5.305 | 1.00 | 79.15 | C |
| ATOM | 1918 | CZ3 | TRP | C | 28 | 35.280 | −9.435 | 6.233 | 1.00 | 83.88 | C |
| ATOM | 1919 | CH2 | TRP | C | 28 | 34.924 | −9.473 | 7.592 | 1.00 | 87.39 | C |
| ATOM | 1920 | CZ2 | TRP | C | 28 | 33.928 | −10.291 | 8.047 | 1.00 | 70.20 | C |
| ATOM | 1921 | C | TRP | C | 28 | 30.255 | −12.260 | 3.575 | 1.00 | 84.57 | C |
| ATOM | 1922 | O | TRP | C | 28 | 29.825 | −13.398 | 3.365 | 1.00 | 89.52 | O |
| ATOM | 1923 | N | CYS | C | 29 | 29.664 | −11.404 | 4.405 | 1.00 | 66.24 | N |
| ATOM | 1924 | CA | CYS | C | 29 | 28.488 | −11.755 | 5.210 | 1.00 | 68.33 | C |
| ATOM | 1925 | CB | CYS | C | 29 | 28.270 | −10.673 | 6.284 | 1.00 | 71.12 | C |
| ATOM | 1926 | SG | CYS | C | 29 | 26.883 | −10.861 | 7.466 | 1.00 | 64.13 | S |
| ATOM | 1927 | C | CYS | C | 29 | 27.231 | −11.922 | 4.346 | 1.00 | 72.44 | C |
| ATOM | 1928 | O | CYS | C | 29 | 26.824 | −10.996 | 3.644 | 1.00 | 77.46 | O |
| ATOM | 1929 | N | GLY | C | 30 | 26.619 | −13.100 | 4.407 | 1.00 | 85.61 | N |
| ATOM | 1930 | CA | GLY | C | 30 | 25.427 | −13.389 | 3.625 | 1.00 | 86.62 | C |
| ATOM | 1931 | C | GLY | C | 30 | 24.290 | −12.417 | 3.880 | 1.00 | 87.50 | C |
| ATOM | 1932 | O | GLY | C | 30 | 23.877 | −11.685 | 2.971 | 1.00 | 85.78 | O |
| ATOM | 1933 | N | PRO | C | 31 | 23.764 | −12.414 | 5.120 | 1.00 | 62.79 | N |
| ATOM | 1934 | CA | PRO | C | 31 | 22.663 | −11.525 | 5.515 | 1.00 | 54.32 | C |
| ATOM | 1935 | CB | PRO | C | 31 | 22.524 | −11.786 | 7.013 | 1.00 | 43.61 | C |
| ATOM | 1936 | CG | PRO | C | 31 | 22.960 | −13.198 | 7.174 | 1.00 | 47.24 | C |
| ATOM | 1937 | CD | PRO | C | 31 | 24.074 | −13.401 | 6.169 | 1.00 | 53.87 | C |
| ATOM | 1938 | C | PRO | C | 31 | 23.001 | −10.069 | 5.278 | 1.00 | 60.54 | C |
| ATOM | 1939 | O | PRO | C | 31 | 22.108 | −9.263 | 5.037 | 1.00 | 66.01 | O |
| ATOM | 1940 | N | CYS | C | 32 | 24.278 | −9.730 | 5.352 | 1.00 | 68.34 | N |
| ATOM | 1941 | CA | CYS | C | 32 | 24.673 | −8.344 | 5.186 | 1.00 | 74.16 | C |
| ATOM | 1942 | CB | CYS | C | 32 | 26.145 | −8.150 | 5.563 | 1.00 | 68.53 | C |
| ATOM | 1943 | SG | CYS | C | 32 | 26.568 | −8.861 | 7.178 | 1.00 | 64.47 | S |
| ATOM | 1944 | C | CYS | C | 32 | 24.422 | −8.012 | 3.733 | 1.00 | 74.06 | C |
| ATOM | 1945 | O | CYS | C | 32 | 24.146 | −6.862 | 3.380 | 1.00 | 75.00 | O |
| ATOM | 1946 | N | ARG | C | 33 | 24.495 | −9.046 | 2.897 | 1.00 | 57.32 | N |
| ATOM | 1947 | CA | ARG | C | 33 | 24.183 | −8.909 | 1.483 | 1.00 | 68.62 | C |
| ATOM | 1948 | CB | ARG | C | 33 | 24.930 | −9.955 | 0.656 | 1.00 | 63.33 | C |
| ATOM | 1949 | CG | ARG | C | 33 | 26.180 | −9.427 | −0.031 | 1.00 | 52.87 | C |
| ATOM | 1950 | CD | ARG | C | 33 | 27.163 | −10.567 | −0.262 | 1.00 | 46.97 | C |
| ATOM | 1951 | NE | ARG | C | 33 | 26.458 | −11.845 | −0.244 | 1.00 | 61.48 | N |
| ATOM | 1952 | CZ | ARG | C | 33 | 27.033 | −13.031 | −0.060 | 1.00 | 68.09 | C |
| ATOM | 1953 | NH1 | ARG | C | 33 | 28.346 | −13.124 | 0.132 | 1.00 | 72.02 | N |
| ATOM | 1954 | NH2 | ARG | C | 33 | 26.290 | −14.133 | −0.069 | 1.00 | 70.32 | N |
| ATOM | 1955 | C | ARG | C | 33 | 22.680 | −9.005 | 1.232 | 1.00 | 70.27 | C |
| ATOM | 1956 | O | ARG | C | 33 | 22.152 | −8.293 | 0.385 | 1.00 | 61.48 | O |
| ATOM | 1957 | N | MET | C | 34 | 21.992 | −9.874 | 1.976 | 1.00 | 104.03 | N |
| ATOM | 1958 | CA | MET | C | 34 | 20.550 | −10.060 | 1.783 | 1.00 | 104.88 | C |
| ATOM | 1959 | CB | MET | C | 34 | 20.071 | −11.406 | 2.340 | 1.00 | 97.45 | C |
| ATOM | 1960 | CG | MET | C | 34 | 19.071 | −12.113 | 1.413 | 1.00 | 115.64 | C |
| ATOM | 1961 | SD | MET | C | 34 | 17.985 | −13.340 | 2.195 | 1.00 | 147.26 | S |
| ATOM | 1962 | CE | MET | C | 34 | 16.736 | −12.316 | 2.992 | 1.00 | 106.94 | C |
| ATOM | 1963 | C | MET | C | 34 | 19.727 | −8.930 | 2.391 | 1.00 | 101.70 | C |
| ATOM | 1964 | O | MET | C | 34 | 18.544 | −9.092 | 2.667 | 1.00 | 99.87 | O |
| ATOM | 1965 | N | ILE | C | 35 | 20.360 | −7.784 | 2.603 | 1.00 | 74.35 | N |
| ATOM | 1966 | CA | ILE | C | 35 | 19.678 | −6.627 | 3.170 | 1.00 | 69.12 | C |
| ATOM | 1967 | CB | ILE | C | 35 | 19.783 | −6.582 | 4.701 | 1.00 | 66.74 | C |
| ATOM | 1968 | CG1 | ILE | C | 35 | 21.187 | −6.164 | 5.136 | 1.00 | 74.90 | C |
| ATOM | 1969 | CD1 | ILE | C | 35 | 21.366 | −6.105 | 6.644 | 1.00 | 55.25 | C |
| ATOM | 1970 | CG2 | ILE | C | 35 | 19.431 | −7.925 | 5.324 | 1.00 | 71.04 | C |
| ATOM | 1971 | C | ILE | C | 35 | 20.295 | −5.367 | 2.611 | 1.00 | 70.92 | C |
| ATOM | 1972 | O | ILE | C | 35 | 19.731 | −4.282 | 2.731 | 1.00 | 71.07 | O |
| ATOM | 1973 | N | ALA | C | 36 | 21.471 | −5.512 | 2.011 | 1.00 | 69.67 | N |
| ATOM | 1974 | CA | ALA | C | 36 | 22.057 | −4.393 | 1.295 | 1.00 | 79.08 | C |
| ATOM | 1975 | CB | ALA | C | 36 | 23.270 | −4.825 | 0.476 | 1.00 | 75.52 | C |
| ATOM | 1976 | C | ALA | C | 36 | 20.972 | −3.774 | 0.408 | 1.00 | 81.00 | C |
| ATOM | 1977 | O | ALA | C | 36 | 20.647 | −2.594 | 0.564 | 1.00 | 80.69 | O |
| ATOM | 1978 | N | PRO | C | 37 | 20.378 | −4.576 | −0.497 | 1.00 | 80.07 | N |
| ATOM | 1979 | CA | PRO | C | 37 | 19.283 | −4.083 | −1.344 | 1.00 | 87.49 | C |
| ATOM | 1980 | CB | PRO | C | 37 | 18.774 | −5.358 | −2.044 | 1.00 | 84.80 | C |
| ATOM | 1981 | CG | PRO | C | 37 | 19.318 | −6.500 | −1.241 | 1.00 | 82.18 | C |
| ATOM | 1982 | CD | PRO | C | 37 | 20.643 | −6.003 | −0.746 | 1.00 | 81.04 | C |
| ATOM | 1983 | C | PRO | C | 37 | 18.142 | −3.407 | −0.561 | 1.00 | 79.73 | C |
| ATOM | 1984 | O | PRO | C | 37 | 17.813 | −2.252 | −0.868 | 1.00 | 69.36 | O |
| ATOM | 1985 | N | ILE | C | 38 | 17.553 | −4.109 | 0.412 | 1.00 | 61.47 | N |
| ATOM | 1986 | CA | ILE | C | 38 | 16.492 | −3.532 | 1.254 | 1.00 | 61.66 | C |
| ATOM | 1987 | CB | ILE | C | 38 | 16.168 | −4.415 | 2.458 | 1.00 | 53.33 | C |
| ATOM | 1988 | CG1 | ILE | C | 38 | 15.565 | −5.750 | 2.031 | 1.00 | 34.59 | C |
| ATOM | 1989 | CD1 | ILE | C | 38 | 16.601 | −6.761 | 1.640 | 1.00 | 55.36 | C |
| ATOM | 1990 | CG2 | ILE | C | 38 | 15.227 | −3.673 | 3.410 | 1.00 | 66.48 | C |
| ATOM | 1991 | C | ILE | C | 38 | 16.813 | −2.143 | 1.833 | 1.00 | 69.25 | C |
| ATOM | 1992 | O | ILE | C | 38 | 16.019 | −1.205 | 1.723 | 1.00 | 66.90 | O |
| ATOM | 1993 | N | ILE | C | 39 | 17.960 | −2.020 | 2.490 | 1.00 | 80.75 | N |
| ATOM | 1994 | CA | ILE | C | 39 | 18.367 | −0.725 | 3.010 | 1.00 | 79.60 | C |
| ATOM | 1995 | CB | ILE | C | 39 | 19.772 | −0.784 | 3.651 | 1.00 | 85.93 | C |
| ATOM | 1996 | CG1 | ILE | C | 39 | 19.838 | −1.919 | 4.674 | 1.00 | 84.47 | C |
| ATOM | 1997 | CD1 | ILE | C | 39 | 18.688 | −1.916 | 5.653 | 1.00 | 83.32 | C |
| ATOM | 1998 | CG2 | ILE | C | 39 | 20.146 | 0.557 | 4.293 | 1.00 | 70.33 | C |
| ATOM | 1999 | C | ILE | C | 39 | 18.348 | 0.287 | 1.870 | 1.00 | 75.91 | C |
| ATOM | 2000 | O | ILE | C | 39 | 17.867 | 1.402 | 2.027 | 1.00 | 73.40 | O |
| ATOM | 2001 | N | GLU | C | 40 | 18.852 | −0.112 | 0.710 | 1.00 | 126.73 | N |
| ATOM | 2002 | CA | GLU | C | 40 | 18.983 | 0.821 | −0.403 | 1.00 | 136.48 | C |
| ATOM | 2003 | CB | GLU | C | 40 | 19.900 | 0.252 | −1.490 | 1.00 | 138.06 | C |
| ATOM | 2004 | CG | GLU | C | 40 | 21.377 | 0.652 | −1.356 | 1.00 | 142.34 | C |
| ATOM | 2005 | CD | GLU | C | 40 | 22.119 | −0.097 | −0.255 | 1.00 | 155.00 | C |
| ATOM | 2006 | OE1 | GLU | C | 40 | 21.568 | −0.244 | 0.858 | 1.00 | 146.66 | O |
| ATOM | 2007 | OE2 | GLU | C | 40 | 23.263 | −0.537 | −0.508 | 1.00 | 150.93 | O |
| ATOM | 2008 | C | GLU | C | 40 | 17.632 | 1.253 | −0.983 | 1.00 | 136.96 | C |
| ATOM | 2009 | O | GLU | C | 40 | 17.514 | 2.345 | −1.545 | 1.00 | 134.09 | O |
| ATOM | 2010 | N | GLU | C | 41 | 16.623 | 0.393 | −0.850 | 1.00 | 107.75 | N |
| ATOM | 2011 | CA | GLU | C | 41 | 15.249 | 0.761 | −1.193 | 1.00 | 105.66 | C |
| ATOM | 2012 | CB | GLU | C | 41 | 14.312 | −0.452 | −1.138 | 1.00 | 102.74 | C |
| ATOM | 2013 | CG | GLU | C | 41 | 14.639 | −1.618 | −2.056 | 1.00 | 99.38 | C |
| ATOM | 2014 | CD | GLU | C | 41 | 13.728 | −2.816 | −1.796 | 1.00 | 109.61 | C |
| ATOM | 2015 | OE1 | GLU | C | 41 | 13.994 | −3.910 | −2.343 | 1.00 | 110.54 | O |
| ATOM | 2016 | OE2 | GLU | C | 41 | 12.745 | −2.663 | −1.034 | 1.00 | 111.88 | O |
| ATOM | 2017 | C | GLU | C | 41 | 14.761 | 1.771 | −0.170 | 1.00 | 106.25 | C |
| ATOM | 2018 | O | GLU | C | 41 | 14.408 | 2.902 | −0.504 | 1.00 | 100.33 | O |
| ATOM | 2019 | N | LEU | C | 42 | 14.754 | 1.339 | 1.089 | 1.00 | 88.35 | N |
| ATOM | 2020 | CA | LEU | C | 42 | 14.297 | 2.167 | 2.192 | 1.00 | 80.06 | C |
| ATOM | 2021 | CB | LEU | C | 42 | 14.401 | 1.407 | 3.516 | 1.00 | 83.80 | C |
| ATOM | 2022 | CG | LEU | C | 42 | 13.336 | 0.331 | 3.790 | 1.00 | 93.85 | C |
| ATOM | 2023 | CD1 | LEU | C | 42 | 13.274 | −0.718 | 2.678 | 1.00 | 92.86 | C |
| ATOM | 2024 | CD2 | LEU | C | 42 | 13.549 | −0.338 | 5.163 | 1.00 | 88.25 | C |
| ATOM | 2025 | C | LEU | C | 42 | 15.100 | 3.462 | 2.218 | 1.00 | 79.12 | C |
| ATOM | 2026 | O | LEU | C | 42 | 14.741 | 4.422 | 2.904 | 1.00 | 79.02 | O |
| ATOM | 2027 | N | ALA | C | 43 | 16.171 | 3.490 | 1.431 | 1.00 | 103.95 | N |
| ATOM | 2028 | CA | ALA | C | 43 | 17.008 | 4.674 | 1.306 | 1.00 | 103.40 | C |
| ATOM | 2029 | CB | ALA | C | 43 | 18.368 | 4.307 | 0.730 | 1.00 | 100.87 | C |
| ATOM | 2030 | C | ALA | C | 43 | 16.331 | 5.760 | 0.467 | 1.00 | 109.15 | C |
| ATOM | 2031 | O | ALA | C | 43 | 16.618 | 6.949 | 0.639 | 1.00 | 107.59 | O |
| ATOM | 2032 | N | GLU | C | 44 | 15.431 | 5.358 | −0.429 | 1.00 | 96.88 | N |
| ATOM | 2033 | CA | GLU | C | 44 | 14.668 | 6.327 | −1.212 | 1.00 | 94.95 | C |
| ATOM | 2034 | CB | GLU | C | 44 | 14.508 | 5.881 | −2.674 | 1.00 | 103.63 | C |
| ATOM | 2035 | CG | GLU | C | 44 | 13.945 | 4.466 | −2.880 | 1.00 | 110.41 | C |
| ATOM | 2036 | CD | GLU | C | 44 | 12.438 | 4.440 | −3.155 | 1.00 | 111.87 | C |
| ATOM | 2037 | OE2 | GLU | C | 44 | 11.666 | 4.056 | −2.245 | 1.00 | 112.81 | O |
| ATOM | 2038 | OE1 | GLU | C | 44 | 12.023 | 4.782 | −4.285 | 1.00 | 102.44 | O |
| ATOM | 2039 | C | GLU | C | 44 | 13.310 | 6.600 | −0.573 | 1.00 | 94.85 | C |
| ATOM | 2040 | O | GLU | C | 44 | 12.957 | 7.756 | −0.332 | 1.00 | 91.64 | O |
| ATOM | 2041 | N | GLU | C | 45 | 12.560 | 5.536 | −0.286 | 1.00 | 75.83 | N |
| ATOM | 2042 | CA | GLU | C | 45 | 11.252 | 5.672 | 0.339 | 1.00 | 80.99 | C |
| ATOM | 2043 | CB | GLU | C | 45 | 10.699 | 4.297 | 0.747 | 1.00 | 86.09 | C |
| ATOM | 2044 | CG | GLU | C | 45 | 9.165 | 4.183 | 0.775 | 1.00 | 90.28 | C |
| ATOM | 2045 | CD | GLU | C | 45 | 8.522 | 4.812 | 2.011 | 1.00 | 91.99 | C |
| ATOM | 2046 | OE2 | GLU | C | 45 | 8.039 | 5.965 | 1.916 | 1.00 | 88.00 | O |
| ATOM | 2047 | OE1 | GLU | C | 45 | 8.499 | 4.156 | 3.078 | 1.00 | 87.14 | O |
| ATOM | 2048 | C | GLU | C | 45 | 11.409 | 6.604 | 1.542 | 1.00 | 85.18 | C |
| ATOM | 2049 | O | GLU | C | 45 | 10.460 | 7.264 | 1.975 | 1.00 | 84.68 | O |
| ATOM | 2050 | N | TYR | C | 46 | 12.627 | 6.652 | 2.072 | 1.00 | 93.10 | N |
| ATOM | 2051 | CA | TYR | C | 46 | 12.976 | 7.593 | 3.121 | 1.00 | 87.83 | C |
| ATOM | 2052 | CB | TYR | C | 46 | 13.237 | 6.859 | 4.429 | 1.00 | 86.56 | C |
| ATOM | 2053 | CG | TYR | C | 46 | 12.061 | 6.025 | 4.870 | 1.00 | 88.69 | C |
| ATOM | 2054 | CD1 | TYR | C | 46 | 10.902 | 6.623 | 5.343 | 1.00 | 84.82 | C |
| ATOM | 2055 | CE1 | TYR | C | 46 | 9.817 | 5.864 | 5.741 | 1.00 | 86.73 | C |
| ATOM | 2056 | CZ | TYR | C | 46 | 9.885 | 4.486 | 5.663 | 1.00 | 93.03 | C |
| ATOM | 2057 | OH | TYR | C | 46 | 8.812 | 3.715 | 6.058 | 1.00 | 88.79 | O |
| ATOM | 2058 | CE2 | TYR | C | 46 | 11.027 | 3.871 | 5.190 | 1.00 | 90.00 | C |
| ATOM | 2059 | CD2 | TYR | C | 46 | 12.102 | 4.639 | 4.795 | 1.00 | 87.39 | C |
| ATOM | 2060 | C | TYR | C | 46 | 14.191 | 8.379 | 2.690 | 1.00 | 82.88 | C |
| ATOM | 2061 | O | TYR | C | 46 | 15.289 | 8.168 | 3.184 | 1.00 | 85.71 | O |
| ATOM | 2062 | N | ALA | C | 47 | 13.984 | 9.260 | 1.723 | 1.00 | 73.83 | N |
| ATOM | 2063 | CA | ALA | C | 47 | 15.049 | 10.116 | 1.240 | 1.00 | 73.14 | C |
| ATOM | 2064 | CB | ALA | C | 47 | 15.120 | 10.074 | −0.263 | 1.00 | 75.07 | C |
| ATOM | 2065 | C | ALA | C | 47 | 14.736 | 11.516 | 1.710 | 1.00 | 75.13 | C |
| ATOM | 2066 | O | ALA | C | 47 | 13.572 | 11.919 | 1.741 | 1.00 | 75.12 | O |
| ATOM | 2067 | N | GLY | C | 48 | 15.769 | 12.264 | 2.077 | 1.00 | 62.09 | N |
| ATOM | 2068 | CA | GLY | C | 48 | 15.565 | 13.583 | 2.649 | 1.00 | 61.50 | C |
| ATOM | 2069 | C | GLY | C | 48 | 14.788 | 13.520 | 3.958 | 1.00 | 61.88 | C |
| ATOM | 2070 | O | GLY | C | 48 | 14.425 | 14.549 | 4.527 | 1.00 | 59.02 | O |
| ATOM | 2071 | N | LYS | C | 49 | 14.526 | 12.311 | 4.440 | 1.00 | 89.30 | N |
| ATOM | 2072 | CA | LYS | C | 49 | 13.863 | 12.150 | 5.729 | 1.00 | 98.01 | C |
| ATOM | 2073 | CB | LYS | C | 49 | 12.453 | 11.579 | 5.549 | 1.00 | 97.68 | C |
| ATOM | 2074 | CG | LYS | C | 49 | 12.325 | 10.486 | 4.496 | 1.00 | 101.42 | C |
| ATOM | 2075 | CD | LYS | C | 49 | 10.855 | 10.151 | 4.254 | 1.00 | 98.99 | C |
| ATOM | 2076 | CE | LYS | C | 49 | 10.048 | 11.409 | 3.952 | 1.00 | 90.65 | C |
| ATOM | 2077 | NZ | LYS | C | 49 | 8.692 | 11.354 | 4.552 | 1.00 | 85.11 | N |
| ATOM | 2078 | C | LYS | C | 49 | 14.682 | 11.299 | 6.702 | 1.00 | 100.75 | C |
| ATOM | 2079 | O | LYS | C | 49 | 14.617 | 11.486 | 7.920 | 1.00 | 97.70 | O |
| ATOM | 2080 | N | VAL | C | 50 | 15.450 | 10.365 | 6.152 | 1.00 | 99.20 | N |
| ATOM | 2081 | CA | VAL | C | 50 | 16.301 | 9.508 | 6.959 | 1.00 | 93.53 | C |
| ATOM | 2082 | CB | VAL | C | 50 | 15.601 | 8.195 | 7.325 | 1.00 | 98.91 | C |
| ATOM | 2083 | CG1 | VAL | C | 50 | 16.601 | 7.222 | 7.945 | 1.00 | 94.88 | C |
| ATOM | 2084 | CG2 | VAL | C | 50 | 14.422 | 8.456 | 8.269 | 1.00 | 101.35 | C |
| ATOM | 2085 | C | VAL | C | 50 | 17.595 | 9.179 | 6.237 | 1.00 | 90.14 | C |
| ATOM | 2086 | O | VAL | C | 50 | 17.589 | 8.888 | 5.047 | 1.00 | 95.63 | O |
| ATOM | 2087 | N | VAL | C | 51 | 18.705 | 9.220 | 6.964 | 1.00 | 62.53 | N |
| ATOM | 2088 | CA | VAL | C | 51 | 20.001 | 8.911 | 6.371 | 1.00 | 59.94 | C |
| ATOM | 2089 | CB | VAL | C | 51 | 21.131 | 9.680 | 7.036 | 1.00 | 46.71 | C |
| ATOM | 2090 | CG1 | VAL | C | 51 | 22.427 | 9.368 | 6.320 | 1.00 | 52.06 | C |
| ATOM | 2091 | CG2 | VAL | C | 51 | 20.834 | 11.179 | 7.035 | 1.00 | 43.27 | C |
| ATOM | 2092 | C | VAL | C | 51 | 20.344 | 7.449 | 6.529 | 1.00 | 54.52 | C |
| ATOM | 2093 | O | VAL | C | 51 | 20.038 | 6.857 | 7.547 | 1.00 | 55.20 | O |
| ATOM | 2094 | N | PHE | C | 52 | 20.977 | 6.866 | 5.521 | 1.00 | 66.58 | N |
| ATOM | 2095 | CA | PHE | C | 52 | 21.508 | 5.523 | 5.657 | 1.00 | 67.85 | C |
| ATOM | 2096 | CB | PHE | C | 52 | 20.813 | 4.553 | 4.710 | 1.00 | 65.84 | C |
| ATOM | 2097 | CG | PHE | C | 52 | 19.361 | 4.348 | 5.017 | 1.00 | 73.14 | C |
| ATOM | 2098 | CD2 | PHE | C | 52 | 18.925 | 3.185 | 5.626 | 1.00 | 77.89 | C |
| ATOM | 2099 | CE2 | PHE | C | 52 | 17.574 | 2.992 | 5.909 | 1.00 | 84.94 | C |
| ATOM | 2100 | CZ | PHE | C | 52 | 16.650 | 3.975 | 5.582 | 1.00 | 81.32 | C |
| ATOM | 2101 | CE1 | PHE | C | 52 | 17.081 | 5.142 | 4.978 | 1.00 | 78.53 | C |
| ATOM | 2102 | CD1 | PHE | C | 52 | 18.425 | 5.322 | 4.694 | 1.00 | 76.89 | C |
| ATOM | 2103 | C | PHE | C | 52 | 23.001 | 5.550 | 5.385 | 1.00 | 75.88 | C |
| ATOM | 2104 | O | PHE | C | 52 | 23.433 | 5.726 | 4.243 | 1.00 | 74.55 | O |
| ATOM | 2105 | N | GLY | C | 53 | 23.789 | 5.410 | 6.446 | 1.00 | 69.77 | N |
| ATOM | 2106 | CA | GLY | C | 53 | 25.224 | 5.278 | 6.304 | 1.00 | 59.28 | C |
| ATOM | 2107 | C | GLY | C | 53 | 25.518 | 3.801 | 6.340 | 1.00 | 52.61 | C |
| ATOM | 2108 | O | GLY | C | 53 | 24.671 | 2.997 | 6.705 | 1.00 | 58.22 | O |
| ATOM | 2109 | N | LYS | C | 54 | 26.712 | 3.422 | 5.941 | 1.00 | 51.28 | N |
| ATOM | 2110 | CA | LYS | C | 54 | 27.121 | 2.067 | 6.211 | 1.00 | 54.13 | C |
| ATOM | 2111 | CB | LYS | C | 54 | 26.710 | 1.124 | 5.097 | 1.00 | 48.13 | C |
| ATOM | 2112 | CG | LYS | C | 54 | 27.343 | 1.434 | 3.781 | 1.00 | 50.56 | C |
| ATOM | 2113 | CD | LYS | C | 54 | 26.991 | 0.357 | 2.778 | 1.00 | 53.71 | C |
| ATOM | 2114 | CE | LYS | C | 54 | 27.604 | 0.675 | 1.418 | 1.00 | 70.19 | C |
| ATOM | 2115 | NZ | LYS | C | 54 | 27.612 | 2.155 | 1.143 | 1.00 | 78.27 | N |
| ATOM | 2116 | C | LYS | C | 54 | 28.610 | 2.021 | 6.486 | 1.00 | 52.48 | C |
| ATOM | 2117 | O | LYS | C | 54 | 29.383 | 2.870 | 6.020 | 1.00 | 54.45 | O |
| ATOM | 2118 | N | VAL | C | 55 | 28.996 | 1.032 | 7.278 | 1.00 | 42.53 | N |
| ATOM | 2119 | CA | VAL | C | 55 | 30.330 | 1.011 | 7.842 | 1.00 | 42.46 | C |
| ATOM | 2120 | CB | VAL | C | 55 | 30.322 | 1.252 | 9.357 | 1.00 | 30.49 | C |
| ATOM | 2121 | CG1 | VAL | C | 55 | 31.648 | 0.827 | 9.962 | 1.00 | 39.78 | C |
| ATOM | 2122 | CG2 | VAL | C | 55 | 30.063 | 2.716 | 9.644 | 1.00 | 30.77 | C |
| ATOM | 2123 | C | VAL | C | 55 | 30.853 | −0.348 | 7.596 | 1.00 | 39.90 | C |
| ATOM | 2124 | O | VAL | C | 55 | 30.352 | −1.302 | 8.186 | 1.00 | 41.46 | O |
| ATOM | 2125 | N | ASN | C | 56 | 31.827 | −0.439 | 6.691 | 1.00 | 46.31 | N |
| ATOM | 2126 | CA | ASN | C | 56 | 32.483 | −1.708 | 6.423 | 1.00 | 52.50 | C |
| ATOM | 2127 | CB | ASN | C | 56 | 33.275 | −1.682 | 5.115 | 1.00 | 45.91 | C |
| ATOM | 2128 | CG | ASN | C | 56 | 33.782 | −3.056 | 4.739 | 1.00 | 56.04 | C |
| ATOM | 2129 | OD1 | ASN | C | 56 | 34.504 | −3.671 | 5.529 | 1.00 | 59.69 | O |
| ATOM | 2130 | ND2 | ASN | C | 56 | 33.364 | −3.579 | 3.563 | 1.00 | 45.20 | N |
| ATOM | 2131 | C | ASN | C | 56 | 33.411 | −1.995 | 7.588 | 1.00 | 50.45 | C |
| ATOM | 2132 | O | ASN | C | 56 | 34.331 | −1.200 | 7.852 | 1.00 | 41.60 | O |
| ATOM | 2133 | N | VAL | C | 57 | 33.164 | −3.110 | 8.284 | 1.00 | 39.33 | N |
| ATOM | 2134 | CA | VAL | C | 57 | 33.858 | −3.388 | 9.547 | 1.00 | 47.47 | C |
| ATOM | 2135 | CB | VAL | C | 57 | 33.145 | −4.439 | 10.415 | 1.00 | 43.33 | C |
| ATOM | 2136 | CG1 | VAL | C | 57 | 31.744 | −3.936 | 10.879 | 1.00 | 47.81 | C |
| ATOM | 2137 | CG2 | VAL | C | 57 | 33.073 | −5.762 | 9.666 | 1.00 | 38.58 | C |
| ATOM | 2138 | C | VAL | C | 57 | 35.278 | −3.881 | 9.328 | 1.00 | 56.89 | C |
| ATOM | 2139 | O | VAL | C | 57 | 36.016 | −4.107 | 10.295 | 1.00 | 58.38 | O |
| ATOM | 2140 | N | ASP | C | 58 | 35.646 | −4.061 | 8.061 | 1.00 | 55.13 | N |
| ATOM | 2141 | CA | ASP | C | 58 | 36.979 | −4.514 | 7.689 | 1.00 | 54.15 | C |
| ATOM | 2142 | CB | ASP | C | 58 | 36.939 | −5.180 | 6.309 | 1.00 | 59.24 | C |
| ATOM | 2143 | CG | ASP | C | 58 | 36.766 | −6.690 | 6.376 | 1.00 | 64.76 | C |
| ATOM | 2144 | OD1 | ASP | C | 58 | 36.534 | −7.230 | 7.482 | 1.00 | 54.14 | O |
| ATOM | 2145 | OD2 | ASP | C | 58 | 36.843 | −7.332 | 5.298 | 1.00 | 63.35 | O |
| ATOM | 2146 | C | ASP | C | 58 | 37.952 | −3.351 | 7.635 | 1.00 | 52.58 | C |
| ATOM | 2147 | O | ASP | C | 58 | 39.093 | −3.458 | 8.071 | 1.00 | 57.81 | O |
| ATOM | 2148 | N | GLU | C | 59 | 37.491 | −2.248 | 7.073 | 1.00 | 51.55 | N |
| ATOM | 2149 | CA | GLU | C | 59 | 38.357 | −1.119 | 6.834 | 1.00 | 52.89 | C |
| ATOM | 2150 | CB | GLU | C | 59 | 38.110 | −0.502 | 5.450 | 1.00 | 59.39 | C |
| ATOM | 2151 | CG | GLU | C | 59 | 38.312 | −1.475 | 4.290 | 1.00 | 69.54 | C |
| ATOM | 2152 | CD | GLU | C | 59 | 39.691 | −2.136 | 4.300 | 1.00 | 74.29 | C |
| ATOM | 2153 | OE2 | GLU | C | 59 | 39.746 | −3.392 | 4.355 | 1.00 | 64.82 | O |
| ATOM | 2154 | OE1 | GLU | C | 59 | 40.710 | −1.401 | 4.259 | 1.00 | 76.06 | O |
| ATOM | 2155 | C | GLU | C | 59 | 38.133 | −0.100 | 7.913 | 1.00 | 52.19 | C |
| ATOM | 2156 | O | GLU | C | 59 | 38.734 | 0.972 | 7.910 | 1.00 | 67.23 | O |
| ATOM | 2157 | N | ASN | C | 60 | 37.243 | −0.422 | 8.836 | 1.00 | 43.23 | N |
| ATOM | 2158 | CA | ASN | C | 60 | 37.122 | 0.381 | 10.041 | 1.00 | 42.67 | C |
| ATOM | 2159 | CB | ASN | C | 60 | 36.126 | 1.552 | 9.892 | 1.00 | 39.53 | C |
| ATOM | 2160 | CG | ASN | C | 60 | 36.145 | 2.198 | 8.495 | 1.00 | 49.81 | C |
| ATOM | 2161 | OD1 | ASN | C | 60 | 36.509 | 3.369 | 8.337 | 1.00 | 49.02 | O |
| ATOM | 2162 | ND2 | ASN | C | 60 | 35.703 | 1.440 | 7.484 | 1.00 | 43.51 | N |
| ATOM | 2163 | C | ASN | C | 60 | 36.716 | −0.506 | 11.196 | 1.00 | 41.06 | C |
| ATOM | 2164 | O | ASN | C | 60 | 35.657 | −0.292 | 11.786 | 1.00 | 43.11 | O |
| ATOM | 2165 | N | PRO | C | 61 | 37.562 | −1.489 | 11.531 | 1.00 | 43.68 | N |
| ATOM | 2166 | CA | PRO | C | 61 | 37.363 | −2.368 | 12.693 | 1.00 | 40.85 | C |
| ATOM | 2167 | CB | PRO | C | 61 | 38.539 | −3.344 | 12.601 | 1.00 | 46.90 | C |
| ATOM | 2168 | CG | PRO | C | 61 | 39.596 | −2.600 | 11.856 | 1.00 | 48.05 | C |
| ATOM | 2169 | CD | PRO | C | 61 | 38.884 | −1.675 | 10.902 | 1.00 | 52.02 | C |
| ATOM | 2170 | C | PRO | C | 61 | 37.380 | −1.608 | 14.022 | 1.00 | 40.26 | C |
| ATOM | 2171 | O | PRO | C | 61 | 36.805 | −2.060 | 15.019 | 1.00 | 42.23 | O |
| ATOM | 2172 | N | GLU | C | 62 | 38.011 | −0.446 | 14.041 | 1.00 | 37.89 | N |
| ATOM | 2173 | CA | GLU | C | 62 | 38.011 | 0.345 | 15.263 | 1.00 | 41.07 | C |
| ATOM | 2174 | CB | GLU | C | 62 | 39.132 | 1.387 | 15.286 | 1.00 | 45.15 | C |
| ATOM | 2175 | CG | GLU | C | 62 | 39.009 | 2.467 | 14.234 | 1.00 | 53.87 | C |
| ATOM | 2176 | CD | GLU | C | 62 | 39.409 | 1.974 | 12.842 | 1.00 | 66.10 | C |
| ATOM | 2177 | OE1 | GLU | C | 62 | 40.147 | 0.964 | 12.743 | 1.00 | 62.85 | O |
| ATOM | 2178 | OE2 | GLU | C | 62 | 38.977 | 2.588 | 11.842 | 1.00 | 67.46 | O |
| ATOM | 2179 | C | GLU | C | 62 | 36.691 | 1.023 | 15.570 | 1.00 | 49.39 | C |
| ATOM | 2180 | O | GLU | C | 62 | 36.336 | 1.148 | 16.733 | 1.00 | 52.80 | O |
| ATOM | 2181 | N | ILE | C | 63 | 35.965 | 1.475 | 14.549 | 1.00 | 50.07 | N |
| ATOM | 2182 | CA | ILE | C | 63 | 34.678 | 2.132 | 14.783 | 1.00 | 47.59 | C |
| ATOM | 2183 | CB | ILE | C | 63 | 34.108 | 2.714 | 13.484 | 1.00 | 56.25 | C |
| ATOM | 2184 | CG1 | ILE | C | 63 | 35.209 | 3.411 | 12.690 | 1.00 | 54.63 | C |
| ATOM | 2185 | CD1 | ILE | C | 63 | 34.690 | 4.469 | 11.743 | 1.00 | 58.91 | C |
| ATOM | 2186 | CG2 | ILE | C | 63 | 32.957 | 3.666 | 13.774 | 1.00 | 48.01 | C |
| ATOM | 2187 | C | ILE | C | 63 | 33.689 | 1.119 | 15.353 | 1.00 | 42.05 | C |
| ATOM | 2188 | O | ILE | C | 63 | 32.973 | 1.380 | 16.329 | 1.00 | 45.48 | O |
| ATOM | 2189 | N | ALA | C | 64 | 33.664 | −0.052 | 14.735 | 1.00 | 37.78 | N |
| ATOM | 2190 | CA | ALA | C | 64 | 32.924 | −1.171 | 15.274 | 1.00 | 38.10 | C |
| ATOM | 2191 | CB | ALA | C | 64 | 33.054 | −2.362 | 14.352 | 1.00 | 30.52 | C |
| ATOM | 2192 | C | ALA | C | 64 | 33.377 | −1.530 | 16.705 | 1.00 | 41.18 | C |
| ATOM | 2193 | O | ALA | C | 64 | 32.528 | −1.823 | 17.550 | 1.00 | 37.48 | O |
| ATOM | 2194 | N | ALA | C | 65 | 34.695 | −1.512 | 16.983 | 1.00 | 37.79 | N |
| ATOM | 2195 | CA | ALA | C | 65 | 35.183 | −1.780 | 18.364 | 1.00 | 39.04 | C |
| ATOM | 2196 | CB | ALA | C | 65 | 36.719 | −1.648 | 18.516 | 1.00 | 33.90 | C |
| ATOM | 2197 | C | ALA | C | 65 | 34.510 | −0.821 | 19.317 | 1.00 | 35.16 | C |
| ATOM | 2198 | O | ALA | C | 65 | 34.026 | −1.247 | 20.357 | 1.00 | 33.90 | O |
| ATOM | 2199 | N | LYS | C | 66 | 34.466 | 0.461 | 18.928 | 1.00 | 39.82 | N |
| ATOM | 2200 | C | LYS | C | 66 | 32.459 | 1.277 | 20.199 | 1.00 | 47.86 | C |
| ATOM | 2201 | O | LYS | C | 66 | 32.122 | 1.455 | 21.360 | 1.00 | 47.09 | O |
| ATOM | 2202 | CA | ALYS | C | 66 | 33.911 | 1.539 | 19.757 | 1.00 | 42.27 | C |
| ATOM | 2203 | CB | ALYS | C | 66 | 34.012 | 2.868 | 19.002 | 1.00 | 43.14 | C |
| ATOM | 2204 | CG | ALYS | C | 66 | 33.833 | 4.146 | 19.831 | 1.00 | 43.31 | C |
| ATOM | 2205 | CD | ALYS | C | 66 | 34.353 | 5.346 | 19.026 | 1.00 | 45.93 | C |
| ATOM | 2206 | CE | ALYS | C | 66 | 34.414 | 6.633 | 19.820 | 1.00 | 44.79 | C |
| ATOM | 2207 | NZ | ALYS | C | 66 | 33.119 | 6.876 | 20.459 | 1.00 | 52.79 | N |
| ATOM | 2208 | CA | BLYS | C | 66 | 33.902 | 1.532 | 19.753 | 0.00 | 42.47 | C |
| ATOM | 2209 | CB | BLYS | C | 66 | 34.011 | 2.870 | 19.011 | 0.00 | 43.32 | C |
| ATOM | 2210 | CG | BLYS | C | 66 | 33.579 | 4.098 | 19.805 | 0.00 | 43.74 | C |
| ATOM | 2211 | CD | BLYS | C | 66 | 34.001 | 5.381 | 19.089 | 0.00 | 46.14 | C |
| ATOM | 2212 | CE | BLYS | C | 66 | 33.703 | 6.622 | 19.919 | 0.00 | 46.58 | C |
| ATOM | 2213 | NZ | BLYS | C | 66 | 34.420 | 7.830 | 19.412 | 0.00 | 45.62 | N |
| ATOM | 2214 | N | TYR | C | 67 | 31.604 | 0.855 | 19.270 | 1.00 | 41.70 | N |
| ATOM | 2215 | CA | TYR | C | 67 | 30.190 | 0.604 | 19.574 | 1.00 | 42.10 | C |
| ATOM | 2216 | CB | TYR | C | 67 | 29.282 | 1.101 | 18.413 | 1.00 | 40.46 | C |
| ATOM | 2217 | CG | TYR | C | 67 | 29.451 | 2.577 | 18.193 | 1.00 | 36.76 | C |
| ATOM | 2218 | CD1 | TYR | C | 67 | 28.753 | 3.495 | 18.958 | 1.00 | 37.38 | C |
| ATOM | 2219 | CE1 | TYR | C | 67 | 28.948 | 4.848 | 18.793 | 1.00 | 33.25 | C |
| ATOM | 2220 | CZ | TYR | C | 67 | 29.859 | 5.280 | 17.871 | 1.00 | 36.05 | C |
| ATOM | 2221 | OH | TYR | C | 67 | 30.087 | 6.614 | 17.698 | 1.00 | 52.57 | O |
| ATOM | 2222 | CE2 | TYR | C | 67 | 30.546 | 4.399 | 17.104 | 1.00 | 34.03 | C |
| ATOM | 2223 | CD2 | TYR | C | 67 | 30.349 | 3.054 | 17.270 | 1.00 | 35.02 | C |
| ATOM | 2224 | C | TYR | C | 67 | 29.953 | −0.876 | 19.850 | 1.00 | 44.78 | C |
| ATOM | 2225 | O | TYR | C | 67 | 28.871 | −1.408 | 19.599 | 1.00 | 39.42 | O |
| ATOM | 2226 | N | GLY | C | 68 | 30.979 | −1.552 | 20.345 | 1.00 | 40.15 | N |
| ATOM | 2227 | CA | GLY | C | 68 | 30.867 | −2.983 | 20.570 | 1.00 | 39.35 | C |
| ATOM | 2228 | C | GLY | C | 68 | 29.913 | −3.666 | 19.601 | 1.00 | 43.60 | C |
| ATOM | 2229 | O | GLY | C | 68 | 28.904 | −4.214 | 19.998 | 1.00 | 44.26 | O |
| ATOM | 2230 | N | ILE | C | 69 | 30.222 | −3.643 | 18.313 | 1.00 | 46.69 | N |
| ATOM | 2231 | CA | ILE | C | 69 | 29.421 | −4.419 | 17.379 | 1.00 | 45.48 | C |
| ATOM | 2232 | CB | ILE | C | 69 | 29.466 | −3.866 | 15.954 | 1.00 | 44.44 | C |
| ATOM | 2233 | CG1 | ILE | C | 69 | 29.130 | −2.383 | 15.957 | 1.00 | 41.83 | C |
| ATOM | 2234 | CD1 | ILE | C | 69 | 27.781 | −2.109 | 16.539 | 1.00 | 42.63 | C |
| ATOM | 2235 | CG2 | ILE | C | 69 | 28.476 | −4.608 | 15.087 | 1.00 | 34.26 | C |
| ATOM | 2236 | C | ILE | C | 69 | 29.888 | −5.861 | 17.379 | 1.00 | 43.90 | C |
| ATOM | 2237 | O | ILE | C | 69 | 30.832 | −6.223 | 16.679 | 1.00 | 43.22 | O |
| ATOM | 2238 | N | MET | C | 70 | 29.208 | −6.688 | 18.159 | 1.00 | 46.82 | N |
| ATOM | 2239 | CA | MET | C | 70 | 29.577 | −8.093 | 18.267 | 1.00 | 46.35 | C |
| ATOM | 2240 | CB | MET | C | 70 | 29.263 | −8.610 | 19.668 | 1.00 | 42.63 | C |
| ATOM | 2241 | CG | MET | C | 70 | 29.900 | −7.765 | 20.758 | 1.00 | 54.97 | C |
| ATOM | 2242 | SD | MET | C | 70 | 31.665 | −7.406 | 20.509 | 1.00 | 56.66 | S |
| ATOM | 2243 | CE | MET | C | 70 | 32.323 | −9.066 | 20.516 | 1.00 | 57.88 | C |
| ATOM | 2244 | C | MET | C | 70 | 28.967 | −9.015 | 17.218 | 1.00 | 50.60 | C |
| ATOM | 2245 | O | MET | C | 70 | 29.147 | −10.228 | 17.289 | 1.00 | 54.74 | O |
| ATOM | 2246 | N | SER | C | 71 | 28.241 | −8.451 | 16.257 | 1.00 | 58.73 | N |
| ATOM | 2247 | CA | SER | C | 71 | 27.664 | −9.254 | 15.177 | 1.00 | 60.33 | C |
| ATOM | 2248 | CB | SER | C | 71 | 26.575 | −10.184 | 15.702 | 1.00 | 59.93 | C |
| ATOM | 2249 | OG | SER | C | 71 | 25.440 | −9.431 | 16.077 | 1.00 | 72.31 | O |
| ATOM | 2250 | C | SER | C | 71 | 27.094 | −8.417 | 14.034 | 1.00 | 62.52 | C |
| ATOM | 2251 | O | SER | C | 71 | 26.563 | −7.327 | 14.241 | 1.00 | 55.88 | O |
| ATOM | 2252 | N | ILE | C | 72 | 27.210 | −8.951 | 12.824 | 1.00 | 55.25 | N |
| ATOM | 2253 | CA | ILE | C | 72 | 26.654 | −8.316 | 11.641 | 1.00 | 54.91 | C |
| ATOM | 2254 | CB | ILE | C | 72 | 27.767 | −7.872 | 10.664 | 1.00 | 49.06 | C |
| ATOM | 2255 | CG1 | ILE | C | 72 | 28.837 | −8.958 | 10.551 | 1.00 | 49.61 | C |
| ATOM | 2256 | CD1 | ILE | C | 72 | 29.856 | −8.744 | 9.459 | 1.00 | 47.30 | C |
| ATOM | 2257 | CG2 | ILE | C | 72 | 28.408 | −6.590 | 11.139 | 1.00 | 50.20 | C |
| ATOM | 2258 | C | ILE | C | 72 | 25.647 | −9.248 | 10.951 | 1.00 | 55.19 | C |
| ATOM | 2259 | O | ILE | C | 72 | 25.835 | −10.463 | 10.896 | 1.00 | 52.21 | O |
| ATOM | 2260 | N | PRO | C | 73 | 24.559 | −8.673 | 10.431 | 1.00 | 71.29 | N |
| ATOM | 2261 | CA | PRO | C | 73 | 24.412 | −7.221 | 10.465 | 1.00 | 64.21 | C |
| ATOM | 2262 | CB | PRO | C | 73 | 23.318 | −6.968 | 9.439 | 1.00 | 63.93 | C |
| ATOM | 2263 | CG | PRO | C | 73 | 22.439 | −8.194 | 9.563 | 1.00 | 68.11 | C |
| ATOM | 2264 | CD | PRO | C | 73 | 23.340 | −9.344 | 9.944 | 1.00 | 71.62 | C |
| ATOM | 2265 | C | PRO | C | 73 | 23.915 | −6.762 | 11.811 | 1.00 | 54.18 | C |
| ATOM | 2266 | O | PRO | C | 73 | 23.480 | −7.548 | 12.663 | 1.00 | 56.58 | O |
| ATOM | 2267 | N | THR | C | 74 | 23.976 | −5.455 | 11.974 | 1.00 | 33.44 | N |
| ATOM | 2268 | CA | THR | C | 74 | 23.498 | −4.782 | 13.157 | 1.00 | 41.33 | C |
| ATOM | 2269 | CB | THR | C | 74 | 24.619 | −4.446 | 14.221 | 1.00 | 45.94 | C |
| ATOM | 2270 | OG1 | THR | C | 74 | 25.076 | −5.618 | 14.911 | 1.00 | 36.18 | O |
| ATOM | 2271 | CG2 | THR | C | 74 | 24.090 | −3.437 | 15.253 | 1.00 | 41.31 | C |
| ATOM | 2272 | C | THR | C | 74 | 23.170 | −3.466 | 12.551 | 1.00 | 42.64 | C |
| ATOM | 2273 | O | THR | C | 74 | 24.043 | −2.808 | 11.989 | 1.00 | 42.88 | O |
| ATOM | 2274 | N | LEU | C | 75 | 21.923 | −3.060 | 12.650 | 1.00 | 60.43 | N |
| ATOM | 2275 | CA | LEU | C | 75 | 21.596 | −1.726 | 12.236 | 1.00 | 61.04 | C |
| ATOM | 2276 | C | LEU | C | 75 | 21.813 | −0.873 | 13.457 | 1.00 | 58.74 | C |
| ATOM | 2277 | O | LEU | C | 75 | 21.518 | −1.308 | 14.573 | 1.00 | 59.77 | O |
| ATOM | 2278 | CB | LEU | C | 75 | 20.134 | −1.672 | 11.833 | 1.00 | 72.15 | C |
| ATOM | 2279 | CG | LEU | C | 75 | 19.867 | −0.895 | 10.557 | 1.00 | 74.20 | C |
| ATOM | 2280 | CD1 | LEU | C | 75 | 20.118 | −1.816 | 9.365 | 1.00 | 64.99 | C |
| ATOM | 2281 | CD2 | LEU | C | 75 | 18.437 | −0.371 | 10.597 | 1.00 | 75.12 | C |
| ATOM | 2282 | N | LEU | C | 76 | 22.324 | 0.336 | 13.268 | 1.00 | 54.05 | N |
| ATOM | 2283 | CA | LEU | C | 76 | 22.394 | 1.280 | 14.385 | 1.00 | 62.87 | C |
| ATOM | 2284 | CB | LEU | C | 76 | 23.845 | 1.610 | 14.763 | 1.00 | 66.29 | C |
| ATOM | 2285 | CG | LEU | C | 76 | 24.281 | 1.300 | 16.194 | 1.00 | 60.93 | C |
| ATOM | 2286 | CD1 | LEU | C | 76 | 24.068 | −0.186 | 16.467 | 1.00 | 53.52 | C |
| ATOM | 2287 | CD2 | LEU | C | 76 | 25.744 | 1.741 | 16.466 | 1.00 | 46.80 | C |
| ATOM | 2288 | C | LEU | C | 76 | 21.631 | 2.562 | 14.075 | 1.00 | 69.02 | C |
| ATOM | 2289 | O | LEU | C | 76 | 21.692 | 3.093 | 12.958 | 1.00 | 61.39 | O |
| ATOM | 2290 | N | PHE | C | 77 | 20.913 | 3.057 | 15.079 | 1.00 | 66.79 | N |
| ATOM | 2291 | CA | PHE | C | 77 | 20.167 | 4.297 | 14.929 | 1.00 | 57.13 | C |
| ATOM | 2292 | CB | PHE | C | 77 | 18.717 | 4.110 | 15.350 | 1.00 | 55.21 | C |
| ATOM | 2293 | CG | PHE | C | 77 | 17.980 | 3.113 | 14.529 | 1.00 | 51.17 | C |
| ATOM | 2294 | CD2 | PHE | C | 77 | 16.923 | 3.511 | 13.723 | 1.00 | 48.29 | C |
| ATOM | 2295 | CE2 | PHE | C | 77 | 16.235 | 2.595 | 12.966 | 1.00 | 55.06 | C |
| ATOM | 2296 | CZ | PHE | C | 77 | 16.596 | 1.248 | 13.006 | 1.00 | 63.34 | C |
| ATOM | 2297 | CE1 | PHE | C | 77 | 17.645 | 0.847 | 13.814 | 1.00 | 64.55 | C |
| ATOM | 2298 | CD1 | PHE | C | 77 | 18.331 | 1.780 | 14.569 | 1.00 | 52.63 | C |
| ATOM | 2299 | C | PHE | C | 77 | 20.761 | 5.391 | 15.770 | 1.00 | 53.08 | C |
| ATOM | 2300 | O | PHE | C | 77 | 20.617 | 5.386 | 16.985 | 1.00 | 55.40 | O |
| ATOM | 2301 | N | PHE | C | 78 | 21.424 | 6.335 | 15.122 | 1.00 | 54.56 | N |
| ATOM | 2302 | CA | PHE | C | 78 | 21.810 | 7.552 | 15.810 | 1.00 | 60.77 | C |
| ATOM | 2303 | CB | PHE | C | 78 | 23.136 | 8.093 | 15.266 | 1.00 | 63.12 | C |
| ATOM | 2304 | CG | PHE | C | 78 | 24.307 | 7.182 | 15.498 | 1.00 | 57.80 | C |
| ATOM | 2305 | CD2 | PHE | C | 78 | 25.465 | 7.668 | 16.081 | 1.00 | 49.21 | C |
| ATOM | 2306 | CE2 | PHE | C | 78 | 26.563 | 6.837 | 16.285 | 1.00 | 49.15 | C |
| ATOM | 2307 | CZ | PHE | C | 78 | 26.492 | 5.498 | 15.906 | 1.00 | 55.56 | C |
| ATOM | 2308 | CE1 | PHE | C | 78 | 25.330 | 5.005 | 15.317 | 1.00 | 56.96 | C |
| ATOM | 2309 | CD1 | PHE | C | 78 | 24.253 | 5.844 | 15.116 | 1.00 | 53.92 | C |
| ATOM | 2310 | C | PHE | C | 78 | 20.708 | 8.596 | 15.638 | 1.00 | 71.43 | C |
| ATOM | 2311 | O | PHE | C | 78 | 20.014 | 8.628 | 14.611 | 1.00 | 61.31 | O |
| ATOM | 2312 | N | LYS | C | 79 | 20.545 | 9.433 | 16.659 | 1.00 | 96.54 | N |
| ATOM | 2313 | CA | LYS | C | 79 | 19.738 | 10.642 | 16.563 | 1.00 | 102.07 | C |
| ATOM | 2314 | CB | LYS | C | 79 | 18.312 | 10.412 | 17.067 | 1.00 | 89.55 | C |
| ATOM | 2315 | CG | LYS | C | 79 | 17.298 | 11.471 | 16.613 | 1.00 | 99.43 | C |
| ATOM | 2316 | CD | LYS | C | 79 | 15.880 | 10.887 | 16.609 | 1.00 | 102.79 | C |
| ATOM | 2317 | CE | LYS | C | 79 | 14.818 | 11.883 | 16.152 | 1.00 | 96.69 | C |
| ATOM | 2318 | NZ | LYS | C | 79 | 13.456 | 11.267 | 16.207 | 1.00 | 87.00 | N |
| ATOM | 2319 | C | LYS | C | 79 | 20.435 | 11.673 | 17.420 | 1.00 | 105.44 | C |
| ATOM | 2320 | O | LYS | C | 79 | 20.819 | 11.380 | 18.552 | 1.00 | 109.55 | O |
| ATOM | 2321 | N | ASN | C | 80 | 20.619 | 12.868 | 16.877 | 1.00 | 80.61 | N |
| ATOM | 2322 | CA | ASN | C | 80 | 21.246 | 13.951 | 17.624 | 1.00 | 86.90 | C |
| ATOM | 2323 | CB | ASN | C | 80 | 20.345 | 14.403 | 18.783 | 1.00 | 88.32 | C |
| ATOM | 2324 | CG | ASN | C | 80 | 18.944 | 14.779 | 18.320 | 1.00 | 83.69 | C |
| ATOM | 2325 | OD1 | ASN | C | 80 | 18.758 | 15.267 | 17.204 | 1.00 | 85.52 | O |
| ATOM | 2326 | ND2 | ASN | C | 80 | 17.951 | 14.548 | 19.175 | 1.00 | 79.77 | N |
| ATOM | 2327 | C | ASN | C | 80 | 22.652 | 13.617 | 18.122 | 1.00 | 82.58 | C |
| ATOM | 2328 | O | ASN | C | 80 | 23.239 | 14.375 | 18.890 | 1.00 | 83.49 | O |
| ATOM | 2329 | N | GLY | C | 81 | 23.192 | 12.488 | 17.671 | 1.00 | 92.00 | N |
| ATOM | 2330 | CA | GLY | C | 81 | 24.546 | 12.093 | 18.025 | 1.00 | 92.08 | C |
| ATOM | 2331 | C | GLY | C | 81 | 24.603 | 11.146 | 19.212 | 1.00 | 80.18 | C |
| ATOM | 2332 | O | GLY | C | 81 | 25.162 | 11.482 | 20.249 | 1.00 | 80.20 | O |
| ATOM | 2333 | N | LYS | C | 82 | 24.032 | 9.959 | 19.041 | 1.00 | 67.40 | N |
| ATOM | 2334 | CA | LYS | C | 82 | 23.853 | 9.004 | 20.120 | 1.00 | 67.23 | C |
| ATOM | 2335 | CB | LYS | C | 82 | 23.504 | 9.732 | 21.413 | 1.00 | 79.38 | C |
| ATOM | 2336 | CG | LYS | C | 82 | 22.227 | 10.565 | 21.322 | 1.00 | 85.22 | C |
| ATOM | 2337 | CD | LYS | C | 82 | 22.245 | 11.686 | 22.346 | 1.00 | 81.09 | C |
| ATOM | 2338 | CE | LYS | C | 82 | 20.935 | 11.772 | 23.092 | 1.00 | 77.88 | C |
| ATOM | 2339 | NZ | LYS | C | 82 | 21.084 | 12.697 | 24.249 | 1.00 | 79.59 | N |
| ATOM | 2340 | C | LYS | C | 82 | 22.741 | 8.015 | 19.779 | 1.00 | 66.98 | C |
| ATOM | 2341 | O | LYS | C | 82 | 21.649 | 8.398 | 19.351 | 1.00 | 68.27 | O |
| ATOM | 2342 | N | VAL | C | 83 | 23.020 | 6.738 | 19.998 | 1.00 | 73.07 | N |
| ATOM | 2343 | CA | VAL | C | 83 | 22.073 | 5.669 | 19.695 | 1.00 | 67.65 | C |
| ATOM | 2344 | CB | VAL | C | 83 | 22.686 | 4.305 | 20.055 | 1.00 | 67.41 | C |
| ATOM | 2345 | CG1 | VAL | C | 83 | 21.844 | 3.159 | 19.497 | 1.00 | 70.44 | C |
| ATOM | 2346 | CG2 | VAL | C | 83 | 24.108 | 4.235 | 19.506 | 1.00 | 57.20 | C |
| ATOM | 2347 | C | VAL | C | 83 | 20.706 | 5.841 | 20.372 | 1.00 | 67.71 | C |
| ATOM | 2348 | O | VAL | C | 83 | 20.551 | 6.646 | 21.289 | 1.00 | 73.66 | O |
| ATOM | 2349 | N | VAL | C | 84 | 19.714 | 5.100 | 19.885 | 1.00 | 64.50 | N |
| ATOM | 2350 | CA | VAL | C | 84 | 18.371 | 5.087 | 20.469 | 1.00 | 65.66 | C |
| ATOM | 2351 | CB | VAL | C | 84 | 17.510 | 6.236 | 19.941 | 1.00 | 64.78 | C |
| ATOM | 2352 | CG1 | VAL | C | 84 | 17.964 | 7.557 | 20.542 | 1.00 | 64.18 | C |
| ATOM | 2353 | CG2 | VAL | C | 84 | 17.568 | 6.272 | 18.426 | 1.00 | 63.80 | C |
| ATOM | 2354 | C | VAL | C | 84 | 17.670 | 3.779 | 20.126 | 1.00 | 66.30 | C |
| ATOM | 2355 | O | VAL | C | 84 | 16.612 | 3.460 | 20.674 | 1.00 | 60.94 | O |
| ATOM | 2356 | N | ASP | C | 85 | 18.262 | 3.049 | 19.183 | 1.00 | 60.89 | N |
| ATOM | 2357 | CA | ASP | C | 85 | 17.813 | 1.704 | 18.842 | 1.00 | 56.32 | C |
| ATOM | 2358 | CB | ASP | C | 85 | 16.472 | 1.724 | 18.112 | 1.00 | 58.62 | C |
| ATOM | 2359 | CG | ASP | C | 85 | 15.565 | 0.565 | 18.522 | 1.00 | 63.73 | C |
| ATOM | 2360 | OD1 | ASP | C | 85 | 15.840 | −0.583 | 18.105 | 1.00 | 64.51 | O |
| ATOM | 2361 | OD2 | ASP | C | 85 | 14.574 | 0.797 | 19.251 | 1.00 | 62.49 | O |
| ATOM | 2362 | C | ASP | C | 85 | 18.864 | 0.947 | 18.039 | 1.00 | 50.54 | C |
| ATOM | 2363 | O | ASP | C | 85 | 19.985 | 1.423 | 17.855 | 1.00 | 55.36 | O |
| ATOM | 2364 | N | GLN | C | 86 | 18.480 | −0.214 | 17.533 | 1.00 | 63.88 | N |
| ATOM | 2365 | CA | GLN | C | 86 | 19.462 | −1.243 | 17.284 | 1.00 | 71.44 | C |
| ATOM | 2366 | CB | GLN | C | 86 | 20.174 | −1.500 | 18.612 | 1.00 | 58.82 | C |
| ATOM | 2367 | CG | GLN | C | 86 | 21.501 | −2.210 | 18.562 | 1.00 | 67.38 | C |
| ATOM | 2368 | CD | GLN | C | 86 | 22.218 | −2.091 | 19.898 | 1.00 | 73.25 | C |
| ATOM | 2369 | OE1 | GLN | C | 86 | 21.575 | −1.845 | 20.926 | 1.00 | 66.33 | O |
| ATOM | 2370 | NE2 | GLN | C | 86 | 23.550 | −2.238 | 19.890 | 1.00 | 61.66 | N |
| ATOM | 2371 | C | GLN | C | 86 | 18.804 | −2.533 | 16.809 | 1.00 | 71.58 | C |
| ATOM | 2372 | O | GLN | C | 86 | 18.269 | −3.304 | 17.606 | 1.00 | 79.93 | O |
| ATOM | 2373 | N | LEU | C | 87 | 18.844 | −2.792 | 15.518 | 1.00 | 55.92 | N |
| ATOM | 2374 | CA | LEU | C | 87 | 18.318 | −4.069 | 15.058 | 1.00 | 67.42 | C |
| ATOM | 2375 | CB | LEU | C | 87 | 17.546 | −3.934 | 13.743 | 1.00 | 66.95 | C |
| ATOM | 2376 | CG | LEU | C | 87 | 16.131 | −3.434 | 14.008 | 1.00 | 75.43 | C |
| ATOM | 2377 | CD1 | LEU | C | 87 | 15.605 | −4.039 | 15.321 | 1.00 | 69.09 | C |
| ATOM | 2378 | CD2 | LEU | C | 87 | 16.120 | −1.918 | 14.070 | 1.00 | 72.19 | C |
| ATOM | 2379 | C | LEU | C | 87 | 19.420 | −5.099 | 14.940 | 1.00 | 64.87 | C |
| ATOM | 2380 | O | LEU | C | 87 | 19.917 | −5.368 | 13.849 | 1.00 | 66.46 | O |
| ATOM | 2381 | N | VAL | C | 88 | 19.800 | −5.679 | 16.068 | 1.00 | 58.03 | N |
| ATOM | 2382 | CA | VAL | C | 88 | 20.835 | −6.701 | 16.055 | 1.00 | 62.36 | C |
| ATOM | 2383 | CB | VAL | C | 88 | 21.331 | −7.009 | 17.472 | 1.00 | 63.43 | C |
| ATOM | 2384 | CG1 | VAL | C | 88 | 22.245 | −8.222 | 17.458 | 1.00 | 65.75 | C |
| ATOM | 2385 | CG2 | VAL | C | 88 | 22.038 | −5.789 | 18.051 | 1.00 | 54.87 | C |
| ATOM | 2386 | C | VAL | C | 88 | 20.364 | −7.984 | 15.366 | 1.00 | 66.90 | C |
| ATOM | 2387 | O | VAL | C | 88 | 19.698 | −8.830 | 15.973 | 1.00 | 68.64 | O |
| ATOM | 2388 | N | GLY | C | 89 | 20.724 | −8.128 | 14.097 | 1.00 | 56.20 | N |
| ATOM | 2389 | CA | GLY | C | 89 | 20.304 | −9.274 | 13.315 | 1.00 | 59.05 | C |
| ATOM | 2390 | C | GLY | C | 89 | 19.596 | −8.846 | 12.044 | 1.00 | 65.04 | C |
| ATOM | 2391 | O | GLY | C | 89 | 18.898 | −7.830 | 12.038 | 1.00 | 68.50 | O |
| ATOM | 2392 | N | ALA | C | 90 | 19.781 | −9.610 | 10.967 | 1.00 | 89.45 | N |
| ATOM | 2393 | CA | ALA | C | 90 | 19.118 | −9.317 | 9.698 | 1.00 | 94.03 | C |
| ATOM | 2394 | CB | ALA | C | 90 | 19.652 | −10.191 | 8.581 | 1.00 | 87.47 | C |
| ATOM | 2395 | C | ALA | C | 90 | 17.638 | −9.532 | 9.860 | 1.00 | 93.83 | C |
| ATOM | 2396 | O | ALA | C | 90 | 17.206 | −10.385 | 10.640 | 1.00 | 90.85 | O |
| ATOM | 2397 | N | ARG | C | 91 | 16.864 | −8.754 | 9.116 | 1.00 | 91.05 | N |
| ATOM | 2398 | CA | ARG | C | 91 | 15.414 | −8.786 | 9.231 | 1.00 | 97.66 | C |
| ATOM | 2399 | CB | ARG | C | 91 | 14.958 | −7.824 | 10.333 | 1.00 | 86.56 | C |
| ATOM | 2400 | CG | ARG | C | 91 | 15.382 | −8.299 | 11.705 | 1.00 | 81.24 | C |
| ATOM | 2401 | CD | ARG | C | 91 | 15.044 | −7.317 | 12.809 | 1.00 | 87.65 | C |
| ATOM | 2402 | NE | ARG | C | 91 | 14.751 | −8.024 | 14.058 | 1.00 | 93.55 | N |
| ATOM | 2403 | CZ | ARG | C | 91 | 15.608 | −8.811 | 14.708 | 1.00 | 87.81 | C |
| ATOM | 2404 | NH1 | ARG | C | 91 | 16.830 | −9.008 | 14.240 | 1.00 | 81.53 | N |
| ATOM | 2405 | NH2 | ARG | C | 91 | 15.241 | −9.405 | 15.833 | 1.00 | 85.17 | N |
| ATOM | 2406 | C | ARG | C | 91 | 14.755 | −8.447 | 7.902 | 1.00 | 98.31 | C |
| ATOM | 2407 | O | ARG | C | 91 | 15.388 | −7.858 | 7.020 | 1.00 | 98.13 | O |
| ATOM | 2408 | N | PRO | C | 92 | 13.485 | −8.844 | 7.749 | 1.00 | 77.00 | N |
| ATOM | 2409 | CA | PRO | C | 92 | 12.648 | −8.524 | 6.580 | 1.00 | 77.89 | C |
| ATOM | 2410 | CB | PRO | C | 92 | 11.404 | −9.396 | 6.784 | 1.00 | 83.19 | C |
| ATOM | 2411 | CG | PRO | C | 92 | 11.399 | −9.744 | 8.261 | 1.00 | 79.58 | C |
| ATOM | 2412 | CD | PRO | C | 92 | 12.830 | −9.774 | 8.688 | 1.00 | 72.09 | C |
| ATOM | 2413 | C | PRO | C | 92 | 12.269 | −7.035 | 6.475 | 1.00 | 78.19 | C |
| ATOM | 2414 | O | PRO | C | 92 | 12.329 | −6.304 | 7.473 | 1.00 | 75.10 | O |
| ATOM | 2415 | N | LYS | C | 93 | 11.859 | −6.604 | 5.279 | 1.00 | 84.79 | N |
| ATOM | 2416 | CA | LYS | C | 93 | 11.629 | −5.186 | 4.993 | 1.00 | 79.09 | C |
| ATOM | 2417 | CB | LYS | C | 93 | 11.009 | −4.999 | 3.612 | 1.00 | 76.22 | C |
| ATOM | 2418 | CG | LYS | C | 93 | 11.088 | −3.551 | 3.138 | 1.00 | 89.03 | C |
| ATOM | 2419 | CD | LYS | C | 93 | 10.760 | −3.395 | 1.660 | 1.00 | 94.28 | C |
| ATOM | 2420 | CE | LYS | C | 93 | 9.254 | −3.314 | 1.426 | 1.00 | 98.15 | C |
| ATOM | 2421 | NZ | LYS | C | 93 | 8.894 | −3.173 | −0.027 | 1.00 | 85.12 | N |
| ATOM | 2422 | C | LYS | C | 93 | 10.814 | −4.389 | 6.027 | 1.00 | 83.56 | C |
| ATOM | 2423 | O | LYS | C | 93 | 11.316 | −3.420 | 6.599 | 1.00 | 81.20 | O |
| ATOM | 2424 | N | GLU | C | 94 | 9.561 | −4.772 | 6.254 | 1.00 | 112.68 | N |
| ATOM | 2425 | CA | GLU | C | 94 | 8.685 | −3.975 | 7.118 | 1.00 | 117.30 | C |
| ATOM | 2426 | CB | GLU | C | 94 | 7.211 | −4.308 | 6.879 | 1.00 | 115.74 | C |
| ATOM | 2427 | CG | GLU | C | 94 | 6.864 | −5.755 | 7.163 | 1.00 | 123.33 | C |
| ATOM | 2428 | CD | GLU | C | 94 | 7.563 | −6.714 | 6.214 | 1.00 | 125.67 | C |
| ATOM | 2429 | OE1 | GLU | C | 94 | 7.525 | −6.465 | 4.989 | 1.00 | 115.14 | O |
| ATOM | 2430 | OE2 | GLU | C | 94 | 8.150 | −7.709 | 6.693 | 1.00 | 125.63 | O |
| ATOM | 2431 | C | GLU | C | 94 | 9.025 | −4.088 | 8.604 | 1.00 | 111.21 | C |
| ATOM | 2432 | O | GLU | C | 94 | 8.791 | −3.145 | 9.365 | 1.00 | 102.74 | O |
| ATOM | 2433 | N | ALA | C | 95 | 9.555 | −5.240 | 9.015 | 1.00 | 121.83 | N |
| ATOM | 2434 | CA | ALA | C | 95 | 10.100 | −5.383 | 10.362 | 1.00 | 121.78 | C |
| ATOM | 2435 | CB | ALA | C | 95 | 10.894 | −6.679 | 10.487 | 1.00 | 110.98 | C |
| ATOM | 2436 | C | ALA | C | 95 | 11.006 | −4.186 | 10.552 | 1.00 | 112.84 | C |
| ATOM | 2437 | O | ALA | C | 95 | 10.872 | −3.406 | 11.500 | 1.00 | 103.60 | O |
| ATOM | 2438 | N | LEU | C | 96 | 11.915 | −4.040 | 9.597 | 1.00 | 70.09 | N |
| ATOM | 2439 | CA | LEU | C | 96 | 12.801 | −2.901 | 9.538 | 1.00 | 76.22 | C |
| ATOM | 2440 | CB | LEU | C | 96 | 13.767 | −3.062 | 8.368 | 1.00 | 80.81 | C |
| ATOM | 2441 | CG | LEU | C | 96 | 14.833 | −1.971 | 8.271 | 1.00 | 87.57 | C |
| ATOM | 2442 | CD1 | LEU | C | 96 | 15.520 | −1.812 | 9.617 | 1.00 | 87.54 | C |
| ATOM | 2443 | CD2 | LEU | C | 96 | 15.842 | −2.295 | 7.181 | 1.00 | 90.71 | C |
| ATOM | 2444 | C | LEU | C | 96 | 12.019 | −1.602 | 9.390 | 1.00 | 79.52 | C |
| ATOM | 2445 | O | LEU | C | 96 | 12.221 | −0.658 | 10.161 | 1.00 | 80.63 | O |
| ATOM | 2446 | N | LYS | C | 97 | 11.128 | −1.563 | 8.400 | 1.00 | 75.39 | N |
| ATOM | 2447 | CA | LYS | C | 97 | 10.359 | −0.365 | 8.095 | 1.00 | 72.02 | C |
| ATOM | 2448 | CB | LYS | C | 97 | 9.346 | −0.643 | 6.969 | 1.00 | 84.24 | C |
| ATOM | 2449 | CG | LYS | C | 97 | 8.993 | 0.578 | 6.085 | 1.00 | 89.21 | C |
| ATOM | 2450 | CD | LYS | C | 97 | 7.952 | 0.259 | 4.996 | 1.00 | 84.81 | C |
| ATOM | 2451 | CE | LYS | C | 97 | 8.520 | −0.643 | 3.898 | 1.00 | 85.94 | C |
| ATOM | 2452 | NZ | LYS | C | 97 | 7.553 | −0.880 | 2.786 | 1.00 | 79.83 | N |
| ATOM | 2453 | C | LYS | C | 97 | 9.665 | 0.165 | 9.352 | 1.00 | 70.03 | C |
| ATOM | 2454 | O | LYS | C | 97 | 9.637 | 1.371 | 9.592 | 1.00 | 72.61 | O |
| ATOM | 2455 | N | GLU | C | 98 | 9.135 | −0.738 | 10.168 | 1.00 | 65.07 | N |
| ATOM | 2456 | CA | GLU | C | 98 | 8.432 | −0.353 | 11.391 | 1.00 | 69.66 | C |
| ATOM | 2457 | CB | GLU | C | 98 | 7.873 | −1.586 | 12.103 | 1.00 | 77.27 | C |
| ATOM | 2458 | CG | GLU | C | 98 | 6.680 | −2.248 | 11.435 | 1.00 | 68.98 | C |
| ATOM | 2459 | CD | GLU | C | 98 | 6.339 | −3.583 | 12.081 | 1.00 | 73.97 | C |
| ATOM | 2460 | OE1 | GLU | C | 98 | 5.920 | −4.511 | 11.348 | 1.00 | 77.99 | O |
| ATOM | 2461 | OE2 | GLU | C | 98 | 6.493 | −3.704 | 13.322 | 1.00 | 67.60 | O |
| ATOM | 2462 | C | GLU | C | 98 | 9.264 | 0.459 | 12.395 | 1.00 | 78.44 | C |
| ATOM | 2463 | O | GLU | C | 98 | 8.808 | 1.506 | 12.868 | 1.00 | 74.73 | O |
| ATOM | 2464 | N | ARG | C | 99 | 10.456 | −0.020 | 12.755 | 1.00 | 80.01 | N |
| ATOM | 2465 | CA | ARG | C | 99 | 11.254 | 0.717 | 13.736 | 1.00 | 77.38 | C |
| ATOM | 2466 | CB | ARG | C | 99 | 12.474 | −0.082 | 14.218 | 1.00 | 76.38 | C |
| ATOM | 2467 | CG | ARG | C | 99 | 12.195 | −0.839 | 15.525 | 1.00 | 78.22 | C |
| ATOM | 2468 | CD | ARG | C | 99 | 13.385 | −1.623 | 16.087 | 1.00 | 81.36 | C |
| ATOM | 2469 | NE | ARG | C | 99 | 12.983 | −2.400 | 17.267 | 1.00 | 81.78 | N |
| ATOM | 2470 | CZ | ARG | C | 99 | 13.817 | −3.048 | 18.082 | 1.00 | 90.20 | C |
| ATOM | 2471 | NH1 | ARG | C | 99 | 15.127 | −3.023 | 17.858 | 1.00 | 85.19 | N |
| ATOM | 2472 | NH2 | ARG | C | 99 | 13.339 | −3.720 | 19.132 | 1.00 | 81.47 | N |
| ATOM | 2473 | C | ARG | C | 99 | 11.643 | 2.076 | 13.178 | 1.00 | 78.21 | C |
| ATOM | 2474 | O | ARG | C | 99 | 11.768 | 3.049 | 13.913 | 1.00 | 77.49 | O |
| ATOM | 2475 | N | ILE | C | 100 | 11.789 | 2.145 | 11.861 | 1.00 | 74.35 | N |
| ATOM | 2476 | CA | ILE | C | 100 | 12.104 | 3.405 | 11.207 | 1.00 | 81.30 | C |
| ATOM | 2477 | CB | ILE | C | 100 | 12.514 | 3.205 | 9.740 | 1.00 | 82.84 | C |
| ATOM | 2478 | CG1 | ILE | C | 100 | 13.518 | 2.060 | 9.630 | 1.00 | 72.62 | C |
| ATOM | 2479 | CD1 | ILE | C | 100 | 13.975 | 1.791 | 8.229 | 1.00 | 87.35 | C |
| ATOM | 2480 | CG2 | ILE | C | 100 | 13.092 | 4.496 | 9.163 | 1.00 | 79.94 | C |
| ATOM | 2481 | C | ILE | C | 100 | 10.915 | 4.345 | 11.270 | 1.00 | 73.62 | C |
| ATOM | 2482 | O | ILE | C | 100 | 11.071 | 5.510 | 11.617 | 1.00 | 80.26 | O |
| ATOM | 2483 | N | LYS | C | 101 | 9.731 | 3.844 | 10.940 | 1.00 | 64.40 | N |
| ATOM | 2484 | CA | LYS | C | 101 | 8.511 | 4.653 | 11.057 | 1.00 | 75.53 | C |
| ATOM | 2485 | CB | LYS | C | 101 | 7.246 | 3.831 | 10.740 | 1.00 | 67.79 | C |
| ATOM | 2486 | CG | LYS | C | 101 | 7.078 | 3.459 | 9.272 | 1.00 | 67.50 | C |
| ATOM | 2487 | CD | LYS | C | 101 | 5.949 | 2.469 | 9.086 | 1.00 | 58.98 | C |
| ATOM | 2488 | CE | LYS | C | 101 | 4.622 | 3.046 | 9.589 | 1.00 | 66.40 | C |
| ATOM | 2489 | NZ | LYS | C | 101 | 3.469 | 2.100 | 9.390 | 1.00 | 52.86 | N |
| ATOM | 2490 | C | LYS | C | 101 | 8.404 | 5.273 | 12.443 | 1.00 | 72.34 | C |
| ATOM | 2491 | O | LYS | C | 101 | 7.765 | 6.308 | 12.630 | 1.00 | 60.93 | O |
| ATOM | 2492 | N | LYS | C | 102 | 9.048 | 4.624 | 13.407 | 1.00 | 72.88 | N |
| ATOM | 2493 | CA | LYS | C | 102 | 9.014 | 5.059 | 14.792 | 1.00 | 68.92 | C |
| ATOM | 2494 | CB | LYS | C | 102 | 9.533 | 3.933 | 15.698 | 1.00 | 71.58 | C |
| ATOM | 2495 | CG | LYS | C | 102 | 9.698 | 4.294 | 17.163 | 1.00 | 70.95 | C |
| ATOM | 2496 | CD | LYS | C | 102 | 8.504 | 5.081 | 17.685 | 1.00 | 81.71 | C |
| ATOM | 2497 | C | LYS | C | 102 | 9.819 | 6.348 | 14.973 | 1.00 | 72.63 | C |
| ATOM | 2498 | O | LYS | C | 102 | 9.405 | 7.255 | 15.699 | 1.00 | 71.22 | O |
| ATOM | 2499 | N | TYR | C | 103 | 10.950 | 6.440 | 14.277 | 1.00 | 79.31 | N |
| ATOM | 2500 | CA | TYR | C | 103 | 11.862 | 7.568 | 14.437 | 1.00 | 77.29 | C |
| ATOM | 2501 | CB | TYR | C | 103 | 13.303 | 7.067 | 14.395 | 1.00 | 70.64 | C |
| ATOM | 2502 | CG | TYR | C | 103 | 13.513 | 5.986 | 15.424 | 1.00 | 65.24 | C |
| ATOM | 2503 | CD1 | TYR | C | 103 | 13.483 | 6.281 | 16.776 | 1.00 | 67.49 | C |
| ATOM | 2504 | CE1 | TYR | C | 103 | 13.642 | 5.293 | 17.726 | 1.00 | 68.13 | C |
| ATOM | 2505 | CZ | TYR | C | 103 | 13.827 | 3.984 | 17.327 | 1.00 | 68.01 | C |
| ATOM | 2506 | OH | TYR | C | 103 | 13.983 | 2.996 | 18.273 | 1.00 | 63.25 | O |
| ATOM | 2507 | CE2 | TYR | C | 103 | 13.856 | 3.666 | 15.988 | 1.00 | 62.76 | C |
| ATOM | 2508 | CD2 | TYR | C | 103 | 13.688 | 4.667 | 15.048 | 1.00 | 66.94 | C |
| ATOM | 2509 | C | TYR | C | 103 | 11.610 | 8.598 | 13.371 | 1.00 | 75.21 | C |
| ATOM | 2510 | O | TYR | C | 103 | 12.472 | 9.416 | 13.069 | 1.00 | 80.48 | O |
| ATOM | 2511 | N | LEU | C | 104 | 10.390 | 8.570 | 12.848 | 1.00 | 122.70 | N |
| ATOM | 2512 | CA | LEU | C | 104 | 10.020 | 9.265 | 11.621 | 1.00 | 125.91 | C |
| ATOM | 2513 | CB | LEU | C | 104 | 8.844 | 8.527 | 10.978 | 1.00 | 123.71 | C |
| ATOM | 2514 | CG | LEU | C | 104 | 8.698 | 8.470 | 9.460 | 1.00 | 129.05 | C |
| ATOM | 2515 | CD1 | LEU | C | 104 | 9.878 | 7.732 | 8.854 | 1.00 | 128.54 | C |
| ATOM | 2516 | CD2 | LEU | C | 104 | 7.382 | 7.790 | 9.089 | 1.00 | 128.89 | C |
| ATOM | 2517 | C | LEU | C | 104 | 9.643 | 10.727 | 11.856 | 1.00 | 135.85 | C |
| ATOM | 2518 | O | LEU | C | 104 | 10.391 | 11.644 | 11.513 | 1.00 | 130.81 | O |
| ATOM | 2519 | OXT | LEU | C | 104 | 8.575 | 11.034 | 12.391 | 1.00 | 137.62 | O |
| TER | | | | | | | | | | | |
| HETATM | 2520 | O | HOH | S | 1 | 25.811 | 2.360 | 24.963 | 1.00 | 45.86 | O |
| HETATM | 2521 | O | HOH | S | 2 | 7.173 | −5.978 | 14.925 | 1.00 | 38.83 | O |
| HETATM | 2522 | O | HOH | S | 3 | 9.962 | 0.261 | 27.317 | 1.00 | 39.71 | O |
| HETATM | 2523 | O | HOH | S | 4 | 24.011 | −15.969 | 22.835 | 1.00 | 47.78 | O |
| HETATM | 2524 | O | HOH | S | 5 | 2.695 | 0.019 | 35.519 | 1.00 | 61.10 | O |
| TER | | | | | | | | | | | |
| END | | | | | | | | | | | |

### REFERENCES

Adam, G. and M. Delbruck, Structural Chemistry and Molecular Biology, ed. A. Rich and N. Davidson. 1968, New York: W. H. Freeman and Co. 198-215.
Ainavarapu, S. R., Wiita, A. P., Huang, H. H. & Fernandez, J. M. A single-molecule assay to directly identify solvent-accessible disulfide bonds and probe their effect on protein folding. J Am Chem Soc 130, 436-7 (2008).
Alegre-Cebollada J, Perez-Jimenez R, Kosuri P, Fernandez J M., Single-molecule force spectroscopy approach to enzyme catalysis, J Biol Chem, 285(25), 18961-6 (2010).
Amer, E. S. and A. Holmgren, Physiological functions of thioredoxin and thioredoxin reductase. Eur J Biochem, 2000. 267(20): p. 6102-9.
Avval, F. Z. and A. Holmgren, Molecular mechanisms of thioredoxin and glutaredoxin as hydrogen donors for Mammalian s phase ribonucleotide reductase. J Biol Chem, 2009. 284(13): p. 8233-40.
Baker-Austin, C. & Dopson, M., Life in acid: pH Homeostasis in acidophiles. Trends Microbiol 15, 165-71 (2007).
Benner, S. A., Sassi, S. O. & Gaucher, E. A. Molecular paleoscience: systems biology from the past. Adv Enzymol Relat Areas Mol Biol 75, 1-132, xi (2007).
Berg, O. G. and C. Blomberg, Association Kinetics with Coupled Diffusion 0.3. Ionic-Strength Dependence of Lac Repressor-Operator Association. Biophysical Chemistry, 1978. 8(4): p. 271-280.
Berg, O. G., R. B. Winter, and P. H. Vonhippel, Diffusion-Driven Mechanisms of Protein Translocation on Nucleic-Acids 0.1. Models and Theory. Biochemistry, 1981. 20(24): p. 6929-6948.
Beynon, R. J. and J. S. Bond, Proteolytic enzymes: a practical approach. 2001, New York: Oxford University Press.
Boussau, B., Blanquart, S., Necsulea, A., Lartillot, N. & Gouy, M. Parallel adaptations to high temperatures in the Archaean eon. Nature 456, 942-5 (2008).
Capitani, G., Markovic-Housley, Z., DelVal, G., Morris, M., Jansonius, J. N. & Schurmann, P., Crystal structures of two functionally different thioredoxins in spinach chloroplasts. J Mol Biol, 2000. 302: p. 135-154.
Carvalho, A. T., et al., Mechanism of thioredoxin-catalyzed disulfide reduction. Activation of the buried thiol and role of the variable active-site residues. J Phys Chem B, 2008. 112(8): p. 2511-23.
Cecconi, C., et al., Protein-DNA chimeras for single molecule mechanical folding studies with the optical tweezers. Eur Biophys J, 2008. 37(6): p. 729-38.
Chang, B. S., Jonsson, K., Kazmi, M. A., Donoghue, M. J. & Sakmar, T. P. Recreating a functional ancestral archosaur visual pigment. Mol Biol Evol 19, 1483-9 (2002).
Chivers, P. T. and R. T. Raines, General acid/base catalysis in the active site of Escherichia coli thioredoxin. Biochemistry, 1997. 36(50): p. 15810-6.
Chivers, P. T., M. C. Laboissiere, and R. T. Raines, The CXXC motif: imperatives for the formation of native disulfide bonds in the cell. EMBO J, 1996. 15(11): p. 2659-67.
Cipriano, D. J. and S. D. Dunn, Tethering polypeptides through bifunctional PEG cross-linking agents to probe protein function: application to ATP synthase. Proteins, 2008. 73(2): p. 458-67.
Corey, D. R., Synthesis of oligonucleotide-peptide and oligonucleotide-protein conjugates. Methods Mol Biol, 2004. 283: p. 197-206.
Crankshaw, M. W. and G. A. Grant, Modification of cysteine. Curr Protoc Protein Sci, 2001. Chapter 15: p. Unit15 1.
del Rio, A., et al., Stretching single talin rod molecules activates vinculin binding. Science, 2009. 323(5914): p. 638-41.
Dyson, H. J. et al., Effects of buried charged groups on cysteine thiol ionization and reactivity in Escherichia coli thioredoxin: structural and functional characterization of mutants of Asp 26 and Lys 57. Biochemistry 36, 2622-36 (1997).
Edgar, R. C. MUSCLE: multiple sequence alignment with high accuracy and high throughput. Nucleic Acids Res 32, 1792-7 (2004).
Fernandez, J. M. & Li, H. Force-clamp spectroscopy monitors the folding trajectory of a single protein. Science 303, 1674-8 (2004).
Florin, E. L. et al. Sensing Specific Molecular-Interactions with the Atomic-Force Microscope. Biosensors & Bioelectronics 10, 895-901 (1995).
Frey, P. A. and A. D. Hegeman, Enzymatic reaction mechanisms. 2007, Oxford: Oxford University Press.
Garcia-Manyes, S., et al., Direct observation of an ensemble of stable collapsed states in the mechanical folding of ubiquitin. Proceedings of the National Academy of Sciences of the United States of America, 2009. 106(26): p. 10534-10539.
Garcia-Manyes, S., L. Dougan, and J. M. Fernandez, Osmolyte-induced separation of the mechanical folding phases of ubiquitin. Proceedings of the National Academy of Sciences of the United States of America, 2009. 106(26): p. 10540-10545.
Garcia-Manyes, S., Liang, J., Szoszkiewicz, R., Kuo, T. L. & Fernandez, J. M. Force-activated reactivity switch in a bimolecular chemical reaction. Nature Chemistry 1, 236-242 (2009).
Gaucher, E. A., et al., Inferring the palaeoenvironment of ancient bacteria on the basis of resurrected proteins. Nature, 2003. 425(6955): p. 285-8.
Gaucher, E. A., Govindarajan, S. & Ganesh, O. K. Palaeotemperature trend for Precambrian life inferred from resurrected proteins. Nature 451, 704-7 (2008).
Gaucher, E. A., Thomson, J. M., Burgan, M. F. & Benner, S. A. Inferring the palaeoenvironment of ancient bacteria on the basis of resurrected proteins. Nature 425, 285-8 (2003).
Godoy-Ruiz, R. et al. Natural selection for kinetic stability is a likely origin of correlations between mutational effects on protein energetics and frequencies of amino acid occurrences in sequence alignments. J Mol Biol 362, 966-78 (2006).
Gogarten-Boekels, M. Hilario, E. & Gogarten J. P., The effects of heavy meteorite bombardment on the early evolution-the emergence for the three domains of life. Orig. Life Evol. Biosph., 25: 251-264 (1995).
Gorman, J., et al., Dynamic basis for one-dimensional DNA scanning by the mismatch repair complex Msh2-Msh6. Mol Cell, 2007. 28(3): p. 359-70.
Green, N. S., E. Reisler, and K. N. Houk, Quantitative evaluation of the lengths of homobifunctional protein cross-linking reagents used as molecular rulers. Protein Sci, 2001. 10(7): p. 1293-304.
Halford, S. E. and J. F. Marko, How do site-specific DNA-binding proteins find their targets? Nucleic Acids Res, 2004. 32(10): p. 3040-52.
Hall, B. G. Simple and accurate estimation of ancestral protein sequences. Proc Natl Acad Sci USA 103, 5431-6 (2006).
Hedges, S. B. & Kumar, S. The Timetree of Life, xxi, 551 p. (Oxford University Press, Oxford, 2009).
Holmgren, A. Thioredoxin. Annu Rev Biochem 54, 237-71 (1985).
Holmgren, A., Reduction of disulfides by thioredoxin. Exceptional reactivity of insulin and suggested functions of thioredoxin in mechanism of hormone action. J Biol Chem, 1979. 254(18): p. 9113-9.
Holmgren, A., Thioredoxin and glutaredoxin systems. J Biol Chem, 1989. 264(24): p. 13963-6.
Holmgren, A., Thioredoxin. Annu Rev Biochem, 1985. 54: p. 237-71.
Holmgren, A., Tryptophan fluorescence study of conformational transitions of the oxidized and reduced form of thioredoxin. J Biol Chem, 1972. 247(7): p. 1992-8.
Holmgren, A., Thioredoxin catalyzes the reduction of insulin disulfides by dithiothreitol and dihydrolipoamide. J Biol Chem 254, 9627-32 (1979)
Ibarra-Molero, B., Loladze, V. V., Makhatadze, G. I. & Sanchez-Ruiz, J. M. Thermal versus guanidine-induced unfolding of ubiquitin. An analysis in terms of the contributions from charge-charge interactions to protein stability. Biochemistry 38, 8138-49 (1999).
Ji, T. H., Bifunctional reagents. Methods Enzymol, 1983. 91: p. 580-609.
Katti, S. K., D. M. LeMaster, and H. Eklund, Crystal structure of thioredoxin from Escherichia coli at 1.68 A resolution. J Mol Biol, 1990. 212(1): p. 167-84.
Kaganman, I., Resurrected Enzymes, Research Highlights, Nature Methods, 8:452 (2011).
Kice, J. L. Nucleophilic Substitution at Different Oxidation States of Sulfur. in Progress in Inorganic Chemistry (ed. Edwards, J. O.) 147-206 (2007).
Knauth, L. P. & Lowe, D. R. High Archean climatic temperature inferred from oxygen isotope geochemistry of cherts in the 3.5 Ga Swaziland Supergroup, South Africa. Geol. Soc. Am. Bull. 115, 566-580 (2003).
Koti Ainavarapu, S. R., Wiita, A. P., Dougan, L., Uggerud, E. & Fernandez, J. M. Single-molecule force spectroscopy measurements of bond elongation during a bimolecular reaction. J Am Chem Soc 130, 6479-87 (2008).
Kumar, J. K., S. Tabor, and C. C. Richardson, Proteomic analysis of thioredoxin-targeted proteins in Escherichia coli. Proc Natl Acad Sci USA, 2004. 101(11): p. 3759-64.
LaMantia, M. L. and W. J. Lennarz, The essential function of yeast protein disulfide isomerase does not reside in its isomerase activity. Cell, 1993. 74(5): p. 899-908.
Lancelin, J. M., Guilhaudis, L., Krimm, I., Blackledge, M. J., Marion, D. & Jacquot, J. P., NMR structures of thioredoxin m from the green alga Chlamydomonas reinhardtii. Proteins 2000. 41: p. 334-349
Liang, J. & Fernandez, J. M. Mechanochemistry: One Bond at a Time. ACS Nano (2009).
Liberles, D. A. Ancestral sequence reconstruction, xiii, 252 p. (Oxford University Press, Oxford; New York, 2007).
Light, A. and H. Janska, Enterokinase (enteropeptidase): comparative aspects. Trends Biochem Sci, 1989. 14(3): p. 110-2.
Lillig, C. H. and A. Holmgren, Thioredoxin and related molecules—from biology to health and disease. Antioxid Redox Signal, 2007. 9(1): p. 25-47.
Liu, R. C., et al., Mechanical Characterization of Protein L in the Low-Force Regime by Electromagnetic Tweezers/Evanescent Nanometry. Biophysical Journal, 2009. 96(9): p. 3810-3821.
Lopez-Otin, C. and J. S. Bond, Proteases: multifunctional enzymes in life and disease. J Biol Chem, 2008. 283(45): p. 30433-7.
Lu, D., et al., Crystal structure of enteropeptidase light chain complexed with an analog of the trypsinogen activation peptide. J Mol Biol, 1999. 292(2): p. 361-73.
Maeda, K., et al., Structural basis for target protein recognition by the protein disulfide reductase thioredoxin. Structure, 2006. 14(11): p. 1701-7.
Martin, J. L., Thioredoxin—a fold for all reasons. Structure, 1995. 3(3): p. 245-50.
Matthias, L. J. and P. J. Hogg, Redox control on the cell surface: implications for HIV-1 entry. Antioxid Redox Signal, 2003. 5(1): p. 133-8.
Matthias, L. J., et al., Disulfide exchange in domain 2 of CD4 is required for entry of HIV-1. Nat Immunol, 2002. 3(8): p. 727-32.
Menzel, U. & Gottschalk, G., The internal pH of Acetobaceterium wieringae and Acetobacter aceti during growth and production of acetic acid. Archives of Microbiology 143, 47-51 (1985).
Milanesi, L., et al., A method for the reversible trapping of proteins in non-native conformations. Biochemistry, 2008. 47(51): p. 13620-34.
Ming, H. et al., Crystal structure of thioredoxin domain of ST2123 from thermophilic archaea Sulfolobus tokodaii strain7. Proteins 69, 204-8 (2007)
Mustacich, D. and G. Powis, Thioredoxin reductase. Biochem J, 2000. 346 Pt 1: p. 1-8.
Nelder, J. A. & Mead, R. A Simplex-Method for Function Minimization. Computer Journal 7, 308-313 (1965).
Nisbet, E. G. & Sleep, N. H. The habitat and nature of early life. Nature 409, 1083-91 (2001).
Peregrin-Alvarez, J. M., Tsoka, S. & Ouzounis, C. A. The phylogenetic extent of metabolic enzymes and pathways. Genome Res 13, 422-7 (2003).
Perez-Jimenez, R. et al. Diversity of chemical mechanisms in thioredoxin catalysis revealed by single-molecule force spectroscopy. Nat Struct Mol Biol 16, 890-6 (2009).
Perez-Jimenez, R. et al. Force-clamp spectroscopy detects residue co-evolution in enzyme catalysis. J Biol. Chem., 283, 27121-27129 (2009).
Perez-Jimenez, R. et al. Single-molecule paleoenzymology probes the chemistry of resurrected enzymes., Nat Struct Mol. Biol. 2011 May; 18(5):592-6. Epub 2011 Apr. 3.
Peterson, F. C., Lytle, B. L., Sampath, S., Vinarov, D., Tyler, E., Shahan, M., Markley, J. L., Volkman, B. F., Solution structure of thioredoxin hl from Arabidopsis thaliana. Protein Sci., 2005. 14: p. 2195-2200
Pollock, D. D. & Chang, B. S. W. in Ancestral sequence reconstruction, pages 85-94 (ed. Liberles. D. A., Oxford University Press, Oxford; New York, 2007).
Powis, G. and W. R. Montfort, Properties and biological activities of thioredoxins Annu Rev Biophys Biomol Struct, 2001. 30: p. 421-55.
Qin, J., Clore, G. M., Kennedy, W. M., Huth, J. R. & Gronenborn, A. M., Solution structure of human thioredoxin in a mixed disulfide intermediate complex with its target peptide from the transcription factor NF kappa B. Structure, 1995. 3: p. 289-297
Qin, J., Clore, G. M., Kennedy, W. P., Kuszewski, J. & Gronenborn, A. M., The solution structure of human thioredoxin complexed with its target from Ref-1 reveals peptide chain reversal. Structure 1996. 4: p. 613-620
Riggs, A. D., Bourgeoi. S, and M. Cohn, Lac Repressor-Operator Interaction 0.3. Kinetic Studies. Journal of Molecular Biology, 1970. 53(3): p. 401-&.
Russell, M. J. & Hall, A. J. The emergence of life from iron monosulphide bubbles at a submarine hydrothermal redox and pH front. J Geol Soc Lond 154, 377-402 (1997).
Sarkar, A., R. B. Robertson, and J. M. Fernandez, Simultaneous atomic force microscope and fluorescence measurements of protein unfolding using a calibrated evanescent wave. Proc Natl Acad Sci USA, 2004. 101(35): p. 12882-6.
Schulte, M. The Emergence of Life on Earth. Oceanography 20, 42-49 (2007).
Smeets, A., Evrard, C., Landtmeters, M., Marchand, C., Knoops, B. & Declercq, J. P., Crystal structures of oxidized and reduced forms of human mitochondrial thioredoxin 2. Protein Sci., 2005. 14: p. 2610-2621
Stanford, N. P., et al., One- and three-dimensional pathways for proteins to reach specific DNA sites. Embo J, 2000. 19(23): p. 6546-57.
Starks, C. M., Francois, J. A., MacArthur, K. M., Heard, B. Z. & Kappock, T. J. Atomic-resolution crystal structure of thioredoxin from the acidophilic bacterium Acetobacter aceti. Protein Sci 16, 92-8 (2007)
Suarez, M. et al., Using multi-objective computational design to extend protein promiscuity. Biophys Chem 147, 13-9 (2010)
Tachibana, C. and T. H. Stevens, The yeast EUG1 gene encodes an endoplasmic reticulum protein that is functionally related to protein disulfide isomerase. Mol Cell Biol, 1992. 12(10): p. 4601-11.
Thomson, J. M. et al. Resurrecting ancestral alcohol dehydrogenases from yeast. Nat Genet. 37, 630-5 (2005).
Thornton, J. W. Resurrecting ancient genes: experimental analysis of extinct molecules. Nat Rev Genet. 5, 366-75 (2004).
Thornton, J. W., Need, E. & Crews, D. Resurrecting the ancestral steroid receptor: ancient origin of estrogen signaling. Science 301, 1714-7 (2003).
Turk, B., Targeting proteases: successes, failures and future prospects. Nat Rev Drug Discov, 2006. 5(9): p. 785-99.
von Hippel, P. H. and O. G. Berg, Facilitated target location in biological systems. J Biol Chem, 1989. 264(2): p. 675-8.
Walker, B. and J. F. Lynas, Strategies for the inhibition of serine proteases. Cell Mol Life Sci, 2001. 58(4): p. 596-624.
Walker, J. C. G. Possible Limits on the Composition of the Archean Ocean. Nature 302, 518-520 (1983).
Wiita, A. P. et al. Probing the chemistry of thioredoxin catalysis with force. Nature 450, 124-7 (2007).
Wiita, A. P., Ainavarapu, S. R., Huang, H. H. & Fernandez, J. M. Force-dependent chemical kinetics of disulfide bond reduction observed with single-molecule techniques. Proc Natl Acad Sci USA 103, 7222-7 (2006).
Wiita, A. P., et al., Probing the chemistry of thioredoxin catalysis with force. Nature, 2007. 450(7166): p. 124-7.
Williams, C. H., et al., Thioredoxin reductase two modes of catalysis have evolved. Eur J Biochem, 2000. 267(20): p. 6110-7.
Windle, H. J., Fox, A., Ni Eidhin, D. & Kelleher, D., The thioredoxin system of Helicobacter pylori. J Biol Chem 275, 5081-9 (2000)
Wynn, R. and F. M. Richards, Chemical modification of protein thiols: formation of mixed disulfides. Methods Enzymol, 1995. 251: p. 351-6.
Xu, S. Z., et al., TRPC channel activation by extracellular thioredoxin. Nature, 2008. 451(7174): p. 69-72.
Yang, Z., Kumar, S. & Nei, M. A new method of inference of ancestral nucleotide and amino acid sequences. Genetics 141, 1641-50 (1995).
Yang, Z. H. PAML: a program package for phylogenetic analysis by maximum likelihood. Comput Appl Biosci 13, 555-556 (1997).
Zalatan, J. G. & Herschlag, D., The far reaches of enzymology. Nat. Chem. Biol., 5:516-520 (2009).

What is claimed is:

1. An isolated polypeptide having a sequence selected from the group consisting of: SEQ ID NO: 1-7, and a variant of any one of SEQ ID NO: 1-7 having at least about 75% identity to SEQ ID NO: 1-7.

2. An isolated polypeptide comprising at least about 10, at least about 20, at least about 30, at least about 50 at least about 60, at least about 70, at least about 80, at least about 90 or at least about 100 consecutive amino acids from any of SEQ ID NOs: 1-7.

3. The isolated polypeptide of claim 1 or 2, wherein the sequence does not have 100% identity with any extant polypeptide.

4. The isolated polypeptide of claim 1, wherein the variant has at least about 85.5%, at least about 90.5%, at least about 92.5%, at least about 95%, about 96%, about 96.5%, about 97%, about 97.5%, about 98%, about 98.5%, about 99%, about 99.5% or about 99.9% amino acid sequence identity to any one of SEQ ID NO: 1-7.

5. The isolated polypeptide of claim 2, wherein the polypeptide has at least about 85.5%, at least about 90.5%, at least about 92.5%, at least about 95%, about 96%, about 96.5%, about 97%, about 97.5%, about 98%, about 98.5%, about 99%, about 99.5% or about 99.9% amino acid sequence identity to any one of SEQ ID NO: 1-7.

6. The isolated polypeptide of any one of claims 1-2, wherein the polypeptide has enzymatic activity.

7. The isolated polypeptide of any one of claims 1-2, wherein the polypeptide has thioredoxin activity.

8. The isolated polypeptide of any one of claims 1-2, wherein the polypeptide is labeled.

9. The isolated polypeptide of claim 8, wherein the label is colorimetric, radioactive, chemiluminescent, or fluorescent.

10. The isolated polypeptide of any one of claims 1-2, wherein the polypeptide is chemically modified.

11. The isolated polypeptide of claim 10, wherein the chemical modification comprises covalent modification of an amino acid.

12. The isolated polypeptide of claim 11, wherein the covalent modification comprises methylation, acetylation, phosphorylation, ubiquitination, sumoylation, citrullination, or ADP ribosylation.

13. An isolated antibody that specifically binds to a polypeptide of any of SEQ ID NO: 1-7.

14. An isolated nucleic acid comprising a nucleic acid sequence which encodes the polypeptide of any of claims 1-2.

15. The sequence of claim 14, wherein the sequence is optimized for expression in a mammalian expression system.

16. The sequence of claim 14, wherein the sequence is optimized for expression in a bacterial expression system.

17. The sequence of claim 16, wherein the bacterial expression system is E. coli.

18. The isolated nucleic acid of claim 14, wherein the isolated nucleic acid is operably linked to one or more control sequences that direct the production of the polypeptide in a suitable expression host.

19. A recombinant expression vector comprising the nucleic acid of claim 18.

20. A recombinant host cell comprising the nucleic acid of claim 14.

21. A method for producing the polypeptide of any one of claims 1-2, the method comprising cultivating a host cell comprising a nucleic acid construct comprising a polynucleotide encoding the polypeptide under conditions suitable for production of the polypeptide; and recovering the polypeptide.

22. A polypeptide produced by the method of claim 21.

23. A method generating a reconstructed ancestral polypeptide having greater activity or stability at low pH than an extant polypeptide, the method comprising
(a) aligning a plurality of sequences corresponding to homologues of the extant polypeptide,
(b) generating a phylogenetic tree of the plurality of sequences corresponding homologues of the extant polypeptide,
(c) using bayesian statistical analysis to generate inferred sequences of one or more ancestral genes encoding a version of the polypeptide that was present in a common ancestor of at least two or more organisms in the phylogenetic tree,
(d) calculating posterior probabilities for all 20 amino acids in each inferred sequence,
(e) generating a reconstructed ancestral polypeptide sequence by assigning to each position in the inferred sequence the amino acid residue having the highest posterior probability for that position and wherein a polypeptide comprising the reconstructed ancestral polypeptide sequence has increased activity or stability at low pH relative to the extant polypeptide.

24. A method generating a reconstructed ancestral polypeptide having greater activity or stability at high temperature than an extant polypeptide, the method comprising
(a) aligning a plurality of sequences corresponding to homologues of the extant polypeptide,
(b) generating a phylogenetic tree of the plurality of sequences corresponding homologues of the extant polypeptide,
(c) using bayesian statistical analysis to generate inferred sequences of one or more ancestral genes encoding a version of the polypeptide that was present in a common ancestor of at least two or more organisms in the phylogenetic tree,
(d) calculating posterior probabilities for all 20 amino acids in each inferred sequence,
(e) generating a reconstructed ancestral polypeptide sequence by assigning to each position in the inferred sequence the amino acid residue having the highest posterior probability for that position and wherein a polypeptide comprising the reconstructed ancestral polypeptide sequence has increased activity or stability at high temperature relative to the extant polypeptide.

25. A method generating a reconstructed ancestral polypeptide having a higher melting temperature than an extant polypeptide, the method comprising
(a) aligning a plurality of sequences corresponding to homologues of the extant polypeptide,
(b) generating a phylogenetic tree of the plurality of sequences corresponding homologues of the extant polypeptide,
(c) using bayesian statistical analysis to generate inferred sequences of one or more ancestral genes encoding a version of the polypeptide that was present in a common ancestor of at least two or more organisms in the phylogenetic tree,
(d) calculating posterior probabilities for all 20 amino acids in each inferred sequence,
(e) generating a reconstructed ancestral polypeptide sequence by assigning to each position in the inferred sequence the amino acid residue having the highest posterior probability for that position and wherein a polypeptide comprising the reconstructed ancestral polypeptide sequence has a higher melting temperature than an extant polypeptide.

26. The method of any one of claim 23, 24 or 25, wherein the extant polypeptide is a thioredoxin polypeptide.

27. A polypeptide generated according to the method of any of claim 23, 24 or 25.
